(19) United States
(12) Patent Application Publication
(10) Pub. No.: US 2026/0209768 A1
(43) Pub. Date: Jul. 23, 2026

(54) GUIDE RNAS THAT TARGET FOXP3 GENE AND METHODS OF USE

(71) Applicant: Life Edit Therapeutics, Inc., Durham, NC (US)

(72) Inventors: Philip Borden, Durham, NC (US); Michael Coyle, Chapel Hill, NC (US); Alexandra Briner Crawley, Cary, NC (US); Drew Kelso, Holly Springs, NC (US); Michael Vanden Oever, New Hill, NC (US)

(73) Assignee: Life Edit Therapeutics, Inc., Durham, NC (US)

(21) Appl. No.: 19/138,521

(22) PCT Filed: Dec. 15, 2023
(86) PCT No.: PCT/IB2023/062825

§ 371 Date: Jun. 12, 2025

Related U.S. Application Data
(60) Provisional application No. 63/387,888, filed on Dec. 16, 2022.

(51) Int. Cl. C12N 15/113; C12N 5/0783; C12N 9/12; C12N 9/22; C12N 9/78; C12Q 1/6869

(52) U.S. Cl. CPC C12N 15/1136; C12N 5/0636; C12N 9/1276; C12N 9/222; C12N 9/78; C12Q 1/6869; C12Y 207/07049; C12Y 305/04; C12N 2310/20; C12N 2310/315; C12N 2310/321; C12N 2310/346; C12N 2310/531

(57) ABSTRACT

Compositions and methods for binding to a target sequence in a forkhead box P3 (FOXP3) gene are provided. Compositions include CRISPR RNAs, guide RNAs, and nucleic acid molecules encoding the same. Vectors and host cells comprising the nucleic acid molecules are also provided. Further provided are RNA-guided nuclease (RGN) systems for binding a target sequence in a FOXP3 gene, wherein the RGN system comprises an RNA-guided nuclease polypeptide and one or more guide RNAs. The compositions find use in cleaving or modifying a target sequence of an FOXP3 gene, and/or modifying the expression of an FOXP3 gene.

### CROSS-REFERENCE TO RELATED APPLICATIONS

This application claims priority to U.S. Provisional Application No. 63/387,888, filed Dec. 16, 2022, which is incorporated by reference herein in its entirety.

### REFERENCE TO A SEQUENCE LISTING SUBMITTED ELECTRONICALLY AS AN XML FILE

The instant application contains a Sequence Listing which has been submitted in xml format via USPTO Patent Center and is hereby incorporated by reference in its entirety. Said xml copy, created on Dec. 7, 2023, is named L103438_1350PCT_0251_7_SL, and is 2.24 MB in size.

### FIELD OF THE INVENTION

The present invention relates to the field of molecular biology and gene editing.

### BACKGROUND OF THE INVENTION

T cells are white blood cells that function in the adaptive immune system to attack and destroy foreign molecules, pathogens, and/or tumors. T cells include cytotoxic T cells which kill their targets, along with helper T cells that help other cells of the immune system. Regulatory T cells (Tregs) are helper T cells that play a role in suppressing or modulating other immune cells. This Treg function is important to ensure that the immune system does not attack ‘self’ molecules of the body and to suppress exaggerated immune responses. Forkhead box P3 (Foxp3) is a transcription factor associated with Tregs that regulates Treg development and functions by activating or repressing other genes. The ability to manipulate expression of Foxp3 would be invaluable in controlling the function of a T cell to either encourage immune suppression in an inflammatory or autoimmune setting or to reduce immune suppression in a tumor microenvironment.

Targeted genome editing or modification is rapidly becoming an important tool for basic and applied research, as it allows modification of genomes such as cutting nucleic acids, deleting nucleic acids, inserting nucleic acids, substituting nucleotides in nucleic acids, and regulating gene expression at specific locations in a genome, along with many other possible modifications. Initial efforts in genome editing involved designing nucleases, proteins that are able to edit nucleic acids, to recognize and bind specifically to a target nucleic acid sequence to be edited. However, engineering nucleases takes considerable time and experimentation to obtain ones effective for editing of a particular sequence. Genome editing systems that use RNA-guided nucleases, such as the Clustered Regularly Interspaced Short Palindromic Repeats (CRISPR)-associated (Cas) proteins of the CRISPR-Cas bacterial system, function by complexing a nuclease with a guide RNA. The hybridization of the guide RNA to a particular target sequence allows editing at a specific location in a genome. Thus, genome editing systems that use RNA-guided nucleases can be less costly and more efficient for editing of genome sequences, as nucleic acids typically can be easier to design and re-design as compared to a nuclease.

Thus, regulation of expression of Foxp3 would benefit from development of RNA-guided nuclease systems that are able to target specific regions of the FOXP3 gene for binding, cleavage, and/or modification.

### BRIEF SUMMARY OF THE INVENTION

Compositions and methods for binding a target sequence in the forkhead box P3 (FOXP3) gene are provided. The compositions find use in modifying the FOXP3 gene at specific regions. Compositions comprise CRISPR RNAs (crRNAs), trans-activating CRISPR RNAs (tracrRNAs), single guide RNAs (sgRNAs), dual guide RNA (dgRNAs), RNA-guided nuclease (RGN) polypeptides, nucleic acid molecules encoding the same, compositions comprising the same, and vectors and host cells comprising the nucleic acid molecules. Also provided are RGN systems and ribonucleoprotein complexes for binding a target sequence in the FOXP3 gene, wherein the RGN system and ribonucleoprotein complex comprises an RGN polypeptide and one or more guide RNAs. Thus, methods disclosed herein are drawn to binding a target sequence in the FOXP3 gene, and in some embodiments, cleaving or modifying the target sequence in the FOXP3 gene. The FOXP3 gene can be modified, for example, to be knocked out as a result of non-homologous end joining after cleavage of a target sequence.

In one aspect, the present disclosure provides a guide RNA (gRNA) comprising a CRISPR RNA (crRNA) and a trans-activating CRISPR RNA (tracrRNA), wherein the crRNA comprises (i) a crRNA repeat; and (ii) a spacer, wherein the tracrRNA comprises: (iii) an anti-repeat; and (iv) a tail, wherein the gRNA comprises a first stem loop formed by hybridization of the crRNA repeat and the anti-repeat, wherein the spacer hybridizes to a target sequence in a forkhead box P3 (FOXP3) gene, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214. In some aspects, the target sequence in a FOXP3 gene that the spacer hybridides to comprises a target strand and a non-target strand.

In some embodiments of the above aspect, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides. In some embodiments of the above aspect, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

In some embodiments of the above aspect, the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA. In some embodiments, the linker has a nucleotide sequence set forth as AAAG, GAAA, ACUU, or CAAAGG. In some embodiments, the linker has a nucleotide sequence set forth as AAAG. In some embodiments of the above aspect, the backbone of the sgRNA comprises a total length of at least 85, 90, 95, 100, 105, 110, 115, or 120 nucleotides. In some embodiments of the above aspect, the backbone of the sgRNA comprises a total length of at most 85, 90, 95, 100, 105, 110, 115, or 120 nucleotides. In some embodiments of the above aspect, the backbone of the sgRNA comprises a total length of 86 to 98 nucleotides. In some embodiments of the above aspect, the backbone of the sgRNA comprises a total length of 94 nucleotides. In some embodiments of the above aspect, the backbone of the sgRNA has a nucleotide sequence having at least 80% sequence identity, at least 90% sequence identity, at least 95% sequence identity, or 100% sequence identity to any one of SEQ ID NOs: 563-573.

In some embodiments of the above aspect, the first stem loop comprises a first stem and a second stem, and wherein the first stem of the first stem loop comprises a total length of at least 3, 4, 5, 6, 7, 8, 9, 10, or 11 base pairs (bp). In some embodiments of the above aspect, the first stem of the first stem loop comprises a total length of at most 3, 4, 5, 6, 7, 8, 9, 10, or 11 bp. In some embodiments, the first stem of the first stem loop comprises a total length of 6 bp. In some embodiments, the first stem of the first stem loop comprises a total length of 3 bp.

In some embodiments of the above aspect, the tail of the tracrRNA comprises a total length of at least 1, 2, 3, 4, 5, 6, or 7 nucleotides. In some embodiments of the above aspect, the tail of the tracrRNA comprises a total length of at most 1, 2, 3, 4, 5, 6, or 7 nucleotides. In some embodiments, the tail of the tracrRNA comprises a total length of 3 nucleotides. In some embodiments, the tail of the tracrRNA comprises a total length of 1 nucleotide.

In some embodiments of the above aspect, the gRNA further comprises a second stem loop most proximal to the tail, wherein the second stem loop comprises a first stem and a second stem. In some embodiments of the above aspect, the first stem of the second stem loop comprises a total length of at least 1, 2, 3, 4, 5, or 6 bp. In some embodiments of the above aspect, the first stem of the second stem loop comprises a total length of at most 1, 2, 3, 4, 5, or 6 bp. In some embodiments, the first stem of the second stem loop comprises a total length of 5 bp.

In some embodiments of the above aspect, the first stem of the first stem loop comprises a total length of 6 bp, the tail of the tracrRNA comprises a total length of 3 nucleotides, and the first stem of the second stem loop comprises a total length of 5 bp.

In some embodiments of the above aspect, the gRNA is a dual guide RNA (dgRNA). In some embodiments of the above aspect, the crRNA repeat of the dgRNA comprises a total length of at least 10, 11, 12, 13, 14, 15, 16, 17, 18, 19, 20, or 21 nucleotides. In some embodiments of the above aspect, the crRNA repeat of the dgRNA comprises a total length of at most 10, 11, 12, 13, 14, 15, 16, 17, 18, 19, 20, or 21 nucleotides. In some embodiments, the crRNA repeat of the dgRNA comprises a total length of 13 nucleotides. In some embodiments, the crRNA repeat of the dgRNA comprises a total length of 16 nucleotides. In some embodiments, the crRNA repeat of the dgRNA comprises a total length of 21 nucleotides. In some embodiments of the above aspect, the tracrRNA of the dgRNA comprises a total length of at least 65, 70, 75, 80, or 85 nucleotides. In some embodiments of the above aspect, the tracrRNA of the dgRNA comprises a total length of at most 65, 70, 75, 80, or 85 nucleotides. In some embodiments, the tracrRNA of the dgRNA comprises a total length of 74 nucleotides. In some embodiments, the tracrRNA of the dgRNA comprises a total length of 77 nucleotides.

In some embodiments of the above aspect, the gRNA comprises a total length of at least 85, 90, 95, 100, 105, 110, 115, 120, 125, 130, or 135 nucleotides. In some embodiments of the above aspect, the gRNA comprises a total length of at most 85, 90, 95, 100, 105, 110, 115, 120, 125, 130, or 135 nucleotides. In some embodiments of the above aspect, the gRNA comprises a total length of 106 to 135 nucleotides. In some embodiments of the above aspect, the gRNA comprises a total length of 117 to 119 nucleotides.

In some embodiments of the above aspect, the gRNA is capable of targeting a bound RNA-guided nuclease (RGN) polypeptide to the target sequence in the FOXP3 gene. In some embodiments of the above aspect, the RGN polypeptide is capable of recognizing a consensus protospacer adjacent motif (PAM) having the nucleotide sequence set forth as NNNNCC. In some embodiments of the above aspect, the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.

In some embodiments of the above aspect, the RGN polypeptide has an amino acid sequence having at least 80% sequence identity to SEQ ID NO: 545; and wherein the target sequence and the spacer are selected from the group consisting of: a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides; b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides; c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides; d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides; e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides. In some embodiments of the above aspect, the spacer has the nucleotide sequence set forth as any one of SEQ ID NOs: 155, 163, 189, 179, 197, and 193.

In some embodiments of the above aspect, the RGN polypeptide has the amino acid sequence set forth as SEQ ID NO: 545. In some embodiments of the above aspect, the crRNA repeat has the nucleotide sequence set forth as SEQ ID NO: 546 or a nucleotide sequence that differs in length and/or sequence from SEQ ID NO: 546 by 1 to 8 nucleotides. In some embodiments of the above aspect, the crRNA repeat has the nucleotide sequence set forth as any one of SEQ ID NOs: 546, 549-552, 839, 842, and 845. In some embodiments of the above aspect, the crRNA has the nucleotide sequence set forth as any one of SEQ ID NOs: 574-692. In some embodiments of the above aspect, the tracrRNA has a nucleotide sequence having at least 80% sequence identity, at least 90% sequence identity, or at least 95% sequence identity to SEQ ID NO: 547. In some embodiments, the tracrRNA has a nucleotide sequence set forth as SEQ ID NO: 547. In some embodiments of the above aspect, the tracrRNA has a nucleotide sequence that differs in length from SEQ ID NO: 547 by 1 to 16 nucleotides. In some embodiments, the tracrRNA has a nucleotide sequence that is 8 nucleotides shorter than SEQ ID NO: 547. In some embodiments, the tracrRNA has a nucleotide sequence that is 11 nucleotides shorter than SEQ ID NO: 547. In some embodiments of the above aspect, the tracrRNA has the nucleotide sequence set forth as any one of SEQ ID NOs: 547, 553-562, 840, 842, and 846.

In some embodiments of the above aspect, the gRNA has a nucleotide sequence set forth as any one of SEQ ID NOs: 693-834. In some embodiments, the gRNA has a nucleotide sequence set forth as any one of SEQ ID NOs: 693, 694, 695, 696, 697, and 698.

In some embodiments of the above aspect, the gRNA comprises at least one chemical modification. In some embodiments, the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof. In some embodiments, the BNA comprises a 2′,4′ BNA modification. In some embodiments, the 2′,4′ BNA modification is selected from the group consisting of: locked nucleic acid (LNA) modification, BNANC[N-Me]modification, 2′-O,4′-C-ethylene bridged nucleic acid (2′,4′-ENA) modification, and S-constrained ethyl (cEt) modification. In some embodiments, the 2′,4′ BNA is a LNA modification. In some embodiments, the 2′,4′ BNA is a cEt modification. In some embodiments, the at least one chemical modification comprises a BNA modification, 2′-O-Me modification, PS modification, or a combination thereof.

In some embodiments, the at least one chemical modification comprises 2′-O-methyl 3′phosphorothioate (MS) modifications at the 3 terminal nucleotides at the 5′ end and at the 3 terminal nucleotides at the 3′ end of the gRNA. In some embodiments of the above aspect, the crRNA repeat has the nucleotide sequence set forth as any one of SEQ ID NOs: 940, 942-945, 1228, 1230, and 1232. In some embodiments of the above aspect, the crRNA has the nucleotide sequence set forth as any one of SEQ ID NOs: 967-1085. In some embodiments of the above aspect, the tracrRNA has the nucleotide sequence set forth as any one of SEQ ID NOs: 941, 946-955, 1229, 1231, and 1233. In some embodiments of the above aspect, the gRNA has the nucleotide sequence set forth as any one of SEQ ID NOs: 1086-1227.

In some embodiments of the above aspect, the gRNA further comprises an extension comprising an edit template for reverse transcriptase (RT) editing.

In another aspect, the present disclosure provides a guide RNA (gRNA) comprising a CRISPR RNA (crRNA) and a trans-activating CRISPR RNA (tracrRNA), wherein the crRNA comprises (i) a crRNA repeat; and (ii) a spacer, wherein the tracrRNA comprises: (iii) an anti-repeat; and (iv) a tail, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.

In some embodiments of the above gRNA aspect, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213. In some embodiments of the above gRNA aspect, the spacer is capable of hybridizing to a target sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

In another aspect, the present disclosure provides a nucleic acid molecule comprising a CRISPR RNA (crRNA) or encoding a crRNA, wherein the crRNA comprises a spacer and a crRNA repeat, wherein the spacer hybridizes to a target sequence in a forkhead box P3 (FOXP3) gene, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

In some embodiments of the nucleic acid molecule aspect, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides. In some embodiments, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

In some embodiments of the nucleic acid molecule aspect, the crRNA is capable of binding a trans-activating CRISPR RNA (tracrRNA) to form a guide RNA (gRNA), wherein the tracrRNA comprises an anti-repeat and a tail. In some embodiments of the nucleic acid molecule aspect, the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA. In some embodiments, the backbone of the sgRNA comprises a total length of 86 to 98 nucleotides. In some embodiments, the backbone of the sgRNA comprises a total length of 94 nucleotides. In some embodiments, the backbone of the sgRNA has a nucleotide sequence having at least 80% sequence identity, at least 90% sequence identity, at least 95% sequence identity, or 100% sequence identity to any one of SEQ ID NOs: 563-573.

In some embodiments of the nucleic acid molecule aspect, the gRNA comprises a first stem loop comprising a first stem and a second stem formed by hybridization of the crRNA repeat and the anti-repeat, wherein the first stem of the first stem loop comprises a total length of at least 3, 4, 5, 6, 7, 8, 9, 10, or 11 bp. In some embodiments of the nucleic acid molecule aspect, the gRNA comprises a first stem loop comprising a first stem and a second stem formed by hybridization of the crRNA repeat and the anti-repeat, wherein the first stem of the first stem loop comprises a total length of at most 3, 4, 5, 6, 7, 8, 9, 10, or 11 bp. In some embodiments, the first stem of the first stem loop comprises a total length of 6 bp. In some embodiments, the first stem of the first stem loop comprises a total length of 3 bp.

In some embodiments of the nucleic acid molecule aspect, the tail of the tracrRNA comprises a total length of at least 1, 2, 3, 4, 5, 6, or 7 nucleotides. In some embodiments of the nucleic acid molecule aspect, the tail of the tracrRNA comprises a total length of at most 1, 2, 3, 4, 5, 6, or 7 nucleotides. In some embodiments, the tail of the tracrRNA comprises a total length of 3 nucleotides. In some embodiments, the tail of the tracrRNA comprises a total length of 1 nucleotide.

In some embodiments of the nucleic acid molecule aspect, the gRNA further comprises a second stem loop most proximal to the tail, wherein the second stem loop comprises a first stem and a second stem. In some embodiments of the nucleic acid molecule aspect, the first stem of the second stem loop comprises a total length of at least 1, 2, 3, 4, 5, or 6 bp. In some embodiments of the nucleic acid molecule aspect, the first stem of the second stem loop comprises a total length of at most 1, 2, 3, 4, 5, or 6 bp. In some embodiments, the first stem of the second stem loop comprises a total length of 5 bp.

In some embodiments of the nucleic acid molecule aspect, the first stem of the first stem loop comprises a total length of 6 bp, the tail of the tracrRNA comprises a total length of 3 nucleotides, and the first stem of the second stem loop comprises a total length of 5 bp.

In some embodiments of the nucleic acid molecule aspect, the gRNA is a dual guide RNA (dgRNA). In some embodiments of the nucleic acid molecule aspect, the crRNA repeat comprises a total length of at least 10, 11, 12, 13, 14, 15, 16, 17, 18, 19, 20, or 21 nucleotides. In some embodiments of the nucleic acid molecule aspect, the crRNA repeat comprises a total length of at most 10, 11, 12, 13, 14, 15, 16, 17, 18, 19, 20, or 21 nucleotides. In some embodiments, the crRNA repeat comprises a total length of 13 nucleotides. In some embodiments, the crRNA repeat comprises a total length of 16 nucleotides. In some embodiments, the crRNA repeat comprises a total length of 21 nucleotides. In some embodiments of the nucleic acid molecule aspect, the tracrRNA comprises a total length of at least 65, 70, 75, 80, or 85 nucleotides. In some embodiments of the nucleic acid molecule aspect, the tracrRNA comprises a total length of at most 65, 70, 75, 80, or 85 nucleotides. In some embodiments, the tracrRNA comprises a total length of 74 nucleotides. In some embodiments, the tracrRNA comprises a total length of 77 nucleotides.

In some embodiments of the nucleic acid molecule aspect, the gRNA comprises a total length of at least 85, 90, 95, 100, 105, 110, 115, 120, 125, 130, or 135 nucleotides. In some embodiments of the nucleic acid molecule aspect, the gRNA comprises a total length of at most 85, 90, 95, 100, 105, 110, 115, 120, 125, 130, or 135 nucleotides. In some embodiments, the gRNA comprises a total length of 106 to 135 nucleotides. In some embodiments, the gRNA comprises a total length of 117 to 119 nucleotides. In some embodiments, the gRNA is capable of targeting a bound RNA-guided nuclease (RGN) polypeptide to a target sequence.

In some embodiments of the nucleic acid molecule aspect, the gRNA is capable of binding to an RGN polypeptide capable of recognizing a consensus protospacer adjacent motif (PAM) having the nucleotide sequence set forth as NNNNCC. In some embodiments, the gRNA is capable of binding to an RGN polypeptide capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.

In some embodiments of the nucleic acid molecule aspect, the RGN polypeptide comprises an amino acid sequence having at least 80% sequence identity to SEQ ID NO: 545; and wherein the target sequence and the spacer are selected from the group consisting of: a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides; b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides; c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides; d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides; e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides. In some embodiments of the above aspect, the spacer has the nucleotide sequence set forth as any one of SEQ ID NOs: 155, 163, 189, 179, 197, and 193.

In some embodiments of the nucleic acid molecule aspect, the RGN polypeptide comprises an amino acid sequence set forth as SEQ ID NO: 545. In some embodiments of the nucleic acid molecule aspect, the crRNA repeat has the nucleotide sequence set forth as SEQ ID NO: 546 or a nucleotide sequence that differs in length and/or sequence from SEQ ID NO: 546 by 1 to 8 nucleotides. In some embodiments of the nucleic acid molecule aspect, the crRNA repeat has the nucleotide sequence set forth as any one of SEQ ID NOs: 546, 549-552, 839, 842, and 845. In some embodiments of the nucleic acid molecule aspect, the crRNA has the nucleotide sequence set forth as any one of SEQ ID NOs: 574-692. In some embodiments of the nucleic acid molecule aspect, the tracrRNA has a nucleotide sequence having at least 80% sequence identity, at least 90% sequence identity, or at least 95% sequence identity to SEQ ID NO: 547. In some embodiments, the tracrRNA has a nucleotide sequence set forth as SEQ ID NO: 547. In some embodiments of the nucleic acid molecule aspect, the tracrRNA has a nucleotide sequence that differs in length from SEQ ID NO: 547 by 1 to 16 nucleotides. In some embodiments, the tracrRNA has a nucleotide sequence that is 8 nucleotides shorter than SEQ ID NO: 547. In some embodiments, the tracrRNA has a nucleotide sequence that is 11 nucleotides shorter than SEQ ID NO: 547. In some embodiments of the nucleic acid molecule aspect, the tracrRNA has the nucleotide sequence set forth as any one of SEQ ID NOs: 547, 553-562, 840, 842, and 846.

In some embodiments of the nucleic acid molecule aspect, the gRNA has a nucleotide sequence set forth as any one of SEQ ID NOs: 693-834. In some embodiments, the gRNA has a nucleotide sequence set forth as any one of SEQ ID NOs: 693, 694, 695, 696, 697, and 698.

In some embodiments of the nucleic acid molecule aspect, the gRNA comprises at least one chemical modification. In some embodiments, the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof. In some embodiments, the BNA comprises a 2′,4′ BNA modification. In some embodiments, the 2′,4′ BNA modification is selected from the group consisting of: locked nucleic acid (LNA) modification, BNANC[N-Me]modification, 2′-0,4′-C-ethylene bridged nucleic acid (2′,4′-ENA) modification, and S-constrained ethyl (cEt) modification. In some embodiments, the 2′,4′ BNA is a LNA modification. In some embodiments, the 2′,4′ BNA is a cEt modification. In some embodiments, the at least one chemical modification comprises a BNA modification, 2′-O-Me modification, PS modification, or a combination thereof.

In some embodiments, the at least one chemical modification comprises 2′-O-methyl 3′phosphorothioate (MS) modifications at the 3 terminal nucleotides at the 5′ end and at the 3 terminal nucleotides at the 3′ end of the gRNA. In some embodiments of the nucleic acid molecule aspect, the crRNA repeat has the nucleotide sequence set forth as any one of SEQ ID NOs: 940, 942-945, 1228, 1230, and 1232. In some embodiments of the nucleic acid molecule aspect, the crRNA has the nucleotide sequence set forth as any one of SEQ ID NOs: 967-1085. In some embodiments of the nucleic acid molecule aspect, the tracrRNA has the nucleotide sequence set forth as any one of SEQ ID NOs: 941, 946-955, 1229, 1231, and 1233. In some embodiments of the nucleic acid molecule aspect, the gRNA has the nucleotide sequence set forth as any one of SEQ ID NOs: 1086-1227.

In some embodiments of the nucleic acid molecule aspect, the gRNA further comprises an extension comprising an edit template for reverse transcriptase (RT) editing.

In yet another aspect, the present disclosure provides a nucleic acid molecule comprising a CRISPR RNA (crRNA) or encoding a crRNA, wherein the crRNA comprises a spacer and a crRNA repeat, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.

In some embodiments of the above nucleic acid molecule aspect, the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

In some embodiments of the above nucleic acid molecule aspect, the spacer is capable of hybridizing to a target sequence, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

In still another aspect, the present disclosure provides a vector comprising the nucleic acid molecule as described hereinabove, wherein the nucleic acid molecule comprises a polynucleotide encoding the crRNA. In some embodiments of the vector aspect, the nucleic acid molecule further comprises a heterologous promoter operably linked to the polynucleotide encoding the crRNA. In some embodiments, the heterologous promoter is an RNA polymerase III (pol III) promoter. In some embodiments of the vector aspect, the vector further comprises a nucleic acid molecule encoding an RGN polypeptide, wherein the crRNA is capable of binding a tracrRNA to form a guide RNA, wherein the guide RNA is capable of binding to the RGN polypeptide. In some embodiments, the vector further comprises a promoter operably linked to the nucleic acid molecule encoding the RGN polypeptide.

In yet another aspect, the present disclosure provides a vector comprising the nucleic acid molecule as described hereinabove, wherein the nucleic acid molecule comprises a polynucleotide encoding the crRNA, and wherein the vector further comprises a polynucleotide encoding the tracrRNA. In some embodiments of the vector aspect, the polynucleotide encoding the crRNA and the polynucleotide encoding the tracrRNA are operably linked to the same promoter and are encoded as a sgRNA. In some embodiments of the vector aspect, the polynucleotide encoding the crRNA and the polynucleotide encoding the tracrRNA are operably linked to separate promoters. In some embodiments of the vector aspect, the vector further comprises a nucleic acid molecule encoding an RGN polypeptide, wherein the crRNA is capable of binding the tracrRNA to form a guide RNA, wherein the guide RNA is capable of binding to the RGN polypeptide. In some embodiments, the vector further comprises a promoter operably linked to the nucleic acid molecule encoding the RGN polypeptide.

In another aspect, the present disclosure provides a cell comprising the gRNA, the nucleic acid molecule, or the vector as described hereinabove.

In another aspect, the present disclosure provides an RNA-guided nuclease (RGN) system for binding a target sequence in a forkhead box P3 (FOXP3) gene, wherein the RGN system comprises: a) one or more gRNAs as described hereinabove, or one or more polynucleotides comprising one or more nucleotide sequences encoding the one or more gRNAs as described hereinabove; and b) an RGN polypeptide, or a polynucleotide comprising a nucleotide sequence encoding the RGN polypeptide; wherein the one or more guide RNAs are capable of forming a complex with the RGN polypeptide to direct the RGN polypeptide to bind to the target sequence.

In some embodiments of the RGN system aspect, the RGN polypeptide is capable of recognizing a consensus protospacer adjacent motif (PAM) having the nucleotide sequence set forth as NNNNCC. In some embodiments of the RGN system aspect, the RGN polypeptide is capable of recognizing a full PAM having a nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments of the RGN system aspect, the RGN polypeptide comprises an amino acid sequence set forth as SEQ ID NO: 545.

In some embodiments of the RGN system aspect, the polynucleotide comprising a nucleotide sequence encoding the RGN polypeptide comprises an mRNA. In some embodiments of the RGN system aspect, the polynucleotide comprising a nucleotide sequence encoding the RGN polypeptide is codon optimized for expression in a mammalian cell. In some embodiments of the RGN system aspect, at least one of the one or more nucleotide sequences encoding the one or more gRNAs and the nucleotide sequence encoding the RGN polypeptide is operably linked to a promoter heterologous to the nucleotide sequence. In some embodiments of the RGN system aspect, the one or more nucleotide sequences encoding the one or more gRNAs and the nucleotide sequence encoding the RGN polypeptide are located on one vector. In some embodiments of the RGN system aspect, the RGN polypeptide is nuclease inactive or is a nickase. In some embodiments of the RGN system aspect, the RGN polypeptide is fused to a base-editing polypeptide. In some embodiments, the base-editing polypeptide comprises a deaminase. In some embodiments of the RGN system aspect, the RGN polypeptide is fused to a RT editing polypeptide. In some embodiments, the RT editing polypeptide comprises a DNA polymerase. In some embodiments, the DNA polymerase comprises a reverse transcriptase. In some embodiments of the RGN system aspect, the gRNA further comprises an extension comprising an edit template for RT editing. In some embodiments of the above aspect, the RGN polypeptide comprises one or more nuclear localization signals.

In still another aspect, the present disclosure provides a ribonucleoprotein (RNP) complex comprising the one or more gRNA and the RGN polypeptide of the RGN system as described hereinabove.

In still another aspect, the present disclosure provides a cell comprising the RGN system or the RNP complex as described hereinabove. In some embodiments, the cell is a eukaryotic cell. In some embodiments, the eukaryotic cell is a mammalian cell. In some embodiments, the mammalian cell is a human cell. In some embodiments, the mammalian cell or human cell is a T cell or an induced pluripotent stem cell.

In another aspect, the present disclosure provides a method for binding a target sequence within a FOXP3 gene, comprising delivering the RGN system or the RNP complex as described hereinabove to the target sequence or a cell comprising the target sequence. In some embodiments of the method for binding a target sequence within a FOXP3 gene aspect, cleavage or modification of the target sequence occurs.

In another aspect, the present disclosure provides a method for assembling an RNA-guided nuclease (RGN) ribonucleoprotein complex, the method comprising combining under conditions suitable for formation of the complex: a) the guide RNA as described hereinabove; and b) an RGN polypeptide that binds the guide RNA. In some embodiments of the above aspect, the RGN polypeptide is capable of recognizing a consensus protospacer adjacent motif (PAM) having the nucleotide sequence set forth as NNNNCC. In some embodiments of the method for assembling an RGN ribonucleoprotein complex aspect, the complex directs cleavage of the target sequence. In some embodiments, the cleavage generates a double-stranded break. In some embodiments, wherein the cleavage generates a single-stranded break.

In another aspect, the present disclosure provides a method for binding a target sequence within a FOXP3 gene, the method comprising: a) combining under conditions suitable for formation of a ribonucleoprotein (RNP) complex: i) the guide RNA as described hereinabove; and ii) an RGN polypeptide that binds the guide RNA; thereby assembling an RNP complex; and b) contacting the target sequence or a cell comprising the target sequence with the assembled RNP complex; thereby directing binding of the RNP complex to the target sequence. In some embodiments of the above aspect, the RGN polypeptide is capable of recognizing a consensus protospacer adjacent motif (PAM) having the nucleotide sequence set forth as NNNNCC. In some embodiments of the method for binding a target sequence within a FOXP3 gene aspect, the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments of the method for binding a target sequence within a FOXP3 gene aspect, the RGN polypeptide comprises an amino acid sequence set forth as SEQ ID NO: 545.

In some embodiments of the method for binding a target sequence within a FOXP3 gene aspect, the method is performed in vitro or ex vivo. In some embodiments of the method for binding a target sequence within a FOXP3 gene aspect, the RGN polypeptide is capable of cleaving the target sequence, thereby allowing for the cleaving and/or modifying of the target sequence. In some embodiments, the cleaving generates a double-stranded break. In some embodiments, the cleaving generates a single-stranded break. In some embodiments, the cleaving results in insertion of a heterologous sequence within the target sequence.

In some embodiments of the method for binding a target sequence within a FOXP3 gene aspect, the RGN polypeptide is nuclease inactive or is a nickase. In some embodiments of the method for binding a target sequence within a FOXP3 gene aspect, the RGN polypeptide is fused to a base-editing polypeptide. In some embodiments, the base-editing polypeptide comprises a deaminase. In some embodiments of the method for binding a target sequence within a FOXP3 gene aspect, the RGN polypeptide is fused to a RT editing polypeptide. In some embodiments, the RT editing polypeptide comprises a DNA polymerase. In some embodiments, the DNA polymerase comprises a reverse transcriptase. In some embodiments of the method for binding a target sequence within a FOXP3 gene aspect, the gRNA further comprises an extension comprising an edit template for RT editing.

In a further aspect, the present disclosure provides a method for modulating expression of a forkhead box P3 (FOXP3) gene in a population of cells, comprising delivering the RGN system described hereinabove or the RNP complex described hereinabove to the population of cells, wherein the population of cells comprises the target sequence, and wherein FOXP3 gene expression is modulated as compared to FOXP3 gene expression in a control population of cells.

In some embodiments of the method for modulating expression of a FOXP3 gene aspect, cleavage or modification of the target sequence occurs. In some embodiments, cleavage or modification of the target sequence is detected by sequencing. In some embodiments, FOXP3 gene expression is measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof.

In some embodiments of the method for modulating expression of a FOXP3 gene aspect, FOXP3 gene expression is decreased. In some embodiments, the decrease in FOXP3 gene expression comprises decrease in FOXP3 mRNA and/or Foxp3 protein level.

In some embodiments of the method for modulating expression of a FOXP3 gene aspect, cleavage or modification of the target sequence occurs at a rate of 40% to 100%. In some embodiments, cleavage or modification of the target sequence occurs at a rate of 80% to 100%.

In some embodiments of the method for modulating expression of a FOXP3 gene aspect, the control population of cells has not been subjected to the delivering.

In some embodiments of the method for modulating expression of a FOXP3 gene aspect, the population of cells comprises T cells.

### BRIEF DESCRIPTION OF THE FIGURES

FIG. 1 shows that increasing spacer length improves editing of APG07433.1 guide RNAs targeting forkhead box P3 (FOXP3) gene.

FIG. 2 shows gene editing rate (as % insertions/deletions (indel)) for multiple FOXP3 guide RNAs over a tested dose curve of guide RNA:RGN protein. For each delivery format, the guides are from left to right: SGN 3378, SGN 3379, SGN 3381, SGN3383, and SGN3384.

FIG. 3 shows consistent editing of FOXP3 guide RNAs at higher doses of ribonucleoprotein (RNP) complex of guide RNA and APG07433.1 RGN. For each guide used, the dose of RNP complex and RGN protein:guide RNA ratio are from left to right: 90 pmol 1:2, 90 pmol 1:3, 120 pmol 1:2, and 120 pmol 1:3.

FIG. 4 shows multiple guide RNAs having >70% editing at FOXP3 in cells from different donors. For each guide used, the donor and RGN protein:guide RNA ratio are from left to right: Donor 1 (F) 1:2, Donor 1 (F) 1:3, Donor 2 (M) 1:2, Donor 2 (M) 1:3, Donor 3 (F) 1:2, and Donor 3 (F) 1:3.

FIG. 5 shows multiple guide RNAs having >70% editing at FOXP3 in cells from different donors and across a range of RNP complex doses. For each guide used, the dose of RNP complex is from left to right: 20 pmol, 40 pmol, 60 pmol, and 80 pmol.

FIG. 6 shows performance of guide RNAs in FOXP3 editing as ratio of editing of guide RNAs with backbone variants and various spacer lengths to guide RNA with native backbone and 25 nt spacer (‘original backbone (135 bp)’). The M backbone has: a deletion of 10 nt in the first stem of stem loop 1 formed by hybridization of the crRNA repeat and anti-repeat; a deletion of 2 nt in stem loop 3 most proximal to the tail of the guide RNA; and a deletion of 4 nt from the tail of the guide RNA; as compared to the native APG07433.1 backbone. The 98bblen has a deletion of 12 nt in the first stem of stem loop 1; the 98bblen_-6 tail has a deletion of 12 nt in the first stem of stem loop 1 and a deletion of 6 nt from the tail; the 98bblen_-6 tail_-2hairpin has a deletion of 12 nt in the first stem of stem loop 1, a deletion of 6 nt from the tail, and a deletion of 2 nt from stem loop 3. The 96bblen has a deletion of 14 nt in the first stem of stem loop 1; the 96bblen_-6 tail has a deletion of 14 nt in the first stem of stem loop 1 and a deletion of 6 nt from the tail; the 96bblen_-6 tail_-2hairpin has a deletion of 14 nt in the first stem of stem loop 1, a deletion of 6 nt from the tail, and a deletion of 2 nt from stem loop 3. The 94bblen has a deletion of 16 nt in the first stem of stem loop 1; the 94bblen_-6 tail has a deletion of 16 nt in the first stem of stem loop 1 and a deletion of 6 nt from the tail; the 94bblen_-6 tail_-2hairpin has a deletion of 16 nt in the first stem of stem loop 1, a deletion of 6 nt from the tail, and a deletion of 2 nt from stem loop 3. For each backbone variant, the guides are from left to right: SGN 3378, SGN 3381, SGN3382, and SGN3384 (indicated as ‘1-4’ in the graph).

FIG. 7 shows performance of guide RNAs in FOXP3 editing as percent editing of each guide RNA. The backbone variants are as described in FIG. 6. For each backbone variant, the guides are from left to right: SGN 3378, SGN 3381, SGN3382, and SGN3384 (indicated as ‘1-4’ in the graph).

FIG. 8 shows that the ‘M’ and 94 nt length backbones yielded high gene editing across a number of FOXP3 targets and was dependent upon spacer length. For each guide spacer-backbone format, the guides are from left to right: SGN 3378, SGN 3381, SGN3382, SGN3384, and SGN 5073. The M and 94 nt length backbones are as described in FIG. 6. The gene editing rates for guide RNAs with M or nt length backbones are compared to that for a guide RNA with native backbone and 25 nt spacer (‘original backbone (135 bp)’).

FIG. 9 shows that truncated guide RNAs (shortened in spacer and/or backbone) were effective at editing multiple FOXP3 target sites across a dose range of RNP complex of guide RNA and APG07433.1 RGN and across multiple donors. For each guide used, the dose of RNP complex is from left to right: 20 pmol, 40 pmol, 60 pmol, and 80 pmol.

FIG. 10 shows that most truncated guide RNAs showed equal or slightly improved editing as compared to the original guide RNA with native backbone and 25 nt spacer. For each guide used, the dose of RNP complex is from left to right: 20 pmol, 40 pmol, 60 pmol, and 80 pmol.

FIG. 11 shows that cell viability was at or above 80% for most samples, across multiple donors, and across a dose range of RNP complex of guide RNA and APG07433.1 RGN. For each guide used, the dose of RNP complex is from left to right: 20 pmol, 40 pmol, 60 pmol, and 80 pmol. For the ‘Donor Controls’ graph, the donors are from left to right: Donor 1, Donor 2, and Donor 3.

FIGS. 12A and 12B show gene editing rate as percent insertions and deletions (indels) in screens to identify effective FOXP3 guide RNAs in association with APG07433.1 RGN. SGN000754 and SGN000755 are control guide RNAs in FIG. 12A. For each guide in FIG. 12A, the % editing with RNP is on the left, and the % editing with mRNA is on the right. SGN002770-SGN002803 are FOXP3 guide RNAs in FIG. 12A. SGN005050-SGN005104 are FOXP3 guide RNAs in FIG. 12B.

FIG. 13 shows that changes to spacer length can generate a better guide RNA with no significant off-target modifications. For each on target site or predicted in silico off target site, the % indel for edited is on the left, and the % indel for control is on the right.

FIG. 14 shows that 5 of the 6 lead FOXP3 guide RNAs had no significant off-target modifications. For each on target site or predicted in silico off target site, the % indel for edited is on the left, and the % indel for control is on the right. The control indicates conditions without RGN and gRNA, where cells are mixed with nucleofection solution but do not go through the nucleofection process.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

RNA-guided nuclease (RGN) systems allow for the targeted manipulation of specific site(s) within a genome and are useful in the context of gene targeting for therapeutic and research applications. In a variety of organisms, including mammals, RGN systems have been used for genome engineering by stimulating non-homologous end joining and homologous recombination, for example. The compositions and methods described herein are useful for modifying the forkhead box P3 (FOXP3) gene.

The RGN systems disclosed herein can bind, cleave, and/or modify target sequences in the FOXP3 gene. Modification of the FOXP3 gene can include reducing or eliminating expression of FoxP3. The guide RNAs of the disclosed RGN systems can be engineered to be shorter than their native lengths and still maintain editing efficiencies of >60%.

The ability to manipulate expression of Foxp3 would be desirable in controlling the function of a T cell to either encourage immune suppression in an inflammatory or autoimmune setting or to reduce immune suppression in a tumor microenvironment.

### II. Guide RNA

The present disclosure provides guide RNAs, components thereof, and polynucleotides encoding the same that target an associated RNA-guided nuclease (RGN) to a target nucleotide sequence in the FOXP3 gene. The term “guide RNA” is known in the art and generally refers to an RNA molecule (or a group of RNA molecules collectively) that can bind to an RNA-guided nuclease (RGN) and aid in targeting the RGN to a specific location within a target polynucleotide (e.g., a DNA or an mRNA molecule). The guide RNA can comprise a nucleotide sequence (i.e., a spacer) having sufficient complementarity with a target nucleotide sequence to hybridize with the target sequence and direct sequence-specific binding of an RGN to the target nucleotide sequence. In some embodiments, when the target nucleotide sequence is double-stranded as is the case with DNA, the target nucleotide sequence comprises a non-target strand (which comprises the PAM sequence) and the target strand, which hybridizes with the spacer of the guide RNA. In these embodiments, the guide RNA has sufficient complementarity with the target strand of a double-stranded target sequence (e.g., target DNA sequence of a FOXP3 gene) such that the guide RNA hybridizes with the target strand and directs sequence-specific binding of an associated RGN to the target sequence (e.g., target DNA sequence of a FOXP3 gene). Therefore, in some embodiments, a guide RNA includes a spacer that is identical to the sequence of the non-target strand except that uracil (U) replaces thymidine (T) in the guide RNA.

An RGN's respective guide RNA is one or more RNA molecules (generally, one or two), that can bind to the RGN and guide the RGN to bind to a particular target sequence, and in those embodiments wherein the RGN has nickase or nuclease activity, also cleave the target strand and/or the non-target strand. In general, a guide RNA comprises a CRISPR RNA (crRNA) and a trans-activating CRISPR RNA (tracrRNA).

The term “guide RNA” also encompasses, collectively, a group of two or more RNA molecules, where the crRNA and the tracrRNA are located in separate RNA molecules. Native guide RNAs that comprise both a crRNA and a tracrRNA generally comprise two separate RNA molecules that hybridize to each other through the repeat sequence of the crRNA and the anti-repeat sequence of the tracrRNA. In certain embodiments, the crRNA and tracrRNA are linked together by a multi-nucleotide linker (e.g., a four-nucleotide linker) to form a single guide RNA molecule, wherein the crRNA and the tracrRNA hybridize to each other through the repeat sequence of the crRNA and the anti-repeat sequence of the tracrRNA. Thus, a guide RNA encompasses a single-guide RNA (sgRNA), where the crRNA and the tracrRNA are located in the same RNA molecule or strand. A total length of a guide RNA refers to the length of the spacer and backbone in a sgRNA, or length of the crRNA and tracrRNA in a dgRNA.

A guide RNA of the disclosure can comprise at least one chemical modification. The at least one chemical modification includes: a bridged nucleic acid (BNA) modification; 2-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2-O-methyl 3′phosphorothioate (MS) modification; 2-O-methyl 3′thiophosphonoacetate (MSP) modification; 2-O-methyl 3′phosphonoacetate (MP) modification; and phosphorothioate (PS) modification; or a combination thereof. In some embodiments, the BNA comprises a 2′,4′ BNA modification. In some embodiments, the 2′,4′ BNA modification is selected from the group consisting of: locked nucleic acid (LNA) modification, BNA^{NC}[N-Me]modification, 2′-O,4′-C-ethylene bridged nucleic acid (2′,4′-ENA) modification, and S-constrained ethyl (cEt) modification. In some embodiments, the 2′,4′ BNA is a LNA modification. In some embodiments, the 2′,4′ BNA is a cEt modification. In some embodiments, the at least one chemical modification comprises a BNA modification, 2′-O-Me modification, or PS modification. Chemical modifications of spacers, crRNA repeats, crRNAs, tracrRNAs, and guide RNAs are described in International application no. PCT/IB2023/058418, filed Aug. 25, 2023, which is hereby incorporated by reference in its entirety herein. The at least one chemical modification can comprise 2′-O-methyl 3′phosphorothioate (MS) modifications at the 3 terminal nucleotides at the 5′ region and at the 3 terminal nucleotides at the 3′ region of the guide RNA. As used herein, a “5′ region” of an RNA molecule disclosed herein includes the first nucleotide, the first 2 nucleotides, the first 3 nucleotides, the first 4 nucleotides, or the first 5 nucleotides of the 5′ end of the RNA molecule. As used herein, a “3′ region” of an RNA molecule disclosed herein includes the first nucleotide, the first 2 nucleotides, the first 3 nucleotides, the first 4 nucleotides, or the first 5 nucleotides of the 3′ end of the RNA molecule. In some embodiments, a 3′ region of a crRNA in the context of a single guide RNA includes the first nucleotide, the first 2 nucleotides, the first 3 nucleotides, the first 4 nucleotides, or the first 5 nucleotides from the tracrRNA or the linker that joins the crRNA and the tracrRNA of the single guide RNA.

As used herein, the term “crRNA” refers to an RNA molecule or portion thereof that includes a spacer, which is the nucleotide sequence that hybridizes with the target strand of a target sequence, and a CRISPR repeat (i.e. a crRNA repeat) that comprises a nucleotide sequence that forms a structure, either on its own or in concert with a hybridized tracrRNA, that is recognized by the RGN molecule. As used herein, the term “tracrRNA” or “transactivating crRNA” refers to an RNA molecule that comprises an anti-repeat sequence that has sufficient complementarity to hybridize to at least a portion of the CRISPR repeat of a crRNA to form a structure that is recognized by an RGN molecule. In some embodiments, additional secondary structure(s) (e.g., stem-loops) within the tracrRNA molecule is required for binding to an RGN.

The present invention provides CRISPR RNAs (crRNAs) or polynucleotides encoding CRISPR RNAs that target an associated RGN to a target sequence in the FOXP3 gene. A crRNA comprises a spacer and a CRISPR repeat. The “spacer” has a nucleotide sequence that directly hybridizes with the non-target strand of a target sequence (e.g., target DNA sequence in the FOXP3 gene) of interest. The spacer is engineered to have full or partial complementarity with the target strand of a target sequence of interest. In some embodiments, the spacer can comprise from about 8 nucleotides to about 30 nucleotides, or more. For example, the spacer can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer is 8, 9, 10, 11, 12, 13, 14, 15, 16, 17, 18, 19, 20, 21, 22, 23, 24, 25, 26, 27, 28, 29, 30, or more nucleotides in length. In some embodiments, the spacer is about 10 to about 26 nucleotides in length, or about 12 to about 30 nucleotides in length. In some embodiments, the spacer is about 30 nucleotides in length. In embodiments, the spacer is 30 nucleotides in length. In some embodiments, the degree of complementarity between a spacer and the target strand of a target sequence (e.g., target DNA sequence), when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In embodiments, the degree of complementarity between a spacer and the target strand of a target sequence (e.g., target DNA sequence), when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more. The spacer can be identical in sequence to the non-target strand of a target sequence. In some of those embodiments wherein the target sequence is a target DNA sequence, the spacer can be identical in sequence to the non-target strand of the target DNA sequence, with the exception of the thymidines (Ts) in the target strand being replaced by uracils (Us) in the spacer. In some embodiments, the spacer is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24). A spacer can comprise at least one chemical modification. In some embodiments, a spacer as part of a guide RNA comprises 2′-O-methyl 3′phosphorothioate (MS) modifications at the 3 terminal nucleotides at the 5′ region of the spacer.

The presently disclosed crRNAs comprise a spacer capable of targeting a bound RGN polypeptide to a target sequence in the forkhead box P3 (FOXP3) gene, wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214. In some embodiments, a spacer of the disclosure has a nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 or a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 5 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 4 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 3 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 2 nucleotides.

In some embodiments, the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 nucleotide.

In some embodiments, a spacer of the disclosure has a nucleotide sequence set forth as: UGCCAGGCCUGGGGUUGGGCAUC (SEQ ID NO: 155), or a nucleotide sequence that differs in length and/or sequence from SEQ ID NO: 155 by 1 to 5 nucleotides. In some embodiments, the spacer has a nucleotide sequence that differs in length and/or sequence from SEQ ID NO: 155 by 5 nucleotides. In some embodiments, the spacer has a nucleotide sequence that differs in length and/or sequence from SEQ ID NO: 155 by 4 nucleotides. In some embodiments, the spacer has a nucleotide sequence that differs in length and/or sequence from SEQ ID NO: 155 by 3 nucleotides. In some embodiments, the spacer has a nucleotide sequence that differs in length and/or sequence from SEQ ID NO: 155 by 2 nucleotides. In some embodiments, the spacer has a nucleotide sequence that differs in length and/or sequence from SEQ ID NO: 155 by 1 nucleotide.

In some embodiments, a spacer of the disclosure has a nucleotide sequence set forth as: CAGGUCUGAGGCUUUGGGUGCAG (SEQ ID NO: 163), or a nucleotide sequence that differs in length and/or sequence from SEQ ID NO: 163 by 1 to 5 nucleotides. In some embodiments, the spacer has a nucleotide sequence that differs in length and/or sequence from SEQ ID NO: 163 by 5 nucleotides. In some embodiments, the spacer has a nucleotide sequence that differs in length and/or sequence from SEQ ID NO: 163 by 4 nucleotides. In some embodiments, the spacer has a nucleotide sequence that differs in length and/or sequence from SEQ ID NO: 163 by 3 nucleotides. In some embodiments, the spacer has a nucleotide sequence that differs in length and/or sequence from SEQ ID NO: 163 by 2 nucleotides. In some embodiments, the spacer has a nucleotide sequence that differs in length and/or sequence from SEQ ID NO: 163 by 1 nucleotide.

In some embodiments, a spacer of the disclosure has a nucleotide sequence set forth as: UCGAAGAUCUCGGCCCUGGAAGG (SEQ ID NO: 179), or a nucleotide sequence that differs in length and/or sequence from SEQ ID NO: 179 by 1 to 5 nucleotides. In some embodiments, the spacer has a nucleotide sequence that differs in length and/or sequence from SEQ ID NO: 179 by 5 nucleotides. In some embodiments, the spacer has a nucleotide sequence that differs in length and/or sequence from SEQ ID NO: 179 by 4 nucleotides. In some embodiments, the spacer has a nucleotide sequence that differs in length and/or sequence from SEQ ID NO: 179 by 3 nucleotides. In some embodiments, the spacer has a nucleotide sequence that differs in length and/or sequence from SEQ ID NO: 179 by 2 nucleotides. In some embodiments, the spacer has a nucleotide sequence that differs in length and/or sequence from SEQ ID NO: 179 by 1 nucleotide.

In some embodiments, a spacer of the disclosure has a nucleotide sequence set forth as: UCUCGGCCCUGGAAGGUUCCCCCUG (SEQ ID NO: 189), or a nucleotide sequence that differs in length and/or sequence from SEQ ID NO: 189 by 1 to 5 nucleotides. In some embodiments, the spacer has a nucleotide sequence that differs in length and/or sequence from SEQ ID NO: 189 by 5 nucleotides. In some embodiments, the spacer has a nucleotide sequence that differs in length and/or sequence from SEQ ID NO: 189 by 4 nucleotides. In some embodiments, the spacer has a nucleotide sequence that differs in length and/or sequence from SEQ ID NO: 189 by 3 nucleotides. In some embodiments, the spacer has a nucleotide sequence that differs in length and/or sequence from SEQ ID NO: 189 by 2 nucleotides. In some embodiments, the spacer has a nucleotide sequence that differs in length and/or sequence from SEQ ID NO: 189 by 1 nucleotide.

In some embodiments, a spacer of the disclosure has a nucleotide sequence set forth as: GGUUCAAGGAAGAAGAGGAGGCA (SEQ ID NO: 197), or a nucleotide sequence that differs in length and/or sequence from SEQ ID NO: 197 by 1 to 5 nucleotides. In some embodiments, the spacer has a nucleotide sequence that differs in length and/or sequence from SEQ ID NO: 197 by 5 nucleotides. In some embodiments, the spacer has a nucleotide sequence that differs in length and/or sequence from SEQ ID NO: 197 by 4 nucleotides. In some embodiments, the spacer has a nucleotide sequence that differs in length and/or sequence from SEQ ID NO: 197 by 3 nucleotides. In some embodiments, the spacer has a nucleotide sequence that differs in length and/or sequence from SEQ ID NO: 197 by 2 nucleotides. In some embodiments, the spacer has a nucleotide sequence that differs in length and/or sequence from SEQ ID NO: 197 by 1 nucleotide.

In some embodiments, a spacer of the disclosure has a nucleotide sequence set forth as: GGGGUUCAAGGAAGAAGAGGAGGCA (SEQ ID NO: 193), or a nucleotide sequence that differs in length and/or sequence from SEQ ID NO: 193 by 1 to 5 nucleotides. In some embodiments, the spacer has a nucleotide sequence that differs in length and/or sequence from SEQ ID NO: 193 by 5 nucleotides. In some embodiments, the spacer has a nucleotide sequence that differs in length and/or sequence from SEQ ID NO: 193 by 4 nucleotides. In some embodiments, the spacer has a nucleotide sequence that differs in length and/or sequence from SEQ ID NO: 193 by 3 nucleotides. In some embodiments, the spacer has a nucleotide sequence that differs in length and/or sequence from SEQ ID NO: 193 by 2 nucleotides. In some embodiments, the spacer has a nucleotide sequence that differs in length and/or sequence from SEQ ID NO: 193 by 1 nucleotide.

Along with a spacer, crRNAs further comprise a CRISPR RNA repeat. The CRISPR RNA repeat comprises a nucleotide sequence that forms a structure, either on its own or in concert with a hybridized tracrRNA, that is recognized by the RGN molecule. In some embodiments, the CRISPR RNA repeat can comprise from about 8 nucleotides to about 30 nucleotides, or more. For example, the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat is 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA antirepeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the degree of complementarity between a CRISPR repeat and its corresponding tracrRNA antirepeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

The CRISPR repeat can comprise the nucleotide sequence of any one of SEQ ID NOs: 546, 549-552, 839, 842, and 845, or an active variant or fragment thereof that when comprised within a guide RNA, is capable of directing the sequence-specific binding of an associated RNA-guided nuclease provided herein to a presently disclosed target DNA sequence within the FOXP3 gene. In some embodiments, an active CRISPR repeat variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to a nucleotide sequence set forth as any one of SEQ ID NOs: 546, 549-552, 839, 842, and 845. In some embodiments, an active CRISPR repeat fragment comprises at least 5, 6, 7, 8, 9, 10, 11, 12, 13, 14, 15, 16, 17, 18, 19, 20, 21, or 22 contiguous nucleotides of a nucleotide sequence set forth as any one of SEQ ID NOs: 546, 549-552, 839, 842, and 845. In some embodiments, the CRISPR repeat comprises a nucleotide sequence that differs in length and/or sequence from SEQ ID NO: 546 by 1 to 8 nucleotides. In some embodiments, the CRISPR repeat comprises a nucleotide sequence that differs in length and/or sequence from SEQ ID NO: 546 by 8 nucleotides. In some embodiments, the CRISPR repeat comprises a nucleotide sequence that differs in length and/or sequence from SEQ ID NO: 546 by 7 nucleotides. In some embodiments, the CRISPR repeat comprises a nucleotide sequence that differs in length and/or sequence from SEQ ID NO: 546 by 6 nucleotides. In some embodiments, the CRISPR repeat comprises a nucleotide sequence that differs in length and/or sequence from SEQ ID NO: 546 by 5 nucleotides. In some embodiments, the CRISPR repeat comprises a nucleotide sequence that differs in length and/or sequence from SEQ ID NO: 546 by 4 nucleotides. In some embodiments, the CRISPR repeat comprises a nucleotide sequence that differs in length and/or sequence from SEQ ID NO: 546 by 3 nucleotides. In some embodiments, the CRISPR repeat comprises a nucleotide sequence that differs in length and/or sequence from SEQ ID NO: 546 by 2 nucleotides. In some embodiments, the CRISPR repeat comprises a nucleotide sequence that differs in length and/or sequence from SEQ ID NO: 546 by 1 nucleotide. In some embodiments, the CRISPR repeat comprises the nucleotide sequence set forth as: GUCAUAGUUCCAUUAAAGCCA (SEQ ID NO: 546). A CRISPR repeat can comprise at least one chemical modification. In some embodiments, a CRISPR repeat as part of a guide RNA comprises 2′-O-methyl 3′phosphorothioate (MS) modifications at the 3 terminal nucleotides at the 3′ region of the CRISPR repeat. CRISPR repeats comprising 2′-O-methyl 3′phosphorothioate (MS) modifications at the 3 terminal nucleotides at the 3′ region of the CRISPR repeat can have nucleotide sequences set forth as any one of SEQ ID NOs: 940, 942-945, 1228, 1230, and 1232.

The crRNA can be an engineered sequence that is not naturally occurring. In some embodiments, the specific CRISPR repeat is not linked to the engineered spacer in nature and the CRISPR repeat is considered heterologous to the spacer. In some embodiments, the spacer is an engineered sequence that is not naturally occurring.

In some embodiments, the crRNA has the sequence set forth as any one of SEQ ID NOs: 574-692. A crRNA can comprise at least one chemical modification. In some embodiments, a crRNA of the disclosure can comprise 2′-O-methyl 3′phosphorothioate (MS) modifications at the 3 terminal nucleotides at the 5′ region and at the 3 terminal nucleotides at the 3′ region of the crRNA. crRNAs comprising 2′-O-methyl 3′phosphorothioate (MS) modifications at the 3 terminal nucleotides at the 5′ region and at the 3 terminal nucleotides at the 3′ region of the crRNA can have nucleotide sequences set forth as any one of SEQ ID NOs: 967-1085.

Generally, the presently disclosed guide RNAs comprise a crRNA and a trans-activating CRISPR RNA (tracrRNA), while some presently disclosed compositions and methods utilize RGN polypeptides that do not require a tracrRNA. A tracrRNA molecule comprises a nucleotide sequence comprising a region, referred to herein as the anti-repeat, that has sufficient complementarity to hybridize to a crRNA repeat. In some embodiments, the tracrRNA molecule further comprises a region with secondary structure (e.g., stem-loop). In some embodiments, secondary structure includes nucleotides that are in one of two states, paired or unpaired, where nucleotide or base pairing includes base-base hydrogen bonding interactions (e.g., adenine (A) pairs with uracil (U), cytosine (C) pairs with guanine (G)) between two complementary nucleic acid strands to form a helix. In some embodiments, the combination of one or more helical elements interspersed with unpaired, single-stranded nucleotides constitutes an RNA structure.

A “stem loop” as used herein refers to a form of secondary structure comprising at least one “stem” and at least one “loop”, “bulge”, or “bubble” found in polynucleotides. A stem loop can form intramolecularly (within one molecule, e.g., within a tracrRNA or a sgRNA) or intermolecularly (between two distinct nucleic acids, e.g., in a dual guide RNA by the crRNA repeat of a crRNA and the anti-repeat of a tracrRNA). Stem loops are created when there is at least some complementarity between two nucleic acid sequences to form a paired double helix. The paired double helix region with full complementarity or sometimes including a G:U wobble base pair (or I:U, I:A, or I:C, where I refers to inosine) is referred to as a “stem”. The term “loop”, “bulge”, or “bubble” refers to a single stranded region within the “stem loop” structure where there is no complementarity between nucleotides, excluding G:U wobble base pairs (or I:U, I:A, or I:C, where I refers to inosine). Thus, “loops”, “bulges” and “bubbles” include nucleotides that are not paired. In some embodiments, a “loop” is distinguished from a “bulge” or “bubble” by being located at one end of the “stem loop” structure, while a “bulge” or a “bubble” is located between two “stems” in the “stem loop” structure.

In certain embodiments, a stem loop structure comprises a stem and a loop at one end of the stem. In some embodiments, a stem loop structure comprises a first stem and a second stem with a bubble in between the stems. In some embodiments, a stem loop structure comprises a loop, multiple stems and multiple bubbles in between the stems. In this circumstance, the bubbles in the order of closeness to the loop are referred to as a “first bubble”, a “second bubble”, a “third bubble”, etc., and the stems in the order of closeness to the loop are referred to as a “first stem”, a “second stem”, a “third stem”, etc. In embodiments of dgRNA, the stem loop formed by the crRNA repeat of a crRNA and the anti-repeat of a tracrRNA does not include a loop, and thus the bubbles in the order of closeness to the 5′ end of the tracrRNA (or 3′ end of the crRNA) are referred to as a “first bubble”, a “second bubble”, a “third bubble”, etc., and the stems in the order of closeness to the 5′ end of the tracrRNA (or 3′ end of the crRNA) are referred to as a “first stem”, a “second stem”, a “third stem”, etc.

The term “first stem of a crRNA repeat of a crRNA”, “first stem of a crRNA repeat”, or “first stem of a crRNA” means the region in the crRNA repeat of the crRNA that forms the first stem of a stem loop structure when hybridizing with an anti-repeat of a tracrRNA. The term “second stem of a crRNA repeat of a crRNA”, “second stem of a crRNA repeat”, or “second stem of a crRNA” means the region in the crRNA repeat of the crRNA that forms the second stem of a stem loop structure when hybridizing with an anti-repeat of a tracrRNA. Similarly, the term “first stem of an anti-repeat of a tracrRNA”, “first stem of an anti-repeat”, or “first stem of a tracrRNA” means the region in the anti-repeat of the tracrRNA that forms the first stem of a stem loop structure when hybridizing with a crRNA repeat of a crRNA. The term “second stem of an anti-repeat of a tracrRNA”, “second stem of an anti-repeat”, or “second stem of a tracrRNA” means the region in the anti-repeat of the tracrRNA that forms the second stem of a stem loop structure when hybridizing with a crRNA repeat of a crRNA.

In some embodiments, a stem loop formed intramolecularly is a hairpin stem loop. Base pairings occur in the stem part of a stem loop and typically involve guanine-cytosine base pairing and adenine-uracil(thymidine) base pairing, although guanine-uracil base pairing is possible. Base stacking interactions promote helix formation. The loop part of a stem loop includes bases that are not paired. In some embodiments, a loop is the point at which a nucleic acid strand turns back on itself for nucleotide pairing to create a stem. In some embodiments, loops that are less than three bases long are sterically impossible and do not form. In some embodiments, optimal loop length is about 4-8 bases long. Common loops with four nucleotide sequences such as GAAA, AAAG, ACUU, or UUCG are known as the “tetraloop” and are particularly stable due to the base-stacking interactions of its component nucleotides.

In some embodiments, the region of the tracrRNA that is fully or partially complementary to a crRNA repeat is at the 5′ end of the molecule and the 3′ end of the tracrRNA comprises secondary structure. This region of secondary structure generally comprises several hairpin structures, including the nexus hairpin, which is found adjacent to the anti-repeat. The nexus forms the core of the interactions between the guide RNA and the RGN, and is at the intersection between the guide RNA, the RGN, and the target sequence. The nexus hairpin often has a conserved nucleotide sequence in the base of the hairpin stem, with the motif UNANNC found in many nexus hairpins in tracrRNAs. In embodiments, guide RNAs or RGN systems of the disclosure use tracrRNAs that comprise non-canonical sequences in the base of the hairpin stem of their nexus hairpins, including UNANNG and CNANNC. In some embodiments, a guide RNA or an RGN system of the disclosure uses a tracrRNA that includes, in the base of the nexus hairpin stem, the non-canonical sequence of UNANNG. In some embodiments, a guide RNA or an RGN system of the disclosure uses a tracrRNA that includes, in the base of the nexus hairpin stem, the non-canonical sequence of CNANNC. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

A tracrRNA of the disclosure can include a tail. The term “tail” as used herein refers to the non-complementary region closest to the 3′ end (e.g., within twelve, eleven, ten, nine, eight, seven, six, five nucleotides from the 3′ end) of a tracrRNA of the disclosure. In some embodiments, a tail of a tracrRNA includes 1-12, 1-8, 1-7, or 1-6 nucleotides from the 3′ end of the tracrRNA. In some embodiments, a tail of a tracrRNA includes 1, 2, 3, 4, 5, 6, 7, 8, 9, 10, 11, 12, or more nucleotides from the 3′ end of the tracrRNA.

A tracrRNA of the disclosure can include additional hairpin or stem loop structures in addition to the nexus hairpin. In some embodiments, a tracrRNA includes at least one stem loop. In some embodiments, a tracrRNA includes at least one stem loop proximal to the anti-repeat and at least one stem loop proximal to the 3′ end of the tracrRNA. “Proximal” refers to being within 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides of a region or an end of a nucleic acid molecule. In certain embodiments, “proximal” refers to being within 1 nucleotide, 2 nucleotides, 3 nucleotides, 4 nucleotides, 5 nucleotides, or 6 nucleotides of a region or an end of a nucleic acid molecule. “Most proximal” refers to being the nearest to a region or to an end of a nucleic acid molecule. For example, a stem loop most proximal to the tail of a tracrRNA is the first stem loop nearest the tail of the tracrRNA. “Distal” refers to being at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more away from a region or an end of a nucleic acid molecule. In some embodiments, “distal” refers to being at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more away from a structure of a nucleic acid molecule (e.g., bubble, loop). For example, nucleotides of the first stem of the anti-repeat of a dual guide RNA distal to the first bubble of the stem loop is nearer to the 3′ terminal nucleotide of the crRNA and the 5′ terminal nucleotide of the tracrRNA than they are to the first bubble. A tracrRNA also forms secondary structure upon hybridizing with its corresponding crRNA. The anti-repeat region of a tracrRNA is fully or partially complementary to the crRNA repeat of a crRNA. In some embodiments, a portion of the anti-repeat of a tracrRNA and a portion of a crRNA repeat hybridize and form a stem. In some embodiments, the crRNA:tracrRNA stem includes at least one nucleotide pair (i.e. base pair) because these portions of the anti-repeat and crRNA repeat are complementary. As described elsewhere herein, a portion of the anti-repeat of a tracrRNA forming a first stem is the first stem of the anti-repeat, a portion of the anti-repeat of a tracrRNA forming a second stem is the second stem of the anti-repeat, a portion of the anti-repeat of a tracrRNA forming a third stem is the third stem of the anti-repeat, etc. As described elsewhere herein, a portion of the crRNA repeat of a crRNA forming a first stem is the first stem of the crRNA repeat, a portion of the crRNA repeat of a crRNA forming a second stem is the second stem of the crRNA repeat, a portion of the crRNA repeat of a crRNA forming a third stem is the third stem of the crRNA repeat, etc. In some embodiments, a portion of the anti-repeat of a tracrRNA and a portion of the crRNA repeat are not complementary with each other and thus do not hybridize to form base pairs. In some embodiments, the region of non-complementarity between the anti-repeat and the crRNA repeat forms a bulge or a bubble. In some embodiments, hybridization of the anti-repeat of a tracrRNA and the crRNA repeat of a crRNA forms a secondary structure that includes at least one stem. In some embodiments, hybridization of the anti-repeat of a tracrRNA and the crRNA repeat of a crRNA forms a secondary structure that includes at least one bubble. In some embodiments, hybridization of the anti-repeat of a tracrRNA and the crRNA repeat of a crRNA forms a secondary structure that includes at least one stem and at least one bubble. In some embodiments, hybridization of the anti-repeat of a tracrRNA and the crRNA repeat of a crRNA forms a secondary structure that includes two stems and one bubble in between.

In some embodiments, the anti-repeat of the tracrRNA that is fully or partially complementary to the CRISPR repeat comprises from about 8 nucleotides to about 30 nucleotides, or more. For example, the region of base pairing between the tracrRNA anti-repeat and the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the region of base pairing between the tracrRNA anti-repeat and the CRISPR repeat is 6, 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA anti-repeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat and its corresponding tracrRNA anti-repeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the entire tracrRNA can comprise from about 60 nucleotides to more than about 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210, or more nucleotides in length. In some embodiments, the tracrRNA is 60, 65, 70, 75, 80, 85, 90, 95, 100, 105, 110, 115, 120, 125, 130, 135, 140, 150, 160, 170, 180, 190, 200, 210 or more nucleotides in length. In some embodiments, the tracrRNA is about 70 to about 105 nucleotides in length, including about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, and about 105 nucleotides in length. In embodiments, the tracrRNA is 70 to 105 nucleotides in length, including 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, and 105 nucleotides in length.

In some embodiments, the tracrRNA comprises the nucleotide sequence set forth as any one of SEQ ID NOs: 547, 553-562, 840, 842, and 846, or an active variant or fragment thereof that when comprised within a guide RNA is capable of directing the sequence-specific binding of an associated RNA-guided nuclease provided herein to a presently disclosed target sequence within the FOXP3 gene. In some embodiments, an active tracrRNA sequence variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as any one of SEQ ID NOs: 547, 553-562, 840, 842, and 846. In some embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as any one of SEQ ID NOs: 547, 553-562, 840, 842, and 846. An active tracrRNA sequence fragment differs in length from SEQ ID NO: 547 by 1 to 16 nucleotides. In some embodiments, an active tracrRNA has a nucleotide sequence that is 8 nucleotides shorter than the nucleotide sequence set forth as SEQ ID NO: 547. In some embodiments, an active tracrRNA has a nucleotide sequence that is 11 nucleotides shorter than the nucleotide sequence set forth as SEQ ID NO: 547. An active tracrRNA sequence fragment can comprise the nucleotide sequence set forth as any one of SEQ ID NOs: 547, 553-562, 840, 842, and 846. In some embodiments, an active tracrRNA has at least 80% sequence identity, at least 90% sequence identity, or at least 95% sequence identity to a nucleotide sequence set forth as SEQ ID NO: 547. In some embodiments, an active tracrRNA has the nucleotide sequence set forth as:

(SEQ ID NO: 547)
UGGCUUUGAUGUUUCUAUGAUAAGGGUUUCGACCCGUGGCGUCGGGGAU
CGCCUGCCCAUUGAAAUGGGCUUCUCCCCAUUUAUU.

A tracrRNA can comprise at least one chemical modification. In some embodiments, a tracrRNA of the disclosure can comprise 2′-O-methyl 3′phosphorothioate (MS) modifications at the 3 terminal nucleotides at the 5′ end and at the 3 terminal nucleotides at the 3′ end of the tracrRNA. TracrRNAs comprising 2′-O-methyl 3′phosphorothioate (MS) modifications at the 3 terminal nucleotides at the 5′ end and at the 3 terminal nucleotides at the 3′ end of the tracrRNA can have nucleotide sequences set forth as any one of SEQ ID NOs: 941, 946-955, 1229, 1231, and 1233.

Two polynucleotide sequences can be considered to be substantially complementary when the two sequences hybridize to each other under stringent conditions. The term “hybridize” refers to one molecule binding or associating with another molecule, or regions of one molecule binding or associating with each other. A spacer of a guide RNA and its target sequence are considered to be substantially complementary when the two sequences hybridize to each other sufficiently to allow for the localization to the target sequence of an RGN bound to the guide RNA. Likewise, an RGN is considered to bind to a particular target sequence in a sequence-specific manner if the guide RNA bound to the RGN binds to a target sequence under normal experimental or in vivo conditions. The term “sequence specific” can also refer to the binding of a RGN polypeptide to a target sequence at a greater affinity than binding to a randomized background sequence.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The guide RNA can be a single guide RNA (sgRNA) or a dual-guide RNA (dgRNA). A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat of the crRNA and at least a portion of the tracrRNA (i.e., the anti repeat), which may be fully or partially complementary to the CRISPR repeat of the crRNA. In embodiments wherein the guide RNA is a single guide RNA, the crRNA and tracrRNA are separated by a linker nucleotide sequence. In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In some embodiments, the linker nucleotide sequence of a single guide RNA is at least 4 nucleotides in length. In certain embodiments, the linker nucleotide sequence of a single guide RNA is 4 nucleotides in length. In some embodiments, the linker nucleotide sequence includes a nucleotide sequence set forth as any of AAAG, GAAA, ACUU, and CAAAGG. In certain embodiments, the linker nucleotide sequence includes a nucleotide sequence set forth as AAAG. In some embodiments, the linker nucleotide sequence includes a nucleotide sequence set forth as GAAA.

In some embodiments, the linker nucleotide sequence includes a nucleotide sequence set forth as ACUU. In some embodiments, the linker nucleotide sequence includes a nucleotide sequence set forth as CAAAGG.

The single guide RNA or dual-guide RNA can be synthesized chemically or via in vitro transcription. Assays for determining sequence-specific binding between an RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA:RGN complex is captured via the detectable label (e.g., with streptavidin beads). A control guide RNA with an unrelated sequence or structure to the guide RNA can be used as a negative control for non-specific binding of the RGN to RNA. In some embodiments, the guide RNA includes any one of SEQ ID NOs: 693-834.

In some embodiments, the guide RNA has at least 80% sequence identity, at least 90% sequence identity, or at least 95% sequence identity, to a nucleotide sequence set forth as SEQ ID NO: 693. In some embodiments, the guide RNA has the nucleotide sequence set forth as:

(SEQ ID NO: 693)
UGCCAGGCCUGGGGUUGGGCAUCGUCAUAGUUCCAUUAAAAAGUUGAUG
UUUCUAUGAUAAGGGUUUCGACCCGUGGCGUCGGGGAUCGCCUGCCCUU
GAAAGGGCUUCUCCCCAUU.

In some embodiments, the guide RNA has at least 80% sequence identity, at least 90% sequence identity, or at least 95% sequence identity, to a nucleotide sequence set forth as SEQ ID NO: 694. In some embodiments, the guide RNA has the nucleotide sequence set forth as:

(SEQ ID NO: 694)
CAGGUCUGAGGCUUUGGGUGCAGGUCAUAGUUCCAUUAAAAAGUUGAUG
UUUCUAUGAUAAGGGUUUCGACCCGUGGCGUCGGGGAUCGCCUGCCCUU
GAAAGGGCUUCUCCCCAUU.

In some embodiments, the guide RNA has at least 80% sequence identity, at least 90% sequence identity, or at least 95% sequence identity, to a nucleotide sequence set forth as SEQ ID NO: 695. In some embodiments, the guide RNA has the nucleotide sequence set forth as:

(SEQ ID NO: 695)
UCGAAGAUCUCGGCCCUGGAAGGGUCAUAGUUCCAUUAAAAAGUUGAUG
UUUCUAUGAUAAGGGUUUCGACCCGUGGCGUCGGGGAUCGCCUGCCCUU
GAAAGGGCUUCUCCCCAUU.

In some embodiments, the guide RNA has at least 80% sequence identity, at least 90% sequence identity, or at least 95% sequence identity, to a nucleotide sequence set forth as SEQ ID NO: 696. In some embodiments, the guide RNA has the nucleotide sequence set forth as:

(SEQ ID NO: 696)
UCUCGGCCCUGGAAGGUUCCCCCUGGUCAUAGUUCCAUAAAGAUGUUUC
UAUGAUAAGGGUUUCGACCCGUGGCGUCGGGGAUCGCCUGCCCAUUGAA
AUGGGCUUCUCCCCAUUUAUU.

In some embodiments, the guide RNA has at least 80% sequence identity, at least 90% sequence identity, or at least 95% sequence identity, to a nucleotide sequence set forth as SEQ ID NO: 697. In some embodiments, the guide RNA has the nucleotide sequence set forth as:

(SEQ ID NO: 697)
GGUUCAAGGAAGAAGAGGAGGCAGUCAUAGUUCCAUUAAAAAGUUGAUG
UUUCUAUGAUAAGGGUUUCGACCCGUGGCGUCGGGGAUCGCCUGCCCUU
GAAAGGGCUUCUCCCCAUU.

In some embodiments, the guide RNA has at least 80% sequence identity, at least 90% sequence identity, or at least 95% sequence identity, to a nucleotide sequence set forth as SEQ ID NO: 698. In some embodiments, the guide RNA has the nucleotide sequence set forth as:

(SEQ ID NO: 698)
GGGGUUCAAGGAAGAAGAGGAGGCAGUCAUAGUUCCAUAAAGAUGUUUC
UAUGAUAAGGGUUUCGACCCGUGGCGUCGGGGAUCGCCUGCCCAUUGAA
AUGGGCUUCUCCCCAUUUAUU.

A guide RNA of the disclosure can comprise at least one chemical modification. In a single guide RNA format, the at least one chemical modification can comprise 2′-O-methyl 3′phosphorothioate (MS) modifications at the 3 terminal nucleotides at the 5′ region and at the 3 terminal nucleotides at the 3′ region of the single guide RNA. In a dual guide RNA format, the at least one chemical modification can comprise 2′-O-methyl 3′phosphorothioate (MS) modifications at the 3 terminal nucleotides at the 5′ region and at the 3 terminal nucleotides at the 3′ region of the crRNA, and can comprise 2′-O-methyl 3′phosphorothioate (MS) modifications at the 3 terminal nucleotides at the 5′ region and/or at the 3 terminal nucleotides at the 3′ region of the tracrRNA. MS modified guide RNAs can have nucleotide sequences set forth as any one of SEQ ID NOs: 1086-1227.

The guide RNA can be introduced into a target cell or embryo as an RNA molecule. The guide RNA can be transcribed in vitro or chemically synthesized. In some embodiments, a nucleotide sequence encoding the guide RNA is introduced into the cell or embryo. In some embodiments, the nucleotide sequence encoding the guide RNA is operably linked to a promoter (e.g., an RNA polymerase III promoter). The promoter can be a native promoter or heterologous to the guide RNA-encoding nucleotide sequence.

In some embodiments, the guide RNA can be introduced into a target cell or embryo as a ribonucleoprotein complex, as described herein, wherein the guide RNA is bound to an RGN polypeptide.

The guide RNA directs an associated RGN to a particular target nucleotide sequence of interest through hybridization of the guide RNA to the target sequence of interest. The target sequence can be bound (and in some embodiments, cleaved) by an RNA-guided nuclease in vitro or in a cell. A target sequence can comprise DNA, RNA, or a combination of both and can be single-stranded or double-stranded. A target sequence can be genomic DNA (i.e., chromosomal DNA), plasmid DNA, or an RNA molecule (e.g., messenger RNA, ribosomal RNA, transfer RNA, micro RNA, small interfering RNA). In those embodiments wherein the target sequence is a chromosomal sequence, the chromosomal sequence can be a nuclear or mitochondrial chromosomal sequence. In the presently disclosed compositions and methods, the target sequence is within a target nucleic acid molecule that is double-stranded (e.g., a target DNA sequence). More specifically, the target sequence is within the FOXP3 gene. In some embodiments, the target sequence is unique in the target genome. In some embodiments, the target sequence comprises the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

The target sequence is adjacent to a protospacer adjacent motif (PAM) and the non-target strand of the target sequence is the strand that comprises the PAM. The PAM is immediately adjacent to the target sequence and often comprises Ns, where each “N” represents any nucleotide. In some embodiments, the PAM comprises about 1 to about 10 Ns, including about 1, about 2, about 3, about 4, about 5, about 6, about 7, about 8, about 9, or about 10 Ns. In certain embodiments, a PAM comprises 1 to 10 Ns, including 1, 2, 3, 4, 5, 6, 7, 8, 9, or 10 Ns. The PAM can be 5′ or 3′ of the target sequence on its non-target strand. In some embodiments, the PAM is 3′ of the target sequence on its non-target strand for the presently disclosed guide RNAs and RGN systems. Generally, the PAM is a consensus sequence of about 3-4 nucleotides, but in certain embodiments it can be 2, 3, 4, 5, 6, 7, 8, 9, or more nucleotides in length.

In some embodiments, a PAM sequence adjacent to a presently disclosed target sequence on its non-target strand comprises the consensus sequence set forth as any one of the PAM sequences in Table 1. In some embodiments, a PAM sequence adjacent to the presently disclosed target sequence on its non-target strand includes the sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments, the PAM sequence is 3′ of the target sequence on its non-target strand.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell or embryo.

Upon recognizing its corresponding PAM sequence, the RGN can cleave one or both strands of a target sequence at a specific cleavage site. As used herein, a cleavage site is made up of the two particular nucleotides within a target sequence between which the target strand, non-target strand, or both strands of a target sequence are cleaved by an RGN. The cleavage site can comprise the 1^{st} and 2^{nd}, 2^{nd} and 3^{rd}, 3^{rd} and 4^{th}, 4^{th} and 5^{th}, 5^{th} and 6^{th}, 7^{th} and 8^{th}, 8^{th} and 9^{th} nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. As RGNs can cleave a target sequence resulting in staggered ends, in certain embodiments, the cleavage site is defined based on the distance of the two nucleotides from the PAM on the non-target strand of the target sequence and, for the target strand, the distance of the two nucleotides from the complement of the PAM.

### III. Length Modifications to Guide RNA

The guide RNAs disclosed herein that are effective in targeting an associated RNA-guided nuclease (RGN) to a target nucleotide sequence in the FOXP3 gene can be engineered to be shorter than their corresponding native guide RNAs but have comparable efficiencies as their corresponding native guide RNAs in gene editing. A native guide RNA includes a guide RNA that is naturally occurring, for example, a guide RNA from an organism. A guide RNA that is engineered to be shorter than its native guide RNA length can be as effective as its non-engineered counterpart in its ability to bind an associated RGN and cleave and/or modify a target sequence.

A modification (e.g., deletion, truncation) “within” a region of a RNA molecule of the disclosure includes all nucleotides and phosphate backbone in that region, including the first and last nucleotide positions that are considered part of that region.

In some embodiments, a spacer, a crRNA repeat, a crRNA, an anti-repeat, a tracrRNA, a backbone, and/or a guide RNA of the present disclosure are engineered to be truncated or shortened. In some embodiments, a truncated spacer, truncated crRNA repeat, truncated crRNA, truncated anti-repeat, truncated tracrRNA, truncated backbone, and/or truncated guide RNA maintains or enhances gene editing efficiency as compared to the same spacer, crRNA repeat, crRNA, anti-repeat, tracrRNA, backbone, and/or guide RNA prior to its engineering. “Truncation” and “deletion” in the context of engineering a spacer, crRNA repeat, crRNA, anti-repeat, tracrRNA, backbone, or guide RNA, are used interchangeably herein and refer to removal of at least one nucleotide from a reference spacer, crRNA repeat, crRNA, anti-repeat, tracrRNA, backbone, or guide RNA, which might be naturally occurring or synthetic.

An engineered spacer can comprise a truncation of 1 nucleotide (nt), 2 nt, 3 nt, 4 nt, or 5 nt, as compared to the same spacer prior to its engineering. An engineered spacer can comprise a truncation of 1 nt, as compared to the spacer prior to its engineering. An engineered spacer can comprise a truncation of 2 nt, as compared to the spacer prior to its engineering. An engineered spacer can comprise a truncation of 3 nt, as compared to the spacer prior to its engineering. An engineered spacer can comprise a truncation of 4 nt, as compared to the spacer prior to its engineering. An engineered spacer can comprise a truncation of 5 nt, as compared to the spacer prior to its engineering. In some embodiments, a spacer of the disclosure has a nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213. In some embodiments, a spacer as part of a guide RNA comprises 2′-O-methyl 3′phosphorothioate (MS) modifications at the 3 terminal nucleotides at the 5′ region of the spacer.

An engineered crRNA repeat can comprise a truncation of 1 nt, 2 nt, 3 nt, 4 nt, 5 nt, 6 nt, 7 nt, 8 nt, 9 nt, or 10 nt, as compared to the crRNA repeat prior to its engineering. An engineered crRNA repeat can comprise a truncation of 1 nt, 2 nt, 3 nt, 4 nt, 5 nt, 6 nt, 7 nt, 8 nt, 9 nt, or 10 nt from its 3′ terminus as compared to the nucleotide sequence set forth as SEQ ID NO: 546. In some embodiments, an engineered crRNA repeat comprises a truncation of 1 nt from its 3′ terminus as compared to the nucleotide sequence set forth as SEQ ID NO: 546. In some embodiments, an engineered crRNA repeat comprises a truncation of 2 nt from its 3′ terminus as compared to the nucleotide sequence set forth as SEQ ID NO: 546. In some embodiments, an engineered crRNA repeat comprises a truncation of 3 nt from its 3′ terminus as compared to the nucleotide sequence set forth as SEQ ID NO: 546. In some embodiments, an engineered crRNA repeat comprises a truncation of 4 nt from its 3′ terminus as compared to the nucleotide sequence set forth as SEQ ID NO: 546. In some embodiments, an engineered crRNA repeat comprises a truncation of 5 nt from its 3′ terminus as compared to the nucleotide sequence set forth as SEQ ID NO: 546. In some embodiments, an engineered crRNA repeat comprises a truncation of 6 nt from its 3′ terminus as compared to the nucleotide sequence set forth as SEQ ID NO: 546. In some embodiments, an engineered crRNA repeat comprises a truncation of 7 nt from its 3′ terminus as compared to the nucleotide sequence set forth as SEQ ID NO: 546. In some embodiments, an engineered crRNA repeat comprises a truncation of 8 nt from its 3′ terminus as compared to the nucleotide sequence set forth as SEQ ID NO: 546. In some embodiments, an engineered crRNA repeat comprises a truncation of 9 nt from its 3′ terminus as compared to the nucleotide sequence set forth as SEQ ID NO: 546. In some embodiments, an engineered crRNA repeat comprises a truncation of 10 nt from its 3′ terminus as compared to the nucleotide sequence set forth as SEQ ID NO: 546.

In some embodiments, an engineered crRNA repeat has the nucleotide sequence set forth as SEQ ID NO: 546 or that differs in length and/or sequence from SEQ ID NO: 546 by 1 to 8 nucleotides. In some embodiments, an engineered crRNA repeat has a nucleotide sequence that differs in length and/or sequence from SEQ ID NO: 546 by 8 nucleotides. In some embodiments, an engineered crRNA repeat has a nucleotide sequence that differs in length and/or sequence from SEQ ID NO: 546 by 7 nucleotides. In some embodiments, an engineered crRNA repeat has a nucleotide sequence that differs in length and/or sequence from SEQ ID NO: 546 by 6 nucleotides. In some embodiments, an engineered crRNA repeat has a nucleotide sequence that differs in length and/or sequence from SEQ ID NO: 546 by 5 nucleotides. In some embodiments, an engineered crRNA repeat has a nucleotide sequence that differs in length and/or sequence from SEQ ID NO: 546 by 4 nucleotides. In some embodiments, an engineered crRNA repeat has a nucleotide sequence that differs in length and/or sequence from SEQ ID NO: 546 by 3 nucleotides. In some embodiments, an engineered crRNA repeat has a nucleotide sequence that differs in length and/or sequence from SEQ ID NO: 546 by 2 nucleotides. In some embodiments, an engineered crRNA repeat has a nucleotide sequence that differs in length and/or sequence from SEQ ID NO: 546 by 1 nucleotide.

A crRNA repeat can comprise a total length of at least 10, 11, 12, 13, 14, 15, or 16 nucleotides. A crRNA repeat can comprise a total length of at most 10, 11, 12, 13, 14, 15, or 16 nucleotides. In some embodiments, a crRNA repeat can comprise a total length of 13 nucleotides. In some embodiments, a crRNA repeat can comprise a total length of 16 nucleotides. In some embodiments, a crRNA repeat has the nucleotide sequence set forth as any one of SEQ ID NOs: 546, 549-552, 839, 842, and 845. In some embodiments, a crRNA repeat as part of a guide RNA comprises 2′-O-methyl 3′phosphorothioate (MS) modifications at the 3 terminal nucleotides at the 3′ region of the crRNA repeat. MS modified crRNA repeats can have nucleotide sequences set forth as any of SEQ ID NOs: 940, 942-945, 1228, 1230, and 1232.

An engineered crRNA can comprise a truncation of 1 nt, 2 nt, 3 nt, 4 nt, 5 nt, 6 nt, 7 nt, 8 nt, 9 nt, 10 nt, 11 nt, 12 nt, 13 nt, 14 nt, or 15 nt as compared to the crRNA prior to its engineering. An engineered crRNA can comprise a truncation of 1 nt, 2 nt, 3 nt, 4 nt, or 5 nt from its 5′ terminus. In some embodiments, an engineered crRNA comprises a truncation of 1 nt from its 5′ terminus. In some embodiments, an engineered crRNA comprises a truncation of 2 nt from its 5′ terminus. In some embodiments, an engineered crRNA comprises a truncation of 3 nt from its 5′ terminus. An engineered crRNA can comprise a truncation of 1 nt, 2 nt, 3 nt, 4 nt, 5 nt, 6 nt, 7 nt, 8 nt, 9 nt, 10 nt, 11 nt, or 12 nt from its 3′ terminus. In some embodiments, an engineered crRNA comprises a truncation of 5 nt from its 3′ terminus. In some embodiments, an engineered crRNA comprises a truncation of 8 nt from its 3′ terminus.

A crRNA can have a nucleotide sequence having at least 80% sequence identity to any one of SEQ ID NOs: 574-692. In some embodiments, a crRNA has a nucleotide sequence having at least 90% sequence identity to any one of SEQ ID NOs: 574-692. In some embodiments, a crRNA has a nucleotide sequence having at least 95% sequence identity to any one of SEQ ID NOs: 574-692. In some embodiments, a crRNA has a nucleotide sequence having 100% sequence identity to any one of SEQ ID NOs: 574-692. A crRNA of the disclosure can comprise 2′-O-methyl 3′phosphorothioate (MS) modifications at the 3 terminal nucleotides at the 5′ region and at the 3 terminal nucleotides at the 3′ region of the crRNA. MS modified crRNAs can have nucleotide sequences set forth as any of SEQ ID NOs: 967-1085.

An engineered tracrRNA can comprises a truncation of 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, or more, as compared to the same tracrRNA prior to its engineering. In some embodiments, an engineered tracrRNA comprises a deletion of 1 to 12 nucleotides within the first stem of the anti-repeat, as compared to the tracrRNA prior to its engineering. In some embodiments, an engineered tracrRNA comprises a deletion of 1 nt, 2 nt, 3 nt, 4 nt, 5 nt, 6 nt, 7 nt, 8 nt, 9 nt, 10 nt, 11 nt, or 12 nt within the first stem of the anti-repeat, as compared to the tracrRNA prior to its engineering. In some embodiments, an engineered tracrRNA comprises a deletion of 1 nt, 2 nt, 3 nt, 4 nt, 5 nt, 6 nt, 7 nt, 8 nt, or 9 nt within the first stem of the anti-repeat, as compared to the tracrRNA prior to its engineering.

An engineered tracrRNA can comprise a deletion of nucleotides from the tail, as compared to the tracrRNA prior to its engineering. In some embodiments, an engineered tracrRNA comprises a deletion of 1 to 6 nucleotides from the tail, as compared to the tracrRNA prior to its engineering. In some embodiments, an engineered tracrRNA comprises a deletion of 1 nucleotide, 2 nucleotides, 3 nucleotides, 4 nucleotides, 5 nucleotides, or 6 nucleotides from the tail, as compared to the tracrRNA prior to its engineering.

An engineered tracrRNA can comprise a deletion in a stem loop most proximal to the tail, as compared to the tracrRNA prior to its engineering. In some embodiments, an engineered tracrRNA comprises a deletion of 1 to 4 base pairs (bp), or 2 to 8 nt, within the first stem of the stem-loop most proximal to the tail of the tracrRNA, as compared to the tracrRNA prior to its engineering. In some embodiments, an engineered tracrRNA comprises a deletion of 1 to 3 bp, or 2 to 6 nt, within the first stem of the stem-loop most proximal to the tail of the tracrRNA, as compared to the tracrRNA prior to its engineering. In some embodiments, an engineered tracrRNA comprises a deletion of 1 bp (2 nt), 2 bp (4 nt), or 3 bp (6 nt) within the first stem of the stem-loop most proximal to the tail of the tracrRNA, as compared to the tracrRNA prior to its engineering.

As disclosed herein, a tracrRNA can comprise a total length of at least 65, 70, 75, 80, or 85 nucleotides. A tracrRNA can comprise comprises a total length of at most 65, 70, 75, 80, or 85 nucleotides. In some embodiments, a tracrRNA comprises a total length of 74 nucleotides. In some embodiments, a tracrRNA comprises a total length of 77 nucleotides.

A tail of a tracrRNA can comprise a total length of at least 1, 2, 3, 4, 5, 6, or 7 nucleotides. A tail of a tracrRNA can comprise a total length of at most 1, 2, 3, 4, 5, 6, or 7 nucleotides. In some embodiments, a tail of a tracrRNA comprises a total length of 3 nucleotides. In some embodiments, a tail of a tracrRNA comprises a total length of 1 nucleotide.

A tracrRNA can comprise a nucleotide sequence having at least 80% sequence identity to any one of SEQ ID NOs: 547, 553-562, 840, 842, and 846. In some embodiments, a tracrRNA has a nucleotide sequence having at least 90% sequence identity to any one of SEQ ID NOs: 547, 553-562, 840, 842, and 846. In some embodiments, a tracrRNA has a nucleotide sequence having at least 95% sequence identity to any one of SEQ ID NOs: 547, 553-562, 840, 842, and 846. In some embodiments, a tracrRNA has a nucleotide sequence having 100% sequence identity to any one of SEQ ID NOs: 547, 553-562, 840, 842, and 846. In some embodiments, a tracrRNA as part of a guide RNA comprises 2′-O-methyl 3′phosphorothioate (MS) modifications at the 3 terminal nucleotides at the 5′ region of the tracrRNA and at the 3 terminal nucleotides at the 3′ region of the tracrRNA. MS modified tracrRNAs can have nucleotide sequences set forth as any of SEQ ID NOs: 941, 946-955, 1229, 1231, and 1233.

A gRNA of the disclosure includes a sgRNA that comprises a backbone, wherein the backbone of the sgRNA comprises a crRNA repeat and a tracrRNA linked by a nucleotide linker. In some embodiments, the linker has a nucleotide sequence set forth as AAAG, GAAA, ACUU, or CAAAGG. In some embodiments, the linker has the nucleotide sequence set forth as AAAG.

Engineered sgRNA backbones disclosed herein can be 2 to 30 nucleotides shorter, as compared to the backbone prior to its engineering. An engineered sgRNA backbone can be 12 to 24 nucleotides shorter, as compared to the backbone prior to its engineering. In some embodiments, an engineered sgRNA backbone is 2 nucleotides, 4 nucleotides, 6 nucleotides, 8 nucleotides, 10 nucleotides, 12 nucleotides, 14 nucleotides, 16 nucleotides, 18 nucleotides, 20 nucleotides, 22 nucleotides, 24 nucleotides, 26 nucleotides, 28 nucleotides, 30 nucleotides, or more shorter, as compared to the backbone prior to its engineering.

An sgRNA backbone of the disclosure can comprise a total length of at least 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides. An sgRNA backbone of the disclosure can comprise a total length of at most 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides. In some embodiments, the sgRNA backbone comprises a total length of 86 to 98 nucleotides. In some embodiments, the sgRNA backbone comprises a total length of 94 nucleotides.

In some embodiments, a sgRNA has the nucleotide sequence set forth as any one of SEQ ID NOs: 693-834.

An sgRNA backbone of the disclosure can have a nucleotide sequence having at least 80% sequence identity to any one of SEQ ID NOs: 563-573. In some embodiments, an sgRNA backbone has a nucleotide sequence having at least 90% sequence identity to any one of SEQ ID NOs: 563-573. In some embodiments, an sgRNA backbone has a nucleotide sequence having at least 95% sequence identity to any one of SEQ ID NOs: 563-573. In some embodiments, an sgRNA backbone has a nucleotide sequence having 100% sequence identity to any one of SEQ ID NOs: 563-573. In some embodiments, a backbone as part of a guide RNA comprises 2′-O-methyl 3′phosphorothioate (MS) modifications at the 3 terminal nucleotides at the 3′ region of the backbone. MS modified backbones can have nucleotide sequences set forth as any of SEQ ID NOs: 956-966.

A gRNA of the disclosure includes a sgRNA that comprises a spacer and a backbone, wherein the backbone of the sgRNA comprises a crRNA repeat and a tracrRNA linked by a nucleotide linker. In some embodiments, an engineered sgRNA comprises a truncation in the spacer and/or a truncation in the backbone, as compared to the sgRNA prior to its engineering. In some embodiments, an engineered sgRNA comprises a truncation in the spacer, as compared to the sgRNA prior to its engineering. In some embodiments, an engineered sgRNA comprises a truncation in the backbone, as compared to the sgRNA prior to its engineering. In some embodiments, an engineered sgRNA comprises a truncation in the spacer and a truncation in the backbone, as compared to the sgRNA prior to its engineering. In embodiments where an engineered sgRNA comprises a truncation in the backbone, the truncation can be within the first stem of the stem loop formed by hybridization of the crRNA repeat and the anti-repeat, within the first stem of the stem loop most proximal to the tail, and/or within the tail of the tracrRNA.

An engineered sgRNA can comprise a deletion of 1 to 30 total nucleotides, as compared to the sgRNA prior to its engineering. In some embodiments, an engineered sgRNA comprises a deletion of 13 to 25 total nucleotides, as compared to the sgRNA prior to its engineering. In some embodiments, an engineered sgRNA comprises a deletion of 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 total nucleotides, or more, as compared to the sgRNA prior to its engineering.

The first stem of a stem loop formed by hybridization of the crRNA repeat and the anti-repeat of a gRNA can comprise a total length of at least 3, 4, 5, 6, 7, 8, 9, 10, or 11 base pairs (bp), or at least 6, 8, 10, 12, 14, 16, 18, 20, or 22 nt. The first stem of a stem loop formed by hybridization of the crRNA repeat and the anti-repeat of a gRNA can comprise a total length of at most 3, 4, 5, 6, 7, 8, 9, 10, or 11 bp, or at most 6, 8, 10, 12, 14, 16, 18, 20, or 22 nt. In some embodiments, the first stem of a stem loop formed by hybridization of the crRNA repeat and the anti-repeat of a gRNA comprises a total length of 6 bp, or 12 nt. In some embodiments, the first stem of a stem loop formed by hybridization of the crRNA repeat and the anti-repeat of a gRNA comprises a total length of 3 bp, or 6 nt.

The first stem of the stem loop most proximal to the tail in a gRNA can comprise a total length of at least 1, 2, 3, 4, 5, or 6 bp, or at least 2, 4, 6, 8, 10, or 12 nt. The first stem of the stem loop most proximal to the tail in a gRNA can comprise a total length of at most 1, 2, 3, 4, 5, or 6 bp, or at most 2, 4, 6, 8, 10, or 12 nt. In some embodiments, the first stem of the stem loop most proximal to the tail in a gRNA comprises a total length of 5 bp, or 10 nt.

In some embodiments, a gRNA of the disclosure comprises the following: the first stem of the stem loop formed by hybridization of the crRNA repeat and the anti-repeat comprises a total length of 6 bp (12 nt), the tail of the tracrRNA comprises a total length of 3 nucleotides, and the first stem of the stem loop most proximal to the tail comprises a total length of 3 bp (6 nt). In some embodiments, a gRNA of the disclosure comprises a first stem of a stem loop formed by hybridization of the crRNA repeat and the anti-repeat comprising a total length of 13 bp (26 nt).

A total length of a guide RNA can refer to a total length of a sgRNA or of a dgRNA. A gRNA of the disclosure can comprise a total length of at least 85, 90, 95, 100, 105, 110, 115, 120, 125, 130, or 135 nucleotides. A gRNA of the disclosure can comprise a total length of at most 85, 90, 95, 100, 105, 110, 115, 120, 125, 130, or 135 nucleotides. In some embodiments, a gRNA of the disclosure comprises a total length of 106 to 135 nucleotides. In some embodiments, a gRNA of the disclosure comprises a total length of 117 to 119 nucleotides. In embodiments where the gRNA comprises a total length of 117 to 119 nucleotides, the gRNA is a sgRNA. In embodiments where a gRNA comprises a total length of 117 to 119 nucleotides as a sgRNA, the total length of the gRNA as a dgRNA can be 4 to 6 nucleotides fewer, or 111 to 115 nucleotides. In some embodiments, the total length of a gRNA as a dgRNA is 4 to 6 nucleotides fewer, or a number of nucleotides fewer that is equivalent to the length of the linker joining the crRNA and tracrRNA, as compared to the total length of the gRNA as a sgRNA. In some embodiments, a gRNA of the disclosure comprises a total length of 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135 nucleotides, or more. In some embodiments, a sgRNA has the nucleotide sequence set forth as any one of SEQ ID NOs: 693-834.

In some embodiments, a sgRNA has at least 80% sequence identity, at least 90% sequence identity, or at least 95% sequence identity, to a nucleotide sequence set forth as SEQ ID NO: 693. In some embodiments, a sgRNA has the nucleotide sequence set forth as SEQ ID NO: 693. In some embodiments, a sgRNA has at least 80% sequence identity, at least 90% sequence identity, or at least 95% sequence identity, to a nucleotide sequence set forth as SEQ ID NO: 694. In some embodiments, a sgRNA has the nucleotide sequence set forth as SEQ ID NO: 694. In some embodiments, a sgRNA has at least 80% sequence identity, at least 90% sequence identity, or at least 95% sequence identity, to a nucleotide sequence set forth as SEQ ID NO: 695. In some embodiments, a sgRNA has the nucleotide sequence set forth as SEQ ID NO: 695. In some embodiments, a sgRNA has at least 80% sequence identity, at least 90% sequence identity, or at least 95% sequence identity, to a nucleotide sequence set forth as SEQ ID NO: 696. In some embodiments, a sgRNA has the nucleotide sequence set forth as SEQ ID NO: 696. In some embodiments, a sgRNA has at least 80% sequence identity, at least 90% sequence identity, or at least 95% sequence identity, to a nucleotide sequence set forth as SEQ ID NO: 697. In some embodiments, a sgRNA has the nucleotide sequence set forth as SEQ ID NO: 697. In some embodiments, a sgRNA has at least 80% sequence identity, at least 90% sequence identity, or at least 95% sequence identity, to a nucleotide sequence set forth as SEQ ID NO: 698. In some embodiments, a sgRNA has the nucleotide sequence set forth as SEQ ID NO: 698.

A sgRNA of the disclosure can comprise 2′-O-methyl 3′phosphorothioate (MS) modifications at the 3 terminal nucleotides at the 5′ region and at the 3 terminal nucleotides at the 3′ region of the sgRNA. MS modified sgRNAs can have nucleotide sequences set forth as any one of SEQ ID NOs: 1086-1227.

### IV. RNA-Guided Nucleases and Other Nucleases

Provided herein are RNA-guided nuclease systems comprising the presently disclosed guide RNAs targeting the FOXP3 gene. The term RNA-guided nuclease (RGN) refers to a polypeptide that binds to a particular target sequence (e.g., target DNA sequence) in a sequence-specific manner and is directed to the target sequence by a guide RNA molecule that is complexed with the polypeptide and hybridizes with the target strand of the target sequence (e.g., target DNA sequence). Active fragments or variants thereof of naturally-occurring RGNs maintain binding to a target nucleotide sequence in an RNA-guided sequence-specific manner. Although an RGN can be capable of cleaving the target sequence upon binding, the term RGN also encompasses nuclease-dead RGNs that are capable of binding to, but not cleaving, a target sequence. Cleavage of a target strand and/or non-target strand of a target sequence by an RGN can result in a single- or double-stranded break. RGNs only capable of cleaving a single strand of a double-stranded target nucleic acid molecule are referred to herein as nickases.

The presently disclosed RGN systems comprise an RGN that binds to a FOXP3 target sequence disclosed herein. In some embodiments, the RGN recognizes a PAM having a consensus nucleotide sequence including NNNNCC 3′ of the target sequence on its non-target strand (where N is A, C, T/U, or G; R is G or A), and active fragments or variants thereof. In some embodiments, the active fragment or variant of an RGN recognizing such PAM sequences is capable of binding and in some embodiments, cleaving or nicking a target sequence.

In some embodiments, an RGN, or an active variant or fragment thereof, capable of binding a target sequence adjacent to a PAM consensus sequence (i.e., capable of recognizing the PAM consensus sequence) set forth as NNNNCC is used in the presently disclosed compositions and methods. In some embodiments, an RGN, or an active variant or fragment thereof, capable of binding a target sequence adjacent to a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC is used in the presently disclosed compositions and methods. In some embodiments, the PAM sequence is 3′ of the target sequence on its non-target strand. In some embodiments, the RGN binds to a guide RNA having a sequence set forth as any one of SEQ ID NOs: 693-834. In some embodiments, the RGN binds to a guide RNA having at least 80% sequence identity, at least 90% sequence identity, or at least 95% sequence identity to a nucleotide sequence set forth as SEQ ID NO: 693. In some embodiments, the RGN binds to a guide RNA having at least 80% sequence identity, at least 90% sequence identity, or at least 95% sequence identity to a nucleotide sequence set forth as SEQ ID NO: 694. In some embodiments, the RGN binds to a guide RNA having at least 80% sequence identity, at least 90% sequence identity, or at least 95% sequence identity to a nucleotide sequence set forth as SEQ ID NO: 695. In some embodiments, the RGN binds to a guide RNA having at least 80% sequence identity, at least 90% sequence identity, or at least 95% sequence identity to a nucleotide sequence set forth as SEQ ID NO: 696. In some embodiments, the RGN binds to a guide RNA having at least 80% sequence identity, at least 90% sequence identity, or at least 95% sequence identity to a nucleotide sequence set forth as SEQ ID NO: 697. In some embodiments, the RGN binds to a guide RNA having at least 80% sequence identity, at least 90% sequence identity, or at least 95% sequence identity to a nucleotide sequence set forth as SEQ ID NO: 698. In some embodiments, the RGN binds to a guide RNA having a sequence set forth as any one of SEQ ID NOs: 693, 694, 695, 696, 697, and 698. In some embodiments, the RGN binds to a guide RNA comprising a CRISPR repeat set forth as any one of SEQ ID NOs: 546, and 549-552, 839, 842, and 845, or an active variant or fragment thereof, and a tracrRNA set forth as any one of SEQ ID NOs: 547, 553-562, 840, 842, and 846, or an active variant or fragment thereof. In some embodiments, the RGN binds to a guide RNA comprising a CRISPR repeat having the nucleotide sequence set forth as SEQ ID NO: 546 or a nucleotide sequence that differs in length and/or sequence from SEQ ID NO: 546 by 1 to 8 nucleotides. In some embodiments, the RGN binds to a guide RNA comprising a tracrRNA having at least 80% sequence identity, at least 90% sequence identity, or at least 95% sequence identity to a nucleotide sequence set forth as SEQ ID NO: 547. In some embodiments, the RGN binds to a guide RNA comprising a tracrRNA having the nucleotide sequence set forth as SEQ ID NO: 547.

RGNs useful in the presently disclosed compositions and methods can be wild-type RGN sequences derived from bacterial or archaeal species. Alternatively, the RGNs can be variants or fragments of wild-type polypeptides. The wild-type RGN can be modified to alter nuclease activity or alter PAM specificity, for example. In some embodiments, the RGN is not naturally-occurring. RGN systems can be classified into Class 1 or Class 2. The Class 1 and 2 systems are subdivided into types (Types I, II, III, IV, V, VI), with some types further divided into subtypes (e.g., Type II-A, Type II-B, Type II-C, Type V-A, Type V-B). Class 2 systems comprise a single effector nuclease and include Types II, V, and VI.

In certain embodiments, the RGN is a naturally-occurring Type II CRISPR effector protein or an active variant or fragment thereof. As used herein, the term “Type II CRISPR-Cas protein,” “Type II CRISPR effector protein,” or “Type II RNA-guided nuclease” refers to an RGN that requires a trans-activating RNA (tracrRNA) and comprises two nuclease domains (i.e., RuvC and HNH), each of which is responsible for cleaving a single strand of a double-stranded DNA molecule. A representative type II RGN includes a Streptococcus pyogenes Cas9 protein, such as Cas9 (SpCas9 or SpyCas9) or a SpCas9 nickase, the sequences of which are set forth as SEQ ID NOs: 835 and 836, respectively, and are described in U.S. Pat. Nos. 10,000,772 and 8,697,359, each of which is herein incorporated by reference in its entirety. SpCas9 recognizes a NGG PAM sequence 3′ of a target sequence, and some of the disclosed FOXP3 target sequences could be targeted with an SpCas9 associated with its guide RNA, as indicated in Table 2 in the Examples. Another representative Cas9 ortholog that recognizes a NNNNCC PAM sequence 3′ of a target sequence includes a compact, high-accuracy Neisseria meningitidis Cas9 (Nme2Cas9), the sequence of which is set forth as SEQ ID NO: 837 and described in Edraki et al. Mol Cell. 2019 Feb. 21; 73(4):714-726.

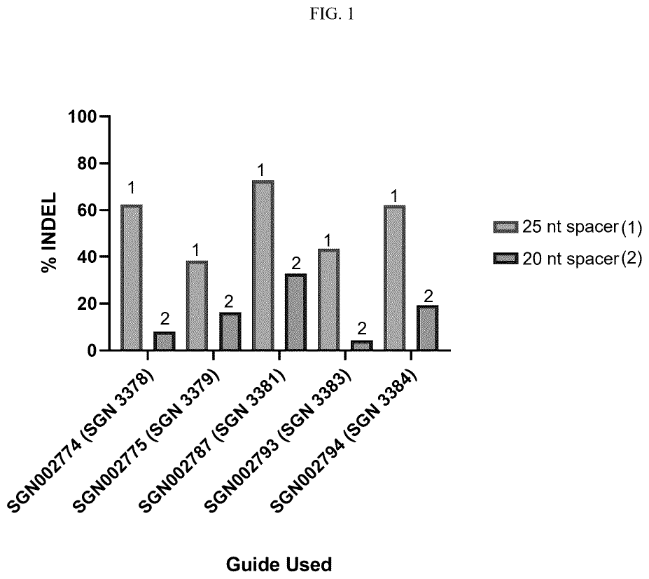

Non-limiting examples of RGN systems useful in the presently disclosed compositions and methods along with corresponding crRNA sequences and tracrRNA sequences (if needed), are presented in Table 1 below and described further in Examples 1-3, and FIGS. 1-14 of the present specification. In certain embodiments, RGN systems of the disclosure comprise an RGN, or a nickase or nuclease-dead variant thereof, listed in Table 1. The guide RNA sequences (crRNA repeat and tracrRNA sequences) that can be used with each RGN of Table 1 are also provided, as well as the consensus PAM sequence (if known). In certain embodiments, an RGN of the disclosure comprises an active variant of an RGN (one able to bind to a nucleic acid molecule in an RNA-guided manner) listed in Table 1 having between 80% and 99% or more sequence identity to any one of the amino acid sequences listed in Table 1, including but not limited to about or more than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In certain embodiments, an RGN of the disclosure comprises an RGN having 80%, 81%, 82%, 83%, 84%, 85%, 86%, 87%, 88%, 89%, 90%, 91%, 92%, 93%, 94%, 95%, 96%, 97%, 98%, 99%, or more sequence identity to an RGN amino acid sequence disclosed in Table 1. In some embodiments, an RGN of the disclosure comprises a fragment of an RGN listed in Table 1 such as one that differs by as few as 1-15 amino acid residues, as few as 1-10, such as 6-10, as few as 5, as few as 4, as few as 3, as few as 2, or as few as 1 amino acid residue. In certain embodiments, the RGN comprises an N-terminal or a C-terminal truncation, which can comprise at least a deletion of 5, 10, 15, 20, 25, 30, 35, 40, 45, 50, 55, 60 amino acids or more from either the N or C terminus of the polypeptide. In some embodiments, the RGN comprises an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more.

TABLE 1
Non-limiting examples of RNA-guided nucleases and corresponding crRNA repeat
sequences, tracrRNA sequences, and PAM sequences.

| | RGN SEQ | crRNA repeat | tracrRNA | PAM |
|---|---|---|---|---|
| RGN Name | ID NO: | SEQ ID NO: | SEQ ID NO: | sequence |
| APG07433.1 | 545 | 546 | 547 | NNNNCC |
| APG05083.1 | 838 | 839 | 840 | NNNNCC |
| APG07513.1 | 841 | 842 | 843 | NNNNCC |
| APG08290.1 | 844 | 845 | 846 | NNRNCC |
| APG05459.1 | 847 | 848 | 849 | NGG |
| APG01688.1 | 850 | 851 | 852 | NNRNNC |
| APG03128 | 853 | 854 | 855 | NNGGNNC |
| APG05733.1 | 856 | 857 | 858 | NNNNCC |
| APG01658.1 | 859 | 860 | 861 | NNGG |
| APG06498.1 | 862 | 863 | 864 | NNNNCC |
| APG06877.1 | 865 | 866 | 867 | NNNNCC |
| APG09053.1 | 868 | 869 | 870 | NGG |
| APG04293.1 | 871 | 872 | 873 | NNNNC |
| APG06646.1 | 874 | 875 | 876 | NNNNC |
| APG02874 | 877 | 878 | 879 | NNNNCC |
| APG03031 | 880 | 881 | 882 | NNNNCC |
| APG09208 | 883 | 884 | 885 | NNNNC |
| APG09344 | 886 | 887 | 888 | NAG |
| APG07991 | 889 | 890 | 891 | NGG |
| APG01868 | 892 | 893 | 894 | NGG |
| APG02998 | 895 | 896 | 897 | NGG |
| APG07433.1 deletion | 898 | 546 | 547 | NNNNCC |
| variant | | | | |
| APG08290.1 deletion | 899 | 845 | 846 | NNRNCC |
| variant | | | | |
| LPG10134 | 900 | 901 | 902 | NGG |
| LPG10136 | 903 | 904 | 905 | NNNNCC |
| LPG10138 | 906 | 907 | 908 | NNNNC |
| LPG10139 | 909 | 910 | 911 | NNNNC |
| LPG10141 | 912 | 913 | 914 | NGGNR |
| LPG10145 | 915 | 916 | 917 | NNGG |
| SpyCas9 | 835 | 918 | 919 | NGG |

N = A, C, T/U, or G; R = G or A

Non-limiting examples of RGNs useful in the presently disclosed methods and compositions include APG07433.1 RNA-guided nuclease, the amino acid sequence of which is set forth as: MRELDYRIGLDIGTNSIGWGVIELSWNKDRERYEKVRIVDQGVRMFDRAEMPKTGASLAEPR RIARSSRRRLNRKSQRKKNIRNLLVQHGVITQEELDSLYPLSKKSMDIWGIRLDGLDRLLNHF EWARLLIHLAQRRGFKSNRKSELKDTETGKVLSSIQLNEKRLSLYRTVGEMWMKDPDFSKY DRKRNSPNEYVFSVSRAELEKEIVTLFAAQRRFQSPYASKDLQETYLQIWTHQLPFASGNAIL NKVGYCSLLKGKERRIPKATYTFQYFSALDQVNRTRLGPDFQPFTKEQREIILNNMFQRTDYY KKKTIPEVTYYDIRKWLELDETIQFKGLNYDPNEELKKIEKKPFINLKAFYEINKVVANYSERT NETFSTLDYDGIGYALTVYKTDKDIRSYLKSSHNLPKRCYDDQLIEELLSLSYTKFGHLSLKAI NHVLSIMQKGNTYKEAVDQLGYDTSGLKKEKRSKFLPPISDEITNPIVKRALTQARKVVNAII RRHGSPHSVHIELARELSKNHDERTKIVSAQDENYKKNKGAISILSEHGILNPTGYDIVRYKL WKEQGERCAYSLKEIPADTFFNELKKERNGAPILEVDHILPYSQSFIDSYHNKVLVYSDENRK KGNRIPYTYFLETNKDWEAFERYVRSNKFFSKKKREYLLKRAYLPRESELIKERHLNDTRYA STFLKNFIEQNLQFKEAEDNPRKRRVQTVNGVITAHFRKRWGLEKDRQETYLHHAMDAIIVA CTDHHMVTRVTEYYQIKESNKSVKKPYFPMPWEGFRDELLSHLASQPIAKKISEELKAGYQS LDYIFVSRMPKRSITGAAHKQTIMRKGGIDKKGKTIIIERLHLKDIKFDENGDFKMVGKEQDM ATYEAIKQRYLEHGKNSKKAFETPLYKPSKKGTGNLIKRVKVEGQAKSFVREVNGGVAQNG DLVRVDLFEKDDKYYMVPIYVPDTVCSELPKKVVASSKGYEQWLTLDNSFTFKFSLYPYDL VRLVKGDEDRFLYFGTLDIDSDRLNFKDVNKPSKKNEYRYSLKTIEDLEKYEVGVLGDLRLV RKETRRNFH (SEQ ID NO: 545), and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 545. In some embodiments, an active fragment of the APG07433.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 545.

In some embodiments, the presently disclosed compositions and methods comprise an RGN capable of binding a target sequence of the disclosure or an RGN having an amino acid sequence set forth as SEQ ID NO: 545, or an active variant or fragment thereof, wherein the RGN is capable of binding a target sequence adjacent to a PAM consensus sequence set forth as NNNNCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

In some embodiments, the presently disclosed compositions and methods comprise an RGN capable of binding a target sequence of the disclosure or an RGN having an amino acid sequence set forth as SEQ ID NO: 545, or an active variant or fragment thereof, wherein the RGN is capable of binding a target sequence adjacent to a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA having at least 80% sequence identity, at least 90% sequence identity, or at least 95% sequence identity to a nucleotide sequence set forth as SEQ ID NO: 693. In some embodiments, the RGN binds to a guide RNA having at least 80% sequence identity, at least 90% sequence identity, or at least 95% sequence identity to a nucleotide sequence set forth as SEQ ID NO: 694. In some embodiments, the RGN binds to a guide RNA having at least 80% sequence identity, at least 90% sequence identity, or at least 95% sequence identity to a nucleotide sequence set forth as SEQ ID NO: 695. In some embodiments, the RGN binds to a guide RNA having at least 80% sequence identity, at least 90% sequence identity, or at least 95% sequence identity to a nucleotide sequence set forth as SEQ ID NO: 696. In some embodiments, the RGN binds to a guide RNA having at least 80% sequence identity, at least 90% sequence identity, or at least 95% sequence identity to a nucleotide sequence set forth as SEQ ID NO: 697. In some embodiments, the RGN binds to a guide RNA having at least 80% sequence identity, at least 90% sequence identity, or at least 95% sequence identity to a nucleotide sequence set forth as SEQ ID NO: 698. In some embodiments, the RGN binds to a guide RNA having a sequence set forth as any one of SEQ ID NOs: 693, 694, 695, 696, 697, and 698. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof. In some embodiments, the RGN binds to a guide RNA comprising a CRISPR repeat set forth as SEQ ID NO: 546, or an active variant or fragment thereof. In some embodiments, the RGN binds to a guide RNA comprising a tracrRNA set forth as SEQ ID NO: 547, or an active variant or fragment thereof.

RGNs useful in the presently disclosed methods and compositions include APG05083.1 RNA-guided nuclease, the amino acid sequence of which is set forth as SEQ ID NO: 838, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 838. In some embodiments, an active fragment of the APG05083.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 838.

In some embodiments, the presently disclosed compositions and methods comprise an RGN capable of binding a target sequence of the disclosure or an RGN having an amino acid sequence set forth as SEQ ID NO: 838, or an active variant or fragment thereof, wherein the RGN is capable of binding a target sequence adjacent to a PAM consensus sequence set forth as NNNNCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

In some embodiments, the presently disclosed compositions and methods comprise an RGN capable of binding a target sequence of the disclosure or an RGN having an amino acid sequence set forth as SEQ ID NO: 838, or an active variant or fragment thereof, wherein the RGN is capable of binding a target sequence adjacent to a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

RGNs useful in the presently disclosed methods and compositions include APG07513.1 RNA-guided nuclease, the amino acid sequence of which is set forth as SEQ ID NO: 841, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 841. In some embodiments, an active fragment of the APG07513.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 841.

In some embodiments, the presently disclosed compositions and methods comprise an RGN capable of binding a target sequence of the disclosure or an RGN having an amino acid sequence set forth as SEQ ID NO: 841, or an active variant or fragment thereof, wherein the RGN is capable of binding a target sequence adjacent to a PAM consensus sequence set forth as NNNNCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

In some embodiments, the presently disclosed compositions and methods comprise an RGN capable of binding a target sequence of the disclosure or an RGN having an amino acid sequence set forth as SEQ ID NO: 841, or an active variant or fragment thereof, wherein the RGN is capable of binding a target sequence adjacent to a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

RGNs useful in the presently disclosed methods and compositions include APG08290.1 RNA-guided nuclease, the amino acid sequence of which is set forth as SEQ ID NO: 844, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 844. In some embodiments, an active fragment of the APG08290.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 844.

In some embodiments, the presently disclosed compositions and methods comprise an RGN capable of binding a target sequence of the disclosure or an RGN having an amino acid sequence set forth as SEQ ID NO: 844, or an active variant or fragment thereof, wherein the RGN is capable of binding a target sequence adjacent to a PAM consensus sequence set forth as NNRNCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

In some embodiments, the presently disclosed compositions and methods comprise an RGN capable of binding a target sequence of the disclosure or an RGN having an amino acid sequence set forth as SEQ ID NO: 844, or an active variant or fragment thereof, wherein the RGN is capable of binding a target sequence adjacent to a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments, the PAM sequence is 3′ of the target sequence on its non-target strand. In some embodiments, the RGN binds to a guide RNA having a sequence set forth as any one of SEQ ID NOs: 693-834, and 1086-1227. In some embodiments, the RGN binds to a guide RNA comprising a CRISPR repeat set forth as any one of SEQ ID NOs: 546, 549-552, 839, 842, 845, 940, 942-945, 1228, 1230, and 1232, or an active variant or fragment thereof, and a tracrRNA set forth as any one of SEQ ID NOs: 547, 553-562, 840, 842, 846, 941, 946-955, 1229, 1231, and 1233, or an active variant or fragment thereof.

In some embodiments, the presently disclosed compositions and methods comprise an RGN capable of binding a target sequence of the disclosure or an RGN having an amino acid sequence set forth as SEQ ID NO: 835, or an active variant or fragment thereof, wherein the RGN is capable of binding a target sequence adjacent to a full PAM sequence set forth as any one of GGGTCCTT, GGGGCCGA, GGGGCCCA, CGGCCCTG, GGGCCCAT, TGGCCC, TGGGCC, GGGCCC, CGGGCC, and AGGGCC. In some embodiments, the PAM sequence is 3′ of the target sequence on its non-target strand. In some embodiments, the RGN binds to a guide RNA comprising a CRISPR repeat set forth as SEQ ID NO: 918, or an active variant or fragment thereof, and a tracrRNA set forth as SEQ ID NO: 919, or an active variant or fragment thereof.

In some embodiments, the presently disclosed compositions and methods comprise an RGN capable of binding a target sequence of the disclosure or an RGN having an amino acid sequence set forth as SEQ ID NO: 915, or an active variant or fragment thereof, wherein the RGN is capable of binding a target sequence adjacent to a full PAM sequence set forth as any one of TCGGCCCT, CAGGCCTG, TCGGCC, and CGGGCC. In some embodiments, the PAM sequence is 3′ of the target sequence on its non-target strand. In some embodiments, the RGN binds to a guide RNA comprising a CRISPR repeat set forth as SEQ ID NO: 916, or an active variant or fragment thereof, and a tracrRNA set forth as SEQ ID NO: 917, or an active variant or fragment thereof.

According to the present invention, the presently disclosed target sequences within the FOXP3 gene are bound by an RGN. The target strand of the target sequence hybridizes with the guide RNA associated with the RGN. The target strand and/or the non-target strand of the target sequence (e.g., target DNA sequence) can then be subsequently cleaved by the RGN if the polypeptide possesses nuclease activity. The terms “cleave” or “cleavage” refer to the hydrolysis of at least one phosphodiester bond within the backbone of one or both strands of a double-stranded target sequence (e.g., target DNA sequence) that can result in either single-stranded or double-stranded breaks within the target DNA sequence. The cleavage of a presently disclosed target sequence can result in staggered breaks or blunt ends.

In some embodiments, the RGN used in the presently disclosed compositions and methods functions as a nickase, only cleaving a single strand of a double-stranded target sequence (e.g., target DNA sequence). Such RGNs have a single functioning nuclease domain. In some embodiments, the nickase is capable of cleaving the target strand or the non-target strand of the double-stranded target sequence (e.g., target DNA sequence). In embodiments where a nickase is used, in order to effect a double-stranded cleavage of a target sequence within the FOXP3 gene, two nickases are needed, each of which nicks a single strand within the target sequence. In some embodiments, additional nuclease domains have been mutated such that the nuclease activity is reduced or eliminated.

In some embodiments, the RGN lacks nuclease activity altogether and is referred to herein as nuclease-dead or nuclease inactive. Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated by reference in its entirety.

In some embodiments, nucleases other than RGNs are used in the presently disclosed compositions and methods. These nucleases can bind to additional target sequences of the FOXP3 gene distinct from the presently disclosed target sequences. As used herein, the term “nuclease” refers to an enzyme that catalyzes the cleavage of phosphodiester bonds between nucleotides in a nucleic acid molecule. In general, the nuclease is an endonuclease, which is capable of cleaving phosphodiester bonds between nucleotides within a nucleic acid molecule. In some embodiments, the sequence-specific nuclease is selected from the group consisting of a meganuclease, a zinc finger nuclease, a TAL-effector DNA binding domain-nuclease fusion protein (TALEN), and an RNA-guided nuclease (RGN) or variants thereof wherein the nuclease activity has been reduced or inhibited.

As used herein, the term “meganuclease” or “homing endonuclease” refers to endonucleases that bind a recognition site within double-stranded DNA that is 12 to 40 bp in length. Non-limiting examples of meganucleases are those that belong to the LAGLIDADG family that comprise the conserved amino acid motif LAGLIDADG (SEQ ID NO: 921). The term “meganuclease” can refer to a dimeric or single-chain meganuclease.

As used herein, the term “zinc finger nuclease” or “ZFN” refers to a chimeric protein comprising a zinc finger DNA-binding domain and a nuclease domain.

As used herein, the term “TAL-effector DNA binding domain-nuclease fusion protein” or “TALEN” refers to a chimeric protein comprising a TAL effector DNA-binding domain and a nuclease domain.

RGNs or nucleases (such as meganucleases, zinc finger nucleases, or TALENs) that lack nuclease activity and therefore, function as a DNA-binding polypeptide, can be used to deliver a fused polypeptide, polynucleotide, or small molecule payload to a particular genomic location. In some embodiments, the RGN polypeptide, guide RNA, or nuclease can be fused to a detectable label to allow for detection of a particular sequence. The detectable label or purification tag can be located at the N-terminus, the C-terminus, or an internal location of the RNA-guided nuclease, either directly or indirectly via a linker peptide. In some embodiments, the RGN component of the fusion protein is a nuclease-dead RGN. In some embodiments, the RGN component of the fusion protein is an RGN with nickase activity.

A detectable label is a molecule that can be visualized or otherwise observed. The detectable label may be fused to the RGN as a fusion protein (e.g., fluorescent protein) or may be a small molecule conjugated to the RGN polypeptide that can be detected visually or by other means. Detectable labels that can be fused to the presently disclosed RGNs as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

RGN polypeptides can also comprise a purification tag, which is any molecule that can be utilized to isolate a protein or fused protein from a mixture (e.g., biological sample, culture medium). Non-limiting examples of purification tags include biotin, myc, maltose binding protein (MBP), glutathione-S-transferase (GST), and 3× FLAG tag.

Alternatively, nuclease-dead RGNs can be targeted to the FOXP3 gene to alter the expression of the gene. In some embodiments, the binding of a nuclease-dead RGN to a target sequence within the FOXP3 gene results in the reduction in expression of FOXP3 by interfering with the binding of RNA polymerase or transcription factors within the targeted genomic region. In some embodiments, the RGN (e.g., a nuclease-dead RGN) or its complexed guide RNA further comprises an expression modulator that, upon binding to a target sequence within the FOXP3 gene, serves to either repress or activate the expression of the target gene.

In some embodiments, the expression modulator comprises a transcriptional repressor domain, which interacts with transcriptional control elements and/or transcriptional regulatory proteins, such as RNA polymerases and transcription factors, to reduce or terminate transcription of the FOXP3 gene. Transcriptional repressor domains are known in the art and include, but are not limited to, Sp1-like repressors, IκB, and Krüppel associated box (KRAB) domains.

In some embodiments, the expression modulator comprises a transcriptional activation domain, which interacts with transcriptional control elements and/or transcriptional regulatory proteins, such as RNA polymerases and transcription factors, to increase or activate transcription of the FOXP3 gene. Transcriptional activation domains are known in the art and include, but are not limited to, a herpes simplex virus VP16 activation domain and an NFAT activation domain.

In some embodiments, the expression modulator modulates the expression of the FOXP3 sequence through epigenetic mechanisms. In some embodiments, an epigenetic modulator covalently modifies DNA or histone proteins to alter histone structure and/or chromosomal structure without altering the DNA sequence, leading to changes in gene expression (e.g., upregulation or downregulation). Non-limiting examples of epigenetic modifications include acetylation or methylation of lysine residues, arginine methylation, serine and threonine phosphorylation, and lysine ubiquitination and sumoylation of histone proteins, and methylation and hydroxymethylation of cytosine residues in DNA. Non-limiting examples of epigenetic modulators include histone acetyltransferases, histone deacetylases, histone methyltransferases, histone demethylases, DNA methyltransferases, and DNA demethylases.

The nuclease-dead RGNs or an RGN with nickase activity can be targeted to particular genomic locations to modify the sequence of a target polynucleotide through fusion to a base-editing polypeptide, for example a deaminase polypeptide or active variant or fragment thereof, that directly chemically modifies (e.g., deaminates) a nucleobase, resulting in conversion from one nucleobase to another. The base-editing polypeptide can be fused to the RGN at its amino-terminal (N-terminal) or carboxy-terminal (C-terminal) end. Additionally, the base-editing polypeptide may be fused to the RGN via a peptide linker. Fusions of base-editing polypeptides and RGNs are described in International Appl. No. PCT/IB2023/061192, filed Nov. 6, 2023, which is herein incorporated by reference in its entirety. A non-limiting example of a deaminase polypeptide that is useful for such compositions and methods includes a cytosine deaminase or an adenosine deaminase (such as the adenosine deaminase base editor described in Gaudelli et al. (2017) Nature 551:464-471, U.S. Publ. Nos. 2017/0121693 and 2018/0073012, and International Publ. No. WO 2018/027078, or any of the deaminases disclosed in International Publ. No. WO 2020/139783, International Publ. No. WO 2022/056254, International Appl. No. PCT/US2022/021271, filed Mar. 22, 2022, and International Appl. No. PCT/IB2023/061192, filed Nov. 6, 2023, each of which is herein incorporated by reference in its entirety). In some embodiments, the deaminase polypeptide that is useful for such presently disclosed compositions and methods is a deaminase disclosed in Table 17 of International Publ. No. WO 2020/139783, which is incorporated herein by reference in its entirety.

Further, it is known in the art that certain fusion proteins between an RGN and a base-editing enzyme (e.g., cytosine deaminase) may also comprise at least one uracil stabilizing polypeptide that increases the mutation rate of a cytidine, deoxycytidine, or cytosine to a thymidine, deoxythymidine, or thymine in a nucleic acid molecule by a deaminase. Non-limiting examples of uracil stabilizing polypeptides include those disclosed in PCT Publication No. WO 2021/217002 and PCT Publication No. WO 2022/015969, each of which is herein incorporated by reference in its entirety. The disclosed uracil stabilizing polypeptides include USP2, and a uracil glycosylase inhibitor (UGI) domain, which may increase base editing efficiency. Therefore, a fusion protein may comprise an RGN described herein or variant thereof, a deaminase, and optionally at least one uracil stabilizing polypeptide, such as UGI or USP2. In embodiments, the RGN that is fused to the base-editing polypeptide is a nickase that cleaves the DNA strand that is not acted upon by the base-editing polypeptide (e.g., deaminase).

An RGN may be fused to a reverse transcriptase (RT) editing polypeptide (also referred to as prime editing polypeptide). RT editing (also referred to as prime editing) is a versatile and precise genome editing method that directly writes new genetic information into a specified DNA site using a nucleic acid programmable DNA binding protein working in association with a polymerase (described in, e.g., U.S. Pat. No. 11,447,770B1; WO2021072328; WO2021226558; WO2020156575; WO2021042047; U.S. Ser. No. 11/193,123; each incorporated by reference in its entirety herein). The RT editing system uses an RGN that is a nickase, and the system is programmed with a RT editing guide RNA. The RT editing guide RNA is a guide RNA that both specifies the target sequence and provides the template for polymerization of the replacement strand containing the edit by way of an extension engineered onto the guide RNA (e.g., at the 5′ or 3′ end, or at an internal portion of the guide RNA). The RGN nickase/RT editing polypeptide fusion is guided to the target sequence by the RT editing guide RNA and nicks the non-target strand upstream of sequence to be edited and upstream of the PAM, creating a 3′ flap on the non-target strand. The RT editing guide RNA includes a primer binding site (PBS) that is complementary to the 3′ flap of the non-target strand. In some embodiments, a PBS is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In certain embodiments, the RT editing guide RNA comprises a PBS that is at least 5 (e.g., at least 5, 6, 7, 8, 9, 10, 11, 12, 13, 14, 15, 16, 17, 28, 19, or 20) nucleotides in length. In some embodiments, the RT editing guide RNA may comprise a PBS that is at least 8 nucleotides in length. Hybridrization of the PBS and 3′ flap of the non-target strand allows polymerization of the replacement strand containing the edit using the extension of the RT editing guide RNA as template. The extension of the RT editing guide RNA can be formed from RNA or DNA. In the case of an RNA extension, the polymerase of the RT editor can be an RNA-dependent DNA polymerase (such as a reverse transcriptase). In the case of a DNA extension, the polymerase of the RT editor may be a DNA-dependent DNA polymerase.

The replacement strand containing the desired edit (e.g., a single nucleobase substitution) shares the same sequence as the non-target strand of the target sequence to be edited (with the exception that it includes the desired edit). Through DNA repair and/or replication machinery, the non-target strand of the target sequence is replaced by the newly synthesized replacement strand containing the desired edit. In some cases, RT editing may be thought of as a “search-and-replace” genome editing technology since the RT editors not only search and locate the desired target sequence to be edited, but at the same time, encode a replacement strand containing a desired edit which is installed in place of the corresponding non-target strand of the target sequence. Thus, in some embodiments, a guide RNA of the disclosure comprises an extension comprising an edit template for RT editing. In some embodiments, a RT editing polypeptide that can be fused to an RGN includes a DNA polymerase. In certain embodiments, the DNA polymerase is a reverse transcriptase. In certain embodiments, the RGN is a nickase.

RGNs or other nucleases that are fused to a polypeptide or domain can be separated or joined by a linker. The term “linker,” as used herein, refers to a chemical group or a molecule linking two molecules or moieties, e.g., a binding domain and a cleavage domain of a nuclease. In some embodiments, a linker joins a gRNA binding domain of an RGN and a detectable label or epigenetic modulator. In some embodiments, a linker joins a nuclease-dead RGN and a detectable label or epigenetic modulator. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The presently disclosed compositions and methods can utilize RGNs or other nucleases comprising at least one nuclear localization signal (NLS) to enhance transport of the RGN to the nucleus of a cell. Nuclear localization signals are known in the art and generally comprise a stretch of basic amino acids (see, e.g., Lange et al., J. Biol. Chem. (2007) 282:5101-5105). In some embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleoplasmin, and c-Myc (see, e.g., Ray et al. (2015) Bioconjug Chem 26(6):1004-7). In embodiments, the RGN comprises the NLS sequence set forth as SEQ ID NO: 922 or 923. The RGN or other nuclease can comprise one or more NLS sequences at its N-terminus, C-terminus, or both the N-terminus and C-terminus. For example, the RGN can comprise two NLS sequences at the N-terminal region and four NLS sequences at the C-terminal region.

In some embodiments, the presently disclosed compositions and methods utilize RGNs or other nucleases comprising at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal and/or cell-penetrating domain can be located at the N-terminus, the C-terminus, or in an internal location of the RGN or other nuclease.

V. Polynucleotides Encoding RNA-Guided Nucleases, Single Guide RNAs, CRISPR RNAs, and or tracrRNAs

The present disclosure provides polynucleotides comprising or encoding the presently disclosed RGNs, crRNAs, tracrRNAs, and/or sgRNAs. Presently disclosed polynucleotides include those comprising or encoding a crRNA comprising a spacer capable of targeting a bound RGN to a target sequence in the FOXP3 gene having the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

The use of the term “polynucleotide” or “nucleic acid molecule” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, and the like.

In some of those embodiments wherein the presently disclosed compositions and methods comprise a nucleic acid molecule encoding an RGN, the nucleic acid molecule is an mRNA (messenger RNA) molecule. An mRNA refers to any polynucleotide which encodes a polypeptide of interest and which is capable of being translated to produce the encoded polypeptide of interest in vitro, in vivo, in situ, or ex vivo. In some embodiments, the basic components of an mRNA molecule include at least a coding region, a 5′UTR, a 3′UTR, a 5′ cap and a poly-A tail. In some embodiments, an mRNA encoding an RGN useful in the presently disclosed methods and compositions can include one or more structural and/or chemical modifications or alterations which impart useful properties to the polynucleotide. For instance, a useful property of an mRNA includes the lack of a substantial induction of the innate immune response of a cell into which the mRNA is introduced. A “structural” feature or modification is one in which two or more linked nucleotides are inserted, deleted, duplicated, inverted or randomized in an mRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. Chemical modifications to mRNA can involve inclusion of 5-methylcytosine, N1-methyl-pseudouridine, pseudouridine, 2-thiouridine, 4-thiouridine, 5-methoxyuridine, 2′Fluoroguanosine, 2′Fluorouridine, 5-bromouridine, 5-(2-carbomethoxyvinyl) uridine, 5-[3(1-E-propenylamino)]uridine, α-thiocytidine, N6-methyladenosine, 5-methylcytidine, N4-acetylcytidine, 5-formylcytidine, or combinations thereof, in an mRNA.

The nucleic acid molecules encoding RGNs can be codon optimized for expression in an organism of interest (e.g., mammal). A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Gaspar et al. (2012) Bioinformatics 28(20): 2683-2684; Komar et al. (1998) Biol. Chem. 379(10): 1295-1300; and Inouye et al. (2015) Protein Expr. Purif 109: 47-54). Non-limiting examples of codon-optimized coding sequences for RGNs useful in the presently disclosed compositions and methods include SEQ ID NO: 548.

Polynucleotides encoding the RGNs, crRNAs, tracrRNAs, and/or sgRNAs provided herein can be provided in expression cassettes for in vitro expression or expression in a cell, embryo, or organism of interest. The cassette will include 5′ and 3′ regulatory sequences operably linked to a polynucleotide encoding an RGN, a crRNA, a tracrRNA, and/or an sgRNA provided herein that allows for expression of the polynucleotide. The cassette may additionally contain at least one additional gene or genetic element to be co-transformed into the organism. Where additional genes or elements are included, the components are operably linked. The term “operably linked” is intended to mean a functional linkage between two or more elements. For example, an operable linkage between a promoter and a coding region of interest (e.g., region coding for an RGN, a crRNA, a tracrRNA, and/or an sgRNA) is a functional link that allows for expression of the coding region of interest. Operably linked elements may be contiguous or non-contiguous. When used to refer to the joining of two protein coding regions, by operably linked or “operably fused” is intended that the coding regions are in the same reading frame. In some embodiments, polypeptides that are “operably fused” means that the structure and/or biological activity of each individual peptide is also present in the fusion. Alternatively, the additional gene(s) or element(s) can be provided on multiple expression cassettes. For example, the nucleotide sequence encoding a presently disclosed RGN can be present on one expression cassette, whereas the nucleotide sequence encoding a crRNA, a tracrRNA, or a complete guide RNA can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette will include in the 5′-3′ direction of transcription, a transcriptional (and, in some embodiments, translational) initiation region (i.e., a promoter), an RGN-, crRNA-, tracrRNA-and/or sgRNA-encoding polynucleotide of the disclosure, and a transcriptional (and in some embodiments, translational) termination region (i.e., termination region) functional in the organism of interest. The promoters of the disclosure are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions include ones from simian virus (SV40), human growth hormone (hGH), bovine growth hormone (BGH), and rabbit beta-globin (rbGlob). See also Proudfoot (1991) Cell 64:671-674; Munroe et al. (1990) Gene 91:151-158; Schek et al. (1992) Molecular and Cellular Biology 12(12):5386-5393; Gil and Proudfoot (1987) Cell 49(3):399-406; Goodwin and Rottman (1992) The Journal of Biological Chemistry 267(23):16330-16334; and Lanoix and Acheson (1988) EMBO J. 7(8): 2515-2522.

Additional regulatory signals include, but are not limited to, transcriptional initiation start sites, operators, activators, enhancers, other regulatory elements, ribosomal binding sites, an initiation codon, termination signals, and the like. See, for example, Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest.

Exemplary constitutive promoters for expression in cells of the present disclosure include: an SV40 early promoter; a mouse mammary tumor virus long terminal repeat (LTR) promoter; adenovirus major late promoter (Ad MLP); a herpes simplex virus (HSV) promoter; a cytomegalovirus (CMV) promoter such as the CMV immediate early promoter region (CMVIE); a rous sarcoma virus (RSV) promoter; a human ubiquitin C promoter (UBC); a human U6 small nuclear promoter (U6); an enhanced U6 promoter; a human H1 promoter from RNA polymerase III (H1); a human elongation factor 1α promoter (EF1A); a human beta-actin promoter (ACTB); a human or mouse phosphoglycerate kinase 1 promoter (PGK); a chicken β-Actin promoter coupled with CMV early enhancer (CAGG); a yeast transcription elongation factor promoter (TEF1); and the like. See, for example, Miyagishi et al. (2002) Nature Biotechnology 20:497-500; Xia et al. (2003) Nucleic Acids Res. 31(17):e100-e100; Pasleau et al. (1985) Gene 38:227-232; Martin-Gallardo et al. (1988) Gene 70: 51-56; Oellig and Seliger (1990) J Neurosci Res 26: 390-396; Manthorpe et al. (1993) Hum Gene Ther 4: 419-431; Yew et al. (1997) Hum Gene Ther 8: 575-584; Xu et al. (2001) Gene 272: 149-156; Nguyen et al. (2008) J Surg Res 148: 60-66; Costa et al. (2005) Nat Meth. 2:259-260; Lam and Truong (2020) ACS Synth. Biol. 9(10):2625-2631.

Examples of inducible promoters include: stress-regulated promoters such as Hsp70 and Hsp90 promoters (Wurm et al. (1986) Proc. Natl. Acad. Sci. USA. 83:5414-5418; Nover L. Heat Shock Response. CRC Press; Boca Raton, FL, USA: 1991); metal-regulated promoters (Mayo et al. (1982) Cell. 29:99-108; Searle et al. (1985) Mol. Cell. Biol. 5:1480-1489); hormone-responsive promoters including a glucocorticoid-responsive promoter (Hynes et al. (1981) Proc. Natl. Acad. Sci. USA. 78:2038-2042; Klock et al. (1987) Nature. 329:734-736). Chemically regulated promoters from prokaryotes that have been used include isopropyl-beta-D-thiogalactopyranoside (IPTG)-regulated promoters, lactose-regulated promoters, and tetracycline-reulated promoters (see, for example, Gossen et al. (1993) Trends Biochem Sci. 18:471-475; Gossen and Bujard (1992) Proc. Natl Acad. Sci. USA 89:5547-5551; Zhou et al. (2006) Gene Ther. 13:1382-1390). Inducible expression can be obtained using operator systems including AlcR/acetaldehyde, ArgR/L-arginine, BirA/biotinyl-AMP, CymR/cumate, EthR/2-phenylethylbutyrate, HdnoR/6-hydroxynicotine, HucR/uric acid, MphR(A)/macrolides, PIP/Streptogramins, Rex/NADH, RheA/heat, ScbR/SCB1, TraR/3-oxo-C8-HSL, and TtgR/phloretin; see, for example, U.S. Pat. No. 8,728,759B2; U.S. Pat. No. 7,745,592B2; Weber and Fussenegger (2004) Methods Mol. Biol. 267:451-466; Hartenbach et al. (2007) Nucleic Acids Res. 35:e136; Weber et al. (2009) Metab. Eng. 11:117-124; Weber et al. (2008) Proc. Natl. Acad. Sci. USA. 105:9994-9998; Malphettes et al. (2005) Nucleic Acids Res. 33:e107; Kemmer et al. (2010) Nat. Biotechnol. 28:355-360; Weber et al. (2002) Nat. Biotechnol. 20:901-907; Fussenegger et al. (2000) Nat. Biotechnol. 18:1203-1208; Weber et al. (2006) Metab. Eng. 8:273-280; Weber et al. (2003) Nucleic Acids Res. 31:e69; Weber et al. (2003) Nucleic Acids Res. 31:e71; Neddermann et al. (2003) EMBO Rep. 4:159-165; and Gitzinger et al. (2009) Proc. Natl. Acad. Sci. USA. 106:10638-10643. Inducible expression can be obtained using protein-protein interaction systems including: rapamycin-induced interaction between FKBP12 (FK506 binding protein 12) and mTOR (Rivera et al. (1996) Nat. Med. 2:1028-1032; Belshaw et al. (1996) Proc. Natl. Acad. Sci. USA. 93:4604-46077); abscisic acid (ABA)-regulated interaction between PYL1 (abscisic acid receptor) and ABIl (protein phosphatase 2C56) (Liang et al. (2011) Sci. Signal. 4(164):rs2-rs2); and light-induced protein-protein interaction systems (Wang et al. (2012) Nat. Methods. 9:266-269; Yamada et al. (2018) Cell. Rep. 25:487-500).

Tissue-specific or tissue-preferred promoters can be utilized to target expression of an expression construct within a particular tissue. In embodiments, the tissue-specific or tissue-preferred promoters are active in mammalian tissue. Examples of tissue-specific or tissue-preferred promoters include promoters that initiate transcription preferentially in certain tissues, such as the heart, CNS, or eye. A “tissue specific” promoter is a promoter that initiates transcription only in certain tissues. Unlike constitutive expression of genes, tissue-specific expression is the result of several interacting levels of gene regulation. As such, promoters from homologous or closely related species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding an RGN, crRNA, tracrRNA, and/or sgRNA comprise a cell type-specific promoter. A “cell type specific” promoter is a promoter that primarily drives expression in certain cell types in one or more organs. Some examples of cells in which cell type specific promoters may be primarily active include, for example, a cytotoxic T cell, a regulatory T cell, or a stem cell. The nucleic acid molecules can also include cell type preferred promoters. A “cell type preferred” promoter is a promoter that primarily drives expression mostly, but not necessarily entirely or solely in certain cell types in one or more organs. Some examples of cells in which cell type preferred promoters may be preferentially active include, for example, lymphocyte, neuron, adipocyte, cardiomyocyte, smooth muscle cell, and photoreceptor cell.

The nucleic acid sequences encoding the RGNs, crRNAs, tracrRNAs, and/or sgRNAs can be operably linked to a promoter sequence that is recognized by a phage RNA polymerase for example, for in vitro mRNA synthesis. In some embodiments, the in vitro-transcribed RNA can be purified for use in the methods described herein. For example, the promoter sequence can be a T7, T3, or SP6 promoter sequence or a variation of a T7, T3, or SP6 promoter sequence. In some embodiments, the expressed protein and/or RNAs can be purified for use in the methods of genome modification described herein.

In embodiments, the polynucleotide encoding the RGN, crRNA, tracrRNA, and/or sgRNA also can be linked to a polyadenylation signal (e.g., SV40 polyA signal and other signals functional in plants) and/or at least one transcriptional termination sequence. Additionally, the sequence encoding the RGN also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the RGN, crRNA, tracrRNA, and/or sgRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT). Marker genes can include genes that allow selection for growth on a particular nutrient or substance, such as dihydrofolate reductase (DHFR; Simonsen and Levinson (1983) Proc. Natl. Acad. Sci. USA. 80:2495-2499), histidinol dehydrogenase (hisD; Hartman and Mulligan (1988) Proc. Natl. Acad. Sci. USA. 85:8047-8051), puromycin-N-acetyl transferase (PAC or puro; de la Luna et al. (1988) Gene 62:121-126), thymidine kinase (TK; Littlefield (1964) Science 145:709-710), and xanthine-guanine phosphoribosyltransferase (XGPRT or gpt; Mulligan and Berg (1981) Proc. Natl. Acad. Sci. USA. 78:2072-2076).

In some embodiments, the expression cassette or vector comprising the sequence encoding the RGN polypeptide can further comprise a sequence encoding a crRNA and/or a tracrRNA, or the crRNA and tracrRNA combined to create an sgRNA. The sequence(s) encoding the crRNA and/or tracrRNA can be operably linked to at least one transcriptional control sequence for expression of the crRNA and/or tracrRNA in the organism or host cell of interest. For example, the polynucleotide encoding the crRNA and/or tracrRNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters, such as the human U6 promoter set forth as SEQ ID NO: 924, as well as the promoters disclosed in U.S. Provisional Appl. No. 63/209,660, filed Jun. 11, 2021, and International Application No. PCT/US2022/032940, filed Jun. 10, 2022, each of which is herein incorporated by reference in its entirety, including promoters set forth herein as SEQ ID NOs: 925-934.

As indicated, expression constructs comprising nucleotide sequences encoding an RGN, a crRNA, a tracrRNA, and/or an sgRNA can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the disclosure do not require a particular method for introducing a nucleotide construct to a host organism, only that the nucleotide construct gains access to the interior of at least one cell of the host organism. The host cell can be a eukaryotic or prokaryotic cell. In some embodiments, the eukaryotic host cell is a mammalian cell, an avian cell, or an insect cell. In some embodiments, the eukaryotic cell that comprises or expresses a presently disclosed crRNA, tracrRNA, sgRNA, and/or RGN or that has been modified by a presently disclosed RGN system is a human cell. In some embodiments, the eukaryotic cell that comprises or expresses a presently disclosed crRNA, tracrRNA, sgRNA, and/or RGN or that has been modified by a presently disclosed RGN system is a stem cell, including an induced pluripotent stem cell. In some embodiments, the mammalian or human cell that comprises or expresses a presently disclosed crRNA, tracrRNA, sgRNA, and/or RGN or that has been modified by a presently disclosed RGN system is a lymphocyte. In some embodiments, the lymphocyte includes a cytotoxic T cell or a regulatory T cell.

Methods for introducing nucleotide constructs into host cells are known in the art including, but not limited to, stable transformation methods, transient transformation methods, and virus-mediated methods.

The presently disclosed methods can result in a transformed organism or cell line derived from these transformed cells.

“Transgenic organisms” or “transformed organisms” or “stably transformed” organisms or cells or tissues refers to organisms that have incorporated or integrated a polynucleotide encoding an RGN, a crRNA, a tracrRNA, and/or an sgRNA of the disclosure. It is recognized that other exogenous or endogenous nucleic acid sequences or DNA fragments may also be incorporated into the host cell. Transformation of a host cell may be performed by infection, conjugation, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, a crRNA, a tracrRNA, and/or an sgRNA includes retroviral, lentiviral, adenoviral, and adeno-associated viral mediated introduction and expression.

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

In some embodiments, cells that have been transformed may be introduced into an organism. These cells could have originated from the organism, wherein the cells are transformed in an ex vivo approach. These cells can be autologous (originated and returned to the same subject), allogeneic (the donor and recipient subjects are of the same species). In general, the donor and recipient of allogeneic cells are a complete or partial HLA match.

The polynucleotides encoding the RGNs, crRNAs, tracrRNAs, and/or sgRNAs or comprising the crRNAs, tracrRNAs, and/or sgRNAs can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The polynucleotides encoding the RGNs, crRNAs, tracrRNAs, and/or sgRNAs or comprising the crRNAs, tracrRNAs, and/or sgRNAs can be used to transform any eukaryotic species, including but not limited to animals (e.g., mammals, humans, insects, fish, birds, and reptiles), fungi, amoeba, algae, and yeast.

Conventional viral and non-viral based gene transfer methods can be used to introduce nucleic acids in mammalian, insect, or avian cells or target tissues. Such methods can be used to administer nucleic acids encoding components of an RGN system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256: 808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., J. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more nucleic acid molecules or vectors described herein. In some embodiments, a cell is transfected as it naturally occurs in a subject. In some embodiments, a cell that is transfected is taken from a subject. In embodiments, the cell is derived from cells taken from a subject, such as a cell line. In some embodiments, the cell line may be mammalian, insect, or avian cells. A wide variety of cell lines for tissue culture are known in the art. Examples of cell lines include, but are not limited to, C8161, CCRF-CEM, MOLT, mIMCD-3, NHDF, HeLaS3, Huh1, Huh4, Huh7, HUVEC, HASMC, HEKn, HEKa, MiaPaCell, Panel, PC-3, TFl, CTLL-2, CIR, Rat6, CVI, RPTE, AlO, T24, 182, A375, ARH-77, Calul, SW480, SW620, SKOV3, SK-UT, CaCo2, P388D1, SEM-K2, WEHI-231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-Ll, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10Tl/2, C6/36, Cal-27, CHO, CHO-7, CHO-IR, CHO-K1, CHO-K2, CHO-T, CHO Dhfr−/−, COR-L23, COR-L23/CPR, COR-L235010, CORL23/R23, COS-7, COV-434, CML Tl, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, lurkat, lY cells, K562 cells, Ku812, KCL22, KGl, KYOl, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more nucleic acid molecules or vectors described herein is used to establish a new cell line comprising one or more vector-derived sequences. In some embodiments, a cell transiently transfected with the components of an RGN system as described herein (such as by transient transfection of one or more vectors, or transfection with RNA), and modified through the activity of an RGN system, is used to establish a new cell line comprising cells containing the modification but lacking any other exogenous sequence.

In some embodiments, one or more nucleic acid molecules or vectors described herein are used to produce a non-human transgenic animal. In some embodiments, the transgenic animal is a mammal, such as a mouse, rat, hamster, rabbit, cow, or pig. In some embodiments, the transgenic animal is a bird, such as a chicken or a duck. In some embodiments, the transgenic animal is an insect, such as a mosquito or a tick.

### VI. Variants and Fragments of Polypeptides and Polynucleotides

The present disclosure provides active variants and fragments of the presently disclosed crRNAs, tracrRNAs, sgRNA backbones, sgRNAs, and RGNs. An active variant or fragment of a naturally-occurring (i.e., wild-type) RGN binds to a target sequence described herein within the FOXP3 gene in an RNA-guided sequence-specific manner. In some embodiments, a target sequence described herein includes a target strand having the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214. In some embodiments, the disclosure provides active variants and fragments of an RGN having an amino acid sequence set forth as SEQ ID NO: 545, as well as active variants and fragments of naturally-occurring CRISPR repeats, including sequences set forth as SEQ ID NOs: 546, 549-552, 839, 842, 845, 940, 942-945, 1228, 1230, and 1232, active variants and fragments of naturally-occurring tracrRNAs, such as any one of the sequences set forth as SEQ ID NOs: 547, 553-562, 840, 842, 846, 941, 946-955, 1229, 1231, and 1233, and active variants and fragments of sgRNAs, such as any one of the sequences set forth as SEQ ID NOs: 693-834, and 1086-1227, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring RGN polypeptides, such as those disclosed herein, will retain sequence-specific, RNA-guided DNA-binding activity. In embodiments, fragments and variants of naturally-occurring RGN polypeptides, such as those disclosed herein, retain nuclease activity (single-stranded or double-stranded).

Fragments and variants of naturally-occurring CRISPR repeats, such as those disclosed herein, will retain the ability, when part of a guide RNA (comprising a tracrRNA), to bind to and guide an RNA-guided nuclease (complexed with the guide RNA) to a target sequence in a sequence-specific manner.

Fragments and variants of naturally-occurring tracrRNAs, such as those disclosed herein, will retain the ability, when part of a guide RNA (comprising a CRISPR RNA), to guide an RNA-guided nuclease (complexed with the guide RNA) to a target sequence in a sequence-specific manner.

Fragments and variants of sgRNA backbones, such as those disclosed herein, will retain the ability, when part of a guide RNA, to guide an RNA-guided nuclease (complexed with the guide RNA) to a target sequence in a sequence-specific manner.

Fragments and variants of sgRNAs, such as those disclosed herein, will retain the ability to guide an RNA-guided nuclease (complexed with the sgRNA) to a target sequence in a sequence-specific manner.

The term “fragment” refers to a portion of a polynucleotide or polypeptide sequence of the disclosure. “Fragments” or “biologically active portions” include polynucleotides comprising a sufficient number of contiguous nucleotides to retain the biological activity (i.e., binding to and directing an RGN in a sequence-specific manner to a target sequence when comprised within a guide RNA). “Fragments” or “biologically active portions” include polypeptides comprising a sufficient number of contiguous amino acid residues to retain the biological activity (i.e., binding to a target sequence in a sequence-specific manner when complexed with a guide RNA). Fragments of the RGN proteins include those that are shorter than the full-length sequences due to the use of an alternate downstream start site. A biologically active portion of an RGN protein can be a polypeptide that comprises, for example, 10, 25, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700 or more contiguous amino acid residues of an RGN that binds a target nucleotide sequence disclosed herein or of SEQ ID NO: 545. Such biologically active portions can be prepared by recombinant techniques and evaluated for sequence-specific, RNA-guided DNA-binding activity. A biologically active fragment of a CRISPR repeat sequence can comprise at least 8 contiguous nucleotides of any one of SEQ ID NOs: 546, 549-552, 839, 842, 845, 940, 942-945, 1228, 1230, and 1232. A biologically active portion of a CRISPR repeat sequence can be a polynucleotide that comprises, for example, 8, 9, 10, 11, 12, or 13 contiguous nucleotides of any one of SEQ ID NOs: 546, 549-552, 839, 842, 845, 940, 942-945, 1228, 1230, and 1232. A biologically active fragment of a crRNA sequence can comprise at least 20 contiguous nucleotides of any one of SEQ ID NOs: 574-692, and 967-1085. A biologically active portion of a crRNA can be a polynucleotide that comprises, for example, 20, 25, 30, 35, 40 or more contiguous nucleotides of any one of SEQ ID NOs: 574-692, and 967-1085. A biologically active portion of a tracrRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80 or more contiguous nucleotides of any one of SEQ ID NOs: 547, 553-562, 840, 842, 846, 941, 946-955, 1229, 1231, and 1233. A biologically active portion of a sgRNA backbone can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more contiguous nucleotides of any one of SEQ ID NOs: 563-573, and 956-966. A biologically active portion of a sgRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more contiguous nucleotides of any one of SEQ ID NOs: 693-834, and 1086-1227.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In certain embodiments, the presently disclosed polynucleotides encode an RNA-guided nuclease polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence encoding an RGN that binds a target sequence disclosed herein or an amino acid sequence set forth as SEQ ID NO: 545.

A biologically active variant of an RGN polypeptide of the disclosure may differ by as few as about 1-15 amino acid residues, as few as about 1-10, such as about 6-10, as few as 5, as few as 4, as few as 3, as few as 2, or as few as 1 amino acid residue. In some embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700 amino acids or more from either the N or C terminus of the polypeptide.

In some embodiments, the presently disclosed polynucleotides comprise or encode a crRNA repeat comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 546, 549-552, 839, 842, 845, 940, 942-945, 1228, 1230, and 1232.

In some embodiments, the presently disclosed polynucleotides comprise or encode a crRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 574-692, and 967-1085.

The presently disclosed polynucleotides can comprise or encode a tracrRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 547, 553-562, 840, 842, 846, 941, 946-955, 1229, 1231, and 1233.

The presently disclosed polynucleotides can comprise or encode an sgRNA backbone comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 563-573, and 956-966.

The presently disclosed polynucleotides can comprise or encode an sgRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 693-834, and 1086-1227.

Biologically active variants of a CRISPR repeat, crRNA, tracrRNA, sgRNA backbone, or sgRNA of the disclosure may differ by as few as about 1-15 nucleotides, as few as about 1-10, such as about 6-10, as few as 5, as few as 4, as few as 3, as few as 2, or as few as 1 nucleotide. In some embodiments, the polynucleotides can comprise a 5′ or 3′ truncation, which can comprise at least a deletion of5, 10, 15, 20, 25, 30, 35, 40, 45, 50, 55, 60, 65, 70, 75, 80, 90, 95, 100, 105, 110 nucleotides or more from either the 5′ or 3′ end of the polynucleotide.

It is recognized that modifications may be made to the RGN polypeptides, CRISPR repeats, crRNAs, tracrRNAs, sgRNA backbones, and sgRNAs provided herein, creating variant proteins and polynucleotides. Changes designed by man may be introduced through the application of site-directed mutagenesis techniques. Alternatively, native, as yet-unknown, or as yet unidentified polynucleotides and/or polypeptides structurally and/or functionally-related to the sequences disclosed herein may also be identified that fall within the scope of the present disclosure. Conservative amino acid substitutions may be made in non-conserved regions that do not alter the function of the RGN proteins. Alternatively, modifications may be made that improve the activity of the RGN.

Variant polynucleotides and proteins also encompass sequences and proteins derived from a mutagenic and recombinogenic procedure such as DNA shuffling. With such a procedure, one or more different RGN proteins disclosed herein (e.g., SEQ ID NO: 545) is manipulated to create a new RGN protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the RGN sequences provided herein and other known RGN genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. It is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Protein sequences that differ by such conservative substitutions are said to have “sequence similarity” or “similarity”. Means for measuring sequence similarity are well known to those of skill in the art. Typically, this involves scoring a conservative substitution as a partial rather than a full mismatch. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VII. RGN Systems and Ribonucleoprotein Complexes for Binding a Target Sequence of Interest and Methods of Making the Same

The present disclosure provides a RGN system for binding a target sequence in the FOXP3 gene. As used herein, an RGN system comprises at least one RGN polypeptide or a polynucleotide comprising a nucleotide sequence encoding the RGN polypeptide and one or more guide RNAs. The one or more guide RNAs are capable of forming a complex with the RGN polypeptide (ribonucleoprotein complex). The presently disclosed RGN systems comprise: a) one or more guide RNAs, or one or more polynucleotides comprising one or more nucleotide sequences encoding the one or more guide RNAs; and b) an RGN polypeptide or a polynucleotide comprising a nucleotide sequence encoding the RGN polypeptide. The one or more guide RNAs are capable of targeting a bound RGN polypeptide to a target sequence. The one or more guide RNAs are capable of forming a complex with the RGN polypeptide to direct the RGN polypeptide to bind to the target sequence in the FOXP3 gene. The guide RNA hybridizes to the target strand of a target sequence in the FOXP3 gene and also forms a complex with the RGN polypeptide, thereby directing the RGN polypeptide to bind to the target sequence. In some embodiments, the target sequence is set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

In some embodiments, the target sequence within the FOXP3 gene has the nucleotide sequence set forth as: TGCCAGGCCTGGGGTTGGGCATC (SEQ ID NO: 156). In some embodiments, the target sequence within the FOXP3 gene has the nucleotide sequence set forth as: CAGGTCTGAGGCTTTGGGTGCAG (SEQ ID NO: 164). In some embodiments, the target sequence within the FOXP3 gene has the nucleotide sequence set forth as: TCGAAGATCTCGGCCCTGGAAGG (SEQ ID NO: 180). In some embodiments, the target sequence within the FOXP3 gene has the nucleotide sequence set forth as: TCTCGGCCCTGGAAGGTTCCCCCTG (SEQ ID NO: 190). In some embodiments, the target sequence within the FOXP3 gene has the nucleotide sequence set forth as: GGTTCAAGGAAGAAGAGGAGGCA (SEQ ID NO: 198). In some embodiments, the target sequence within the FOXP3 gene has the nucleotide sequence set forth as:

(SEQ ID NO: 194)
GGGGTTCAAGGAAGAAGAGGAGGCA.

In some embodiments, the RGN is capable of recognizing a consensus PAM sequence set forth as NNNNCC. In some embodiments, the RGN is capable of recognizing a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. In some embodiments, the RGN comprises an amino acid sequence set forth as SEQ ID NO: 545, or an active variant or fragment thereof. In some embodiments, the RGN comprises an amino acid sequence having at least 80% sequence identity to SEQ ID NO: 545. In some embodiments, the guide RNA comprises a CRISPR repeat sequence comprising the nucleotide sequence set forth as any one of SEQ ID NOs: 546, 549-552, 839, 842, 845, 940, 942-945, 1228, 1230, and 1232, or an active variant or fragment thereof. In some embodiments, the guide RNA comprises a CRISPR repeat having the nucleotide sequence set forth as SEQ ID NO: 546, or an active variant or fragment thereof. In some embodiments, the guide RNA comprises a crRNA comprising the nucleotide sequence set forth as any one of SEQ ID NOs: 574-692, and 967-1085, or an active variant or fragment thereof. In some embodiments, the guide RNA comprises a tracrRNA comprising the nucleotide sequence set forth as any one of SEQ ID NOs: 547, 553-562, 840, 842, 846, 941, 946-955, 1229, 1231, and 1233, or an active variant or fragment thereof. In some embodiments, the guide RNA comprises a tracrRNA having the nucleotide sequence set forth as SEQ ID NO: 547, or an active variant or fragment thereof. In some embodiments, the guide RNA comprises an sgRNA backbone comprising any one of the nucleotide sequences set forth as SEQ ID NOs: 563-573, and 956-966. In some embodiments, the guide RNA comprises an sgRNA comprising any one of the nucleotide sequences set forth as SEQ ID NOs: 693-834, and 1086-1227, or an active variant or fragment thereof. In some embodiments, the guide RNA comprises a sgRNA having the nucleotide sequence set forth as SEQ ID NO: 693, or an active variant or fragment thereof. In some embodiments, the guide RNA comprises a sgRNA having the nucleotide sequence set forth as SEQ ID NO: 694, or an active variant or fragment thereof. In some embodiments, the guide RNA comprises a sgRNA having the nucleotide sequence set forth as SEQ ID NO: 695, or an active variant or fragment thereof. In some embodiments, the guide RNA comprises a sgRNA having the nucleotide sequence set forth as SEQ ID NO: 696, or an active variant or fragment thereof. In some embodiments, the guide RNA comprises a sgRNA having the nucleotide sequence set forth as SEQ ID NO: 697, or an active variant or fragment thereof. In some embodiments, the guide RNA comprises a sgRNA having the nucleotide sequence set forth as SEQ ID NO: 698, or an active variant or fragment thereof. The guide RNA of the system can be a single guide RNA or a dual-guide RNA. In some embodiments, the system comprises an RNA-guided nuclease that is heterologous to the guide RNA, wherein the RGN and guide RNA are not found complexed to one another (i.e., bound to one another) in nature.

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and an RNA-guided nuclease as the protein component. Such ribonucleoprotein complexes can be purified from a cell or organism that naturally expresses an RGN polypeptide and has been engineered to express a particular guide RNA that is specific for a target sequence of interest (e.g., a target sequence in the FOXP3 gene). Alternatively, the ribonucleoprotein complex can be purified from a cell or organism that has been transformed with polynucleotides (e.g., an mRNA) that encode an RGN polypeptide and a guide RNA and cultured under conditions to allow for the expression of the RGN polypeptide and guide RNA. In some embodiments, the ribonucleoprotein complex is purified from a cell or organism that has been transformed with a polynucleotide (e.g., an mRNA) that encodes an RGN polypeptide and wherein a synthetically derived gRNA has been introduced. Thus, methods are provided for making an RGN polypeptide or an RGN ribonucleoprotein complex. Such methods comprise culturing a cell comprising a nucleotide sequence encoding an RGN polypeptide, and in some embodiments a nucleotide sequence encoding a guide RNA, under conditions in which the RGN polypeptide (and in some embodiments, the guide RNA) is expressed. The RGN polypeptide or RGN ribonucleoprotein can then be purified from a lysate of the cultured cells. In some embodiments, the nucleotide sequence encoding an RGN polypeptide includes a mRNA (messenger RNA). In some embodiments, methods for assembling an RNP complex comprise combining one or more of the presently disclosed guide RNAs and one or more of the presently disclosed RGN polypeptides under conditions suitable for formation of the RNP complex.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide is recombinantly produced and comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG (e.g., 3× FLAG tag), HA, nus, Softag 1, Softag 3, Strep, SBP, Glu-Glu, HSV, KT3, S, S1, T7, V5, VSV-G, 6×His, 10×His, biotin carboxyl carrier protein (BCCP), and calmodulin. Generally, the tagged RGN polypeptide or RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals. Similarly, an “isolated” polynucleotide or nucleic acid molecule is removed from its naturally occurring environment. An isolated polynucleotide is substantially free of chemical precursors or other chemicals when chemically synthesized or has been removed from a genomic locus via the breaking of phosphodiester bonds. An isolated polynucleotide can be part of a vector, a composition of matter or can be contained within a cell so long as the cell is not the original environment of the polynucleotide.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, contacting”, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

Some aspects of this disclosure provide kits comprising one or more elements of an RGN system described herein, including: guide RNAs (i.e. crRNAs, tracrRNAs, and/or sgRNAs), RGNs, and/or polynucleotides encoding the same; cells; and complete RGN systems, and in some embodiments another type of nuclease. In some embodiments, the kit includes suitable reagents, buffers, and/or instructions for using one or more elements of an RGN system, e.g., for in vitro or in vivo nucleic acid editing. Reagents may be provided in any suitable container, such as a vial, a bottle, or a tube. Reagents may be used in a process utilizing one or more of the elements of an RGN system. For example, restriction enzymes may be included for cloning of a polynucleotide encoding an RGN or a guide RNA into a vector. In some embodiments, the kit includes instructions regarding the design and use of suitable guide RNAs (i.e. crRNAs, tracrRNAs, and/or sgRNAs) for targeted editing of a nucleic acid sequence. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

A kit including one or more elements of an RGN system of the disclosure has utility in a wide variety of applications including modifying (e.g., deleting, inserting, translocating, inactivating, activating) a target polynucleotide in a multiplicity of cell types.

In some embodiments, a kit of the disclosure includes a kit including a pharmaceutical composition described herein. In some embodiments, a kit may include: (a) a container containing a composition of the disclosure in lyophilized form and (b) a second container containing an acceptable diluent (e.g., sterile water) for injection. An acceptable diluent can be used for reconstitution or dilution of the lyophilized compound of the disclosure. Optionally associated with such container(s) can be a notice in the form prescribed by a governmental agency regulating the manufacture, use or sale of biological products.

### VII. Methods of Binding, Cleaving, or Modifying a Target Sequence

The present disclosure provides methods for binding, cleaving, and/or modifying a target sequence in the FOXP3 gene. The methods include delivering an RGN system comprising at least one guide RNA or a polynucleotide encoding the same, and at least one RGN polypeptide or a polynucleotide encoding the same to the target sequence or a cell or embryo comprising the target sequence. In some embodiments, the target sequence within the FOXP3 gene has a nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214. In some embodiments, the target sequence within the FOXP3 gene has the nucleotide sequence set forth as SEQ ID NO: 156. In some embodiments, the target sequence within the FOXP3 gene has the nucleotide sequence set forth as SEQ ID NO: 164. In some embodiments, the target sequence within the FOXP3 gene has the nucleotide sequence set forth as SEQ ID NO: 180. In some embodiments, the target sequence within the FOXP3 gene has the nucleotide sequence set forth as SEQ ID NO: 190. In some embodiments, the target sequence within the FOXP3 gene has the nucleotide sequence set forth as SEQ ID NO: 198. In some embodiments, the target sequence within the FOXP3 gene has the nucleotide sequence set forth as SEQ ID NO: 194.

In some embodiments, the RGN is capable of recognizing a consensus PAM sequence set forth as NNNNCC. In some embodiments, the RGN is capable of recognizing a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. The RGN can comprise an amino acid sequence set forth as SEQ ID NO: 545, or an active variant or fragment thereof. In some embodiments, the RGN comprises an amino acid sequence having at least 80% sequence identity to SEQ ID NO: 545. The guide RNA can comprise a CRISPR repeat sequence comprising the nucleotide sequence set forth as any one of SEQ ID NOs: 546, 549-552, 839, 842, 845, 940, 942-945, 1228, 1230, and 1232, or an active variant or fragment thereof. In some embodiments, the guide RNA comprises a CRISPR repeat having the nucleotide sequence set forth as SEQ ID NO: 546, or an active variant or fragment thereof. The guide RNA can comprise a crRNA comprising the nucleotide sequence set forth as any one of SEQ ID NOs: 574-692, and 967-1085, or an active variant or fragment thereof. The guide RNA can comprise a tracrRNA comprising the nucleotide sequence set forth as any one of SEQ ID NOs: 547, 553-562, 840, 842, 846, 941, 946-955, 1229, 1231, and 1233, or an active variant or fragment thereof. In some embodiments, the guide RNA comprises a tracrRNA having the nucleotide sequence set forth as SEQ ID NO: 547, or an active variant or fragment thereof. The guide RNA can comprise an sgRNA backbone comprising any one of the nucleotide sequences set forth as SEQ ID NOs: 563-573, and 956-966, or an active variant or fragment thereof. The guide RNA can comprise an sgRNA comprising any one of the nucleotide sequences set forth as SEQ ID NOs: 693-834, and 1086-1227, or an active variant or fragment thereof. In some embodiments, the guide RNA comprises a sgRNA having the nucleotide sequence set forth as SEQ ID NO: 693, or an active variant or fragment thereof. In some embodiments, the guide RNA comprises a sgRNA having the nucleotide sequence set forth as SEQ ID NO: 694, or an active variant or fragment thereof. In some embodiments, the guide RNA comprises a sgRNA having the nucleotide sequence set forth as SEQ ID NO: 695, or an active variant or fragment thereof. In some embodiments, the guide RNA comprises a sgRNA having the nucleotide sequence set forth as SEQ ID NO: 696, or an active variant or fragment thereof. In some embodiments, the guide RNA comprises a sgRNA having the nucleotide sequence set forth as SEQ ID NO: 697, or an active variant or fragment thereof. In some embodiments, the guide RNA comprises a sgRNA having the nucleotide sequence set forth as SEQ ID NO: 698, or an active variant or fragment thereof. The guide RNA of the system can be a single guide RNA or a dual-guide RNA.

The RGN of the system may be a nuclease dead RGN, have nickase activity, or may be a fusion polypeptide. In some embodiments, the RGN fusion protein comprises a polypeptide that recruits members of a functional nucleic acid repair complex, such as a member of the nucleotide excision repair (NER) or transcription coupled-nucleotide excision repair (TC-NER) pathway (Wei et al., 2015, PNAS USA 112(27):E3495-504; Troelstra et al., 1992, Cell 71:939-953; Marnef et al., 2017, J Mol Biol 429(9):1277-1288), as described in U.S. Provisional Application No. 62/966,203, which was filed on Jan. 27, 2020, and is incorporated by reference in its entirety. In some embodiments, the RGN fusion protein comprises CSB (van den Boom et al., 2004, J Cell Biol 166(1):27-36; van Gool et al., 1997, EMBO J 16(19):5955-65; an example of which is set forth as SEQ ID NO: 935), which is a member of the TC-NER (nucleotide excision repair) pathway and functions in the recruitment of other members. In further embodiments, the RGN fusion protein comprises an active domain of CSB, such as the acidic domain of CSB which comprises amino acid residues 356-394 of SEQ ID NO: 935 (Teng et al., 2018, Nat Commun 9(1):4115).

In certain embodiments, the RGN and/or guide RNA is heterologous to the cell or embryo to which the RGN and/or guide RNA (or polynucleotide(s) encoding at least one of the RGN and guide RNA) are introduced.

In embodiments wherein the method comprises delivering a polynucleotide encoding a guide RNA and/or an RGN polypeptide, the cell or embryo can then be cultured under conditions in which the guide RNA and/or RGN polypeptide are expressed. In some embodiments, the method comprises contacting a target nucleic acid molecule with an RGN ribonucleoprotein complex. The RGN ribonucleoprotein complex may comprise an RGN that is nuclease dead or has nickase activity. In some embodiments, the method comprises introducing into a cell or embryo comprising a target nucleic acid molecule an RGN ribonucleoprotein complex. The RGN ribonucleoprotein complex can be one that has been purified from a biological sample, recombinantly produced and subsequently purified, or in vitro-assembled as described herein. In embodiments wherein the RGN ribonucleoprotein complex that is contacted with the target nucleic acid molecule, or cell or embryo, has been assembled in vitro, the method can further comprise the in vitro assembly of the complex prior to contact with the target nucleic acid molecule, cell or embryo.

A purified or in vitro assembled RGN ribonucleoprotein complex can be introduced into a cell or embryo using any method known in the art, including, but not limited to electroporation. Alternatively, an RGN polypeptide and/or polynucleotide encoding or comprising the guide RNA can be introduced into a cell or embryo using any method known in the art (e.g., electroporation).

Upon delivery to or contact with the target nucleic acid molecule or cell or embryo comprising the target nucleic acid molecule, the guide RNA directs the RGN to bind to the target sequence within the target nucleic acid molecule in a sequence-specific manner. In those embodiments wherein the RGN has nuclease activity, the RGN polypeptide cleaves the target sequence upon binding. The target sequence can subsequently be modified via endogenous repair mechanisms, such as non-homologous end joining, or homology-directed repair with a provided donor polynucleotide.

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target nucleic acid molecule comprising a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of only one RGN and only one of the presently disclosed guide RNAs. In some embodiments, the methods involve the use of a single type of RGN complexed with more than one guide RNA. In some embodiments, the methods involve the use of two types of RGNs, each complexed with a guide RNA. The more than one guide RNA can target different regions of a single gene or can target multiple genes. For example, a first guide RNA can target exon 1 in the FOXP3 gene and a second guide RNA can target intron 1 in the FOXP3 gene.

In those embodiments wherein a donor polynucleotide is not provided, a double-stranded break introduced by an RGN polypeptide can be repaired by a non-homologous end-joining (NHEJ) repair process. Due to the error-prone nature of NHEJ, repair of the double-stranded break can result in a mutation to the target sequence. In certain embodiments, a “mutation” in reference to a nucleic acid molecule refers to a change in the nucleotide sequence of the nucleic acid molecule, which can be a deletion, insertion, or substitution of one or more nucleotides, or a combination thereof. Mutation of the target nucleic acid molecule comprising a target sequence can result in the expression of an altered protein product or inactivation of a coding sequence.

The methods can comprise integrating a donor polynucleotide into the FOXP3 gene using an RGN system of the disclosure. In those embodiments wherein a donor polynucleotide is present, the donor sequence in the donor polynucleotide can be integrated into or exchanged with the target nucleotide sequence during the course of repair of the introduced double-stranded break, resulting in the introduction of the exogenous donor sequence. A donor polynucleotide thus comprises a donor sequence that is desired to be introduced into a target sequence of interest (e.g., a target sequence in the FOXP3 gene). In some embodiments, the donor sequence alters the original target nucleotide sequence such that the newly integrated donor sequence will not be recognized and cleaved by the RGN. Integration of the donor sequence can be enhanced by the inclusion within the donor polynucleotide of flanking sequences, referred to herein as “homology arms” that have substantial sequence identity with the sequences flanking the target nucleotide sequence, allowing for a homology-directed repair process. In some embodiments, homology arms have a length of at least 50 base pairs, at least 100 base pairs, and up to 2000 base pairs or more, and have at least 90%, at least 95%, or more, sequence homology to their corresponding sequence within the target nucleotide sequence.

In those embodiments wherein the RGN polypeptide introduces double-stranded staggered breaks, the donor polynucleotide can comprise a donor sequence flanked by compatible overhangs, allowing for direct ligation of the donor sequence to the cleaved target nucleotide sequence comprising overhangs by a non-homologous repair process during repair of the double-stranded break.

In those embodiments wherein the method involves the use of an RGN that is a nickase (i.e., is only able to cleave a single strand of a double-stranded polynucleotide), the method can comprise introducing two RGN nickases that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide.

In some embodiments, a method is provided for binding a target nucleotide sequence and detecting the target sequence, wherein the method comprises introducing into a cell or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN and further comprises a detectable label, and the method further comprises detecting the detectable label. The detectable label may be fused to the RGN as a fusion protein (e.g., fluorescent protein) or may be a small molecule conjugated to or incorporated within the RGN polypeptide that can be detected visually or by other means.

Also provided herein are methods for modulating the expression of a FOXP3 gene. In some embodiments, the methods comprise modulating expression of a FOXP3 gene in a population of cells. In some embodiments, the population of cells comprises T cells. The method can comprise comprising delivering an RGN system or an RNP complex described herein to the population of cells, wherein the population of cells comprises a target sequence within the FOXP3 gene, and wherein FOXP3 gene expression is modulated as compared to FOXP3 gene expression in a control population of cells. In some embodiments, cleavage or modification of the target sequence occurs. Cleavage or modification of the target sequence can be detected by sequencing. FOXP3 gene expression can be measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof. In some embodiments, FOXP3 gene expression is decreased. The decrease in FOXP3 gene expression can comprise a decrease in FOXP3 mRNA level and/or Foxp3 protein level. In some embodiments, the decrease in FOXP3 mRNA level and/or Foxp3 protein level is due to cleavage of the FOXP3 gene by an RGN system of the disclosure. Cleavage or modification of the target sequence can occur at a rate of 40% to 100%, or 60% to 99%, or 70% to 90%. In some embodiments, cleavage or modification of the target sequence can occur at a rate of at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, cleavage or modification of the target sequence occurs at a rate of 80% to 100%. The control population of cells can include a population of cells that has not been subjected to the delivering.

In some embodiments, methods for modulating the expression of a FOXP3 gene comprise introducing into a cell or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN. In some embodiments, the nuclease-dead RGN is a fusion protein comprising an expression modulator as described herein.

The methods can comprise activation of the FOXP3 gene using an RGN system of the disclosure. In some embodiments, an RGN system can be targeted to the FOXP3 gene to increase or activate expression of the gene. In some embodiments, the increase or activation of the FOXP3 gene is effected by the RGN system directly and in other embodiments the increase or activation of the FOXP3 gene is effected via integration of a donor polynucleotide. The RGN (e.g., a nuclease-dead RGN) or its complexed guide RNA can be operably fused to an expression modulator such that binding of the RGN/guide RNA complex to a target sequence within the FOXP3 gene serves to increase or activate expression of the FOXP3 gene. In some embodiments, the expression modulator comprises a transcriptional activation domain, which interacts with transcriptional control elements and/or transcriptional regulatory proteins, such as RNA polymerases and transcription factors, to increase or activate transcription of the FOXP3 gene. Transcriptional activation domains are known in the art and include, but are not limited to, a herpes simplex virus VP16 activation domain and an NFAT activation domain.

One of ordinary skill in the art will appreciate that any of the presently disclosed methods can be used to target a single target sequence or multiple target sequences in the FOXP3 gene. Thus, methods comprise the use of a single RGN polypeptide in combination with multiple, distinct guide RNAs, which can target multiple, distinct sequences within the FOXP3 gene.

In some embodiments, methods of the disclosure are performed ex vivo or in vitro. In some embodiments, methods of the disclosure do not include methods for treatment of the human or animal body by therapy. In some embodiments, methods of the disclosure do not include methods that comprise a process for modifying the germ line genetic identity of human beings or does not comprise a use of human embryos for industrial or commercial purposes.

### IX. Cells Comprising a Polynucleotide Genetic Modification

Provided herein are cells and organisms comprising a target sequence in the FOXP3 gene that has been modified using a process mediated by an RGN, crRNA, tracrRNA, and/or sgRNA as described herein. In some embodiments, the RGN is capable of recognizing a consensus PAM sequence set forth as NNNNCC. In some embodiments, the RGN is capable of recognizing a full PAM sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC. The RGN can comprise an amino acid sequence set forth as SEQ ID NO: 545, or an active variant or fragment thereof. In some embodiments, the RGN comprises an amino acid sequence having at least 80% sequence identity to SEQ ID NO: 545. The guide RNA can comprise a CRISPR repeat sequence comprising the nucleotide sequence set forth as any one of SEQ ID NOs: 546, 549-552, 839, 842, 845, 940, 942-945, 1228, 1230, and 1232, or an active variant or fragment thereof. In some embodiments, the guide RNA comprises a CRISPR repeat having the nucleotide sequence set forth as SEQ ID NO: 546, or an active variant or fragment thereof. The guide RNA can comprise a crRNA comprising the nucleotide sequence set forth as any one of SEQ ID NOs: 574-692, and 967-1085, or an active variant or fragment thereof. The guide RNA can comprise a tracrRNA comprising the nucleotide sequence set forth as any one of SEQ ID NOs: 547, 553-562, 840, 842, 846, 941, 946-955, 1229, 1231, and 1233, or an active variant or fragment thereof. In some embodiments, the guide RNA comprises a tracrRNA having the nucleotide sequence set forth as SEQ ID NO: 547, or an active variant or fragment thereof. The guide RNA can comprise an sgRNA backbone comprising the nucleotide sequence set forth as any one of SEQ ID NOs: 563-573, and 956-966, or an active variant or fragment thereof. The guide RNA can comprise an sgRNA comprising the nucleotide sequences set forth as any one of SEQ ID NOs: 693-834, and 967-1085, or an active variant or fragment thereof. In some embodiments, the guide RNA comprises a sgRNA having the nucleotide sequence set forth as SEQ ID NO: 693, or an active variant or fragment thereof. In some embodiments, the guide RNA comprises a sgRNA having the nucleotide sequence set forth as SEQ ID NO: 694, or an active variant or fragment thereof. In some embodiments, the guide RNA comprises a sgRNA having the nucleotide sequence set forth as SEQ ID NO: 695, or an active variant or fragment thereof. In some embodiments, the guide RNA comprises a sgRNA having the nucleotide sequence set forth as SEQ ID NO: 696, or an active variant or fragment thereof. In some embodiments, the guide RNA comprises a sgRNA having the nucleotide sequence set forth as SEQ ID NO: 697, or an active variant or fragment thereof. In some embodiments, the guide RNA comprises a sgRNA having the nucleotide sequence set forth as SEQ ID NO: 698, or an active variant or fragment thereof. The guide RNA of the system can be a single guide RNA or a dual-guide RNA.

The modified cells can be eukaryotic (e.g., mammalian, insect, avian cell) or prokaryotic. Prokaryotic cells can be from species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

Eukaryotic cells can include cells from animals (e.g., mammals, insects, fish, birds, and reptiles), fungi, amoeba, algae, and yeast. In some embodiments, the cell that is modified by the presently disclosed methods include lymphocytes. In some embodiments, lymphocytes include cytotoxic T cells or regulatory T cells. Cytotoxic T cells recognize and destroy infected, damaged, or cancerous cells and can be identified by various markers including CD8; CD45; CD54; tumor necrosis factor (TNF) alpha, interferon (IFN) gamma, IL-2 CXCR3, and/or TBX21 for Tc1; IL-4, IL-5, CCR4, and/or GATA3 for Tc2; IL-9, IL-10, and/or IRF4 for Tc9; and CCR6, KLRB1, IL-17, IRF4, and/or RORC for Tc17. Regulatory T cells modulate or suppress immune responses by, for example, secreting anti-inflammatory cytokines, expressing inhibitory proteins, and/or inducing apoptosis of effector T cells by cytokine deprivation, and can be identified by various markers including FoxP3, IL-2 receptor alpha (IL2RA or CD25), STAT5A, CTLA4, IL-10, and/or transforming growth factor (TGF) beta. Also provided are embryos comprising at least one FOXP3 gene that has been modified by a process utilizing an RGN, crRNA, tracrRNA, and/or sgRNA as described herein. The genetically modified cells, organisms, and embryos can be heterozygous or homozygous for the modified FOXP3 gene.

In some embodiments, the chromosomal modification of the cell, organism, or embryo can result in downregulation or abolishment of expression of the FOXP3 mRNA or protein encoded by the FOXP3 gene. In embodiments, the chromosomal modification results in the production of a FOXP3 mRNA that has decreased translation of the Foxp3 protein as compared to a FOXP3 mRNA transcribed from a wild-type FOXP3 gene of a cell, organism, or embryo that has not undergone chromosomal modification. In some embodiments, the chromosomal modification results in the production of a variant Foxp3 protein product that is less stable or reduced in expression as compared to a Foxp3 protein encoded by a wild-type FOXP3 gene of a cell, organism or embryo that has not undergone chromosomal modification. In some embodiments, the expressed variant Foxp3 protein can have at least one amino acid substitution and/or the addition or deletion of at least one amino acid. The variant Foxp3 protein encoded by the altered chromosomal sequence can exhibit modified characteristics or activities when compared to the wild-type Foxp3 protein, including but not limited to altered ability to activate or repress Foxp3 target genes.

Cells that have been modified may be introduced into an organism. These cells could have originated from the same organism (e.g., person) in the case of autologous cellular transplants, wherein the cells are modified in an ex vivo approach. Alternatively, the cells originated from another organism within the same species (e.g., another person) in the case of allogeneic cellular transplants.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

Non-limiting embodiments include:
1. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
   wherein the tracrRNA comprises:
(iii) an anti-repeat; and
(iv) a tail,
   wherein the spacer is capable of hybridizing to a target sequence in a forkhead box P3 (FOXP3) gene,
   wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.
2. The gRNA of embodiment 1, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.
3. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 5 nucleotides.
4. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 4 nucleotides.
5. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 3 nucleotides.
6. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 2 nucleotides.
7. The gRNA of embodiment 2, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 nucleotide.
8. The gRNA of embodiment 1, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.
9. The gRNA of any one of embodiments 1-8, wherein the crRNA repeat has the nucleotide sequence set forth as SEQ ID NO: 546 or a nucleotide sequence that differs in length and/or sequence from SEQ ID NO: 546 by 1 to 8 nucleotides.
10. The gRNA of embodiment 9, wherein the crRNA repeat has a nucleotide sequence that differs in length and/or sequence from SEQ ID NO: 546 by 8 nucleotides.
11. The gRNA of embodiment 9, wherein the crRNA repeat has a nucleotide sequence that differs in length and/or sequence from SEQ ID NO: 546 by 7 nucleotides.
12. The gRNA of embodiment 9, wherein the crRNA repeat has a nucleotide sequence that differs in length and/or sequence from SEQ ID NO: 546 by 6 nucleotides.
13. The gRNA of embodiment 9, wherein the crRNA repeat has a nucleotide sequence that differs in length and/or sequence from SEQ ID NO: 546 by 5 nucleotides.
14. The gRNA of embodiment 9, wherein the crRNA repeat has a nucleotide sequence that differs in length and/or sequence from SEQ ID NO: 546 by 4 nucleotides.
15. The gRNA of embodiment 9, wherein the crRNA repeat has a nucleotide sequence that differs in length and/or sequence from SEQ ID NO: 546 by 3 nucleotides.
16. The gRNA of embodiment 9, wherein the crRNA repeat has a nucleotide sequence that differs in length and/or sequence from SEQ ID NO: 546 by 2 nucleotides.
17. The gRNA of embodiment 9, wherein the crRNA repeat has a nucleotide sequence that differs in length and/or sequence from SEQ ID NO: 546 by 1 nucleotide.
18. The gRNA of embodiment 4, wherein the crRNA repeat has the nucleotide sequence set forth as any one of SEQ ID NOs: 546, 549-552, 839, 842, and 845.
19. The gRNA of any one of embodiments 1-9, wherein the crRNA has a nucleotide sequence having at least 80% sequence identity to SEQ ID NOs: 574-692.
20. The gRNA of embodiment 19, wherein the crRNA has a nucleotide sequence having at least 90% sequence identity to any one of SEQ ID NOs: 574-692.
21. The gRNA of embodiment 19, wherein the crRNA has a nucleotide sequence having at least 95% sequence identity to any one of SEQ ID NOs: 574-692.
22. The gRNA of embodiment 19, wherein the crRNA has the nucleotide sequence set forth as any one of SEQ ID NOs: 574-692.
23. The gRNA of any one of embodiments 1-9, wherein the tracrRNA has a nucleotide sequence having at least 80% sequence identity to SEQ ID NO: 547.
24. The gRNA of embodiment 23, wherein the tracrRNA has a nucleotide sequence having at least 90% sequence identity to SEQ ID NO: 547.
25. The gRNA of embodiment 23, wherein the tracrRNA has a nucleotide sequence having at least 95% sequence identity to SEQ ID NO: 547.
26. The gRNA of any one of embodiments 1-9, wherein the tracrRNA has a nucleotide sequence that differs in length from SEQ ID NO: 547 by 1 to 16 nucleotides.
27. The gRNA of embodiment 26, wherein the tracrRNA has a nucleotide sequence that is 8 nucleotides shorter than SEQ ID NO: 547.
28. The gRNA of embodiment 26, wherein the tracrRNA has a nucleotide sequence that is 11 nucleotides shorter than SEQ ID NO: 547.
29. The gRNA of any one of embodiments 23-28, wherein the tracrRNA has the nucleotide sequence set forth as any one of SEQ ID NOs: 547, 553-562, 840, 842, and 846.
30. The gRNA of any one of embodiments 1-8, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
31. The gRNA of embodiment 30, wherein the linker has a nucleotide sequence set forth as AAAG, GAAA, ACUU, or CAAAGG.
32. The gRNA of embodiment 31, wherein the linker has a nucleotide sequence set forth as AAAG.
33. The gRNA of any one of embodiments 30-32, wherein the backbone of the sgRNA comprises a total length of at least 85, 90, 95, 100, 105, 110, 115, or 120 nucleotides.
34. The gRNA of any one of embodiments 30-32, wherein the backbone of the sgRNA comprises a total length of at most 85, 90, 95, 100, 105, 110, 115, or 120 nucleotides.
35. The gRNA of any one of embodiments 30-32, wherein the backbone of the sgRNA comprises a total length of 86 to 98 nucleotides.
36. The gRNA of any one of embodiments 30-32, wherein the backbone of the sgRNA comprises a total length of 94 nucleotides.
37. The gRNA of any one of embodiments 30-32, wherein the backbone of the sgRNA has a nucleotide sequence having at least 80% sequence identity to any one of SEQ ID NOs: 563-573.
38. The gRNA of embodiment 37, wherein the sgRNA backbone has a nucleotide sequence having at least 90% sequence identity to any one of SEQ ID NOs: 563-573.
39. The gRNA of embodiment 37, wherein the sgRNA backbone has a nucleotide sequence having at least 95% sequence identity to any one of SEQ ID NOs: 563-573.
40. The gRNA of embodiment 37, wherein the sgRNA backbone has the nucleotide sequence set forth as any one of SEQ ID NOs: 563-573.
41. The gRNA of any one of embodiments 1-8, wherein the gRNA comprises a first stem loop formed by hybridization of the crRNA repeat and the anti-repeat, wherein the first stem loop comprises a first stem and a second stem, and wherein the first stem of the first stem loop comprises a total length of at least 3, 4, 5, 6, 7, 8, 9, 10, or 11 base pairs (bp).
42. The gRNA of any one of embodiments 1-8, wherein the gRNA comprises a first stem loop formed by hybridization of the crRNA repeat and the anti-repeat, wherein the first stem loop comprises a first stem and a second stem, and wherein the first stem of the first stem loop comprises a total length of at most 3, 4, 5, 6, 7, 8, 9, 10, or 11 bp.
43. The gRNA of embodiment 41 or 42, wherein the first stem of the first stem loop comprises a total length of 6 bp.
44. The gRNA of embodiment 41 or 42, wherein the first stem of the first stem loop comprises a total length of 3 bp.
45. The gRNA of any one of embodiments 1-8, wherein the tail of the tracrRNA comprises a total length of at least 1, 2, 3, 4, 5, 6, or 7 nucleotides.
46. The gRNA of any one of embodiments 1-8, wherein the tail of the tracrRNA comprises a total length of at most 1, 2, 3, 4, 5, 6, or 7 nucleotides.
47. The gRNA of embodiment 45 or 46, wherein the tail of the tracrRNA comprises a total length of 3 nucleotides.
48. The gRNA of embodiment 45 or 46, wherein the tail of the tracrRNA comprises a total length of 1 nucleotide.
49. The gRNA of embodiment 41 or 42, wherein the gRNA further comprises a second stem loop most proximal to the tail, wherein the second stem loop comprises a first stem and a second stem.
50. The gRNA of embodiment 49, wherein the first stem of the second stem loop comprises a total length of at least 1, 2, 3, 4, 5, or 6 bp.
51. The gRNA of embodiment 49, wherein the first stem of the second stem loop comprises a total length of at most 1, 2, 3, 4, 5, or 6 bp.
52. The gRNA of embodiment 50 or 51, wherein the first stem of the second stem loop comprises a total length of 5 bp.
53. The gRNA of any one of embodiments 49-52, wherein the first stem of the first stem loop comprises a total length of 6 bp, the tail of the tracrRNA comprises a total length of 3 nucleotides, and the first stem of the second stem loop comprises a total length of 5 bp.
54. The gRNA of any one of embodiments 1-8, wherein the gRNA is a dual guide RNA (dgRNA).
55. The gRNA of embodiment 54, wherein the crRNA repeat of the dgRNA comprises a total length of at least 10, 11, 12, 13, 14, 15, 16, 17, 18, 19, 20, or 21 nucleotides.
56. The gRNA of embodiment 54, wherein the crRNA repeat of the dgRNA comprises a total length of at most 10, 11, 12, 13, 14, 15, 16, 17, 18, 19, 20, or 21 nucleotides.
57. The gRNA of embodiment 55 or 56, wherein the crRNA repeat of the dgRNA comprises a total length of 13 nucleotides.
58. The gRNA of embodiment 55 or 56, wherein the crRNA repeat of the dgRNA comprises a total length of 16 nucleotides.
59. The gRNA of embodiment 55 or 56, wherein the crRNA repeat of the dgRNA comprises a total length of 21 nucleotides.
60. The gRNA of embodiment 54, wherein the tracrRNA of the dgRNA comprises a total length of at least 65, 70, 75, 80, or 85 nucleotides.
61. The gRNA of embodiment 54, wherein the tracrRNA of the dgRNA comprises a total length of at most 65, 70, 75, 80, or 85 nucleotides.
62. The gRNA of embodiment 60 or 61, wherein the tracrRNA of the dgRNA comprises a total length of 74 nucleotides.
63. The gRNA of embodiment 60 or 61, wherein the tracrRNA of the dgRNA comprises a total length of 77 nucleotides.
64. The gRNA of any one of embodiments 1-63, wherein the gRNA comprises a total length of at least 85, 90, 95, 100, 105, 110, 115, 120, 125, 130, or 135 nucleotides.
65. The gRNA of any one of embodiments 1-63, wherein the gRNA comprises a total length of at most 85, 90, 95, 100, 105, 110, 115, 120, 125, 130, or 135 nucleotides.
66. The gRNA of any one of embodiments 1-63, wherein the gRNA comprises a total length of 106 to 135 nucleotides.
67. The gRNA of embodiment 66, wherein the gRNA comprises a total length of 117 to 119 nucleotides.
68. The gRNA of any one of embodiments 1-67, wherein the gRNA is capable of targeting a bound RNA-guided nuclease (RGN) polypeptide to the target sequence.
69. The gRNA of embodiment 68, wherein the RGN polypeptide is capable of recognizing a consensus protospacer adjacent motif (PAM) having the nucleotide sequence set forth as NNNNCC.
70. The gRNA of embodiment 69, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.
71. The gRNA of any one of embodiments 68-70, wherein the RGN polypeptide has an amino acid sequence having at least 80% sequence identity to SEQ ID NO: 545; and wherein the target sequence and the spacer are selected from the group consisting of:
a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides;
b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides;
c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides;
d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides;
e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and
f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides.
72. The gRNA of embodiment 71, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 5 nucleotides;
b) a target sequence having the nucleotide sequence set forth as SEQ ID NO: 164 and a spacer having a nucleotide sequence that differs in length and/or sequence from SEQ ID NO: 163 by 5 nucleotides;
c) a target sequence having the nucleotide sequence set forth as SEQ ID NO: 190 and a spacer having a nucleotide sequence that differs in length and/or sequence from SEQ ID NO: 189 by 5 nucleotides;
d) a target sequence having the nucleotide sequence set forth as SEQ ID NO: 180 and a spacer having a nucleotide sequence that differs in length and/or sequence from SEQ ID NO: 179 by 5 nucleotides;
e) a target sequence having the nucleotide sequence set forth as SEQ ID NO: 198 and a spacer having a nucleotide sequence that differs in length and/or sequence from SEQ ID NO: 197 by 5 nucleotides; and
f) a target sequence having the nucleotide sequence set forth as SEQ ID NO: 194 and a spacer having a nucleotide sequence that differs in length and/or sequence from SEQ ID NO: 193 by 5 nucleotides.
73. The gRNA of embodiment 71, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 4 nucleotides;
b) a target sequence having the nucleotide sequence set forth as SEQ ID NO: 164 and a spacer having a nucleotide sequence that differs in length and/or sequence from SEQ ID NO: 163 by 4 nucleotides;
c) a target sequence having the nucleotide sequence set forth as SEQ ID NO: 190 and a spacer having a nucleotide sequence that differs in length and/or sequence from SEQ ID NO: 189 by 4 nucleotides;
d) a target sequence having the nucleotide sequence set forth as SEQ ID NO: 180 and a spacer having a nucleotide sequence that differs in length and/or sequence from SEQ ID NO: 179 by 4 nucleotides;
e) a target sequence having the nucleotide sequence set forth as SEQ ID NO: 198 and a spacer having a nucleotide sequence that differs in length and/or sequence from SEQ ID NO: 197 by 4 nucleotides; and
f) a target sequence having the nucleotide sequence set forth as SEQ ID NO: 194 and a spacer having a nucleotide sequence that differs in length and/or sequence from SEQ ID NO: 193 by 4 nucleotides.
74. The gRNA of embodiment 71, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 3 nucleotides;
b) a target sequence having the nucleotide sequence set forth as SEQ ID NO: 164 and a spacer having a nucleotide sequence that differs in length and/or sequence from SEQ ID NO: 163 by 3 nucleotides;
c) a target sequence having the nucleotide sequence set forth as SEQ ID NO: 190 and a spacer having a nucleotide sequence that differs in length and/or sequence from SEQ ID NO: 189 by 3 nucleotides;
d) a target sequence having the nucleotide sequence set forth as SEQ ID NO: 180 and a spacer having a nucleotide sequence that differs in length and/or sequence from SEQ ID NO: 179 by 3 nucleotides;
e) a target sequence having the nucleotide sequence set forth as SEQ ID NO: 198 and a spacer having a nucleotide sequence that differs in length and/or sequence from SEQ ID NO: 197 by 3 nucleotides; and
f) a target sequence having the nucleotide sequence set forth as SEQ ID NO: 194 and a spacer having a nucleotide sequence that differs in length and/or sequence from SEQ ID NO: 193 by 3 nucleotides.
75. The gRNA of embodiment 71, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 2 nucleotides;
b) a target sequence having the nucleotide sequence set forth as SEQ ID NO: 164 and a spacer having a nucleotide sequence that differs in length and/or sequence from SEQ ID NO: 163 by 2 nucleotides;
c) a target sequence having the nucleotide sequence set forth as SEQ ID NO: 190 and a spacer having a nucleotide sequence that differs in length and/or sequence from SEQ ID NO: 189 by 2 nucleotides;
d) a target sequence having the nucleotide sequence set forth as SEQ ID NO: 180 and a spacer having a nucleotide sequence that differs in length and/or sequence from SEQ ID NO: 179 by 2 nucleotides;
e) a target sequence having the nucleotide sequence set forth as SEQ ID NO: 198 and a spacer having a nucleotide sequence that differs in length and/or sequence from SEQ ID NO: 197 by 2 nucleotides; and
f) a target sequence having the nucleotide sequence set forth as SEQ ID NO: 194 and a spacer having a nucleotide sequence that differs in length and/or sequence from SEQ ID NO: 193 by 2 nucleotides.
76. The gRNA of embodiment 71, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 1 nucleotide;
b) a target sequence having the nucleotide sequence set forth as SEQ ID NO: 164 and a spacer having a nucleotide sequence that differs in length and/or sequence from SEQ ID NO: 163 by 1 nucleotide;
c) a target sequence having the nucleotide sequence set forth as SEQ ID NO: 190 and a spacer having a nucleotide sequence that differs in length and/or sequence from SEQ ID NO: 189 by 1 nucleotide;
d) a target sequence having the nucleotide sequence set forth as SEQ ID NO: 180 and a spacer having a nucleotide sequence that differs in length and/or sequence from SEQ ID NO: 179 by 1 nucleotide;
e) a target sequence having the nucleotide sequence set forth as SEQ ID NO: 198 and a spacer having a nucleotide sequence that differs in length and/or sequence from SEQ ID NO: 197 by 1 nucleotide; and
f) a target sequence having the nucleotide sequence set forth as SEQ ID NO: 194 and a spacer having a nucleotide sequence that differs in length and/or sequence from SEQ ID NO: 193 by 1 nucleotide.
77. The gRNA of embodiment 71, wherein the spacer has the nucleotide sequence set forth as any one of SEQ ID NOs: 155, 163, 189, 179, 197, and 193.
78. The gRNA of any one of embodiments 71-77, wherein the RGN polypeptide has an amino acid sequence having at least 90% sequence identity to SEQ ID NO: 545.
79. The gRNA of any one of embodiments 71-77, wherein the RGN polypeptide has an amino acid sequence having at least 95% sequence identity to SEQ ID NO: 545.
80. The gRNA of any one of embodiments 71-77, wherein the RGN polypeptide has the amino acid sequence set forth as SEQ ID NO: 545.
81. The gRNA of any one of embodiments 68-80, wherein the gRNA has a nucleotide sequence having at least 80% sequence identity to any one of SEQ ID NOs: 693-834.
82. The gRNA of embodiment 81, wherein the gRNA has a nucleotide sequence having at least 90% sequence identity to any one of SEQ ID NOs: 693-834.
83. The gRNA of embodiment 81, wherein the gRNA has a nucleotide sequence having at least 95% sequence identity to any one of SEQ ID NOs: 693-834.
84. The gRNA of embodiment 81, wherein the gRNA has a nucleotide sequence set forth as any one of SEQ ID NOs: 693-834.
85. The gRNA of embodiment 84, wherein the gRNA has a nucleotide sequence set forth as any one of SEQ ID NOs: 693, 694, 695, 696, 697, and 698.
86. The gRNA of embodiment 70, wherein the RGN polypeptide is capable of recognizing a full PAM having the nucleotide sequence set forth as any one of GGGTCCTT, GGGGCCGA, GGGGCCCA, CGGCCCTG, GGGCCCAT, TGGCCC, TGGGCC, GGGCCC, CGGGCC, and AGGGCC.
87. The gRNA of embodiment 86, wherein the RGN polypeptide has an amino acid sequence having at least 80% sequence identity to SEQ ID NO: 835.
88. The gRNA of embodiment 87, wherein the RGN polypeptide has an amino acid sequence having at least 90% sequence identity to SEQ ID NO: 835.
89. The gRNA of embodiment 87, wherein the RGN polypeptide has an amino acid sequence having at least 95% sequence identity to SEQ ID NO: 835.
90. The gRNA of embodiment 87, wherein the RGN polypeptide has the amino acid sequence set forth as SEQ ID NO: 835.
91. The gRNA of embodiment 70, wherein the RGN polypeptide is capable of recognizing a full PAM having the nucleotide sequence set forth as any one of TCGGCCCT, CAGGCCTG, TCGGCC, and CGGGCC.
92. The gRNA of embodiment 91, wherein the RGN polypeptide has an amino acid sequence having at least 80% sequence identity to SEQ ID NO: 915.
93. The gRNA of embodiment 92, wherein the RGN polypeptide has an amino acid sequence having at least 90% sequence identity to SEQ ID NO: 915.
94. The gRNA of embodiment 92, wherein the RGN polypeptide has an amino acid sequence having at least 95% sequence identity to SEQ ID NO: 915.
95. The gRNA of embodiment 92, wherein the RGN polypeptide has the amino acid sequence set forth as SEQ ID NO: 915.
96. The gRNA of any one of embodiments 1-95, wherein the gRNA comprises at least one chemical modification.
97. The gRNA of embodiment 96, wherein the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.
98. The gRNA of embodiment 97, wherein the at least one chemical modification comprises MS modifications at the 3 terminal nucleotides at the 5′ region and at the 3 terminal nucleotides at the 3′ region of the gRNA.
99. The gRNA of embodiment 98, wherein the crRNA repeat has the nucleotide sequence set forth as any one of SEQ ID NOs: 940, 942-945, 1228, 1230, and 1232.
100. The gRNA of embodiment 98 or 99, wherein the crRNA has the nucleotide sequence set forth as any one of SEQ ID NOs: 967-1085.
101. The gRNA of any one of embodiments 98-100, wherein the tracrRNA has the nucleotide sequence set forth as any one of SEQ ID NOs: 941, 946-955, 1229, 1231, and 1233.
102. The gRNA of any one of embodiments 98-101, wherein the gRNA has the nucleotide sequence set forth as any one of SEQ ID NOs: 1086-1227.
103. The gRNA of embodiment 97, wherein the BNA comprises a 2′,4′ BNA modification.
104. The gRNA of embodiment 103, wherein the 2′,4′ BNA modification is selected from the group consisting of: locked nucleic acid (LNA) modification, BNANC[N-Me]modification, 2′-O,4′-C-ethylene bridged nucleic acid (2′,4′-ENA) modification, and S-constrained ethyl (cEt) modification.
105. The gRNA of embodiment 104, wherein the 2′,4′ BNA is a LNA modification.
106. The gRNA of embodiment 104, wherein the 2′,4′ BNA is a cEt modification.
107. The gRNA of embodiment 97, wherein the at least one chemical modification comprises a BNA modification, 2′-O-Me modification, PS modification, or a combination thereof.
108. The gRNA of any one of embodiments 1-107, wherein the gRNA further comprises an extension comprising an edit template for reverse transcriptase (RT) editing.
109. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
   wherein the tracrRNA comprises:
(iii) an anti-repeat; and
(iv) a tail,
   wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.
110. The gRNA of embodiment 109, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 5 nucleotides.
111. The gRNA of embodiment 109, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 4 nucleotides.
112. The gRNA of embodiment 109, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 3 nucleotides.
113. The gRNA of embodiment 109, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 2 nucleotides.
114. The gRNA of embodiment 109, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 nucleotide.
115. The gRNA of embodiment 109, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.
116. The gRNA of any one of embodiments 109-115, wherein the spacer is capable of hybridizing to a target sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.
117. The gRNA of any one of embodiments 109-116, wherein the crRNA repeat has the nucleotide sequence set forth as SEQ ID NO: 546 or a nucleotide sequence that differs in length and/or sequence from SEQ ID NO: 546 by 1 to 8 nucleotides.
118. The gRNA of embodiment 117, wherein the crRNA repeat has a nucleotide sequence that differs in length and/or sequence from SEQ ID NO: 546 by 8 nucleotides.
119. The gRNA of embodiment 117, wherein the crRNA repeat has a nucleotide sequence that differs in length and/or sequence from SEQ ID NO: 546 by 7 nucleotides.
120. The gRNA of embodiment 117, wherein the crRNA repeat has a nucleotide sequence that differs in length and/or sequence from SEQ ID NO: 546 by 6 nucleotides.
121. The gRNA of embodiment 117, wherein the crRNA repeat has a nucleotide sequence that differs in length and/or sequence from SEQ ID NO: 546 by 5 nucleotides.
122. The gRNA of embodiment 117, wherein the crRNA repeat has a nucleotide sequence that differs in length and/or sequence from SEQ ID NO: 546 by 4 nucleotides.
123. The gRNA of embodiment 117, wherein the crRNA repeat has a nucleotide sequence that differs in length and/or sequence from SEQ ID NO: 546 by 3 nucleotides.
124. The gRNA of embodiment 117, wherein the crRNA repeat has a nucleotide sequence that differs in length and/or sequence from SEQ ID NO: 546 by 2 nucleotides.
125. The gRNA of embodiment 117, wherein the crRNA repeat has a nucleotide sequence that differs in length and/or sequence from SEQ ID NO: 546 by 1 nucleotide.
126. The gRNA of embodiment 117, wherein the crRNA repeat has the nucleotide sequence set forth as any one of SEQ ID NOs: 546, 549-552, 839, 842, and 845.
127. The gRNA of any one of embodiments 109-117, wherein the crRNA has a nucleotide sequence having at least 80% sequence identity to SEQ ID NOs: 574-692.
128. The gRNA of any one of embodiments 109-117, wherein the crRNA has a nucleotide sequence having at least 90% sequence identity to any one of SEQ ID NOs: 574-692.
129. The gRNA of any one of embodiments 109-117, wherein the crRNA has a nucleotide sequence having at least 95% sequence identity to any one of SEQ ID NOs: 574-692.
130. The gRNA of any one of embodiments 109-117, wherein the crRNA has the nucleotide sequence set forth as any one of SEQ ID NOs: 574-692.
131. The gRNA of any one of embodiments 109-117, wherein the tracrRNA has a nucleotide sequence having at least 80% sequence identity to SEQ ID NO: 547.
132. The gRNA of embodiment 131, wherein the tracrRNA has a nucleotide sequence having at least 90% sequence identity to SEQ ID NO: 547.
133. The gRNA of embodiment 131, wherein the tracrRNA has a nucleotide sequence having at least 95% sequence identity to SEQ ID NO: 547.
134. The gRNA of any one of embodiments 109-117, wherein the tracrRNA has a nucleotide sequence that differs in length from SEQ ID NO: 547 by 1 to 16 nucleotides.
135. The gRNA of embodiment 134, wherein the tracrRNA has a nucleotide sequence that is 8 nucleotides shorter than SEQ ID NO: 547.
136. The gRNA of embodiment 134, wherein the tracrRNA has a nucleotide sequence that is 11 nucleotides shorter than SEQ ID NO: 547.
137. The gRNA of any one of embodiments 131-136, wherein the tracrRNA has the nucleotide sequence set forth as any one of SEQ ID NOs: 547, 553-562, 840, 842, and 846.
138. The gRNA of any one of embodiments 109-116, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
139. The gRNA of embodiment 138, wherein the linker has a nucleotide sequence set forth as AAAG, GAAA, ACUU, or CAAAGG.
140. The gRNA of embodiment 139, wherein the linker has a nucleotide sequence set forth as AAAG.
141. The gRNA of any one of embodiments 138-140, wherein the backbone of the sgRNA comprises a total length of at least 85, 90, 95, 100, 105, 110, 115, or 120 nucleotides.
142. The gRNA of any one of embodiments 138-140, wherein the backbone of the sgRNA comprises a total length of at most 85, 90, 95, 100, 105, 110, 115, or 120 nucleotides.
143. The gRNA of any one of embodiments 138-140, wherein the backbone of the sgRNA comprises a total length of 86 to 98 nucleotides.
144. The gRNA of any one of embodiments 138-140, wherein the backbone of the sgRNA comprises a total length of 94 nucleotides.
145. The gRNA of any one of embodiments 138-140, wherein the backbone of the sgRNA has a nucleotide sequence having at least 80% sequence identity to any one of SEQ ID NOs: 563-573.
146. The gRNA of embodiment 145, wherein the sgRNA backbone has a nucleotide sequence having at least 90% sequence identity to any one of SEQ ID NOs: 563-573.
147. The gRNA of embodiment 145, wherein the sgRNA backbone has a nucleotide sequence having at least 95% sequence identity to any one of SEQ ID NOs: 563-573.
148. The gRNA of embodiment 145, wherein the sgRNA backbone has the nucleotide sequence set forth as any one of SEQ ID NOs: 563-573.
149. The gRNA of any one of embodiments 109-116, wherein the gRNA comprises a first stem loop formed by hybridization of the crRNA repeat and the anti-repeat, wherein the first stem loop comprises a first stem and a second stem, and wherein the first stem of the first stem loop comprises a total length of at least 3, 4, 5, 6, 7, 8, 9, 10, or 11 base pairs (bp).
150. The gRNA of any one of embodiments 109-116, wherein the gRNA comprises a first stem loop formed by hybridization of the crRNA repeat and the anti-repeat, wherein the first stem loop comprises a first stem and a second stem, and wherein the first stem of the first stem loop comprises a total length of at most 3, 4, 5, 6, 7, 8, 9, 10, or 11 bp.
151. The gRNA of embodiment 149 or 150, wherein the first stem of the first stem loop comprises a total length of 6 bp.
152. The gRNA of embodiment 149 or 150, wherein the first stem of the first stem loop comprises a total length of 3 bp.
153. The gRNA of any one of embodiments 109-116, wherein the tail of the tracrRNA comprises a total length of at least 1, 2, 3, 4, 5, 6, or 7 nucleotides.
154. The gRNA of any one of embodiments 109-116, wherein the tail of the tracrRNA comprises a total length of at most 1, 2, 3, 4, 5, 6, or 7 nucleotides.
155. The gRNA of embodiment 153 or 154, wherein the tail of the tracrRNA comprises a total length of 3 nucleotides.
156. The gRNA of embodiment 153 or 154, wherein the tail of the tracrRNA comprises a total length of 1 nucleotide.
157. The gRNA of embodiment 149 or 150, wherein the gRNA further comprises a second stem loop most proximal to the tail, wherein the second stem loop comprises a first stem and a second stem.
158. The gRNA of embodiment 157, wherein the first stem of the second stem loop comprises a total length of at least 1, 2, 3, 4, 5, or 6 bp.
159. The gRNA of embodiment 157, wherein the first stem of the second stem loop comprises a total length of at most 1, 2, 3, 4, 5, or 6 bp.
160. The gRNA of embodiment 158 or 159, wherein the first stem of the second stem loop comprises a total length of 5 bp.
161. The gRNA of any one of embodiments 157-160, wherein the first stem of the first stem loop comprises a total length of 6 bp, the tail of the tracrRNA comprises a total length of 3 nucleotides, and the first stem of the second stem loop comprises a total length of 5 bp.
162. The gRNA of any one of embodiments 109-116, wherein the gRNA is a dual guide RNA (dgRNA).
163. The gRNA of embodiment 162, wherein the crRNA repeat of the dgRNA comprises a total length of at least 10, 11, 12, 13, 14, 15, 16, 17, 18, 19, 20, or 21 nucleotides.
164. The gRNA of embodiment 162, wherein the crRNA repeat of the dgRNA comprises a total length of at most 10, 11, 12, 13, 14, 15, 16, 17, 18, 19, 20, or 21 nucleotides.
165. The gRNA of embodiment 163 or 164, wherein the crRNA repeat of the dgRNA comprises a total length of 13 nucleotides.
166. The gRNA of embodiment 163 or 164, wherein the crRNA repeat of the dgRNA comprises a total length of 16 nucleotides.
167. The gRNA of embodiment 163 or 164, wherein the crRNA repeat of the dgRNA comprises a total length of 21 nucleotides.
168. The gRNA of embodiment 162, wherein the tracrRNA of the dgRNA comprises a total length of at least 65, 70, 75, 80, or 85 nucleotides.
169. The gRNA of embodiment 162, wherein the tracrRNA of the dgRNA comprises a total length of at most 65, 70, 75, 80, or 85 nucleotides.
170. The gRNA of embodiment 168 or 169, wherein the tracrRNA of the dgRNA comprises a total length of 74 nucleotides.
171. The gRNA of embodiment 168 or 169, wherein the tracrRNA of the dgRNA comprises a total length of 77 nucleotides.
172. The gRNA of any one of embodiments 109-171, wherein the gRNA comprises a total length of at least 85, 90, 95, 100, 105, 110, 115, 120, 125, 130, or 135 nucleotides.
173. The gRNA of any one of embodiments 109-171, wherein the gRNA comprises a total length of at most 85, 90, 95, 100, 105, 110, 115, 120, 125, 130, or 135 nucleotides.
174. The gRNA of any one of embodiments 109-171, wherein the gRNA comprises a total length of 106 to 135 nucleotides.
175. The gRNA of embodiment 174, wherein the gRNA comprises a total length of 117 to 119 nucleotides.
176. The gRNA of any one of embodiments 109-175, wherein the gRNA is capable of targeting a bound RNA-guided nuclease (RGN) polypeptide to the target sequence.
177. The gRNA of embodiment 176, wherein the RGN polypeptide is capable of recognizing a consensus protospacer adjacent motif (PAM) having the nucleotide sequence set forth as NNNNCC.
178. The gRNA of embodiment 177, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.
179. The gRNA of any one of embodiments 176-178, wherein the RGN polypeptide has an amino acid sequence having at least 80% sequence identity to SEQ ID NO: 545; and wherein the target sequence and the spacer are selected from the group consisting of:
a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides;
b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides;
c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides;
d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides;
e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and
f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides.
180. The gRNA of embodiment 179, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 5 nucleotides;
b) a target sequence having the nucleotide sequence set forth as SEQ ID NO: 164 and a spacer having a nucleotide sequence that differs in length and/or sequence from SEQ ID NO: 163 by 5 nucleotides;
c) a target sequence having the nucleotide sequence set forth as SEQ ID NO: 190 and a spacer having a nucleotide sequence that differs in length and/or sequence from SEQ ID NO: 189 by 5 nucleotides;
d) a target sequence having the nucleotide sequence set forth as SEQ ID NO: 180 and a spacer having a nucleotide sequence that differs in length and/or sequence from SEQ ID NO: 179 by 5 nucleotides;
e) a target sequence having the nucleotide sequence set forth as SEQ ID NO: 198 and a spacer having a nucleotide sequence that differs in length and/or sequence from SEQ ID NO: 197 by 5 nucleotides; and
f) a target sequence having the nucleotide sequence set forth as SEQ ID NO: 194 and a spacer having a nucleotide sequence that differs in length and/or sequence from SEQ ID NO: 193 by 5 nucleotides.
181. The gRNA of embodiment 179, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 4 nucleotides;
b) a target sequence having the nucleotide sequence set forth as SEQ ID NO: 164 and a spacer having a nucleotide sequence that differs in length and/or sequence from SEQ ID NO: 163 by 4 nucleotides;
c) a target sequence having the nucleotide sequence set forth as SEQ ID NO: 190 and a spacer having a nucleotide sequence that differs in length and/or sequence from SEQ ID NO: 189 by 4 nucleotides;
d) a target sequence having the nucleotide sequence set forth as SEQ ID NO: 180 and a spacer having a nucleotide sequence that differs in length and/or sequence from SEQ ID NO: 179 by 4 nucleotides;
e) a target sequence having the nucleotide sequence set forth as SEQ ID NO: 198 and a spacer having a nucleotide sequence that differs in length and/or sequence from SEQ ID NO: 197 by 4 nucleotides; and
f) a target sequence having the nucleotide sequence set forth as SEQ ID NO: 194 and a spacer having a nucleotide sequence that differs in length and/or sequence from SEQ ID NO: 193 by 4 nucleotides.
182. The gRNA of embodiment 179, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 3 nucleotides;
b) a target sequence having the nucleotide sequence set forth as SEQ ID NO: 164 and a spacer having a nucleotide sequence that differs in length and/or sequence from SEQ ID NO: 163 by 3 nucleotides;
c) a target sequence having the nucleotide sequence set forth as SEQ ID NO: 190 and a spacer having a nucleotide sequence that differs in length and/or sequence from SEQ ID NO: 189 by 3 nucleotides;
d) a target sequence having the nucleotide sequence set forth as SEQ ID NO: 180 and a spacer having a nucleotide sequence that differs in length and/or sequence from SEQ ID NO: 179 by 3 nucleotides;
e) a target sequence having the nucleotide sequence set forth as SEQ ID NO: 198 and a spacer having a nucleotide sequence that differs in length and/or sequence from SEQ ID NO: 197 by 3 nucleotides; and
f) a target sequence having the nucleotide sequence set forth as SEQ ID NO: 194 and a spacer having a nucleotide sequence that differs in length and/or sequence from SEQ ID NO: 193 by 3 nucleotides.
183. The gRNA of embodiment 179, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 2 nucleotides;
b) a target sequence having the nucleotide sequence set forth as SEQ ID NO: 164 and a spacer having a nucleotide sequence that differs in length and/or sequence from SEQ ID NO: 163 by 2 nucleotides;
c) a target sequence having the nucleotide sequence set forth as SEQ ID NO: 190 and a spacer having a nucleotide sequence that differs in length and/or sequence from SEQ ID NO: 189 by 2 nucleotides;
d) a target sequence having the nucleotide sequence set forth as SEQ ID NO: 180 and a spacer having a nucleotide sequence that differs in length and/or sequence from SEQ ID NO: 179 by 2 nucleotides;
e) a target sequence having the nucleotide sequence set forth as SEQ ID NO: 198 and a spacer having a nucleotide sequence that differs in length and/or sequence from SEQ ID NO: 197 by 2 nucleotides; and
f) a target sequence having the nucleotide sequence set forth as SEQ ID NO: 194 and a spacer having a nucleotide sequence that differs in length and/or sequence from SEQ ID NO: 193 by 2 nucleotides.
184. The gRNA of embodiment 179, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 1 nucleotide;
b) a target sequence having the nucleotide sequence set forth as SEQ ID NO: 164 and a spacer having a nucleotide sequence that differs in length and/or sequence from SEQ ID NO: 163 by 1 nucleotide;
c) a target sequence having the nucleotide sequence set forth as SEQ ID NO: 190 and a spacer having a nucleotide sequence that differs in length and/or sequence from SEQ ID NO: 189 by 1 nucleotide;
d) a target sequence having the nucleotide sequence set forth as SEQ ID NO: 180 and a spacer having a nucleotide sequence that differs in length and/or sequence from SEQ ID NO: 179 by 1 nucleotide;
e) a target sequence having the nucleotide sequence set forth as SEQ ID NO: 198 and a spacer having a nucleotide sequence that differs in length and/or sequence from SEQ ID NO: 197 by 1 nucleotide; and
f) a target sequence having the nucleotide sequence set forth as SEQ ID NO: 194 and a spacer having a nucleotide sequence that differs in length and/or sequence from SEQ ID NO: 193 by 1 nucleotide.
185. The gRNA of embodiment 179, wherein the spacer has the nucleotide sequence set forth as any one of SEQ ID NOs: 155, 163, 189, 179, 197, and 193.
186. The gRNA of any one of embodiments 179-185, wherein the RGN polypeptide has an amino acid sequence having at least 90% sequence identity to SEQ ID NO: 545.
187. The gRNA of any one of embodiments 179-185, wherein the RGN polypeptide has an amino acid sequence having at least 95% sequence identity to SEQ ID NO: 545.
188. The gRNA of any one of embodiments 179-185, wherein the RGN polypeptide has the amino acid sequence set forth as SEQ ID NO: 545.
189. The gRNA of any one of embodiments 176-188, wherein the gRNA has a nucleotide sequence having at least 80% sequence identity to any one of SEQ ID NOs: 693-834.
190. The gRNA of embodiment 189, wherein the gRNA has a nucleotide sequence having at least 90% sequence identity to any one of SEQ ID NOs: 693-834.
191. The gRNA of embodiment 189, wherein the gRNA has a nucleotide sequence having at least 95% sequence identity to any one of SEQ ID NOs: 693-834.
192. The gRNA of embodiment 189, wherein the gRNA has a nucleotide sequence set forth as any one of SEQ ID NOs: 693-834.
193. The gRNA of embodiment 192, wherein the gRNA has a nucleotide sequence set forth as any one of SEQ ID NOs: 693, 694, 695, 696, 697, and 698.
194. The gRNA of embodiment 178, wherein the RGN polypeptide is capable of recognizing a full PAM having the nucleotide sequence set forth as any one of GGGTCCTT, GGGGCCGA, GGGGCCCA, CGGCCCTG, GGGCCCAT, TGGCCC, TGGGCC, GGGCCC, CGGGCC, and AGGGCC.
195. The gRNA of embodiment 194, wherein the RGN polypeptide has an amino acid sequence having at least 80% sequence identity to SEQ ID NO: 835.
196. The gRNA of embodiment 195, wherein the RGN polypeptide has an amino acid sequence having at least 90% sequence identity to SEQ ID NO: 835.
197. The gRNA of embodiment 195, wherein the RGN polypeptide has an amino acid sequence having at least 95% sequence identity to SEQ ID NO: 835.
198. The gRNA of embodiment 195, wherein the RGN polypeptide has the amino acid sequence set forth as SEQ ID NO: 835.
199. The gRNA of embodiment 178, wherein the RGN polypeptide is capable of recognizing a full PAM having the nucleotide sequence set forth as any one of TCGGCCCT, CAGGCCTG, TCGGCC, and CGGGCC.
200. The gRNA of embodiment 199, wherein the RGN polypeptide has an amino acid sequence having at least 80% sequence identity to SEQ ID NO: 915.
201. The gRNA of embodiment 200, wherein the RGN polypeptide has an amino acid sequence having at least 90% sequence identity to SEQ ID NO: 915.
202. The gRNA of embodiment 200, wherein the RGN polypeptide has an amino acid sequence having at least 95% sequence identity to SEQ ID NO: 915.
203. The gRNA of embodiment 200, wherein the RGN polypeptide has the amino acid sequence set forth as SEQ ID NO: 915.
204. The gRNA of any one of embodiments 109-203, wherein the gRNA comprises at least one chemical modification.
205. The gRNA of embodiment 204, wherein the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.
206. The gRNA of embodiment 205, wherein the at least one chemical modification comprises MS modifications at the 3 terminal nucleotides at the 5′ region and at the 3 terminal nucleotides at the 3′ region of the gRNA.
207. The gRNA of embodiment 206, wherein the crRNA repeat has the nucleotide sequence set forth as any one of SEQ ID NOs: 940, 942-945, 1228, 1230, and 1232.
208. The gRNA of embodiment 206 or 207, wherein the crRNA has the nucleotide sequence set forth as any one of SEQ ID NOs: 967-1085.
209. The gRNA of any one of embodiments 206-208, wherein the tracrRNA has the nucleotide sequence set forth as any one of SEQ ID NOs: 941, 946-955, 1229, 1231, and 1233.
210. The gRNA of any one of embodiments 206-209, wherein the gRNA has the nucleotide sequence set forth as any one of SEQ ID NOs: 1086-1227.
211. The gRNA of embodiment 205, wherein the BNA comprises a 2′,4′ BNA modification.
212. The gRNA of embodiment 211, wherein the 2′,4′ BNA modification is selected from the group consisting of: locked nucleic acid (LNA) modification, BNANC[N-Me]modification, 2′-O,4′-C-ethylene bridged nucleic acid (2′,4′-ENA) modification, and S-constrained ethyl (cEt) modification.
213. The gRNA of embodiment 212, wherein the 2′,4′ BNA is a LNA modification.
214. The gRNA of embodiment 212, wherein the 2′,4′ BNA is a cEt modification.
215. The gRNA of embodiment 205, wherein the at least one chemical modification comprises a BNA modification, 2′-O-Me modification, PS modification, or a combination thereof.
216. The gRNA of any one of embodiments 109-215, wherein the gRNA further comprises an extension comprising an edit template for reverse transcriptase (RT) editing.
217. A nucleic acid molecule comprising a CRISPR RNA (crRNA) or encoding a crRNA, wherein the crRNA comprises a spacer and a crRNA repeat, wherein the spacer is capable of hybridizing to a target sequence, and wherein the target sequence in a forkhead box P3 (FOXP3) gene has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.
218. The nucleic acid molecule of embodiment 217, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.
219. The nucleic acid molecule of embodiment 218, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 5 nucleotides.
220. The nucleic acid molecule of embodiment 218, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 4 nucleotides.
221. The nucleic acid molecule of embodiment 218, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 3 nucleotides.
222. The nucleic acid molecule of embodiment 218, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 2 nucleotides.
223. The nucleic acid molecule of embodiment 218, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 nucleotide.
224. The nucleic acid molecule of embodiment 217, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.
225. The nucleic acid molecule of any one of embodiments 217-224, wherein the crRNA repeat has the nucleotide sequence set forth as SEQ ID NO: 546 or a nucleotide sequence that differs in length and/or sequence from SEQ ID NO: 546 by 1 to 8 nucleotides.
226. The nucleic acid molecule of embodiment 225, wherein the crRNA repeat has a nucleotide sequence that differs in length and/or sequence from SEQ ID NO: 546 by 8 nucleotides.
227. The nucleic acid molecule of embodiment 225, wherein the crRNA repeat has a nucleotide sequence that differs in length and/or sequence from SEQ ID NO: 546 by 7 nucleotides.
228. The nucleic acid molecule of embodiment 225, wherein the crRNA repeat has a nucleotide sequence that differs in length and/or sequence from SEQ ID NO: 546 by 6 nucleotides.
229. The nucleic acid molecule of embodiment 225, wherein the crRNA repeat has a nucleotide sequence that differs in length and/or sequence from SEQ ID NO: 546 by 5 nucleotides.
230. The nucleic acid molecule of embodiment 225, wherein the crRNA repeat has a nucleotide sequence that differs in length and/or sequence from SEQ ID NO: 546 by 4 nucleotides.
231. The nucleic acid molecule of embodiment 225, wherein the crRNA repeat has a nucleotide sequence that differs in length and/or sequence from SEQ ID NO: 546 by 3 nucleotides.
232. The nucleic acid molecule of embodiment 225, wherein the crRNA repeat has a nucleotide sequence that differs in length and/or sequence from SEQ ID NO: 546 by 2 nucleotides.
233. The nucleic acid molecule of embodiment 225, wherein the crRNA repeat has a nucleotide sequence that differs in length and/or sequence from SEQ ID NO: 546 by 1 nucleotide.
234. The nucleic acid molecule of embodiment 225, wherein the crRNA repeat has the nucleotide sequence set forth as any one of SEQ ID NOs: 546, 549-552, 839, 842, and 845.
235. The nucleic acid molecule of any one of embodiments 217-225, wherein the crRNA has a nucleotide sequence having at least 80% sequence identity to any one of SEQ ID NOs: 574-692.
236. The nucleic acid molecule of embodiment 235, wherein the crRNA has a nucleotide sequence having at least 90% sequence identity to any one of SEQ ID NOs: 574-692.
237. The nucleic acid molecule of embodiment 236, wherein the crRNA has a nucleotide sequence having at least 95% sequence identity to any one of SEQ ID NOs: 574-692.
238. The nucleic acid molecule of any one of embodiments 217-225, wherein the crRNA has the nucleotide sequence set forth as any one of SEQ ID NOs: 574-692.
239. The nucleic acid molecule of any one of embodiments 217-238, wherein the crRNA is capable of binding a trans-activating CRISPR RNA (tracrRNA) to form a guide RNA (gRNA), wherein the tracrRNA comprises an anti-repeat and a tail.
240. The nucleic acid molecule of embodiment 239, wherein the tracrRNA has a nucleotide sequence having at least 80% sequence identity to SEQ ID NO: 547.
241. The nucleic acid molecule of embodiment 240, wherein the tracrRNA has a nucleotide sequence having at least 90% sequence identity to SEQ ID NO: 547.
242. The nucleic acid molecule of embodiment 240, wherein the tracrRNA has a nucleotide sequence having at least 95% sequence identity to SEQ ID NO: 547.
243. The nucleic acid molecule of embodiment 239, wherein the tracrRNA has a nucleotide sequence that differs in length from SEQ ID NO: 547 by 1 to 16 nucleotides.
244. The nucleic acid molecule of embodiment 243, wherein the tracrRNA has a nucleotide sequence that is 8 nucleotides shorter than SEQ ID NO: 547.
245. The nucleic acid molecule of embodiment 243, wherein the tracrRNA has a nucleotide sequence that is 11 nucleotides shorter than SEQ ID NO: 547.
246. The nucleic acid molecule of any one of embodiments 240-245, wherein the tracrRNA has the nucleotide sequence set forth as any one of SEQ ID NOs: 547, 553-562, 840, 842, and 846.
247. The nucleic acid molecule of embodiment 239, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
248. The nucleic acid molecule of embodiment 247, wherein the backbone of the sgRNA comprises a total length of 86 to 98 nucleotides.
249. The nucleic acid molecule of embodiment 247, wherein the backbone of the sgRNA comprises a total length of 94 nucleotides.
250. The nucleic acid molecule of embodiment 247, wherein the backbone of the sgRNA has a nucleotide sequence having at least 80% sequence identity to any one of SEQ ID NOs: 563-573.
251. The nucleic acid molecule of embodiment 250, wherein the backbone of the sgRNA has a nucleotide sequence having at least 90% sequence identity to any one of SEQ ID NOs: 563-573.
252. The nucleic acid molecule of embodiment 250, wherein the backbone of the sgRNA has a nucleotide sequence having at least 95% sequence identity to any one of SEQ ID NOs: 563-573.
253. The nucleic acid molecule of embodiment 250, wherein the backbone of the sgRNA has the nucleotide sequence set forth as any one of SEQ ID NOs: 563-573.
254. The nucleic acid molecule of any one of embodiments 239-253, wherein the gRNA comprises a first stem loop comprising a first stem and a second stem formed by hybridization of the crRNA repeat and the anti-repeat, wherein the first stem of the first stem loop comprises a total length of at least 3, 4, 5, 6, 7, 8, 9, 10, or 11 bp.
255. The nucleic acid molecule of any one of embodiments 239-253, wherein the gRNA comprises a first stem loop comprising a first stem and a second stem formed by hybridization of the crRNA repeat and the anti-repeat, wherein the first stem of the first stem loop comprises a total length of at most 3, 4, 5, 6, 7, 8, 9, 10, or 11 bp.
256. The nucleic acid molecule of embodiment 254 or 255, wherein the first stem of the first stem loop comprises a total length of 6 bp.
257. The nucleic acid molecule of embodiment 254 or 255, wherein the first stem of the first stem loop comprises a total length of 3 bp.
258. The nucleic acid molecule of any one of embodiments 239-257, wherein the tail of the tracrRNA comprises a total length of at least 1, 2, 3, 4, 5, 6, or 7 nucleotides.
259. The nucleic acid molecule of any one of embodiments 239-257, wherein the tail of the tracrRNA comprises a total length of at most 1, 2, 3, 4, 5, 6, or 7 nucleotides.
260. The nucleic acid molecule of embodiment 258 or 259, wherein the tail of the tracrRNA comprises a total length of 3 nucleotides.
261. The nucleic acid molecule of embodiment 258 or 259, wherein the tail of the tracrRNA comprises a total length of 1 nucleotide.
262. The nucleic acid molecule of any one of embodiments 254-261, wherein the gRNA further comprises a second stem loop most proximal to the tail, wherein the second stem loop comprises a first stem and a second stem.
263. The nucleic acid molecule of embodiment 262, wherein the first stem of the second stem loop comprises a total length of at least 1, 2, 3, 4, 5, or 6 bp.
264. The nucleic acid molecule of embodiment 262, wherein the first stem of the second stem loop comprises a total length of at most 1, 2, 3, 4, 5, or 6 bp.
265. The nucleic acid molecule of embodiment 263 or 264, wherein the first stem of the second stem loop comprises a total length of 5 bp.
266. The nucleic acid molecule of any one of embodiments 262-265, wherein the first stem of the first stem loop comprises a total length of 6 bp, the tail of the tracrRNA comprises a total length of 3 nucleotides, and the first stem of the second stem loop comprises a total length of 5 bp.
267. The nucleic acid molecule of embodiment 239, wherein the gRNA is a dual guide RNA (dgRNA).
268. The nucleic acid molecule of embodiment 267, wherein the crRNA repeat comprises a total length of at least 10, 11, 12, 13, 14, 15, 16, 17, 18, 19, 20, or 21 nucleotides.
269. The nucleic acid molecule of embodiment 267, wherein the crRNA repeat comprises a total length of at most 10, 11, 12, 13, 14, 15, 16, 17, 18, 19, 20, or 21 nucleotides.
270. The nucleic acid molecule of embodiment 268 or 269, wherein the crRNA repeat comprises a total length of 13 nucleotides.
271. The nucleic acid molecule of embodiment 268 or 269, wherein the crRNA repeat comprises a total length of 16 nucleotides.
272. The nucleic acid molecule of embodiment 268 or 269, wherein the crRNA repeat of the dgRNA comprises a total length of 21 nucleotides.
273. The nucleic acid molecule of any one of embodiments 267-272, wherein the tracrRNA comprises a total length of at least 65, 70, 75, 80, or 85 nucleotides.
274. The nucleic acid molecule of any one of embodiments 267-272, wherein the tracrRNA comprises a total length of at most 65, 70, 75, 80, or 85 nucleotides.
275. The nucleic acid molecule of embodiment 273 or 274, wherein the tracrRNA comprises a total length of 74 nucleotides.
276. The nucleic acid molecule of embodiment 273 or 274, wherein the tracrRNA comprises a total length of 77 nucleotides.
277. The nucleic acid molecule of any one of embodiments 239-276, wherein the gRNA comprises a total length of at least 85, 90, 95, 100, 105, 110, 115, 120, 125, 130, or 135 nucleotides.
278. The nucleic acid molecule of any one of embodiments 239-276, wherein the gRNA comprises a total length of at most 85, 90, 95, 100, 105, 110, 115, 120, 125, 130, or 135 nucleotides.
279. The nucleic acid molecule of any one of embodiments 239-276, wherein the gRNA comprises a total length of 106 to 135 nucleotides.
280. The nucleic acid molecule of embodiment 279, wherein the gRNA comprises a total length of 117 to 119 nucleotides.
281. The nucleic acid molecule of any one of embodiments 239-280, wherein the gRNA is capable of targeting a bound RNA-guided nuclease (RGN) polypeptide to a target sequence.
282. The nucleic acid molecule of embodiment 281, wherein the RGN polypeptide is capable of recognizing a consensus protospacer adjacent motif (PAM) having the nucleotide sequence set forth as NNNNCC.
283. The nucleic acid molecule of embodiment 282, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.
284. The nucleic acid molecule of any one of embodiments 281-283, wherein the RGN polypeptide comprises an amino acid sequence having at least 80% sequence identity to SEQ ID NO: 545; and wherein the target sequence and the spacer are selected from the group consisting of:
a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides;
b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides;
c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides;
d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides;
e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and
f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides.
285. The nucleic acid molecule of embodiment 284, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 5 nucleotides;
b) a target sequence having the nucleotide sequence set forth as SEQ ID NO: 164 and a spacer having a nucleotide sequence that differs in length and/or sequence from SEQ ID NO: 163 by 5 nucleotides;
c) a target sequence having the nucleotide sequence set forth as SEQ ID NO: 190 and a spacer having a nucleotide sequence that differs in length and/or sequence from SEQ ID NO: 189 by 5 nucleotides;
d) a target sequence having the nucleotide sequence set forth as SEQ ID NO: 180 and a spacer having a nucleotide sequence that differs in length and/or sequence from SEQ ID NO: 179 by 5 nucleotides;
e) a target sequence having the nucleotide sequence set forth as SEQ ID NO: 198 and a spacer having a nucleotide sequence that differs in length and/or sequence from SEQ ID NO: 197 by 5 nucleotides; and
f) a target sequence having the nucleotide sequence set forth as SEQ ID NO: 194 and a spacer having a nucleotide sequence that differs in length and/or sequence from SEQ ID NO: 193 by 5 nucleotides.
286. The nucleic acid molecule of embodiment 284, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 4 nucleotides;
b) a target sequence having the nucleotide sequence set forth as SEQ ID NO: 164 and a spacer having a nucleotide sequence that differs in length and/or sequence from SEQ ID NO: 163 by 4 nucleotides;
c) a target sequence having the nucleotide sequence set forth as SEQ ID NO: 190 and a spacer having a nucleotide sequence that differs in length and/or sequence from SEQ ID NO: 189 by 4 nucleotides;
d) a target sequence having the nucleotide sequence set forth as SEQ ID NO: 180 and a spacer having a nucleotide sequence that differs in length and/or sequence from SEQ ID NO: 179 by 4 nucleotides;
e) a target sequence having the nucleotide sequence set forth as SEQ ID NO: 198 and a spacer having a nucleotide sequence that differs in length and/or sequence from SEQ ID NO: 197 by 4 nucleotides; and
f) a target sequence having the nucleotide sequence set forth as SEQ ID NO: 194 and a spacer having a nucleotide sequence that differs in length and/or sequence from SEQ ID NO: 193 by 4 nucleotides.
287. The nucleic acid molecule of embodiment 284, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 3 nucleotides;
b) a target sequence having the nucleotide sequence set forth as SEQ ID NO: 164 and a spacer having a nucleotide sequence that differs in length and/or sequence from SEQ ID NO: 163 by 3 nucleotides;
c) a target sequence having the nucleotide sequence set forth as SEQ ID NO: 190 and a spacer having a nucleotide sequence that differs in length and/or sequence from SEQ ID NO: 189 by 3 nucleotides;
d) a target sequence having the nucleotide sequence set forth as SEQ ID NO: 180 and a spacer having a nucleotide sequence that differs in length and/or sequence from SEQ ID NO: 179 by 3 nucleotides;
e) a target sequence having the nucleotide sequence set forth as SEQ ID NO: 198 and a spacer having a nucleotide sequence that differs in length and/or sequence from SEQ ID NO: 197 by 3 nucleotides; and
f) a target sequence having the nucleotide sequence set forth as SEQ ID NO: 194 and a spacer having a nucleotide sequence that differs in length and/or sequence from SEQ ID NO: 193 by 3 nucleotides.
288. The nucleic acid molecule of embodiment 284, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 2 nucleotides;
b) a target sequence having the nucleotide sequence set forth as SEQ ID NO: 164 and a spacer having a nucleotide sequence that differs in length and/or sequence from SEQ ID NO: 163 by 2 nucleotides;
c) a target sequence having the nucleotide sequence set forth as SEQ ID NO: 190 and a spacer having a nucleotide sequence that differs in length and/or sequence from SEQ ID NO: 189 by 2 nucleotides;
d) a target sequence having the nucleotide sequence set forth as SEQ ID NO: 180 and a spacer having a nucleotide sequence that differs in length and/or sequence from SEQ ID NO: 179 by 2 nucleotides;
e) a target sequence having the nucleotide sequence set forth as SEQ ID NO: 198 and a spacer having a nucleotide sequence that differs in length and/or sequence from SEQ ID NO: 197 by 2 nucleotides; and
f) a target sequence having the nucleotide sequence set forth as SEQ ID NO: 194 and a spacer having a nucleotide sequence that differs in length and/or sequence from SEQ ID NO: 193 by 2 nucleotides.
289. The nucleic acid molecule of embodiment 284, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 1 nucleotide;
b) a target sequence having the nucleotide sequence set forth as SEQ ID NO: 164 and a spacer having a nucleotide sequence that differs in length and/or sequence from SEQ ID NO: 163 by 1 nucleotide;
c) a target sequence having the nucleotide sequence set forth as SEQ ID NO: 190 and a spacer having a nucleotide sequence that differs in length and/or sequence from SEQ ID NO: 189 by 1 nucleotide;
d) a target sequence having the nucleotide sequence set forth as SEQ ID NO: 180 and a spacer having a nucleotide sequence that differs in length and/or sequence from SEQ ID NO: 179 by 1 nucleotide;
e) a target sequence having the nucleotide sequence set forth as SEQ ID NO: 198 and a spacer having a nucleotide sequence that differs in length and/or sequence from SEQ ID NO: 197 by 1 nucleotide; and
f) a target sequence having the nucleotide sequence set forth as SEQ ID NO: 194 and a spacer having a nucleotide sequence that differs in length and/or sequence from SEQ ID NO: 193 by 1 nucleotide.
290. The nucleic acid molecule of embodiment 284, wherein the spacer has the nucleotide sequence set forth as any one of SEQ ID NOs: 155, 163, 189, 179, 197, and 193.
291. The nucleic acid molecule of any one of embodiments 284-290, wherein the RGN polypeptide has an amino acid sequence having at least 90% sequence identity to SEQ ID NO: 545.
292. The nucleic acid molecule of any one of embodiments 2844-290, wherein the RGN polypeptide has an amino acid sequence having at least 95% sequence identity to SEQ ID NO: 545.
293. The nucleic acid molecule of any one of embodiments 284-290, wherein the RGN polypeptide comprises an amino acid sequence set forth as SEQ ID NO: 545.
294. The nucleic acid molecule of any one of embodiments 281-293, wherein the gRNA has a nucleotide sequence having at least 80% sequence identity to any one of SEQ ID NOs: 693-834.
295. The nucleic acid molecule of embodiment 294, wherein the gRNA has a nucleotide sequence having at least 90% sequence identity to any one of SEQ ID NOs: 693-834.
296. The nucleic acid molecule of embodiment 294, wherein the gRNA has a nucleotide sequence having at least 95% sequence identity to any one of SEQ ID NOs: 693-834.
297. The nucleic acid molecule of embodiment 294, wherein the gRNA has a nucleotide sequence set forth as any one of SEQ ID NOs: 693-834.
298. The nucleic acid molecule of embodiment 297, wherein the gRNA has a nucleotide sequence set forth as any one of SEQ ID NOs: 693, 694, 695, 696, 697, and 698.
299. The nucleic acid molecule of embodiment 283, wherein the RGN polypeptide is capable of recognizing a full PAM having the nucleotide sequence set forth as any one of GGGTCCTT, GGGGCCGA, GGGGCCCA, CGGCCCTG, GGGCCCAT, TGGCCC, TGGGCC, GGGCCC, CGGGCC, and AGGGCC.
300. The nucleic acid molecule of embodiment 299, wherein the RGN polypeptide has an amino acid sequence having at least 80% sequence identity to SEQ ID NO: 835.
301. The nucleic acid molecule of embodiment 300, wherein the RGN polypeptide has an amino acid sequence having at least 90% sequence identity to SEQ ID NO: 835.
302. The nucleic acid molecule of embodiment 300, wherein the RGN polypeptide has an amino acid sequence having at least 95% sequence identity to SEQ ID NO: 835.
303. The nucleic acid molecule of embodiment 300, wherein the RGN polypeptide has the amino acid sequence set forth as SEQ ID NO: 835.
304. The nucleic acid molecule of embodiment 283, wherein the RGN polypeptide is capable of recognizing a full PAM having the nucleotide sequence set forth as any one of TCGGCCCT, CAGGCCTG, TCGGCC, and CGGGCC.
305. The nucleic acid molecule of embodiment 304, wherein the RGN polypeptide has an amino acid sequence having at least 80% sequence identity to SEQ ID NO: 915.
306. The nucleic acid molecule of embodiment 305, wherein the RGN polypeptide has an amino acid sequence having at least 90% sequence identity to SEQ ID NO: 915.
307. The nucleic acid molecule of embodiment 305, wherein the RGN polypeptide has an amino acid sequence having at least 95% sequence identity to SEQ ID NO: 915.
308. The nucleic acid molecule of embodiment 305, wherein the RGN polypeptide has the amino acid sequence set forth as SEQ ID NO: 915.
309. The nucleic acid molecule of any one of embodiments 217-308, wherein the gRNA comprises at least one chemical modification.
310. The nucleic acid molecule of embodiment 309, wherein the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.
311. The nucleic acid molecule of embodiment 310, wherein the at least one chemical modification comprises MS modifications at the 3 terminal nucleotides at the 5′ region and at the 3 terminal nucleotides at the 3′ region of the gRNA.
312. The nucleic acid molecule of embodiment 311, wherein the crRNA repeat has the nucleotide sequence set forth as any one of SEQ ID NOs: 940, 942-945, 1228, 1230, and 1232.
313. The nucleic acid molecule of embodiment 311 or 312, wherein the crRNA has the nucleotide sequence set forth as any one of SEQ ID NOs: 967-1085.
314. The nucleic acid molecule of any one of embodiments 311-313, wherein the tracrRNA has the nucleotide sequence set forth as any one of SEQ ID NOs: 941, 946-955, 1229, 1231, and 1233.
315. The nucleic acid molecule of any one of embodiments 311-314, wherein the gRNA has the nucleotide sequence set forth as any one of SEQ ID NOs: 1086-1227.
316. The nucleic acid molecule of embodiment 310, wherein the BNA comprises a 2′,4′ BNA modification.
317. The nucleic acid molecule of embodiment 316, wherein the 2′,4′ BNA modification is selected from the group consisting of: locked nucleic acid (LNA) modification, BNANC[N-Me]modification, 2′-0,4′-C-ethylene bridged nucleic acid (2′,4′-ENA) modification, and S-constrained ethyl (cEt) modification.
318. The nucleic acid molecule of embodiment 317, wherein the 2′,4′ BNA is a LNA modification.
319. The nucleic acid molecule of embodiment 317, wherein the 2′,4′ BNA is a cEt modification.
320. The nucleic acid molecule of embodiment 310, wherein the at least one chemical modification comprises a BNA modification, 2′-O-Me modification, PS modification, or a combination thereof.
321. The nucleic acid molecule of any one of embodiments 239-320, wherein the gRNA further comprises an extension comprising an edit template for reverse transcriptase editing.
322. A vector comprising the nucleic acid molecule of any one of embodiments 217-238, wherein the nucleic acid molecule comprises a polynucleotide encoding the crRNA.
323. The vector of embodiment 322, wherein the nucleic acid molecule further comprises a heterologous promoter operably linked to the polynucleotide encoding the crRNA.
324. The vector of embodiment 323, wherein the heterologous promoter is an RNA polymerase III (pol III) promoter.
325. The vector of any one of embodiments 322-324, wherein the vector further comprises a nucleic acid molecule encoding an RGN polypeptide.
326. The vector of embodiment 325, wherein the crRNA is capable of binding a tracrRNA to form a guide RNA, and wherein the guide RNA is capable of binding to the RGN polypeptide.
327. The vector of embodiment 325 or 326, wherein the vector further comprises a promoter operably linked to the nucleic acid molecule encoding the RGN polypeptide.
328. A vector comprising the nucleic acid molecule of any one of embodiments 239-321, wherein the nucleic acid molecule comprises a polynucleotide encoding the crRNA, and wherein the vector further comprises a polynucleotide encoding the tracrRNA.
329. The vector of embodiment 328, wherein the polynucleotide encoding the crRNA and the polynucleotide encoding the tracrRNA are operably linked to the same promoter and are encoded as a sgRNA.
330. The vector of embodiment 328, wherein the polynucleotide encoding the crRNA and the polynucleotide encoding the tracrRNA are operably linked to separate promoters.
331. The vector of any one of embodiments 328-330, wherein the vector further comprises a nucleic acid molecule encoding an RGN polypeptide.
332. The vector of embodiment 331, wherein the crRNA is capable of binding the tracrRNA to form a guide RNA, and wherein the guide RNA is capable of binding to the RGN polypeptide.
333. The vector of embodiment 331 or 332, wherein the vector further comprises a promoter operably linked to the nucleic acid molecule encoding the RGN polypeptide.
334. A nucleic acid molecule comprising a CRISPR RNA (crRNA) or encoding a crRNA, wherein the crRNA comprises a spacer and a crRNA repeat, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.
335. The nucleic acid molecule of embodiment 334, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.
336. The nucleic acid molecule of embodiment 335, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 5 nucleotides.
337. The nucleic acid molecule of embodiment 335, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 4 nucleotides.
338. The nucleic acid molecule of embodiment 335, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 3 nucleotides.
339. The nucleic acid molecule of embodiment 335, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 2 nucleotides.
340. The nucleic acid molecule of embodiment 335, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 nucleotide.
341. The nucleic acid molecule of embodiment 334, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.
342. The nucleic acid molecule of any one of embodiments 334-341, wherein the spacer is capable of hybridizing to a target sequence, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.
343. The nucleic acid molecule of any one of embodiments 334-342, wherein the crRNA repeat has the nucleotide sequence set forth as SEQ ID NO: 546 or a nucleotide sequence that differs in length and/or sequence from SEQ ID NO: 546 by 1 to 8 nucleotides.
344. The nucleic acid molecule of embodiment 343, wherein the crRNA repeat has a nucleotide sequence that differs in length and/or sequence from SEQ ID NO: 546 by 8 nucleotides.
345. The nucleic acid molecule of embodiment 343, wherein the crRNA repeat has a nucleotide sequence that differs in length and/or sequence from SEQ ID NO: 546 by 7 nucleotides.
346. The nucleic acid molecule of embodiment 343, wherein the crRNA repeat has a nucleotide sequence that differs in length and/or sequence from SEQ ID NO: 546 by 6 nucleotides.
347. The nucleic acid molecule of embodiment 343, wherein the crRNA repeat has a nucleotide sequence that differs in length and/or sequence from SEQ ID NO: 546 by 5 nucleotides.
348. The nucleic acid molecule of embodiment 343, wherein the crRNA repeat has a nucleotide sequence that differs in length and/or sequence from SEQ ID NO: 546 by 4 nucleotides.
349. The nucleic acid molecule of embodiment 343, wherein the crRNA repeat has a nucleotide sequence that differs in length and/or sequence from SEQ ID NO: 546 by 3 nucleotides.
350. The nucleic acid molecule of embodiment 343, wherein the crRNA repeat has a nucleotide sequence that differs in length and/or sequence from SEQ ID NO: 546 by 2 nucleotides.
351. The nucleic acid molecule of embodiment 343, wherein the crRNA repeat has a nucleotide sequence that differs in length and/or sequence from SEQ ID NO: 546 by 1 nucleotide.
352. The nucleic acid molecule of embodiment 343, wherein the crRNA repeat has the nucleotide sequence set forth as any one of SEQ ID NOs: 546, 549-552, 839, 842, and 845.
353. The nucleic acid molecule of any one of embodiments 334-343, wherein the crRNA has a nucleotide sequence having at least 80% sequence identity to any one of SEQ ID NOs: 574-692.
354. The nucleic acid molecule of embodiment 353, wherein the crRNA has a nucleotide sequence having at least 90% sequence identity to any one of SEQ ID NOs: 574-692.
355. The nucleic acid molecule of embodiment 354, wherein the crRNA has a nucleotide sequence having at least 95% sequence identity to any one of SEQ ID NOs: 574-692.
356. The nucleic acid molecule of any one of embodiments 334-343, wherein the crRNA has the nucleotide sequence set forth as any one of SEQ ID NOs: 574-692.
357. The nucleic acid molecule of any one of embodiments 334-356, wherein the crRNA is capable of binding a trans-activating CRISPR RNA (tracrRNA) to form a guide RNA (gRNA), wherein the tracrRNA comprises an anti-repeat and a tail.
358. The nucleic acid molecule of embodiment 357, wherein the tracrRNA has a nucleotide sequence having at least 80% sequence identity to SEQ ID NO: 547.
359. The nucleic acid molecule of embodiment 358, wherein the tracrRNA has a nucleotide sequence having at least 90% sequence identity to SEQ ID NO: 547.
360. The nucleic acid molecule of embodiment 358, wherein the tracrRNA has a nucleotide sequence having at least 95% sequence identity to SEQ ID NO: 547.
361. The nucleic acid molecule of embodiment 357, wherein the tracrRNA has a nucleotide sequence that differs in length from SEQ ID NO: 547 by 1 to 16 nucleotides.
362. The nucleic acid molecule of embodiment 361, wherein the tracrRNA has a nucleotide sequence that is 8 nucleotides shorter than SEQ ID NO: 547.
363. The nucleic acid molecule of embodiment 361, wherein the tracrRNA has a nucleotide sequence that is 11 nucleotides shorter than SEQ ID NO: 547.
364. The nucleic acid molecule of any one of embodiments 358-363, wherein the tracrRNA has the nucleotide sequence set forth as any one of SEQ ID NOs: 547, 553-562, 840, 842, and 846.
365. The nucleic acid molecule of embodiment 357, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.
366. The nucleic acid molecule of embodiment 365, wherein the backbone of the sgRNA comprises a total length of 86 to 98 nucleotides.
367. The nucleic acid molecule of embodiment 365, wherein the backbone of the sgRNA comprises a total length of 94 nucleotides.
368. The nucleic acid molecule of embodiment 365, wherein the backbone of the sgRNA has a nucleotide sequence having at least 80% sequence identity to any one of SEQ ID NOs: 563-573.
369. The nucleic acid molecule of embodiment 368, wherein the backbone of the sgRNA has a nucleotide sequence having at least 90% sequence identity to any one of SEQ ID NOs: 563-573.
370. The nucleic acid molecule of embodiment 368, wherein the backbone of the sgRNA has a nucleotide sequence having at least 95% sequence identity to any one of SEQ ID NOs: 563-573.
371. The nucleic acid molecule of embodiment 368, wherein the backbone of the sgRNA has the nucleotide sequence set forth as any one of SEQ ID NOs: 563-573.
372. The nucleic acid molecule of any one of embodiments 357-371, wherein the gRNA comprises a first stem loop comprising a first stem and a second stem formed by hybridization of the crRNA repeat and the anti-repeat, wherein the first stem of the first stem loop comprises a total length of at least 3, 4, 5, 6, 7, 8, 9, 10, or 11 bp.
373. The nucleic acid molecule of any one of embodiments 357-371, wherein the gRNA comprises a first stem loop comprising a first stem and a second stem formed by hybridization of the crRNA repeat and the anti-repeat, wherein the first stem of the first stem loop comprises a total length of at most 3, 4, 5, 6, 7, 8, 9, 10, or 11 bp.
374. The nucleic acid molecule of embodiment 372 or 373, wherein the first stem of the first stem loop comprises a total length of 6 bp.
375. The nucleic acid molecule of embodiment 372 or 373, wherein the first stem of the first stem loop comprises a total length of 3 bp.
376. The nucleic acid molecule of any one of embodiments 357-375, wherein the tail of the tracrRNA comprises a total length of at least 1, 2, 3, 4, 5, 6, or 7 nucleotides.
377. The nucleic acid molecule of any one of embodiments 357-375, wherein the tail of the tracrRNA comprises a total length of at most 1, 2, 3, 4, 5, 6, or 7 nucleotides.
378. The nucleic acid molecule of embodiment 376 or 377, wherein the tail of the tracrRNA comprises a total length of 3 nucleotides.
379. The nucleic acid molecule of embodiment 376 or 377, wherein the tail of the tracrRNA comprises a total length of 1 nucleotide.
380. The nucleic acid molecule of any one of embodiments 372-379, wherein the gRNA further comprises a second stem loop most proximal to the tail, wherein the second stem loop comprises a first stem and a second stem.
381. The nucleic acid molecule of embodiment 380, wherein the first stem of the second stem loop comprises a total length of at least 1, 2, 3, 4, 5, or 6 bp.
382. The nucleic acid molecule of embodiment 380, wherein the first stem of the second stem loop comprises a total length of at most 1, 2, 3, 4, 5, or 6 bp.
383. The nucleic acid molecule of embodiment 381 or 382, wherein the first stem of the second stem loop comprises a total length of 5 bp.
384. The nucleic acid molecule of any one of embodiments 380-383, wherein the first stem of the first stem loop comprises a total length of 6 bp, the tail of the tracrRNA comprises a total length of 3 nucleotides, and the first stem of the second stem loop comprises a total length of 5 bp.
385. The nucleic acid molecule of embodiment 357, wherein the gRNA is a dual guide RNA (dgRNA).
386. The nucleic acid molecule of embodiment 385, wherein the crRNA repeat comprises a total length of at least 10, 11, 12, 13, 14, 15, 16, 17, 18, 19, 20, or 21 nucleotides.
387. The nucleic acid molecule of embodiment 385, wherein the crRNA repeat comprises a total length of at most 10, 11, 12, 13, 14, 15, 16, 17, 18, 19, 20, or 21 nucleotides.
388. The nucleic acid molecule of embodiment 386 or 387, wherein the crRNA repeat comprises a total length of 13 nucleotides.
389. The nucleic acid molecule of embodiment 386 or 387, wherein the crRNA repeat comprises a total length of 16 nucleotides.
390. The nucleic acid molecule of embodiment 386 or 387, wherein the crRNA repeat of the dgRNA comprises a total length of 21 nucleotides.
391. The nucleic acid molecule of any one of embodiments 385-390, wherein the tracrRNA comprises a total length of at least 65, 70, 75, 80, or 85 nucleotides.
392. The nucleic acid molecule of any one of embodiments 385-390, wherein the tracrRNA comprises a total length of at most 65, 70, 75, 80, or 85 nucleotides.
393. The nucleic acid molecule of embodiment 391 or 392, wherein the tracrRNA comprises a total length of 74 nucleotides.
394. The nucleic acid molecule of embodiment 391 or 392, wherein the tracrRNA comprises a total length of 77 nucleotides.
395. The nucleic acid molecule of any one of embodiments 357-394, wherein the gRNA comprises a total length of at least 85, 90, 95, 100, 105, 110, 115, 120, 125, 130, or 135 nucleotides.
396. The nucleic acid molecule of any one of embodiments 357-394, wherein the gRNA comprises a total length of at most 85, 90, 95, 100, 105, 110, 115, 120, 125, 130, or 135 nucleotides.
397. The nucleic acid molecule of any one of embodiments 357-394, wherein the gRNA comprises a total length of 106 to 135 nucleotides.
398. The nucleic acid molecule of embodiment 397, wherein the gRNA comprises a total length of 117 to 119 nucleotides.
399. The nucleic acid molecule of any one of embodiments 357-398, wherein the gRNA is capable of targeting a bound RNA-guided nuclease (RGN) polypeptide to a target sequence.
400. The nucleic acid molecule of embodiment 399, wherein the RGN polypeptide is capable of recognizing a consensus protospacer adjacent motif (PAM) having the nucleotide sequence set forth as NNNNCC.
401. The nucleic acid molecule of embodiment 400, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.
402. The nucleic acid molecule of any one of embodiments 399-401, wherein the RGN polypeptide comprises an amino acid sequence having at least 80% sequence identity to SEQ ID NO: 545; and wherein the target sequence and the spacer are selected from the group consisting of:
a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides;
b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides;
c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides;
d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides;
e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and
f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides.
403. The nucleic acid molecule of embodiment 402, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 5 nucleotides;
b) a target sequence having the nucleotide sequence set forth as SEQ ID NO: 164 and a spacer having a nucleotide sequence that differs in length and/or sequence from SEQ ID NO: 163 by 5 nucleotides;
c) a target sequence having the nucleotide sequence set forth as SEQ ID NO: 190 and a spacer having a nucleotide sequence that differs in length and/or sequence from SEQ ID NO: 189 by 5 nucleotides;
d) a target sequence having the nucleotide sequence set forth as SEQ ID NO: 180 and a spacer having a nucleotide sequence that differs in length and/or sequence from SEQ ID NO: 179 by 5 nucleotides;
e) a target sequence having the nucleotide sequence set forth as SEQ ID NO: 198 and a spacer having a nucleotide sequence that differs in length and/or sequence from SEQ ID NO: 197 by 5 nucleotides; and
f) a target sequence having the nucleotide sequence set forth as SEQ ID NO: 194 and a spacer having a nucleotide sequence that differs in length and/or sequence from SEQ ID NO: 193 by 5 nucleotides.
404. The nucleic acid molecule of embodiment 402, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 4 nucleotides;
b) a target sequence having the nucleotide sequence set forth as SEQ ID NO: 164 and a spacer having a nucleotide sequence that differs in length and/or sequence from SEQ ID NO: 163 by 4 nucleotides;
c) a target sequence having the nucleotide sequence set forth as SEQ ID NO: 190 and a spacer having a nucleotide sequence that differs in length and/or sequence from SEQ ID NO: 189 by 4 nucleotides;
d) a target sequence having the nucleotide sequence set forth as SEQ ID NO: 180 and a spacer having a nucleotide sequence that differs in length and/or sequence from SEQ ID NO: 179 by 4 nucleotides;
e) a target sequence having the nucleotide sequence set forth as SEQ ID NO: 198 and a spacer having a nucleotide sequence that differs in length and/or sequence from SEQ ID NO: 197 by 4 nucleotides; and
f) a target sequence having the nucleotide sequence set forth as SEQ ID NO: 194 and a spacer having a nucleotide sequence that differs in length and/or sequence from SEQ ID NO: 193 by 4 nucleotides.
405. The nucleic acid molecule of embodiment 402, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 3 nucleotides;
b) a target sequence having the nucleotide sequence set forth as SEQ ID NO: 164 and a spacer having a nucleotide sequence that differs in length and/or sequence from SEQ ID NO: 163 by 3 nucleotides;
c) a target sequence having the nucleotide sequence set forth as SEQ ID NO: 190 and a spacer having a nucleotide sequence that differs in length and/or sequence from SEQ ID NO: 189 by 3 nucleotides;
d) a target sequence having the nucleotide sequence set forth as SEQ ID NO: 180 and a spacer having a nucleotide sequence that differs in length and/or sequence from SEQ ID NO: 179 by 3 nucleotides;
e) a target sequence having the nucleotide sequence set forth as SEQ ID NO: 198 and a spacer having a nucleotide sequence that differs in length and/or sequence from SEQ ID NO: 197 by 3 nucleotides; and
f) a target sequence having the nucleotide sequence set forth as SEQ ID NO: 194 and a spacer having a nucleotide sequence that differs in length and/or sequence from SEQ ID NO: 193 by 3 nucleotides.
406. The nucleic acid molecule of embodiment 402, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 2 nucleotides;
b) a target sequence having the nucleotide sequence set forth as SEQ ID NO: 164 and a spacer having a nucleotide sequence that differs in length and/or sequence from SEQ ID NO: 163 by 2 nucleotides;
c) a target sequence having the nucleotide sequence set forth as SEQ ID NO: 190 and a spacer having a nucleotide sequence that differs in length and/or sequence from SEQ ID NO: 189 by 2 nucleotides;
d) a target sequence having the nucleotide sequence set forth as SEQ ID NO: 180 and a spacer having a nucleotide sequence that differs in length and/or sequence from SEQ ID NO: 179 by 2 nucleotides;
e) a target sequence having the nucleotide sequence set forth as SEQ ID NO: 198 and a spacer having a nucleotide sequence that differs in length and/or sequence from SEQ ID NO: 197 by 2 nucleotides; and
f) a target sequence having the nucleotide sequence set forth as SEQ ID NO: 194 and a spacer having a nucleotide sequence that differs in length and/or sequence from SEQ ID NO: 193 by 2 nucleotides.
407. The nucleic acid molecule of embodiment 402, wherein the target sequence and the spacer are selected from the group consisting of:
a) a target sequence having the nucleotide sequence set forth as SEQ ID NO: 156 and a spacer having a nucleotide sequence that differs in length and/or sequence from SEQ ID NO: 155 by 1 nucleotide;
b) a target sequence having the nucleotide sequence set forth as SEQ ID NO: 164 and a spacer having a nucleotide sequence that differs in length and/or sequence from SEQ ID NO: 163 by 1 nucleotide;
c) a target sequence having the nucleotide sequence set forth as SEQ ID NO: 190 and a spacer having a nucleotide sequence that differs in length and/or sequence from SEQ ID NO: 189 by 1 nucleotide;
d) a target sequence having the nucleotide sequence set forth as SEQ ID NO: 180 and a spacer having a nucleotide sequence that differs in length and/or sequence from SEQ ID NO: 179 by 1 nucleotide;
e) a target sequence having the nucleotide sequence set forth as SEQ ID NO: 198 and a spacer having a nucleotide sequence that differs in length and/or sequence from SEQ ID NO: 197 by 1 nucleotide; and
f) a target sequence having the nucleotide sequence set forth as SEQ ID NO: 194 and a spacer having a nucleotide sequence that differs in length and/or sequence from SEQ ID NO: 193 by 1 nucleotide.
408. The nucleic acid molecule of embodiment 402, wherein the spacer has the nucleotide sequence set forth as any one of SEQ ID NOs: 155, 163, 189, 179, 197, and 193.
409. The nucleic acid molecule of any one of embodiments 402-408, wherein the RGN polypeptide has an amino acid sequence having at least 90% sequence identity to SEQ ID NO: 545.
410. The nucleic acid molecule of any one of embodiments 402-408, wherein the RGN polypeptide has an amino acid sequence having at least 95% sequence identity to SEQ ID NO: 545.
411. The nucleic acid molecule of any one of embodiments 402-408, wherein the RGN polypeptide comprises an amino acid sequence set forth as SEQ ID NO: 545.
412. The nucleic acid molecule of any one of embodiments 399-411, wherein the gRNA has a nucleotide sequence having at least 80% sequence identity to any one of SEQ ID NOs: 693-834.
413. The nucleic acid molecule of embodiment 412, wherein the gRNA has a nucleotide sequence having at least 90% sequence identity to any one of SEQ ID NOs: 693-834.
414. The nucleic acid molecule of embodiment 412, wherein the gRNA has a nucleotide sequence having at least 95% sequence identity to any one of SEQ ID NOs: 693-834.
415. The nucleic acid molecule of embodiment 412, wherein the gRNA has a nucleotide sequence set forth as any one of SEQ ID NOs: 693-834.
416. The nucleic acid molecule of embodiment 412, wherein the gRNA has a nucleotide sequence set forth as any one of SEQ ID NOs: 693, 694, 695, 696, 697, and 698.
417. The nucleic acid molecule of embodiment 401, wherein the RGN polypeptide is capable of recognizing a full PAM having the nucleotide sequence set forth as any one of GGGTCCTT, GGGGCCGA, GGGGCCCA, CGGCCCTG, GGGCCCAT, TGGCCC, TGGGCC, GGGCCC, CGGGCC, and AGGGCC.
418. The nucleic acid molecule of embodiment 417, wherein the RGN polypeptide has an amino acid sequence having at least 80% sequence identity to SEQ ID NO: 835.
419. The nucleic acid molecule of embodiment 418, wherein the RGN polypeptide has an amino acid sequence having at least 90% sequence identity to SEQ ID NO: 835.
420. The nucleic acid molecule of embodiment 418, wherein the RGN polypeptide has an amino acid sequence having at least 95% sequence identity to SEQ ID NO: 835.
421. The nucleic acid molecule of embodiment 418, wherein the RGN polypeptide has the amino acid sequence set forth as SEQ ID NO: 835.
422. The nucleic acid molecule of embodiment 401, wherein the RGN polypeptide is capable of recognizing a full PAM having the nucleotide sequence set forth as any one of TCGGCCCT, CAGGCCTG, TCGGCC, and CGGGCC.
423. The nucleic acid molecule of embodiment 422, wherein the RGN polypeptide has an amino acid sequence having at least 80% sequence identity to SEQ ID NO: 915.
424. The nucleic acid molecule of embodiment 423, wherein the RGN polypeptide has an amino acid sequence having at least 90% sequence identity to SEQ ID NO: 915.
425. The nucleic acid molecule of embodiment 423, wherein the RGN polypeptide has an amino acid sequence having at least 95% sequence identity to SEQ ID NO: 915.
426. The nucleic acid molecule of embodiment 423, wherein the RGN polypeptide has the amino acid sequence set forth as SEQ ID NO: 915.
427. The nucleic acid molecule of any one of embodiments 357-426, wherein the gRNA comprises at least one chemical modification.
428. The nucleic acid molecule of embodiment 427, wherein the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.
429. The nucleic acid molecule of embodiment 428, wherein the at least one chemical modification comprises MS modifications at the 3 terminal nucleotides at the 5′ region and at the 3 terminal nucleotides at the 3′ region of the gRNA.
430. The nucleic acid molecule of embodiment 429, wherein the crRNA repeat has the nucleotide sequence set forth as any one of SEQ ID NOs: 940, 942-945, 1228, 1230, and 1232.
431. The nucleic acid molecule of embodiment 429 or 430, wherein the crRNA has the nucleotide sequence set forth as any one of SEQ ID NOs: 967-1085.
432. The nucleic acid molecule of any one of embodiments 429-431, wherein the tracrRNA has the nucleotide sequence set forth as any one of SEQ ID NOs: 941, 946-955, 1229, 1231, and 1233.
433. The nucleic acid molecule of any one of embodiments 429-432, wherein the gRNA has the nucleotide sequence set forth as any one of SEQ ID NOs: 1086-1227.
434. The nucleic acid molecule of embodiment 428, wherein the BNA comprises a 2′,4′ BNA modification.
435. The nucleic acid molecule of embodiment 434, wherein the 2′,4′ BNA modification is selected from the group consisting of: locked nucleic acid (LNA) modification, BNANC[N-Me]modification, 2′-0,4′-C-ethylene bridged nucleic acid (2′,4′-ENA) modification, and S-constrained ethyl (cEt) modification.
436. The nucleic acid molecule of embodiment 435, wherein the 2′,4′ BNA is a LNA modification.
437. The nucleic acid molecule of embodiment 435, wherein the 2′,4′ BNA is a cEt modification.
438. The nucleic acid molecule of embodiment 428, wherein the at least one chemical modification comprises a BNA modification, 2′-O-Me modification, PS modification, or a combination thereof.
439. The nucleic acid molecule of any one of embodiments 357-438, wherein the gRNA further comprises an extension comprising an edit template for reverse transcriptase editing.
440. A vector comprising the nucleic acid molecule of any one of embodiments 334-356, wherein the nucleic acid molecule comprises a polynucleotide encoding the crRNA.
441. The vector of embodiment 440, wherein the nucleic acid molecule further comprises a heterologous promoter operably linked to the polynucleotide encoding the crRNA.
442. The vector of embodiment 441, wherein the heterologous promoter is an RNA polymerase III (pol III) promoter.
443. The vector of any one of embodiments 440-442, wherein the vector further comprises a nucleic acid molecule encoding an RGN polypeptide.
444. The vector of embodiment 443, wherein the crRNA is capable of binding a tracrRNA to form a guide RNA, and wherein the guide RNA is capable of binding to the RGN polypeptide.
445. The vector of embodiment 443 or 444, wherein the vector further comprises a promoter operably linked to the nucleic acid molecule encoding the RGN polypeptide.
446. A vector comprising the nucleic acid molecule of any one of embodiments 357-439, wherein the nucleic acid molecule comprises a polynucleotide encoding the crRNA, and wherein the vector further comprises a polynucleotide encoding the tracrRNA.
447. The vector of embodiment 446, wherein the polynucleotide encoding the crRNA and the polynucleotide encoding the tracrRNA are operably linked to the same promoter and are encoded as a sgRNA.
448. The vector of embodiment 446, wherein the polynucleotide encoding the crRNA and the polynucleotide encoding the tracrRNA are operably linked to separate promoters.
449. The vector of any one of embodiments 446-448, wherein the vector further comprises a nucleic acid molecule encoding an RGN polypeptide.
450. The vector of embodiment 449, wherein the crRNA is capable of binding the tracrRNA to form a guide RNA, and wherein the guide RNA is capable of binding to the RGN polypeptide.
451. The vector of embodiment 449 or 450, wherein the vector further comprises a promoter operably linked to the nucleic acid molecule encoding the RGN polypeptide.
452. A cell comprising the gRNA of any one of embodiments 1-216, the nucleic acid molecule of any one of embodiments 217-321 and 334-439, or the vector of any one of embodiments 322-333 and 440-451.
453. An RNA-guided nuclease (RGN) system for binding a target sequence within a forkhead box P3 (FOXP3) gene, wherein the RGN system comprises:
a) one or more guide RNA (gRNA) of any one of embodiments 1-216, or one or more polynucleotides comprising one or more nucleotide sequences encoding the one or more gRNA of any one of embodiments 1-216; and
b) an RGN polypeptide, or a polynucleotide comprising a nucleotide sequence encoding the RGN polypeptide.
454. The RGN system of embodiment 453, wherein the one or more gRNA is capable of forming a complex with the RGN polypeptide to direct the RGN polypeptide to bind to the target sequence.
455. The RGN system of embodiment 453 or 454, wherein the RGN polypeptide is capable of recognizing a consensus protospacer adjacent motif (PAM) having the nucleotide sequence set forth as NNNNCC.
456. The RGN system of embodiment 455, wherein the RGN polypeptide is capable of recognizing a full PAM having a nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.
457. The RGN system of any one of embodiments 453-456, wherein the RGN polypeptide comprises an amino acid sequence having at least 80% sequence identity to SEQ ID NO: 545.
458. The RGN system of embodiment 457, wherein the RGN polypeptide comprises an amino acid sequence having at least 90% sequence identity to SEQ ID NO: 545.
459. The RGN system of embodiment 457, wherein the RGN polypeptide comprises an amino acid sequence having at least 95% sequence identity to SEQ ID NO: 545.
460. The RGN system of embodiment 457, wherein the RGN polypeptide comprises the amino acid sequence set forth as SEQ ID NO: 545.
461. The RGN system of any one of embodiments 453-460, wherein the polynucleotide comprising a nucleotide sequence encoding the RGN polypeptide is codon optimized for expression in a mammalian cell.
462. The RGN system of any one of embodiments 453-461, wherein at least one of the one or more nucleotide sequences encoding the one or more gRNAs and the nucleotide sequence encoding the RGN polypeptide is operably linked to a promoter heterologous to the nucleotide sequence.
463. The RGN system of any one of embodiments 453-462, wherein the one or more nucleotide sequences encoding the one or more gRNAs and the nucleotide sequence encoding the RGN polypeptide are located on one vector.
464. The RGN system of any one of embodiments 453-460, wherein the polynucleotide comprising a nucleotide sequence encoding the RGN polypeptide comprises an mRNA.
465. The RGN system of any one of embodiments 453-464, wherein the RGN polypeptide is nuclease inactive or is a nickase.
466. The RGN system of any one of embodiments 453-465, wherein the RGN polypeptide is fused to a base-editing polypeptide.
467. The RGN system of embodiment 466, wherein the base-editing polypeptide comprises a deaminase.
468. The RGN system of any one of embodiments 453-465, wherein the RGN polypeptide is fused to a reverse transcriptase (RT) editing polypeptide.
469. The RGN system of embodiment 468, wherein the RT editing polypeptide comprises a DNA polymerase.
470. The RGN system of embodiment 469, wherein the DNA polymerase comprises a reverse transcriptase.
471. The RGN system of any one of embodiments 468-470, wherein the gRNA further comprises an extension comprising an edit template for RT editing.
472. The RGN system of any one of embodiments 453-471, wherein the RGN polypeptide comprises one or more nuclear localization signals.
473. A ribonucleoprotein (RNP) complex comprising the one or more gRNA and the RGN polypeptide of the RGN system of any one of embodiments 453-472.
474. A cell comprising the RGN system of any one of embodiments 453-472 or the RNP complex of embodiment 473.
475. The cell of embodiment 474, wherein the cell is a eukaryotic cell.
476. The cell of embodiment 475, wherein the eukaryotic cell is a mammalian cell.
477. The cell of embodiment 476, wherein the mammalian cell is a human cell.
478. The cell of embodiment 476 or 477, wherein the mammalian cell or human cell is a T cell or an induced pluripotent stem cell.
479. A method for binding a target sequence within a FOXP3 gene, comprising delivering the RGN system of any one of embodiments 453-472 or the RNP complex of embodiment 473 to the target sequence or a cell comprising the target sequence.
480. The method of embodiment 479, wherein cleavage or modification of the target sequence occurs.
481. A method for assembling an RNA-guided nuclease (RGN) ribonucleoprotein complex, the method comprising combining under conditions suitable for formation of the complex:
a) the guide RNA of any one of embodiments 1-216; and
b) an RGN polypeptide that binds the guide RNA.
482. The method of embodiment 481, wherein the RGN polypeptide is capable of recognizing a consensus protospacer adjacent motif (PAM) having the nucleotide sequence set forth as NNNNCC.
483. The method of embodiment 481 or 482, wherein the complex directs cleavage of the target sequence.
484. The method of embodiment 483, wherein the cleavage generates a double-stranded break.
485. The method of embodiment 483, wherein the cleavage generates a single-stranded break.
486. A method for binding a target sequence within a FOXP3 gene, the method comprising:
a) combining under conditions suitable for formation of a ribonucleoprotein (RNP) complex:
   i) the guide RNA of any one of embodiments 1-216; and
   ii) an RGN polypeptide that binds the guide RNA; thereby assembling an RNP complex; and
b) contacting the target sequence or a cell comprising the target sequence with the assembled RNP complex.
487. The method of embodiment 486, wherein the guide RNA hybridizes to the target sequence, thereby directing binding of the RNP complex to the target sequence.
488. The method of embodiment 486 or 487, wherein the RGN polypeptide is capable of recognizing a consensus protospacer adjacent motif (PAM) having the nucleotide sequence set forth as NNNNCC.
489. The method of embodiment 488, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.
490. The method of any one of embodiments 486-489, wherein the RGN polypeptide comprises an amino acid sequence having at least 80% sequence identity to SEQ ID NO: 545.
491. The method of embodiment 490, wherein the RGN polypeptide comprises an amino acid sequence having at least 90% sequence identity to SEQ ID NO: 545.
492. The method of embodiment 490, wherein the RGN polypeptide comprises an amino acid sequence having at least 95% sequence identity to SEQ ID NO: 545.
493. The method of embodiment 490, wherein the RGN polypeptide comprises an amino acid sequence set forth as SEQ ID NO: 545.
494. The method of any one of embodiments 486-493, wherein the method is performed in vitro or ex vivo.
495. The method of any one of embodiments 486-494, wherein the RGN polypeptide is capable of cleaving the target sequence, thereby allowing for the cleaving and/or modifying of the target sequence.
496. The method of embodiment 495, wherein the cleaving generates a single-stranded break.
497. The method of embodiment 495, wherein the cleaving generates a double-stranded break.
498. The method of embodiment 495, wherein the cleaving results in insertion of a heterologous sequence within the target sequence.
499. The method of any one of embodiments 486-494, wherein the RGN polypeptide is nuclease inactive or is a nickase.
500. The method of embodiment 499, wherein the RGN polypeptide is fused to a base-editing polypeptide.
501. The method of embodiment 500, wherein the base-editing polypeptide comprises a deaminase.
502. The method of any one of embodiments 486-494, wherein the RGN is fused to a reverse transcriptase (RT) editing polypeptide.
503. The method of embodiment 502, wherein the RT editing polypeptide comprises a DNA polymerase.
504. The method of embodiment 503, wherein the DNA polymerase comprises a reverse transcriptase.
505. The method of any one of embodiments 502-504, wherein the gRNA further comprises an extension comprising an edit template for RT editing.
506. The method of any one of embodiments 486-505, wherein the target sequence is within a cell.
507. The method of embodiment 506, wherein the cell is a eukaryotic cell.
508. The method of embodiment 507, wherein the eukaryotic cell is a mammalian cell.
509. The method of embodiment 508, wherein the mammalian cell is a human cell.
510. The method of embodiment 508 or 509, wherein the mammalian cell or human cell is a T cell or an induced pluripotent stem cell.
511. The method of any one of embodiments 486-510, further comprising selecting a cell comprising a modified target sequence.
512. A cell comprising a modified target sequence obtained according to the method of embodiment 511.
513. The cell of embodiment 512, wherein the cell is a eukaryotic cell.
514. The cell of embodiment 513, wherein the eukaryotic cell is a mammalian cell.
515. The cell of embodiment 514, wherein the mammalian cell is a human cell.
516. The cell of embodiment 514 or 515, wherein the mammalian cell or human cell is a T cell or an induced pluripotent stem cell.
517. A method for producing a genetically modified cell comprising insertions and/or deletions within a forkhead box P3 (FOXP3) gene, wherein the method comprises introducing into a cell the RGN system of any one of embodiments 453-472 or an RNP complex of embodiment 473.
518. The method of embodiment 517, wherein the genetically modified cell has lower levels of Foxp3 protein compared to a cell that has not been genetically modified.
519. The method of embodiment 517 or 518, wherein the cell is a mammalian cell.
520. The method of embodiment 519, wherein the mammalian cell is a human cell.
521. The method of embodiment 519 or 520, wherein the mammalian cell or human cell is a T cell or an induced pluripotent stem cell.
522. A genetically modified cell comprising insertions and/or deletions within a FOXP3 gene produced according to the method of any one of embodiments 517-521.
523. A method for modulating expression of a forkhead box P3 (FOXP3) gene in a population of cells, comprising delivering the RGN system of any one of embodiments 453-472 or the RNP complex of embodiment 473 to the population of cells, wherein the population of cells comprises the target sequence, and wherein FOXP3 gene expression is modulated as compared to FOXP3 gene expression in a control population of cells.
524. The method of embodiment 523, wherein cleavage or modification of the target sequence occurs.
525. The method of embodiment 524, wherein cleavage or modification of the target sequence is detected by sequencing.
526. The method of any one of embodiments 523-525, wherein FOXP3 gene expression is measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof.
527. The method of any one of embodiment 523-526, wherein FOXP3 gene expression is decreased.
528. The method of embodiment 527, wherein the decrease in FOXP3 gene expression comprises decrease in FOXP3 mRNA and/or Foxp3 protein.
529. The method of any one of embodiments 524-528, wherein cleavage or modification of the target sequence occurs at a rate of 40% to 100%.
530. The method of any one of embodiments 524-529, wherein cleavage or modification of the target sequence occurs at a rate of 80% to 100%.
531. The method of any one of embodiments 523-530, wherein the control population of cells has not been subjected to the delivering.
532. The method of any one of embodiments 523-531, wherein the population of cells comprises T cells.
   The following examples are offered by way of illustration and not by way of limitation.

### EXAMPLES

### Example 1. A Screen to Identify Guide RNAs Effective in Targeting a Forkhead Protein P3 (FOXP3) gene for editing

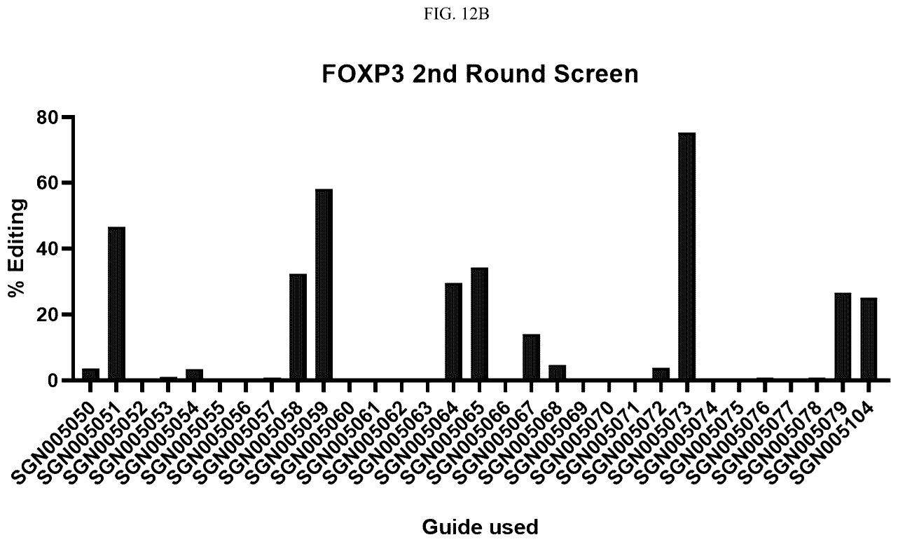

Guide RNAs were screened for their effectiveness in cutting target sequences in the FOXP3 gene in association with the APG07433.1 RGN. T cells were thawed and activated as described in Example 2. Three days after activation, 4 μg of guide RNA and 2 μg of the APG07433.1 RGN were delivered to the T cells via Amaxa nucleofection. The tested guide RNAs, their target sequences, and PAM sequences are listed in Table 2. Table 2 also indicates which FOXP3 target sequences could also be targeted by S. pyogenes Cas9 (SpyCas9) and/or LPG10145 RGN due to the PAM sequences. Two days after lipofection, the genomic DNA (gDNA) was extracted from the cells, and next generation sequencing (NGS) was performed on an amplified fragment of the FOXP3 gene. Table 4 shows the primer sequences used for amplifications. An initial screen using mRNA encoding the APG07433.1 RGN (mRNA) or the APG07433.1 RGN (RNP) showed that using 20 nt spacer lengths yielded low editing (FIG. 12A). A subsequent screen was performed with increased spacer length for top guide RNAs having editing above 5%. Table 3 and FIG. 12B show gene editing as percent insertions and deletions (indels) using FOXP3 guide RNAs with APG07433.1 RGN. Table 5 shows gene editing data for lead FOXP3 guide RNAs as a function of ribonucleoprotein (RNP) dose response. The guide RNAs from the screening that were most effective in targeting FOXP3 for gene editing are listed in Table 6.

TABLE 2
List of FOXP3 guide RNAs screened.

| | | | | | | Targetable |
|---|---|---|---|---|---|---|
| | Guide | | | | | by |
| | RNA | | Target | | Target | SpyCas9* |
| Guide | SEQ | | SEQ | PAM | Length | and/or |
| name | ID NO: | Target Sequence | ID NO: | Sequence | (in nt) | LPG10145 |
| SGN002770 | 699 | TTGGACAAGGACCCGATGCC | 2 | CAACCCCA | 20 | |
| SGN002771 | 700 | TGGACAAGGACCCGATGCCC | 4 | AACCCCAG | 20 | |
| SGN002772 | 701 | TGGGGTTGGGCATCGGGTCC | 6 | TTGTCCAA | 20 | |
| SGN002773 | 702 | AAGGACCCGATGCCCAACCC | 8 | CAGGCCTG | 20 | |
| SGN002774 | 703 | CAGGCCTGGGGTTGGGCATC | 10 | GGGTCCTT | 20 | SpyCas9 |
| SGN002775 | 704 | ATGCCCAACCCCAGGCCTGG | 12 | CAAGCCCT | 20 | |
| SGN002776 | 705 | TGCCCTTGGACAAGGACCCG | 14 | ATGCCCAA | 20 | |
| SGN002777 | 706 | CTTGGACAAGGACCCGATGC | 16 | CCAACCCC | 20 | |
| SGN002778 | 707 | TCCTCTTCTTCCTTGAACCC | 18 | CATGCCAC | 20 | |
| SGN002779 | 708 | TCTTCTTCCTTGAACCCCAT | 20 | GCCACCAT | 20 | |
| SGN002780 | 709 | CGCAGCCTGCCCTTGGACAA | 22 | GGACCCGA | 20 | |
| SGN002781 | 710 | GGAAGGGGCCGAGGGCTTGC | 24 | CAGGCCTG | 20 | |
| SGN002782 | 711 | GGCCTGGCAAGCCCTCGGCC | 26 | CCTTCCTT | 20 | |
| SGN002783 | 712 | GCAAGCCCTCGGCCCCTTCC | 28 | TTGGCCCT | 20 | |
| SGN002784 | 713 | ATGGGCCAAGGGCCAAGGAA | 30 | GGGGCCGA | 20 | SpyCas9 |
| SGN002785 | 714 | CTTGGCCCATCCCCAGGAGC | 32 | CTCGCCCA | 20 | |
| SGN002786 | 715 | CCTCGCCCAGCTGGAGGGCT | 34 | GCACCCAA | 20 | |
| SGN002787 | 716 | GTCTGAGGCTTTGGGTGCAG | 36 | CCCTCCAG | 20 | |
| SGN002788 | 717 | CAGGTCTGAGGCTTTGGGTG | 38 | CAGCCCTC | 20 | |
| SGN002789 | 718 | AAGGTTCCCCCTGGGCCCCG | 40 | GGCCCCCA | 20 | |
| SGN002790 | 719 | GAAGGTTCCCCCTGGGCCCC | 42 | GGGCCCCC | 20 | |
| SGN002791 | 720 | GCCCTGGAAGGTTCCCCCTG | 44 | GGCCCCGG | 20 | |
| SGN002792 | 721 | GGGCCCAGGGGGAACCTTCC | 46 | AGGGCCGA | 20 | |
| SGN002793 | 722 | AGATCTCGGCCCTGGAAGGT | 48 | TCCCCCTG | 20 | |
| SGN002794 | 723 | AAGATCTCGGCCCTGGAAGG | 50 | TTCCCCCT | 20 | |
| SGN002795 | 724 | GAAGATCTCGGCCCTGGAAG | 52 | GTTCCCCC | 20 | |
| SGN002796 | 725 | CGAAGATCTCGGCCCTGGAA | 54 | GGTTCCCC | 20 | |
| SGN002797 | 726 | AGGGCCGAGATCTTCGAGGC | 56 | GGGGCCCA | 20 | SpyCas9 |
| SGN002798 | 727 | GGGCCCCGCCTCGAAGATCT | 58 | CGGCCCTG | 20 | SpyCas9 |
| SGN002799 | 728 | GGGCCGAGATCTTCGAGGCG | 60 | GGGCCCAT | 20 | SpyCas9 |
| SGN002800 | 729 | TGGGCCCCGCCTCGAAGATC | 62 | TCGGCCCT | 20 | LPG10145 |
| SGN002801 | 730 | GAGATCTTCGAGGCGGGGCC | 64 | CATGCCTC | 20 | |
| SGN002802 | 731 | ATCTTCGAGGCGGGGCCCAT | 66 | GCCTCCTC | 20 | |
| SGN002803 | 732 | GCGGGGCCCATGCCTCCTCT | 68 | TCTTCCTT | 20 | |
| SGN003378 | 733 | CTTGCCAGGCCTGGGGTTGG | 70 | GGGTCCTT | 25 | SpyCas9 |
| | | GCATC | | | | |
| SGN003379 | 734 | ACCCGATGCCCAACCCCAGG | 72 | CAAGCCCT | 25 | |
| | | CCTGG | | | | |
| SGN003380 | 735 | GCCAAGGAAGGGGCCGAGG | 74 | CAGGCCTG | 25 | |
| | | GCTTGC | | | | |
| SGN003381 | 736 | AGCAGGTCTGAGGCTTTGGG | 76 | CCCTCCAG | 25 | |
| | | TGCAG | | | | |
| SGN003382 | 737 | TCTCGGCCCTGGAAGGTTCC | 78 | GGCCCCGG | 25 | |
| | | CCCTG | | | | |
| SGN003383 | 738 | CTCGAAGATCTCGGCCCTGG | 80 | CAGGCCTG | 25 | LPG10145 |
| | | AAGGT | | | | |
| SGN003384 | 739 | CCTCGAAGATCTCGGCCCTG | 82 | TTCCCCCT | 25 | |
| | | GAAGG | | | | |
| SGN005042 | 740 | TTGCCAGGCCTGGGGTTGGG | 84 | GGGTCCTT | 24 | SpyCas9 |
| | | CATC | | | | |
| SGN005043 | 741 | GCAGGTCTGAGGCTTTGGGT | 86 | CCCTCCAG | 24 | |
| | | GCAG | | | | |
| SGN005044 | 742 | CTCGAAGATCTCGGCCCTGG | 88 | TTCCCCCT | 24 | |
| | | AAGG | | | | |
| SGN005050 | 743 | AGCCCTCGGCCCCTTCCTTGG | 90 | TGGCCC | 25 | SpyCas9 |
| | | CCCT | | | | |
| SGN005051 | 744 | AGCTGGAGGGCTGCACCCAA | 92 | CAGACC | 25 | |
| | | AGCCT | | | | |
| SGN005052 | 745 | AGGCATGGGCCCCGCCTCGA | 94 | TCGGCC | 25 | |
| | | AGATC | | | | |
| SGN005053 | 746 | CAAGGGCCAAGGAAGGGGC | 96 | CTTGCC | 25 | |
| | | CGAGGG | | | | |
| SGN005054 | 747 | CCAACCCCAGGCCTGGCAAG | 98 | GGCCCC | 25 | |
| | | CCCTC | | | | |
| SGN005055 | 748 | CCCCAGCAGGTCTGAGGCTT | 100 | GCAGCC | 25 | |
| | | TGGGT | | | | |
| SGN005056 | 749 | CCCGATGCCCAACCCCAGGC | 102 | AAGCCC | 25 | |
| | | CTGGC | | | | |
| SGN005057 | 750 | CCGAGATCTTCGAGGCGGGG | 104 | GCCTCC | 25 | |
| | | CCCAT | | | | |
| SGN005058 | 751 | CCTCCTCTTCTTCCTTGAACC | 106 | GCCACC | 25 | |
| | | CCAT | | | | |
| SGN005059 | 752 | CCTGGCAAGCCCTCGGCCCC | 108 | TGGCCC | 25 | SpyCas9 |
| | | TTCCT | | | | |
| SGN005060 | 753 | CGGCCCCTTCCTTGGCCCTTG | 110 | ATCCCC | 25 | |
| | | GCCC | | | | |
| SGN005061 | 754 | CTCGCCCAGCTGGAGGGCTG | 112 | AAAGCC | 25 | |
| | | CACCC | | | | |
| SGN005062 | 755 | CTCGGCCCCTTCCTTGGCCCT | 114 | CCATCC | 25 | |
| | | TGGC | | | | |
| SGN005063 | 756 | CTGGGGGCCCGGGGCCCAGG | 116 | CCTTCC | 25 | |
| | | GGGAA | | | | |
| SGN005064 | 757 | GATCTCGGCCCTGGAAGGTT | 118 | TGGGCC | 25 | SpyCas9 |
| | | CCCCC | | | | |
| SGN005065 | 758 | GCACCCAAAGCCTCAGACCT | 120 | GGGCCC | 25 | SpyCas9 |
| | | GCTGG | | | | |
| SGN005066 | 759 | GCCCAACCCCAGGCCTGGCA | 122 | TCGGCC | 25 | LPG10145 |
| | | AGCCC | | | | |
| SGN005067 | 760 | GCCCTGGAAGGTTCCCCCTG | 124 | CGGGCC | 25 | SpyCas9 |
| | | GGCCC | | | | and |
| | | | | | | LPG10145 |
| SGN005068 | 761 | GGCCCATGCCTCCTCTTCTTC | 126 | AACCCC | 25 | |
| | | CTTG | | | | |
| SGN005069 | 762 | GGCCCTTGGCCCATCCCCAG | 128 | TCGCCC | 25 | |
| | | GAGCC | | | | |
| SGN005070 | 763 | GGGCCGAGATCTTCGAGGCG | 130 | CATGCC | 25 | |
| | | GGGCC | | | | |
| SGN005071 | 764 | GGGCGAGGCTCCTGGGGATG | 132 | AGGGCC | 25 | SpyCas9 |
| | | GGCCA | | | | |
| SGN005072 | 765 | GGGGCCCATGCCTCCTCTTCT | 134 | TGAACC | 25 | |
| | | TCCT | | | | |
| SGN005073 | 766 | GGGGTTCAAGGAAGAAGAG | 136 | TGGGCC | 25 | SpyCas9 |
| | | GAGGCA | | | | |
| SGN005074 | 767 | GGTTCAAGGAAGAAGAGGA | 138 | GGCCCC | 25 | |
| | | GGCATG | | | | |
| SGN005075 | 768 | TCAAGGAAGAAGAGGAGGC | 140 | CCCGCC | 25 | |
| | | ATGGGC | | | | |
| SGN005076 | 769 | TCCAGCTGGGCGAGGCTCCT | 142 | TGGGCC | 25 | SpyCas9 |
| | | GGGGA | | | | |
| SGN005077 | 770 | TCGAGGCGGGGCCCATGCCT | 144 | TCTTCC | 25 | |
| | | CCTCT | | | | |
| SGN005078 | 771 | TGGGTGCAGCCCTCCAGCTG | 146 | GGCTCC | 25 | |
| | | GGCGA | | | | |
| SGN005079 | 772 | TTCCAGGGCCGAGATCTTCG | 148 | GGGCCC | 25 | SpyCas9 |
| | | AGGCG | | | | |
| SGN005104 | 773 | AAGCCTCAGACCTGCTGGGG | 150 | GGGCCC | 25 | SpyCas9 |
| | | GCCCG | | | | |
| SGN005635 | 774 | CTTGCCAGGCCTGGGGTTGG | 152 | GGGTCCTT | 25 | SpyCas9 |
| | | GCATC | | | | |
| SGN005636 | 775 | TTGCCAGGCCTGGGGTTGGG | 154 | GGGTCCTT | 24 | SpyCas9 |
| | | CATC | | | | |
| SGN005637 | 693 | TGCCAGGCCTGGGGTTGGGC | 156 | GGGTCCTT | 23 | SpyCas9 |
| | | ATC | | | | |
| SGN005638 | 776 | GCCAGGCCTGGGGTTGGGCA | 158 | GGGTCCTT | 22 | SpyCas9 |
| | | TC | | | | |
| SGN005639 | 777 | AGCAGGTCTGAGGCTTTGGG | 160 | CCCTCCAG | 25 | |
| | | TGCAG | | | | |
| SGN005640 | 778 | GCAGGTCTGAGGCTTTGGGT | 162 | CCCTCCAG | 24 | |
| | | GCAG | | | | |
| SGN005641 | 694 | CAGGTCTGAGGCTTTGGGTG | 164 | CCCTCCAG | 23 | |
| | | CAG | | | | |
| SGN005642 | 779 | AGGTCTGAGGCTTTGGGTGC | 166 | CCCTCCAG | 22 | |
| | | AG | | | | |
| SGN005643 | 780 | TCTCGGCCCTGGAAGGTTCC | 168 | GGCCCCGG | 25 | |
| | | CCCTG | | | | |
| SGN005644 | 781 | CTCGGCCCTGGAAGGTTCCC | 170 | GGCCCCGG | 24 | |
| | | CCTG | | | | |
| SGN005645 | 782 | TCGGCCCTGGAAGGTTCCCC | 172 | GGCCCCGG | 23 | |
| | | CTG | | | | |
| SGN005646 | 783 | CGGCCCTGGAAGGTTCCCCC | 174 | GGCCCCGG | 22 | |
| | | TG | | | | |
| SGN005647 | 784 | CCTCGAAGATCTCGGCCCTG | 176 | TTCCCCCT | 25 | |
| | | GAAGG | | | | |
| SGN005648 | 785 | CTCGAAGATCTCGGCCCTGG | 178 | TTCCCCCT | 24 | |
| | | AAGG | | | | |
| SGN005649 | 695 | TCGAAGATCTCGGCCCTGGA | 180 | TTCCCCCT | 23 | |
| | | AGG | | | | |
| SGN005650 | 786 | CGAAGATCTCGGCCCTGGAA | 182 | TTCCCCCT | 22 | |
| | | GG | | | | |
| SGN005651 | 787 | CTCGGCCCTGGAAGGTTCCC | 184 | GGCCCCGG | 24 | |
| | | CCTG | | | | |
| SGN005683 | 788 | CTTGCCAGGCCTGGGGTTGG | 186 | GGGTCCTT | 25 | SpyCas9 |
| | | GCATC | | | | |
| SGN005684 | 789 | CTTGCCAGGCCTGGGGTTGG | 186 | GGGTCCTT | 25 | SpyCas9 |
| | | GCATC | | | | |
| SGN005685 | 790 | CTTGCCAGGCCTGGGGTTGG | 186 | GGGTCCTT | 25 | SpyCas9 |
| | | GCATC | | | | |
| SGN005686 | 791 | CTTGCCAGGCCTGGGGTTGG | 186 | GGGTCCTT | 25 | SpyCas9 |
| | | GCATC | | | | |
| SGN005687 | 792 | CTTGCCAGGCCTGGGGTTGG | 186 | GGGTCCTT | 25 | SpyCas9 |
| | | GCATC | | | | |
| SGN005688 | 793 | CTTGCCAGGCCTGGGGTTGG | 186 | GGGTCCTT | 25 | SpyCas9 |
| | | GCATC | | | | |
| SGN005689 | 794 | CTTGCCAGGCCTGGGGTTGG | 186 | GGGTCCTT | 25 | SpyCas9 |
| | | GCATC | | | | |
| SGN005690 | 795 | CTTGCCAGGCCTGGGGTTGG | 186 | GGGTCCTT | 25 | SpyCas9 |
| | | GCATC | | | | |
| SGN005691 | 796 | CTTGCCAGGCCTGGGGTTGG | 186 | GGGTCCTT | 25 | SpyCas9 |
| | | GCATC | | | | |
| SGN005992 | 797 | AGCAGGTCTGAGGCTTTGGG | 188 | CCCTCCAG | 25 | |
| | | TGCAG | | | | |
| SGN005693 | 798 | AGCAGGTCTGAGGCTTTGGG | 188 | CCCTCCAG | 25 | |
| | | TGCAG | | | | |
| SGN005694 | 799 | AGCAGGTCTGAGGCTTTGGG | 188 | CCCTCCAG | 25 | |
| | | TGCAG | | | | |
| SGN005695 | 800 | AGCAGGTCTGAGGCTTTGGG | 188 | CCCTCCAG | 25 | |
| | | TGCAG | | | | |
| SGN005696 | 801 | AGCAGGTCTGAGGCTTTGGG | 188 | CCCTCCAG | 25 | |
| | | TGCAG | | | | |
| SGN005697 | 802 | AGCAGGTCTGAGGCTTTGGG | 188 | CCCTCCAG | 25 | |
| | | TGCAG | | | | |
| SGN005698 | 803 | AGCAGGTCTGAGGCTTTGGG | 188 | CCCTCCAG | 25 | |
| | | TGCAG | | | | |
| SGN005699 | 804 | AGCAGGTCTGAGGCTTTGGG | 188 | CCCTCCAG | 25 | |
| | | TGCAG | | | | |
| SGN005700 | 805 | AGCAGGTCTGAGGCTTTGGG | 188 | CCCTCCAG | 25 | |
| | | TGCAG | | | | |
| SGN005701 | 806 | TCTCGGCCCTGGAAGGTTCC | 190 | GGCCCCGG | 25 | |
| | | CCCTG | | | | |
| SGN005702 | 807 | TCTCGGCCCTGGAAGGTTCC | 190 | GGCCCCGG | 25 | |
| | | CCCTG | | | | |
| SGN005703 | 808 | TCTCGGCCCTGGAAGGTTCC | 190 | GGCCCCGG | 25 | |
| | | CCCTG | | | | |
| SGN005704 | 809 | TCTCGGCCCTGGAAGGTTCC | 190 | GGCCCCGG | 25 | |
| | | CCCTG | | | | |
| SGN005705 | 810 | TCTCGGCCCTGGAAGGTTCC | 190 | GGCCCCGG | 25 | |
| | | CCCTG | | | | |
| SGN005706 | 811 | TCTCGGCCCTGGAAGGTTCC | 190 | GGCCCCGG | 25 | |
| | | CCCTG | | | | |
| SGN005707 | 696 | TCTCGGCCCTGGAAGGTTCC | 190 | GGCCCCGG | 25 | |
| | | CCCTG | | | | |
| SGN005708 | 812 | TCTCGGCCCTGGAAGGTTCC | 190 | GGCCCCGG | 25 | |
| | | CCCTG | | | | |
| SGN005709 | 813 | TCTCGGCCCTGGAAGGTTCC | 190 | GGCCCCGG | 25 | |
| | | CCCTG | | | | |
| SGN005710 | 814 | CCTCGAAGATCTCGGCCCTG | 192 | TTCCCCCT | 25 | |
| | | GAAGG | | | | |
| SGN005711 | 815 | CCTCGAAGATCTCGGCCCTG | 192 | TTCCCCCT | 25 | |
| | | GAAGG | | | | |
| SGN005712 | 816 | CCTCGAAGATCTCGGCCCTG | 192 | TTCCCCCT | 25 | |
| | | GAAGG | | | | |
| SGN005713 | 817 | CCTCGAAGATCTCGGCCCTG | 192 | TTCCCCCT | 25 | |
| | | GAAGG | | | | |
| SGN005714 | 818 | CCTCGAAGATCTCGGCCCTG | 192 | TTCCCCCT | 25 | |
| | | GAAGG | | | | |
| SGN005715 | 819 | CCTCGAAGATCTCGGCCCTG | 192 | TTCCCCCT | 25 | |
| | | GAAGG | | | | |
| SGN005716 | 820 | CCTCGAAGATCTCGGCCCTG | 192 | TTCCCCCT | 25 | |
| | | GAAGG | | | | |
| SGN005717 | 821 | CCTCGAAGATCTCGGCCCTG | 192 | TTCCCCCT | 25 | |
| | | GAAGG | | | | |
| SGN005718 | 822 | CCTCGAAGATCTCGGCCCTG | 192 | TTCCCCCT | 25 | |
| | | GAAGG | | | | |
| SGN006269 | 823 | GGGGTTCAAGGAAGAAGAG | 194 | TGGGCC | 25 | SpyCas9 |
| | | GAGGCA | | | | |
| SGN006270 | 824 | GGGTTCAAGGAAGAAGAGG | 196 | TGGGCC | 24 | SpyCas9 |
| | | AGGCA | | | | |
| SGN006271 | 697 | GGTTCAAGGAAGAAGAGGA | 198 | TGGGCC | 23 | SpyCas9 |
| | | GGCA | | | | |
| SGN006272 | 698 | GGGGTTCAAGGAAGAAGAG | 194 | TGGGCC | 25 | SpyCas9 |
| | | GAGGCA | | | | |
| SGN006273 | 825 | GGGTTCAAGGAAGAAGAGG | 196 | TGGGCC | 24 | SpyCas9 |
| | | AGGCA | | | | |
| SGN006274 | 826 | GGTTCAAGGAAGAAGAGGA | 198 | TGGGCC | 23 | SpyCas9 |
| | | GGCA | | | | |
| SGN006275 | 827 | TTGCCAGGCCTGGGGTTGGG | 200 | GGGTCCTT | 24 | SpyCas9 |
| | | CATC | | | | |
| SGN006276 | 828 | TGCCAGGCCTGGGGTTGGGC | 202 | GGGTCCTT | 23 | SpyCas9 |
| | | ATC | | | | |
| SGN006277 | 829 | GCAGGTCTGAGGCTTTGGGT | 204 | CCCTCCAG | 24 | |
| | | GCAG | | | | |
| SGN006278 | 830 | CAGGTCTGAGGCTTTGGGTG | 206 | CCCTCCAG | 23 | |
| | | CAG | | | | |
| SGN006279 | 831 | CTCGGCCCTGGAAGGTTCCC | 208 | GGCCCCGG | 24 | |
| | | CCTG | | | | |
| SGN006280 | 832 | TCGGCCCTGGAAGGTTCCCC | 210 | GGCCCCGG | 23 | |
| | | CTG | | | | |
| SGN006281 | 833 | CTCGAAGATCTCGGCCCTGG | 212 | TTCCCCCT | 24 | |
| | | AAGG | | | | |
| SGN006282 | 834 | TCGAAGATCTCGGCCCTGGA | 214 | TTCCCCCT | 23 | |
| | | AGG | | | | |

*If the FOXP3 target sequence can be targeted by S. pyogenes Cas9 (SpyCas9) and/or LPG10145
RGN, the respective polypeptide(s) is indicated.

TABLE 3
Gene editing of FOXP3 gene with indicated guide RNA.

| | | Target | Average |
|---|---|---|---|
| Guide | | Length (in | Percent |
| Name | Cell Line | nucleotides) | Indels |
| SGN002770 | PBMC-001 | 20 | 0 |
| SGN002770 | PBMC-003 | 20 | 0 |
| SGN002771 | PBMC-001 | 20 | 0.18 |
| SGN002771 | PBMC-003 | 20 | 0 |
| SGN002772 | PBMC-001 | 20 | 0 |
| SGN002772 | PBMC-003 | 20 | 0 |
| SGN002773 | PBMC-001 | 20 | 1.12 |
| SGN002773 | PBMC-003 | 20 | 0.16 |
| SGN002774 | CD4-002 | 20 | 16.2 |
| SGN002774 | CD4-003 | 20 | 8.15 |
| SGN002774 | PBMC-001 | 20 | 6.74 |
| SGN002774 | PBMC-003 | 20 | 1.17 |
| SGN002774 | TCell-004 | 20 | 16.35 |
| SGN002775 | CD4-002 | 20 | 32.18 |
| SGN002775 | CD4-003 | 20 | 16.39 |
| SGN002775 | PBMC-001 | 20 | 14.7 |
| SGN002775 | PBMC-003 | 20 | 2.88 |
| SGN002775 | TCell-004 | 20 | 32.06 |
| SGN002776 | PBMC-001 | 20 | 0.21 |
| SGN002776 | PBMC-003 | 20 | 0.08 |
| SGN002777 | PBMC-001 | 20 | 0.61 |
| SGN002777 | PBMC-003 | 20 | 0.14 |
| SGN002778 | PBMC-001 | 20 | 0 |
| SGN002778 | PBMC-003 | 20 | 0.3 |
| SGN002779 | PBMC-001 | 20 | 0.42 |
| SGN002779 | PBMC-003 | 20 | 0.13 |
| SGN002780 | PBMC-001 | 20 | 1.15 |
| SGN002780 | PBMC-003 | 20 | 0.07 |
| SGN002781 | CD4-002 | 20 | 15.01 |
| SGN002781 | CD4-003 | 20 | 4.99 |
| SGN002781 | PBMC-001 | 20 | 6.15 |
| SGN002781 | PBMC-003 | 20 | 0.57 |
| SGN002781 | TCell-004 | 20 | 7.4 |
| SGN002782 | PBMC-001 | 20 | 0 |
| SGN002782 | PBMC-003 | 20 | 0 |
| SGN002783 | PBMC-001 | 20 | 0.72 |
| SGN002783 | PBMC-003 | 20 | 0.12 |
| SGN002784 | PBMC-001 | 20 | 0 |
| SGN002784 | PBMC-003 | 20 | 0 |
| SGN002785 | CD4-002 | 20 | 1.81 |
| SGN002785 | PBMC-001 | 20 | 2.25 |
| SGN002785 | PBMC-003 | 20 | 0.13 |
| SGN002785 | TCell-004 | 20 | 1.52 |
| SGN002786 | PBMC-001 | 20 | 0.06 |
| SGN002786 | PBMC-003 | 20 | 0 |
| SGN002787 | CD4-002 | 20 | 56.02 |
| SGN002787 | CD4-003 | 20 | 32.77 |
| SGN002787 | PBMC-001 | 20 | 35.47 |
| SGN002787 | PBMC-003 | 20 | 16.91 |
| SGN002787 | TCell-004 | 20 | 47.68 |
| SGN002788 | CD4-002 | 20 | 6.05 |
| SGN002788 | PBMC-001 | 20 | 5.6 |
| SGN002788 | PBMC-003 | 20 | 0.68 |
| SGN002788 | TCell-004 | 20 | 5.06 |
| SGN002789 | PBMC-001 | 20 | 0.53 |
| SGN002789 | PBMC-003 | 20 | 0.11 |
| SGN002790 | PBMC-001 | 20 | 0.8 |
| SGN002790 | PBMC-003 | 20 | 0.38 |
| SGN002791 | CD4-002 | 20 | 34.21 |
| SGN002791 | CD4-003 | 20 | 14.18 |
| SGN002791 | PBMC-001 | 20 | 31.02 |
| SGN002791 | PBMC-003 | 20 | 1.59 |
| SGN002791 | TCell-004 | 20 | 23.7 |
| SGN002792 | PBMC-001 | 20 | 0.49 |
| SGN002792 | PBMC-003 | 20 | 0.35 |
| SGN002793 | CD4-003 | 20 | 4.39 |
| SGN002793 | PBMC-001 | 20 | 6.81 |
| SGN002793 | PBMC-003 | 20 | 1.49 |
| SGN002793 | TCell-004 | 20 | 6.69 |
| SGN002794 | CD4-002 | 20 | 36.56 |
| SGN002794 | CD4-003 | 20 | 19.34 |
| SGN002794 | PBMC-001 | 20 | 25.49 |
| SGN002794 | PBMC-003 | 20 | 2.42 |
| SGN002794 | TCell-004 | 20 | 42.12 |
| SGN002795 | CD4-002 | 20 | 0.93 |
| SGN002795 | PBMC-001 | 20 | 2.48 |
| SGN002795 | PBMC-003 | 20 | 0.33 |
| SGN002795 | TCell-004 | 20 | 0.65 |
| SGN002796 | PBMC-001 | 20 | 0.1 |
| SGN002796 | PBMC-003 | 20 | 0.38 |
| SGN002797 | PBMC-001 | 20 | 0 |
| SGN002797 | PBMC-003 | 20 | 0 |
| SGN002798 | CD4-002 | 20 | 6.04 |
| SGN002798 | PBMC-001 | 20 | 2.25 |
| SGN002798 | PBMC-003 | 20 | 0.55 |
| SGN002798 | TCell-004 | 20 | 4.91 |
| SGN002799 | PBMC-001 | 20 | 0 |
| SGN002799 | PBMC-003 | 20 | 0 |
| SGN002800 | CD4-002 | 20 | 2.88 |
| SGN002800 | PBMC-001 | 20 | 3.53 |
| SGN002800 | PBMC-003 | 20 | 0.1 |
| SGN002800 | TCell-004 | 20 | 1.55 |
| SGN002801 | PBMC-001 | 20 | 0.39 |
| SGN002801 | PBMC-003 | 20 | 0.04 |
| SGN002802 | PBMC-001 | 20 | 0.15 |
| SGN002802 | PBMC-003 | 20 | 0 |
| SGN002803 | PBMC-001 | 20 | 0 |
| SGN002803 | PBMC-003 | 20 | 0 |
| SGN003378 | CD4-003 | 25 | 62.49 |
| SGN003378 | CD4-004 | 25 | 77.61 |
| SGN003378 | CD4-005 | 25 | 71.92 |
| SGN003378 | CD4-006 | 25 | 62.28 |
| SGN003378 | CD4-007 | 25 | 73.38 |
| SGN003378 | Tcell-001 | 25 | 73.98 |
| SGN003378 | Tcell-002 | 25 | 68.09 |
| SGN003378 | Tcell-003 | 25 | 63.4 |
| SGN003378 | TCell-006 | 25 | 65.68 |
| SGN003378 | TCell-008 | 25 | 73.22 |
| SGN003379 | CD4-003 | 25 | 38.4 |
| SGN003379 | CD4-004 | 25 | 57.38 |
| SGN003379 | CD4-005 | 25 | 54.62 |
| SGN003379 | CD4-006 | 25 | 48.22 |
| SGN003379 | CD4-007 | 25 | 32.01 |
| SGN003379 | TCell-006 | 25 | 61.04 |
| SGN003380 | CD4-003 | 25 | 0 |
| SGN003380 | CD4-004 | 25 | 61.19 |
| SGN003380 | CD4-005 | 25 | 52.98 |
| SGN003380 | CD4-006 | 25 | 48.39 |
| SGN003380 | CD4-007 | 25 | 39.23 |
| SGN003381 | CD4-003 | 25 | 72.69 |
| SGN003381 | CD4-004 | 25 | 80.46 |
| SGN003381 | CD4-005 | 25 | 77.9 |
| SGN003381 | CD4-006 | 25 | 48.57 |
| SGN003381 | CD4-007 | 25 | 84.95 |
| SGN003381 | Tcell-001 | 25 | 87.35 |
| SGN003381 | Tcell-002 | 25 | 80.65 |
| SGN003381 | Tcell-003 | 25 | 75.65 |
| SGN003381 | TCell-006 | 25 | 64.11 |
| SGN003381 | TCell-008 | 25 | 64.94 |
| SGN003382 | CD4-003 | 25 | 0.12 |
| SGN003382 | CD4-004 | 25 | 83.26 |
| SGN003382 | CD4-005 | 25 | 80.11 |
| SGN003382 | CD4-006 | 25 | 71.75 |
| SGN003382 | CD4-007 | 25 | 59.81 |
| SGN003382 | Tcell-001 | 25 | 84.2 |
| SGN003382 | Tcell-002 | 25 | 77.54 |
| SGN003382 | Tcell-003 | 25 | 70.99 |
| SGN003382 | TCell-008 | 25 | 82 |
| SGN003383 | CD4-003 | 25 | 43.57 |
| SGN003383 | CD4-004 | 25 | 32.8 |
| SGN003383 | CD4-005 | 25 | 27.41 |
| SGN003383 | CD4-006 | 25 | 26.98 |
| SGN003383 | CD4-007 | 25 | 17.47 |
| SGN003383 | TCell-006 | 25 | 38.85 |
| SGN003384 | CD4-003 | 25 | 62.24 |
| SGN003384 | CD4-004 | 25 | 76.6 |
| SGN003384 | CD4-005 | 25 | 71.08 |
| SGN003384 | CD4-006 | 25 | 58.19 |
| SGN003384 | CD4-007 | 25 | 84.63 |
| SGN003384 | Tcell-001 | 25 | 68.11 |
| SGN003384 | Tcell-002 | 25 | 61.03 |
| SGN003384 | Tcell-003 | 25 | 51.44 |
| SGN003384 | TCell-006 | 25 | 51.42 |
| SGN003384 | TCell-008 | 25 | 78.37 |
| SGN005042 | TCell-008 | 24 | 30.56 |
| SGN005043 | TCell-008 | 24 | 55.36 |
| SGN005044 | TCell-008 | 24 | 58.66 |
| SGN005050 | CD4-006 | 25 | 3.54 |
| SGN005052 | CD4-006 | 25 | 0 |
| SGN005053 | CD4-006 | 25 | 0.96 |
| SGN005055 | CD4-006 | 25 | 0 |
| SGN005056 | CD4-006 | 25 | 0.26 |
| SGN005058 | CD4-006 | 25 | 32.33 |
| SGN005060 | CD4-006 | 25 | 0 |
| SGN005061 | CD4-006 | 25 | 0.2 |
| SGN005062 | CD4-006 | 25 | 0 |
| SGN005063 | CD4-006 | 25 | 0 |
| SGN005066 | CD4-006 | 25 | 0 |
| SGN005067 | CD4-006 | 25 | 14.06 |
| SGN005068 | CD4-006 | 25 | 4.75 |
| SGN005069 | CD4-006 | 25 | 0 |
| SGN005070 | CD4-006 | 25 | 0 |
| SGN005071 | CD4-006 | 25 | 0 |
| SGN005072 | CD4-006 | 25 | 3.89 |
| SGN005073 | Tcell-001 | 25 | 85.21 |
| SGN005073 | Tcell-002 | 25 | 78 |
| SGN005073 | Tcell-003 | 25 | 74.9 |
| SGN005073 | TCell-008 | 25 | 89.32 |
| SGN005074 | CD4-006 | 25 | 0 |
| SGN005075 | CD4-006 | 25 | 0 |
| SGN005076 | CD4-006 | 25 | 0.92 |
| SGN005104 | CD4-006 | 25 | 25.1 |
| SGN005635 | TCell-008 | 25 | 68.12 |
| SGN005636 | TCell-008 | 24 | 68.31 |
| SGN005637 | Tcell-001 | 23 | 69.79 |
| SGN005637 | Tcell-002 | 23 | 66.45 |
| SGN005637 | Tcell-003 | 23 | 59.77 |
| SGN005637 | TCell-008 | 23 | 76.52 |
| SGN005638 | TCell-008 | 22 | 19.49 |
| SGN005639 | TCell-008 | 25 | 80.37 |
| SGN005640 | TCell-008 | 24 | 82.08 |
| SGN005641 | Tcell-001 | 23 | 91.53 |
| SGN005641 | Tcell-002 | 23 | 89.81 |
| SGN005641 | Tcell-003 | 23 | 85.34 |
| SGN005641 | TCell-008 | 23 | 85.1 |
| SGN005642 | TCell-008 | 22 | 0.08 |
| SGN005643 | TCell-008 | 25 | 79.7 |
| SGN005644 | TCell-008 | 24 | 69.06 |
| SGN005645 | TCell-008 | 23 | 61.67 |
| SGN005646 | TCell-008 | 22 | 46.68 |
| SGN005647 | TCell-008 | 25 | 81.82 |
| SGN005648 | TCell-008 | 24 | 82.13 |
| SGN005649 | Tcell-001 | 23 | 67.15 |
| SGN005649 | Tcell-002 | 23 | 67.06 |
| SGN005649 | Tcell-003 | 23 | 61.17 |
| SGN005649 | TCell-008 | 23 | 84.63 |
| SGN005650 | TCell-008 | 22 | 0.12 |
| SGN005651 | TCell-008 | 24 | 35.55 |
| SGN005683 | TCell-008 | 25 | 59.58 |
| SGN005684 | TCell-008 | 25 | 33.99 |
| SGN005685 | TCell-008 | 25 | 41.82 |
| SGN005686 | TCell-008 | 25 | 49.67 |
| SGN005687 | TCell-008 | 25 | 27.22 |
| SGN005688 | TCell-008 | 25 | 31.47 |
| SGN005689 | TCell-008 | 25 | 71.61 |
| SGN005690 | TCell-008 | 25 | 32.29 |
| SGN005691 | TCell-008 | 25 | 31.5 |
| SGN005692 | TCell-008 | 25 | 79.85 |
| SGN005693 | TCell-008 | 25 | 72.84 |
| SGN005694 | TCell-008 | 25 | 70.33 |
| SGN005695 | TCell-008 | 25 | 67.82 |
| SGN005696 | TCell-008 | 25 | 63.54 |
| SGN005697 | TCell-008 | 25 | 65.89 |
| SGN005698 | TCell-008 | 25 | 80.06 |
| SGN005699 | TCell-008 | 25 | 72.14 |
| SGN005700 | TCell-008 | 25 | 66.12 |
| SGN005701 | TCell-008 | 25 | 73.02 |
| SGN005702 | TCell-008 | 25 | 56.16 |
| SGN005703 | TCell-008 | 25 | 58.73 |
| SGN005704 | TCell-008 | 25 | 70.04 |
| SGN005705 | TCell-008 | 25 | 54.1 |
| SGN005706 | TCell-008 | 25 | 56.93 |
| SGN005707 | Tcell-001 | 25 | 79.46 |
| SGN005707 | Tcell-002 | 25 | 81.41 |
| SGN005707 | Tcell-003 | 25 | 69.26 |
| SGN005707 | TCell-008 | 25 | 80.8 |
| SGN005708 | TCell-008 | 25 | 57.91 |
| SGN005709 | TCell-008 | 25 | 56.8 |
| SGN005710 | TCell-008 | 25 | 63.59 |
| SGN005711 | TCell-008 | 25 | 48.86 |
| SGN005712 | TCell-008 | 25 | 59.33 |
| SGN005713 | TCell-008 | 25 | 51.4 |
| SGN005714 | TCell-008 | 25 | 44.74 |
| SGN005715 | TCell-008 | 25 | 53.37 |
| SGN005716 | TCell-008 | 25 | 61.66 |
| SGN005717 | TCell-008 | 25 | 35.59 |
| SGN005718 | TCell-008 | 25 | 53.17 |
| SGN006269 | TCell-008 | 25 | 87.13 |
| SGN006270 | TCell-008 | 24 | 81.15 |
| SGN006271 | Tcell-001 | 23 | 69.32 |
| SGN006271 | Tcell-002 | 23 | 69.1 |
| SGN006271 | Tcell-003 | 23 | 62.68 |
| SGN006271 | TCell-008 | 23 | 85.53 |
| SGN006272 | Tcell-001 | 25 | 62.34 |
| SGN006272 | Tcell-002 | 25 | 61.39 |
| SGN006272 | Tcell-003 | 25 | 55.22 |
| SGN006272 | TCell-008 | 25 | 82 |
| SGN006273 | TCell-008 | 24 | 71.95 |
| SGN006274 | TCell-008 | 23 | 75.46 |
| SGN006275 | TCell-008 | 24 | 60.63 |
| SGN006276 | TCell-008 | 23 | 64.65 |
| SGN006277 | TCell-008 | 24 | 80.19 |
| SGN006278 | TCell-008 | 23 | 79.39 |
| SGN006279 | TCell-008 | 24 | 57.87 |
| SGN006280 | TCell-008 | 23 | 0.11 |
| SGN006281 | TCell-008 | 24 | 20.06 |
| SGN006282 | TCell-008 | 23 | 45.54 |

TABLE 4
Primer sets for FOXP3 gene amplifications

| Primer Set | Left | Right |
|---|---|---|
| FOXP3-1 | TCGTCGGCAGCGTCAGATGTGTATA | GTCTCGTGGGCTCGGAGATGTGTAT |
| | AGAGACAGGCTTTGACCAGAGG | AAGAGACAGTCAAGGAAGAAGA |
| | AGTGTC (SEQ ID NO: 215) | GGAGGCA (SEQ ID NO: 216) |
| FOXP3-2 | TCGTCGGCAGCGTCAGATGTGTATA | GTCTCGTGGGCTCGGAGATGTGTAT |
| | AGAGACAGCCTATTGTCTACGC | AAGAGACAGCCAGTGCCACAGTA |
| | AGCCTG (SEQ ID NO: 217) | AAGGTC (SEQ ID NO: 218) |
| FOXP3-Exon1 | TCGTCGGCAGCGTCAGATGTGTATS | GTCTCGTGGGCTCGGAGATGTGTAT |
| | AGAGACAGGCTTTGACCAGAGG | AAGAGACAGCCAGTGCCACAGTA |
| | AGTGTC (SEQ ID NO: 219) | AAGGTC (SEQ ID NO: 220) |

TABLE 5
Lead guide RNA dose response data in multiple donors.

| | | RNP | |
|---|---|---|---|
| Guide | | Concentration | Percent |
| Name | Cell Line | (pmol) | Indels |
| SGN005637 | Tcell-001 | 20 | 46.77 |
| SGN005637 | Tcell-001 | 20 | 46.13 |
| SGN005637 | Tcell-001 | 20 | 45.39 |
| SGN005637 | Tcell-001 | 40 | 76.82 |
| SGN005637 | Tcell-001 | 40 | 77.06 |
| SGN005637 | Tcell-001 | 40 | 77.29 |
| SGN005637 | Tcell-001 | 60 | 85.96 |
| SGN005637 | Tcell-001 | 60 | 86.29 |
| SGN005637 | Tcell-001 | 60 | 87.67 |
| SGN005637 | Tcell-001 | 80 | 91.50 |
| SGN005637 | Tcell-001 | 80 | 92.06 |
| SGN005637 | Tcell-001 | 80 | 91.73 |
| SGN005641 | Tcell-001 | 20 | 86.46 |
| SGN005641 | Tcell-001 | 20 | 86.17 |
| SGN005641 | Tcell-001 | 20 | 84.54 |
| SGN005641 | Tcell-001 | 40 | 93.38 |
| SGN005641 | Tcell-001 | 40 | 93.29 |
| SGN005641 | Tcell-001 | 40 | 93.84 |
| SGN005641 | Tcell-001 | 60 | 93.88 |
| SGN005641 | Tcell-001 | 60 | 92.96 |
| SGN005641 | Tcell-001 | 60 | 94.46 |
| SGN005641 | Tcell-001 | 80 | 94.091 |
| SGN005641 | Tcell-001 | 80 | 93.69 |
| SGN005641 | Tcell-001 | 80 | 95.22 |
| SGN005707 | Tcell-001 | 20 | 59.08 |
| SGN005707 | Tcell-001 | 20 | 58.08 |
| SGN005707 | Tcell-001 | 20 | 60.47 |
| SGN005707 | Tcell-001 | 40 | 86.91 |
| SGN005707 | Tcell-001 | 40 | 83.20 |
| SGN005707 | Tcell-001 | 40 | 85.83 |
| SGN005707 | Tcell-001 | 60 | 91.57 |
| SGN005707 | Tcell-001 | 60 | 90.55 |
| SGN005707 | Tcell-001 | 60 | 93.31 |
| SGN005707 | Tcell-001 | 80 | 92.83 |
| SGN005707 | Tcell-001 | 80 | 94.18 |
| SGN005707 | Tcell-001 | 80 | 95.34 |
| SGN005649 | Tcell-001 | 20 | 23.60 |
| SGN005649 | Tcell-001 | 20 | 21.86 |
| SGN005649 | Tcell-001 | 20 | 21.92 |
| SGN005649 | Tcell-001 | 40 | 67.93 |
| SGN005649 | Tcell-001 | 40 | 68.05 |
| SGN005649 | Tcell-001 | 40 | 69.06 |
| SGN005649 | Tcell-001 | 60 | 83.97 |
| SGN005649 | Tcell-001 | 60 | 84.09 |
| SGN005649 | Tcell-001 | 60 | 83.99 |
| SGN005649 | Tcell-001 | 80 | 89.23 |
| SGN005649 | Tcell-001 | 80 | 88.75 |
| SGN005649 | Tcell-001 | 80 | 90.70 |
| SGN006271 | Tcell-001 | 20 | 34.75 |
| SGN006271 | Tcell-001 | 20 | 34.80 |
| SGN006271 | Tcell-001 | 20 | 34.63 |
| SGN006271 | Tcell-001 | 40 | 70.99 |
| SGN006271 | Tcell-001 | 40 | 70.94 |
| SGN006271 | Tcell-001 | 40 | 69.79 |
| SGN006271 | Tcell-001 | 60 | 83.12 |
| SGN006271 | Tcell-001 | 60 | 82.96 |
| SGN006271 | Tcell-001 | 60 | 82.55 |
| SGN006271 | Tcell-001 | 80 | 89.12 |
| SGN006271 | Tcell-001 | 80 | 90.21 |
| SGN006271 | Tcell-001 | 80 | 91.05 |
| SGN006272 | Tcell-001 | 20 | 23.28 |
| SGN006272 | Tcell-001 | 20 | 25.02 |
| SGN006272 | Tcell-001 | 20 | 24.99 |
| SGN006272 | Tcell-001 | 40 | 59.02 |
| SGN006272 | Tcell-001 | 40 | 58.49 |
| SGN006272 | Tcell-001 | 40 | 58.06 |
| SGN006272 | Tcell-001 | 60 | 81.72 |
| SGN006272 | Tcell-001 | 60 | 77.91 |
| SGN006272 | Tcell-001 | 60 | 81.10 |
| SGN006272 | Tcell-001 | 80 | 88.31 |
| SGN006272 | Tcell-001 | 80 | 88.66 |
| SGN006272 | Tcell-001 | 80 | 89.00 |
| SGN003156 | Tcell-002 | 20 | 70.28 |
| SGN005637 | Tcell-002 | 20 | 35.60 |
| SGN005637 | Tcell-002 | 20 | 34.80 |
| SGN005637 | Tcell-002 | 20 | 35.43 |
| SGN005637 | Tcell-002 | 40 | 71.30 |
| SGN005637 | Tcell-002 | 40 | 70.97 |
| SGN005637 | Tcell-002 | 40 | 72.94 |
| SGN005637 | Tcell-002 | 60 | 83.56 |
| SGN005637 | Tcell-002 | 60 | 83.69 |
| SGN005637 | Tcell-002 | 60 | 82.45 |
| SGN005637 | Tcell-002 | 80 | 87.97 |
| SGN005637 | Tcell-002 | 80 | 88.61 |
| SGN005637 | Tcell-002 | 80 | 89.67 |
| SGN005641 | Tcell-002 | 20 | 81.88 |
| SGN005641 | Tcell-002 | 20 | 82.94 |
| SGN005641 | Tcell-002 | 20 | 79.86 |
| SGN005641 | Tcell-002 | 40 | 90.48 |
| SGN005641 | Tcell-002 | 40 | 90.26 |
| SGN005641 | Tcell-002 | 40 | 91.87 |
| SGN005641 | Tcell-002 | 60 | 91.28 |
| SGN005641 | Tcell-002 | 60 | 92.20 |
| SGN005641 | Tcell-002 | 60 | 93.28 |
| SGN005641 | Tcell-002 | 80 | 91.81 |
| SGN005641 | Tcell-002 | 80 | 92.20 |
| SGN005641 | Tcell-002 | 80 | 93.97 |
| SGN005707 | Tcell-002 | 20 | 59.69 |
| SGN005707 | Tcell-002 | 20 | 58.38 |
| SGN005707 | Tcell-002 | 20 | 56.95 |
| SGN005707 | Tcell-002 | 40 | 85.24 |
| SGN005707 | Tcell-002 | 40 | 84.54 |
| SGN005707 | Tcell-002 | 40 | 85.90 |
| SGN005707 | Tcell-002 | 60 | 92.91 |
| SGN005707 | Tcell-002 | 60 | 92.20 |
| SGN005707 | Tcell-002 | 60 | 93.41 |
| SGN005707 | Tcell-002 | 80 | 93.56 |
| SGN005707 | Tcell-002 | 80 | 93.27 |
| SGN005707 | Tcell-002 | 80 | 94.81 |
| SGN005649 | Tcell-002 | 20 | 21.89 |
| SGN005649 | Tcell-002 | 20 | 22.78 |
| SGN005649 | Tcell-002 | 20 | 22.07 |
| SGN005649 | Tcell-002 | 40 | 67.29 |
| SGN005649 | Tcell-002 | 40 | 67.71 |
| SGN005649 | Tcell-002 | 40 | 67.32 |
| SGN005649 | Tcell-002 | 60 | 83.66 |
| SGN005649 | Tcell-002 | 60 | 85.14 |
| SGN005649 | Tcell-002 | 60 | 83.93 |
| SGN005649 | Tcell-002 | 80 | 90.31 |
| SGN005649 | Tcell-002 | 80 | 89.41 |
| SGN005649 | Tcell-002 | 80 | 91.02 |
| SGN006271 | Tcell-002 | 20 | 33.69 |
| SGN006271 | Tcell-002 | 20 | 31.84 |
| SGN006271 | Tcell-002 | 20 | 34.10 |
| SGN006271 | Tcell-002 | 40 | 68.60 |
| SGN006271 | Tcell-002 | 40 | 67.94 |
| SGN006271 | Tcell-002 | 40 | 70.60 |
| SGN006271 | Tcell-002 | 60 | 84.39 |
| SGN006271 | Tcell-002 | 60 | 82.91 |
| SGN006271 | Tcell-002 | 60 | 88.64 |
| SGN006271 | Tcell-002 | 80 | 87.50 |
| SGN006271 | Tcell-002 | 80 | 87.12 |
| SGN006271 | Tcell-002 | 80 | 89.02 |
| SGN006272 | Tcell-002 | 20 | 22.21 |
| SGN006272 | Tcell-002 | 20 | 22.28 |
| SGN006272 | Tcell-002 | 20 | 21.25 |
| SGN006272 | Tcell-002 | 40 | 56.69 |
| SGN006272 | Tcell-002 | 40 | 57.41 |
| SGN006272 | Tcell-002 | 40 | 55.48 |
| SGN006272 | Tcell-002 | 60 | 77.66 |
| SGN006272 | Tcell-002 | 60 | 77.89 |
| SGN006272 | Tcell-002 | 60 | 80.23 |
| SGN006272 | Tcell-002 | 80 | 87.83 |
| SGN006272 | Tcell-002 | 80 | 87.82 |
| SGN006272 | Tcell-002 | 80 | 89.80 |
| SGN005637 | Tcell-003 | 20 | 32.39 |
| SGN005637 | Tcell-003 | 20 | 26.68 |
| SGN005637 | Tcell-003 | 20 | 28.88 |
| SGN005637 | Tcell-003 | 40 | 59.69 |
| SGN005637 | Tcell-003 | 40 | 60.34 |
| SGN005637 | Tcell-003 | 40 | 62.44 |
| SGN005637 | Tcell-003 | 60 | 74.69 |
| SGN005637 | Tcell-003 | 60 | 73.71 |
| SGN005637 | Tcell-003 | 60 | 78.70 |
| SGN005637 | Tcell-003 | 80 | 83.57 |
| SGN005637 | Tcell-003 | 80 | 81.71 |
| SGN005637 | Tcell-003 | 80 | 83.90 |
| SGN005641 | Tcell-003 | 20 | 72.01 |
| SGN005641 | Tcell-003 | 20 | 66.83 |
| SGN005641 | Tcell-003 | 20 | 66.89 |
| SGN005641 | Tcell-003 | 40 | 88.52 |
| SGN005641 | Tcell-003 | 40 | 87.66 |
| SGN005641 | Tcell-003 | 40 | 89.84 |
| SGN005641 | Tcell-003 | 60 | 91.18 |
| SGN005641 | Tcell-003 | 60 | 91.01 |
| SGN005641 | Tcell-003 | 60 | 92.53 |
| SGN005641 | Tcell-003 | 80 | 92.31 |
| SGN005641 | Tcell-003 | 80 | 84.35 |
| SGN005641 | Tcell-003 | 80 | 93.85 |
| SGN005707 | Tcell-003 | 20 | 43.28 |
| SGN005707 | Tcell-003 | 20 | 36.26 |
| SGN005707 | Tcell-003 | 20 | 39.04 |
| SGN005707 | Tcell-003 | 40 | 66.15 |
| SGN005707 | Tcell-003 | 40 | 67.63 |
| SGN005707 | Tcell-003 | 40 | 68.67 |
| SGN005707 | Tcell-003 | 60 | 80.34 |
| SGN005707 | Tcell-003 | 60 | 74.86 |
| SGN005707 | Tcell-003 | 60 | 83.36 |
| SGN005707 | Tcell-003 | 80 | 86.88 |
| SGN005707 | Tcell-003 | 80 | 86.91 |
| SGN005707 | Tcell-003 | 80 | 88.80 |
| SGN005649 | Tcell-003 | 20 | 19.90 |
| SGN005649 | Tcell-003 | 20 | 16.19 |
| SGN005649 | Tcell-003 | 20 | 18.43 |
| SGN005649 | Tcell-003 | 40 | 54.10 |
| SGN005649 | Tcell-003 | 40 | 55.56 |
| SGN005649 | Tcell-003 | 40 | 58.53 |
| SGN005649 | Tcell-003 | 60 | 74.46 |
| SGN005649 | Tcell-003 | 60 | 74.26 |
| SGN005649 | Tcell-003 | 60 | 76.82 |
| SGN005649 | Tcell-003 | 80 | 83.06 |
| SGN005649 | Tcell-003 | 80 | 81.10 |
| SGN005649 | Tcell-003 | 80 | 84.09 |
| SGN006271 | Tcell-003 | 20 | 28.38 |
| SGN006271 | Tcell-003 | 20 | 23.48 |
| SGN006271 | Tcell-003 | 20 | 25.17 |
| SGN006271 | Tcell-003 | 40 | 58.49 |
| SGN006271 | Tcell-003 | 40 | 60.51 |
| SGN006271 | Tcell-003 | 40 | 63.40 |
| SGN006271 | Tcell-003 | 60 | 72.90 |
| SGN006271 | Tcell-003 | 60 | 74.69 |
| SGN006271 | Tcell-003 | 60 | 76.95 |
| SGN006271 | Tcell-003 | 80 | 83.77 |
| SGN006271 | Tcell-003 | 80 | 81.17 |
| SGN006271 | Tcell-003 | 80 | 82.94 |
| SGN006272 | Tcell-003 | 20 | 19.62 |
| SGN006272 | Tcell-003 | 20 | 13.06 |
| SGN006272 | Tcell-003 | 20 | 18.26 |
| SGN006272 | Tcell-003 | 40 | 43.26 |
| SGN006272 | Tcell-003 | 40 | 43.92 |
| SGN006272 | Tcell-003 | 40 | 46.10 |
| SGN006272 | Tcell-003 | 60 | 69.28 |
| SGN006272 | Tcell-003 | 60 | 69.59 |
| SGN006272 | Tcell-003 | 60 | 72.86 |
| SGN006272 | Tcell-003 | 80 | 82.23 |
| SGN006272 | Tcell-003 | 80 | 80.18 |
| SGN006272 | Tcell-003 | 80 | 82.36 |
| SGN005637\hat{ } | Tcell-001 | 0 | 0 |
| SGN005637 | Tcell-002 | 0 | 0 |
| SGN005637 | Tcell-003 | 0 | 0 |

\hat{ }Unedited controls are italicized

TABLE 6
Lead FOXP3 guide RNAs that yield the best gene
editing with APG07433.1 RGN from the screens.

| | Guide | spacer | Target |
|---|---|---|---|
| | RNA SEQ | SEQ ID | SEQ ID |
| Guide Name | ID NO: | NO: | NO: |
| SGN005637 | 693 | 155 | 156 |
| SGN005641 | 694 | 163 | 164 |
| SGN005649 | 695 | 179 | 180 |
| SGN005707 | 696 | 189 | 190 |
| SGN006271 | 697 | 197 | 198 |
| SGN006272 | 698 | 193 | 194 |

### Example 2. Establishing Conditions for Gene Editing of a Forkhead Protein P3 (FOXP3) Gene and Screen to Identify APG07433.1 Guide RNA Backbones Effective for Targeting the FOXP3 Gene

Conditions were established to edit a forkhead protein P3 (FOXP3) gene using an APG07433.1 RGN (SEQ ID NO: 545) and guide RNAs disclosed herein. RGN expression cassettes were produced and introduced into vectors for mammalian expression. The APG07433.1 RGN was codon-optimized for human expression (SEQ ID NO: 548), and operably fused at the 5′ end to an SV40 nuclear localization sequence (NLS; SEQ ID NO: 922) and to 3×FLAG tag (SEQ ID NO: 936), and operably fused at the 3′ end to nucleoplasmin NLS sequences (SEQ ID NO: 923). Two copies of the NLS sequence were used, operably fused in tandem. The construct was then subcloned into a proprietary vector from Trilink Biotechnologies for the purpose of mRNA synthesis (Trilink). The mRNA was synthesized with full substitutions of 5-Methoxyuridine, capped with CleanCap (Trilink), synthesized with an additional 120 polyadenylated tail, and resuspended in 1 mM sodium citrate, pH6.4 (Trilink). Purified mRNA was tested at 2 μg per 1×10⁶ cells per nucleofection for guide screening and optimization purposes.

The plasmid containing the bacterial codon optimized APG07433.1 RGN coding sequence was synthesized and cloned by TWIST bioscience into a pET-29b(+) vector backbone between the NdeI and XhoI restriction sites. A description of the open reading frame (ORF) in this construct from N to C terminus is as follows: 10× polyhistidine (HIS) tag, tobacco etch virus (TEV) protease site, simian virus 40 (SV40) nuclear localization signal (NLS), APG07433.1 RGN, and nucleoplasmin NLS. Purified protein was used in conjunction with guide RNAs listed below for RNP editing. Various doses of protein and guide were tested to determine optimal conditions.

Guide RNAs (gRNAs) were synthesized by Integrated DNA technologies. Guides were synthesized with phosphorothioated 2′-O-methyl modifications to the 5′ terminal 3nt and 3′ terminal 3nt of each guide. Spacer and target sequences for each guide are included in the Sequence Listing and sequence descriptions are in Table 10.

The components described above were introduced into primary human T cells. Three days prior to Amaxa nucleofection, primary human T cells were thawed and activated into a T150 flask containing complete CTS Optimizer T-cell Expansion SFM (Gibco) supplemented with OpTmizer T-Cell Expansion Supplement (2.6% v/v, Gibco), CTS Immune Cell SR (2.5% v/v, Gibco) 1× GlutaMAX Supplement (Gibco) and 1% Penicillin-Streptomycin (Gibco). Base media was also supplemented with recombinant human IL-2 (300 IU/mL, Miltenyi Biotec), human IL-7 (5 ng/mL, Miltenyi Biotec) and human IL-15 (5 ng/mL, Miltenyi Biotec). T cells were activated with anti CD3/CD28 Dynabeads at a ratio of 1:1 bead/cell. Cells were initially seeded at 1×10⁶ cells per ml and grown for 3 days.

After 3 days of activation, Dynabeads were removed using a magnetic stand and 1×10⁶ T cells were nucleofected using the 4D-Nucleofector™ X Unit (program EO-115 for mRNA and EH-115 for RNP) following the manufacturer's instructions. For mRNA delivery, 2 μg of APG07433.1 RGN in an mRNA format and 4 μg of sgRNA were co-transfected with 1×10⁶ T Cells in 20 μl. Various amounts of protein and guide were tested for RNP delivery.

After 96 hours of growth, total genomic DNA was harvested using a genomic DNA isolation kit (Machery-Nagel) according to the manufacturer's instructions. The total genomic DNA was then analyzed to determine the rate of editing for each FOXP3 target. First, oligonucleotides were produced to be used for PCR amplification and subsequent analysis of the amplified FOXP3 target site. Oligonucleotide sequences used are listed in Table 4.

All PCR reactions were performed using 10 μL of 2× Master Mix Phusion High-Fidelity DNA polymerase (Thermo Scientific) in a 20 μL reaction including 0.5 μM of each primer. Large genomic regions encompassing each target gene were first amplified using PCR #1 primers, using a program of: 98° C., 1 min; 30 cycles of [98° C., 10 sec; 62° C., 15 sec; 72° C., 5 min]; 72° C., 5 min; 12° C., forever. One L of this PCR reaction was then further amplified using primers specific for each guide (PCR #2 primers), using a program of: 98° C., 1 min; 35 cycles of [98° C., 10 sec; 67° C., 15 sec; 72° C., 30 sec]; 72° C., 5 min; 12° C., forever. Primers for PCR #2 include Nextera Read 1 and Read 2 Transposase Adapter overhang sequences for Illumina sequencing.

Table 2 lists FOXP3 guide RNAs used in experiments described in the Examples, along with sequence identifiers for the guide RNAs and their target sequences.

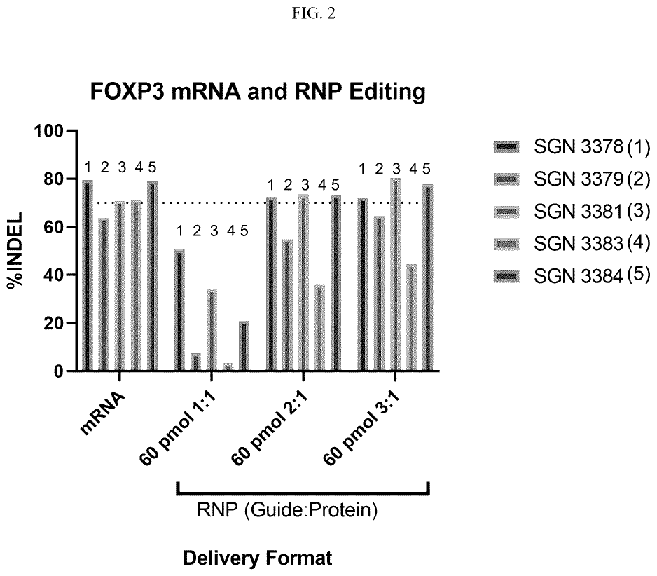

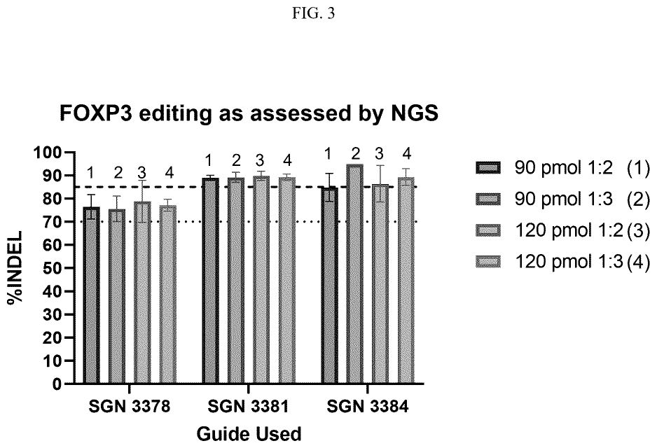

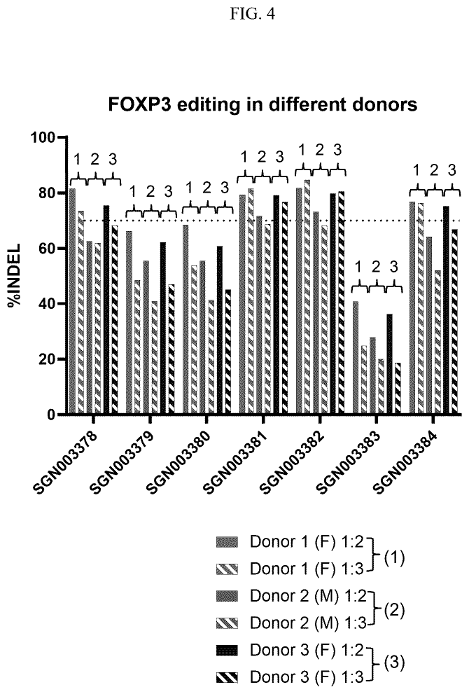

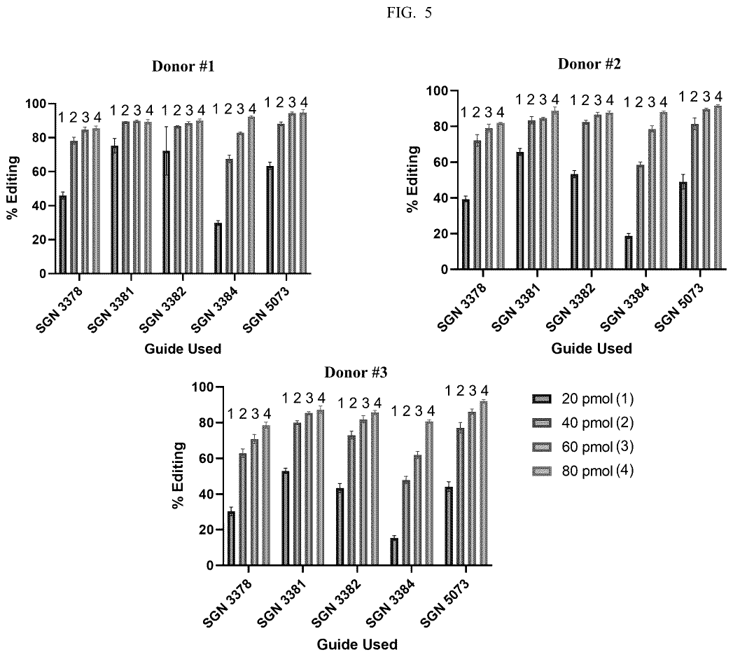

It was determined that increased 25 nt spacer length improved editing relative to 20 nt spacer length (FIG. 1). Multiple guide RNAs showed >70% editing at FOXP3 over a range of guide RNA:RGN protein ratios (FIG. 2). Consistent editing of FOXP3 could be obtained at higher doses of ribonucleoprotein (RNP) complex of guide RNA and APG07433.1 RGN (FIGS. 3 and 5), and multiple guide RNAs showed >70% editing at FOXP3 in cells from different donors (FIGS. 4 and 5).

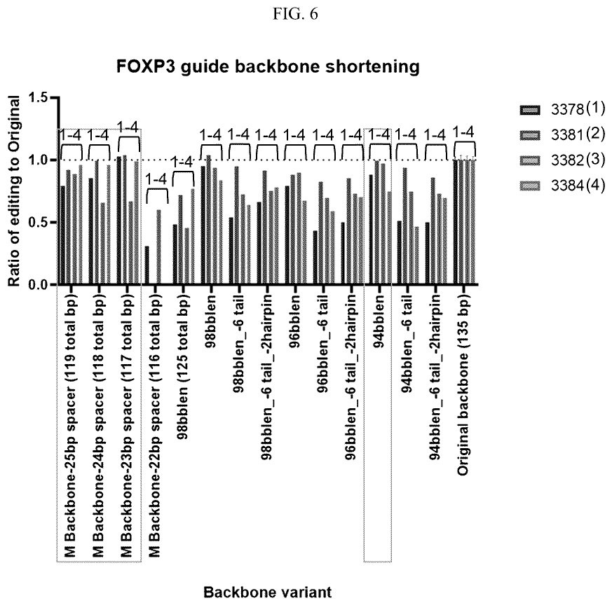

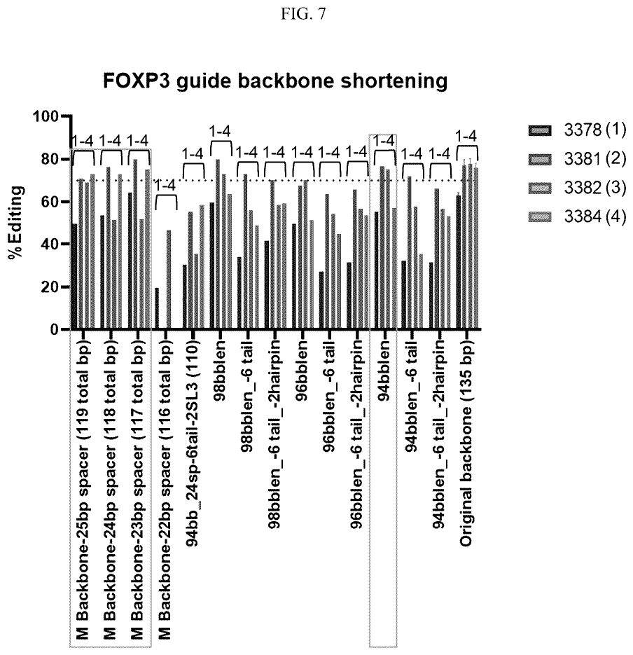

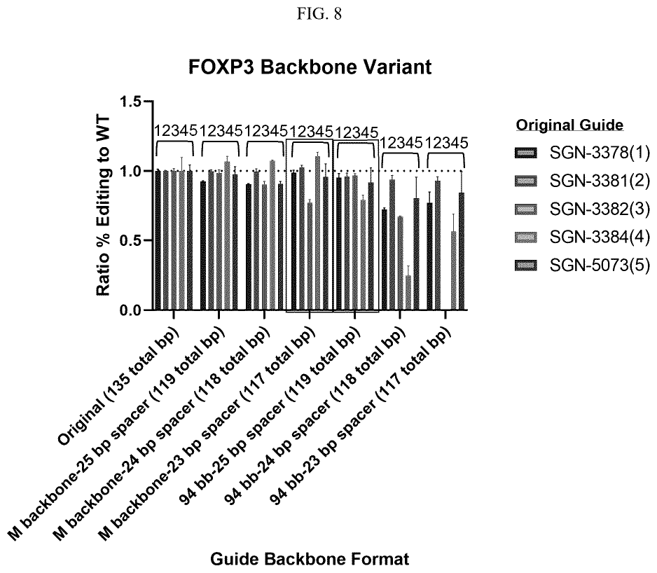

Shortened backbone variants of the native APG07433.1 backbone (native backbone length of 110 nucleotides (nt)) were tested to see which were most effective in editing of the FOXP3 gene. FIGS. 6 and 7 show performance of guide RNAs in FOXP3 editing, either as ratio of editing of guide RNAs with backbone variants and various spacer lengths to guide RNA with native backbone and 25 nt spacer (‘original backbone (135 bp)’)(FIG. 6) or as percent editing of each guide RNA (FIG. 7). Results suggest that the ‘M’ backbone and 94 nt length backbone performed the best, with total guide RNA length at or under 119 nt. The ‘M’ backbone has a deletion of 10 nt in the first stem of stem loop 1 formed by hybridization of the crRNA repeat and anti-repeat, a deletion of 2 nt in stem loop 3 most proximal to the tail of the guide RNA, and a deletion of 4 nt from the tail of the guide RNA. The ‘M’ and 94 nt length backbones yielded high gene editing across a number of FOXP3 targets and was dependent upon spacer length (FIG. 8).

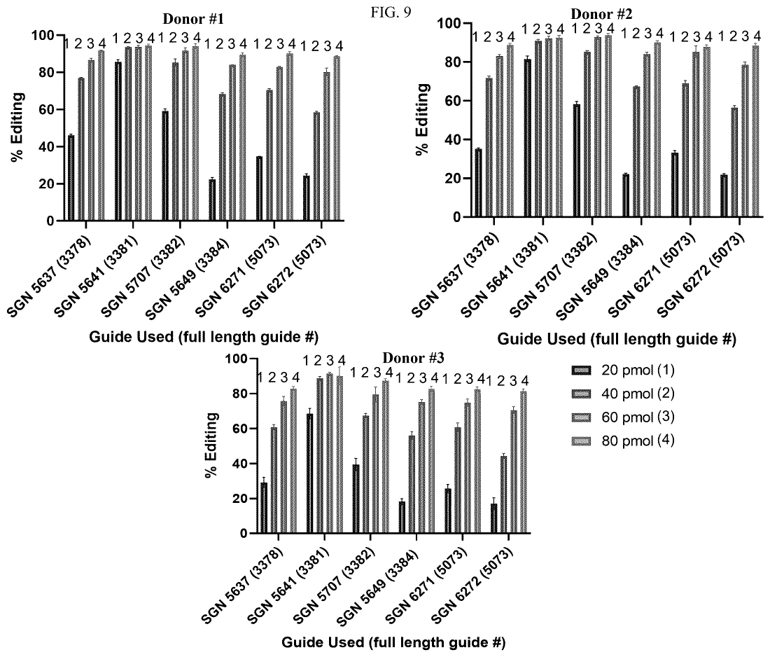

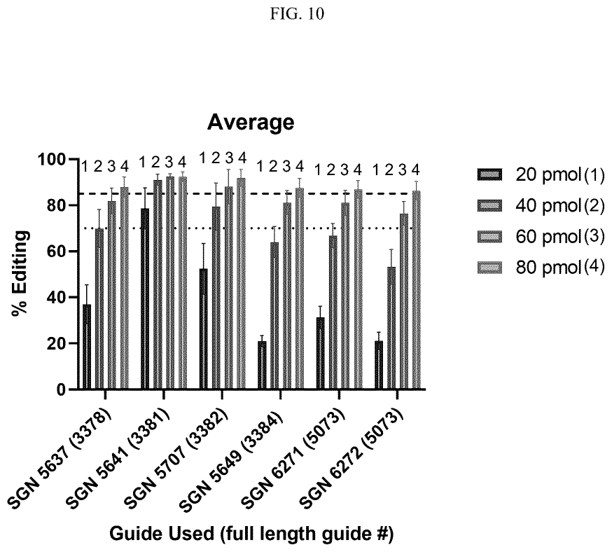

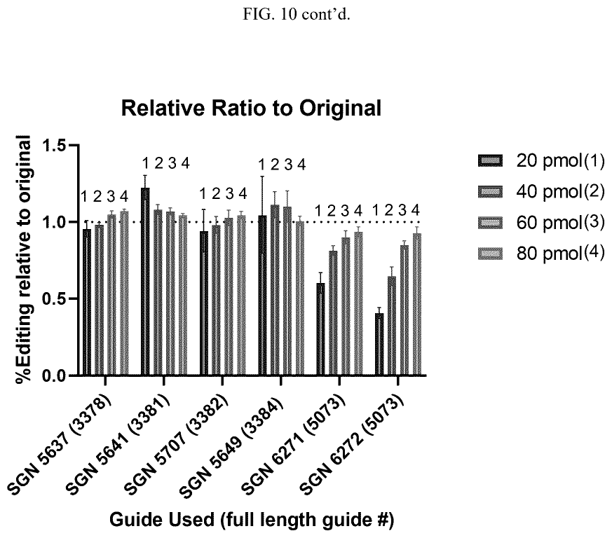

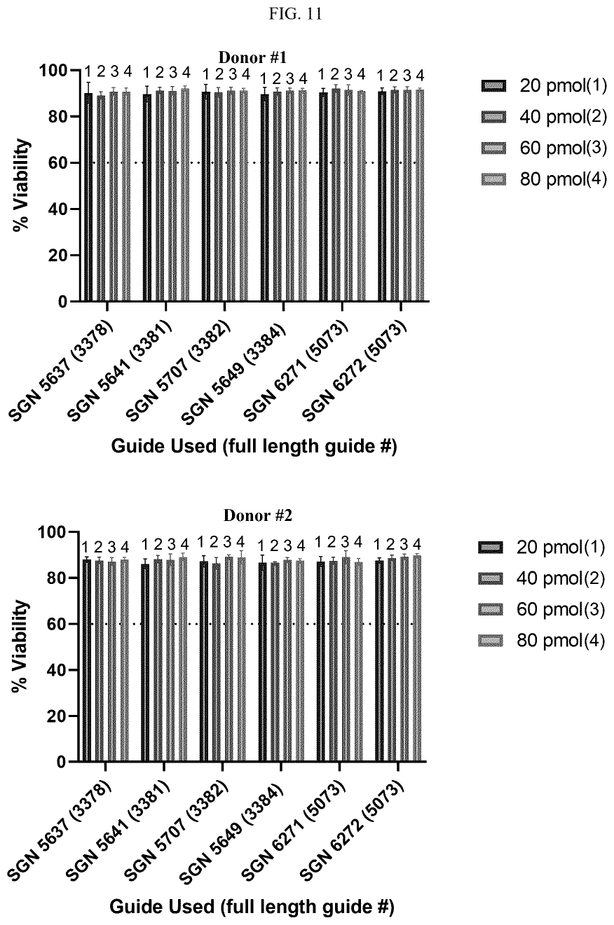

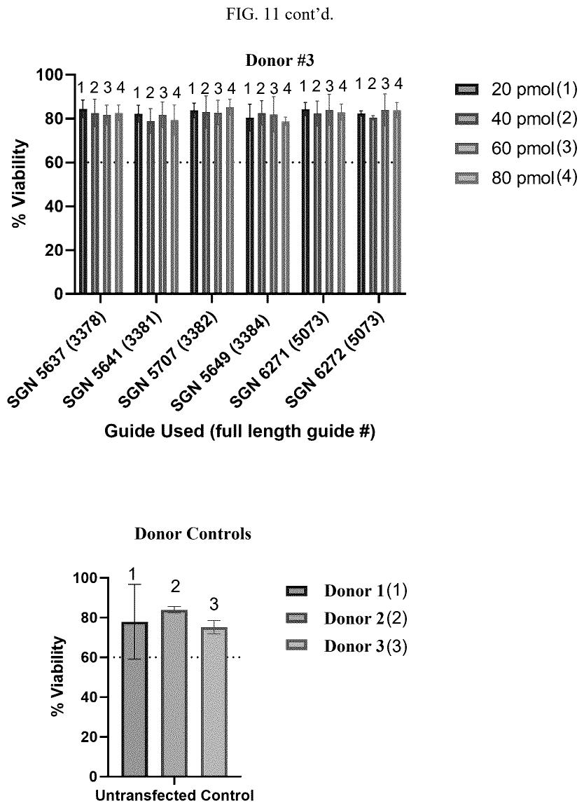

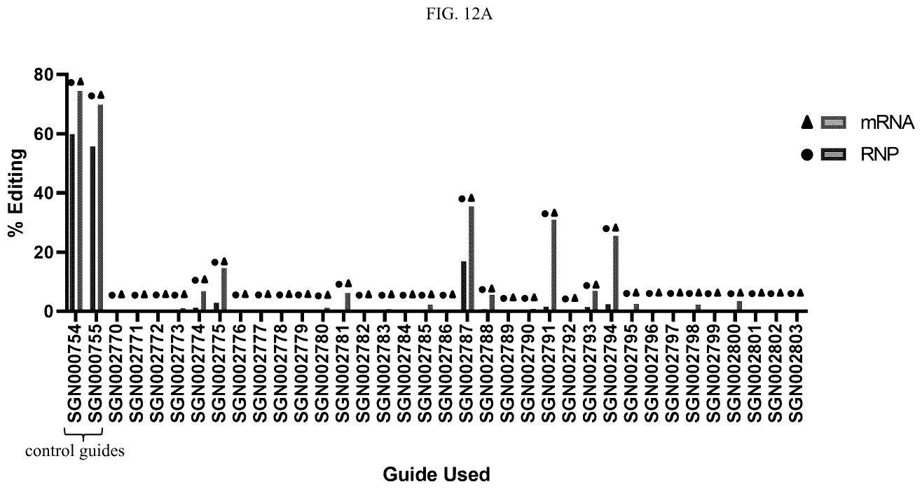

Truncated guide RNAs (shortened in spacer and/or backbone) were effective at editing multiple FOXP3 target sites across a dose range of RNP and across multiple donors (FIG. 9). Most truncated guide RNAs showed equal or slightly improved editing as compared to the original guide RNA with native backbone and 25 nt spacer (FIG. 10). Cell viability was at or above 80% for most samples, across multiple donors, and across a dose range of RNP (FIG. 11).

### Example 3. No Bona Fide Off-Target Gene Editing was Seen for 5 of the 6 Lead FOXP3 Guide RNAs

Bioinformatics was used to identify potential off-target sequences. The criteria for potential off-target site selection included no mismatches in the PAM sequence and 5 or less mismatches in the spacer (which includes RNA and DNA bulges). Table 7 shows predicted off-target sites for some FOXP3 guide RNAs and their spacer lengths. Manipulation of spacer length can alter the predicted off-target sites.

TABLE 7
Bioinformatic identification of potential off-target sites.

| Guide RNA | Spacer | | Shortened | Spacer | |
|---|---|---|---|---|---|
| Name | (nt) | # POTS* | Guide | (bp) | # POTS |
| SGN003378 | 25 | 11 | SGN005637 | 23 | 37 |
| SGN003381 | 25 | 13 | SGN005641 | 23 | 15 |
| SGN003384 | 25 | 4 | SGN005649 | 23 | 1 |
| SGN005073 | 25 | 19 | SGN006271 | 23 | 31 |

*POTS - Predicted Off Target Sites

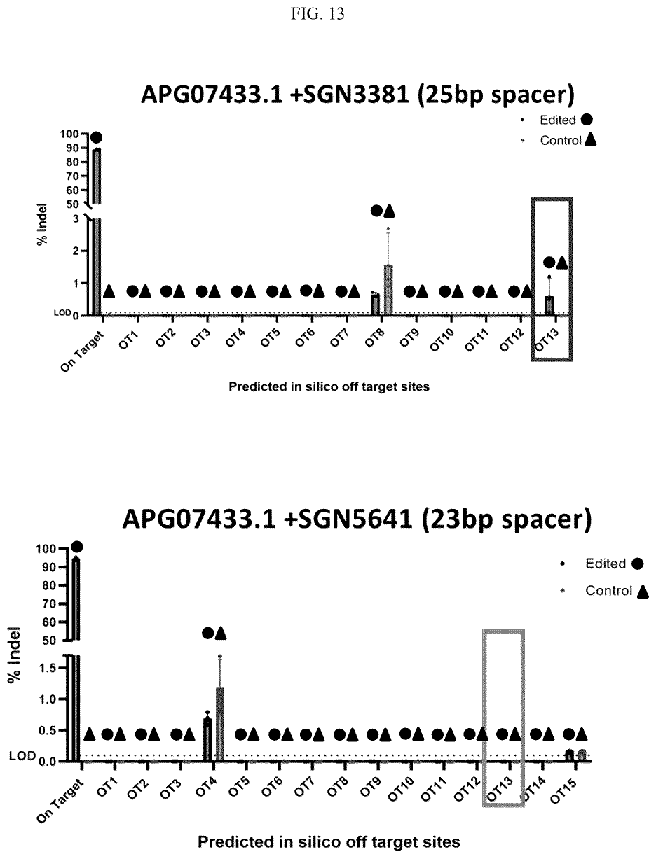

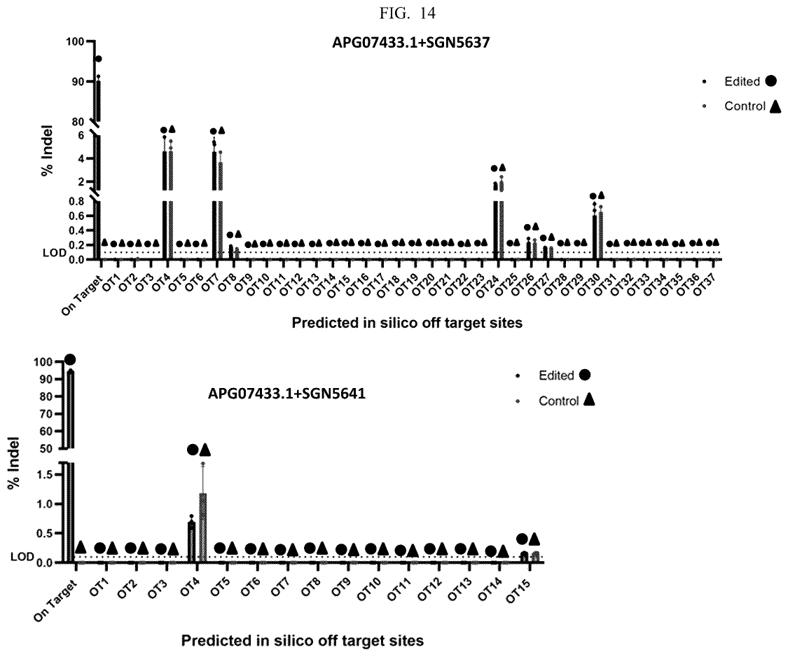

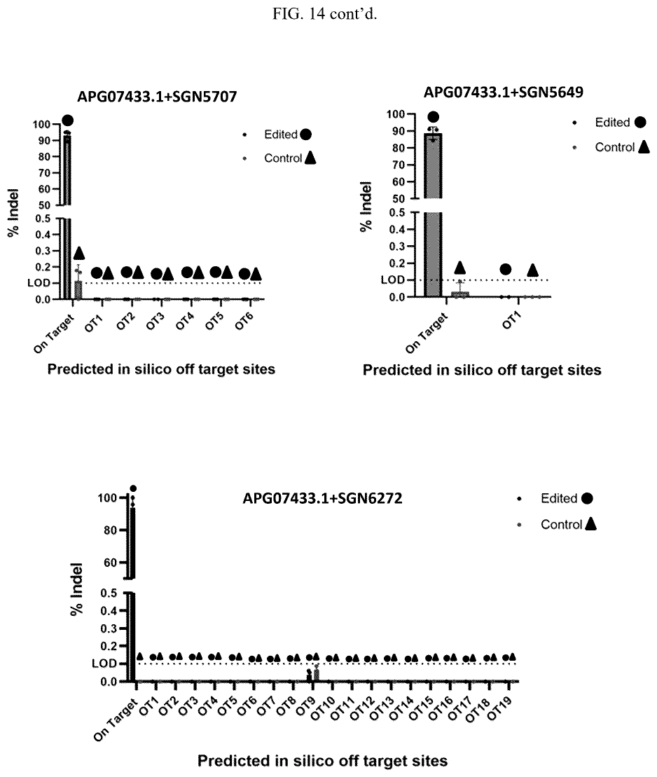

Amplicon sequencing (Amp-Seq) was used to confirm bona fide off-target sites at 0.1% limit of detection. Table 8 shows gene editing rates for off-target sites for the 6 FOXP3 lead guide RNAs. Changes to spacer length can generate a better guide RNA with no bona fide off-targets (FIG. 13). 5 of the 6 lead FOXP3 guide RNAs had no bona fide off-target gene editing (FIG. 14). Table 9 lists the primers used in generating amplicons for Amp-Seq.

TABLE 8
Gene editing of off-target sequences for lead FOXP3 guide RNAs.

| | Off-Target | Cell | Control | Reads | Reads | Percent |
|---|---|---|---|---|---|---|
| Guide | Number | Line | Type | In | Aligned | Indels |
| SGN005637 | 5637-OT1 | Cell line- | | 183475 | 183213 | 0 |
| | | 001 | | | | |
| | | Cell line- | | 158437 | 158096 | 0 |
| | | 002 | | | | |
| | | Cell line- | | 153949 | 153813 | 0 |
| | | 003 | | | | |
| | | Cell line- | Unedited | 180231 | 180046 | 0 |
| | | 001 | Control | | | |
| | | Cell line- | Unedited | 154910 | 154741 | 0 |
| | | 002 | Control | | | |
| | | Cell line- | Unedited | 172080 | 171887 | 0 |
| | | 003 | Control | | | |
| | 5637-OT2 | Cell line- | | 205026 | 203921 | 0 |
| | | 001 | | | | |
| | | Cell line- | | 175120 | 174731 | 0 |
| | | 002 | | | | |
| | | Cell line- | | 160664 | 160434 | 0 |
| | | 003 | | | | |
| | | Cell line- | Unedited | 168512 | 168093 | 0 |
| | | 001 | Control | | | |
| | | Cell line- | Unedited | 183759 | 183047 | 0 |
| | | 002 | Control | | | |
| | | Cell line- | Unedited | 997524 | 996002 | 0.013730398 |
| | | 003 | Control | | | |
| | 5637-OT4 | Cell line- | | 169094 | 168540 | 3.807021882 |
| | | 001 | | | | |
| | | Cell line- | | 214782 | 214213 | 5.856096259 |
| | | 002 | | | | |
| | | Cell line- | | 215959 | 215337 | 4.201692922 |
| | | 003 | | | | |
| | | Cell line- | Unedited | 171152 | 170725 | 3.513581166 |
| | | 001 | Control | | | |
| | | Cell line- | Unedited | 181842 | 181398 | 5.486579301 |
| | | 002 | Control | | | |
| | | Cell line- | Unedited | 276022 | 275290 | 4.906624551 |
| | | 003 | Control | | | |
| | 5637-OT4 | Cell line- | | 150532 | 150146 | 0 |
| | | 001 | | | | |
| | | Cell line- | | 113490 | 113210 | 0 |
| | | 002 | | | | |
| | | Cell line- | | 125827 | 125563 | 0 |
| | | 003 | | | | |
| | | Cell line- | Unedited | 118058 | 117782 | 0 |
| | | 001 | Control | | | |
| | | Cell line- | Unedited | 113270 | 113018 | 0 |
| | | 002 | Control | | | |
| | | Cell line- | Unedited | 125012 | 124731 | 0 |
| | | 003 | Control | | | |
| | 5637-OT6 | Cell line- | | 129523 | 129204 | 0 |
| | | 001 | | | | |
| | | Cell line- | | 97784 | 97569 | 0 |
| | | 002 | | | | |
| | | Cell line- | | 115052 | 114805 | 0 |
| | | 003 | | | | |
| | | Cell line- | Unedited | 119926 | 119607 | 0 |
| | | 001 | Control | | | |
| | | Cell line- | Unedited | 124422 | 124164 | 0 |
| | | 002 | Control | | | |
| | | Cell line- | Unedited | 128489 | 128207 | 0 |
| | | 003 | Control | | | |
| | 5637-OT7 | Cell line- | | 139588 | 137227 | 5.409052530 |
| | | 001 | | | | |
| | | Cell line- | | 120943 | 119438 | 3.134734026 |
| | | 002 | | | | |
| | | Cell line- | | 102928 | 101164 | 5.202627008 |
| | | 003 | | | | |
| | | Cell line- | Unedited | 109478 | 108014 | 2.978107197 |
| | | 001 | Control | | | |
| | | Cell line- | Unedited | 104773 | 103176 | 3.457196142 |
| | | 002 | Control | | | |
| | | Cell line- | Unedited | 113186 | 111336 | 4.535825777 |
| | | 003 | Control | | | |
| | 5637-OT8 | Cell line- | | 175175 | 170318 | 0.186097097 |
| | | 001 | | | | |
| | | Cell line- | | 178505 | 175075 | 0.170383596 |
| | | 002 | | | | |
| | | Cell line- | | 191830 | 185853 | 0.138262302 |
| | | 003 | | | | |
| | | Cell line- | Unedited | 186726 | 180119 | 0.122809611 |
| | | 001 | Control | | | |
| | | Cell line- | Unedited | 179663 | 171404 | 0.127925397 |
| | | 002 | Control | | | |
| | | Cell line- | Unedited | 179494 | 170466 | 0.149519915 |
| | | 003 | Control | | | |
| | 5637-OT9 | Cell line- | | 134410 | 134242 | 0 |
| | | 001 | | | | |
| | | Cell line- | | 171148 | 171001 | 0 |
| | | 002 | | | | |
| | | Cell line- | | 205390 | 205092 | 0 |
| | | 003 | | | | |
| | | Cell line- | Unedited | 187611 | 187383 | 0 |
| | | 001 | Control | | | |
| | | Cell line- | Unedited | 149326 | 148948 | 0 |
| | | 002 | Control | | | |
| | | Cell line- | Unedited | 168484 | 168177 | 0 |
| | | 003 | Control | | | |
| | 5637-OT10 | Cell line- | | 182926 | 182461 | 0 |
| | | 001 | | | | |
| | | Cell line- | | 192336 | 192044 | 0 |
| | | 002 | | | | |
| | | Cell line- | | 170432 | 170212 | 0 |
| | | 003 | | | | |
| | | Cell line- | Unedited | 187470 | 187156 | 0 |
| | | 001 | Control | | | |
| | | Cell line- | Unedited | 177197 | 176960 | 0 |
| | | 002 | Control | | | |
| | | Cell line- | Unedited | 172210 | 171416 | 0 |
| | | 003 | Control | | | |
| | 5637-OT11 | Cell line- | | 177320 | 177171 | 0 |
| | | 001 | | | | |
| | | Cell line- | | 141652 | 141555 | 0 |
| | | 002 | | | | |
| | | Cell line- | | 99883 | 99811 | 0 |
| | | 003 | | | | |
| | | Cell line- | Unedited | 166019 | 165887 | 0 |
| | | 001 | Control | | | |
| | | Cell line- | Unedited | 145248 | 145137 | 0 |
| | | 002 | Control | | | |
| | | Cell line- | Unedited | 171129 | 170963 | 0 |
| | | 003 | Control | | | |
| | 5637-OT12 | Cell line- | | 249585 | 248446 | 0 |
| | | 001 | | | | |
| | | Cell line- | | 254502 | 253493 | 0 |
| | | 002 | | | | |
| | | Cell line- | | 276754 | 275588 | 0 |
| | | 003 | | | | |
| | | Cell line- | Unedited | 604437 | 601835 | 0 |
| | | 001 | Control | | | |
| | | Cell line- | Unedited | 355925 | 354396 | 0 |
| | | 002 | Control | | | |
| | | Cell line- | Unedited | 355563 | 354080 | 0 |
| | | 003 | Control | | | |
| | 5637-OT13 | Cell line- | | 138311 | 137901 | 0 |
| | | 001 | | | | |
| | | Cell line- | | 140195 | 139821 | 0 |
| | | 002 | | | | |
| | | Cell line- | | 145980 | 145484 | 0 |
| | | 003 | | | | |
| | | Cell line- | Unedited | 131439 | 130977 | 0 |
| | | 001 | Control | | | |
| | | Cell line- | Unedited | 145859 | 145385 | 0 |
| | | 002 | Control | | | |
| | | Cell line- | Unedited | 153769 | 153084 | 0 |
| | | 003 | Control | | | |
| | 5637-OT14 | Cell line- | | 144823 | 144608 | 0 |
| | | 001 | | | | |
| | | Cell line- | | 144755 | 144543 | 0 |
| | | 002 | | | | |
| | | Cell line- | | 141346 | 141142 | 0 |
| | | 003 | | | | |
| | | Cell line- | Unedited | 148163 | 147937 | 0 |
| | | 001 | Control | | | |
| | | Cell line- | Unedited | 142585 | 142413 | 0 |
| | | 002 | Control | | | |
| | | Cell line- | Unedited | 180292 | 180025 | 0 |
| | | 003 | Control | | | |
| | 5637-OT15 | Cell line- | | 116580 | 116414 | 0 |
| | | 001 | | | | |
| | | Cell line- | | 118493 | 118285 | 0 |
| | | 002 | | | | |
| | | Cell line- | | 123798 | 123560 | 0 |
| | | 003 | | | | |
| | | Cell line- | Unedited | 118327 | 118102 | 0 |
| | | 001 | Control | | | |
| | | Cell line- | Unedited | 81833 | 81687 | 0 |
| | | 002 | Control | | | |
| | | Cell line- | Unedited | 144217 | 143934 | 0 |
| | | 003 | Control | | | |
| | 5637-OT16 | Cell line- | | 216908 | 216471 | 0 |
| | | 001 | | | | |
| | | Cell line- | | 180970 | 180693 | 0 |
| | | 002 | | | | |
| | | Cell line- | | 192276 | 191983 | 0 |
| | | 003 | | | | |
| | | Cell line- | Unedited | 199088 | 198789 | 0 |
| | | 001 | Control | | | |
| | | Cell line- | Unedited | 220733 | 220386 | 0 |
| | | 002 | Control | | | |
| | | Cell line- | Unedited | 183680 | 183351 | 0 |
| | | 003 | Control | | | |
| | 5637-OT17 | Cell line- | | 174323 | 174016 | 0 |
| | | 001 | | | | |
| | | Cell line- | | 152249 | 152010 | 0 |
| | | 002 | | | | |
| | | Cell line- | | 140102 | 139901 | 0 |
| | | 003 | | | | |
| | | Cell line- | Unedited | 132135 | 131902 | 0 |
| | | 001 | Control | | | |
| | | Cell line- | Unedited | 126047 | 125846 | 0 |
| | | 002 | Control | | | |
| | | Cell line- | Unedited | 119166 | 118965 | 0 |
| | | 003 | Control | | | |
| | 5637-OT18 | Cell line- | | 225970 | 224752 | 0 |
| | | 001 | | | | |
| | | Cell line- | | 274401 | 272327 | 0 |
| | | 002 | | | | |
| | | Cell line- | | 158309 | 157106 | 0 |
| | | 003 | | | | |
| | | Cell line- | Unedited | 175811 | 174931 | 0 |
| | | 001 | Control | | | |
| | | Cell line- | Unedited | 173683 | 172724 | 0 |
| | | 002 | Control | | | |
| | | Cell line- | Unedited | 202250 | 200781 | 0 |
| | | 003 | Control | | | |
| | 5637-OT19 | Cell line- | | 172668 | 172225 | 0 |
| | | 001 | | | | |
| | | Cell line- | | 75022 | 74763 | 0 |
| | | 002 | | | | |
| | | Cell line- | | 129749 | 129475 | 0 |
| | | 003 | | | | |
| | | Cell line- | Unedited | 112676 | 112390 | 0 |
| | | 001 | Control | | | |
| | | Cell line- | Unedited | 115803 | 115536 | 0 |
| | | 002 | Control | | | |
| | | Cell line- | Unedited | 133961 | 133657 | 0 |
| | | 003 | Control | | | |
| | 5637-OT20 | Cell line- | | 153340 | 152497 | 0 |
| | | 001 | | | | |
| | | Cell line- | | 95508 | 95167 | 0 |
| | | 002 | | | | |
| | | Cell line- | | 127598 | 126372 | 0 |
| | | 003 | | | | |
| | | Cell line- | Unedited | 142029 | 141535 | 0 |
| | | 001 | Control | | | |
| | | Cell line- | Unedited | 119733 | 119164 | 0 |
| | | 002 | Control | | | |
| | | Cell line- | Unedited | 123858 | 123134 | 0 |
| | | 003 | Control | | | |
| | 5637-OT21 | Cell line- | | 181618 | 171699 | 0 |
| | | 001 | | | | |
| | | Cell line- | | 131985 | 127256 | 0 |
| | | 002 | | | | |
| | | Cell line- | | 184130 | 178006 | 0 |
| | | 003 | | | | |
| | | Cell line- | Unedited | 186565 | 181107 | 0 |
| | | 001 | Control | | | |
| | | Cell line- | Unedited | 179623 | 172141 | 0 |
| | | 002 | Control | | | |
| | | Cell line- | Unedited | 230916 | 221538 | 0 |
| | | 003 | Control | | | |
| | 5637-OT22 | Cell line- | | 117802 | 115698 | 0 |
| | | 001 | | | | |
| | | Cell line- | | 137524 | 136167 | 0 |
| | | 002 | | | | |
| | | Cell line- | | 123924 | 121936 | 0 |
| | | 003 | | | | |
| | | Cell line- | Unedited | 124209 | 121237 | 0 |
| | | 001 | Control | | | |
| | | Cell line- | Unedited | 130509 | 129299 | 0 |
| | | 002 | Control | | | |
| | | Cell line- | Unedited | 138778 | 136805 | 0 |
| | | 003 | Control | | | |
| | 5637-OT23 | Cell line- | | 160949 | 142952 | 0 |
| | | 001 | | | | |
| | | Cell line- | | 139531 | 125400 | 0 |
| | | 002 | | | | |
| | | Cell line- | | 174062 | 159869 | 0 |
| | | 003 | | | | |
| | | Cell line- | Unedited | 137181 | 126627 | 0 |
| | | 001 | Control | | | |
| | | Cell line- | Unedited | 137392 | 125443 | 0 |
| | | 002 | Control | | | |
| | | Cell line- | Unedited | 129200 | 118287 | 0 |
| | | 003 | Control | | | |
| | 5637-OT24 | Cell line- | | 146038 | 145533 | 1.440182216 |
| | | 001 | | | | |
| | | Cell line- | | 160026 | 159599 | 1.841903794 |
| | | 002 | | | | |
| | | Cell line- | | 135018 | 134623 | 1.693028256 |
| | | 003 | | | | |
| | | Cell line- | Unedited | 153382 | 152932 | 1.223008993 |
| | | 001 | Control | | | |
| | | Cell line- | Unedited | 151764 | 151130 | 1.934019804 |
| | | 002 | Control | | | |
| | | Cell line- | Unedited | 162262 | 161413 | 2.416418829 |
| | | 003 | Control | | | |
| | 5637-OT25 | Cell line- | | 167755 | 167537 | 0 |
| | | 001 | | | | |
| | | Cell line- | | 179831 | 179623 | 0 |
| | | 002 | | | | |
| | | Cell line- | | 198835 | 198577 | 0 |
| | | 003 | | | | |
| | | Cell line- | Unedited | 213451 | 213200 | 0 |
| | | 001 | Control | | | |
| | | Cell line- | Unedited | 192533 | 192282 | 0 |
| | | 002 | Control | | | |
| | | Cell line- | Unedited | 223867 | 223517 | 0 |
| | | 003 | Control | | | |
| | 5637-OT26 | Cell line- | | 188937 | 188686 | 0.184912638 |
| | | 001 | | | | |
| | | Cell line- | | 177710 | 177524 | 0.288859977 |
| | | 002 | | | | |
| | | Cell line- | | 189293 | 189055 | 0.223935231 |
| | | 003 | | | | |
| | | Cell line- | Unedited | 186087 | 185889 | 0.195985044 |
| | | 001 | Control | | | |
| | | Cell line- | Unedited | 192082 | 191858 | 0.216437586 |
| | | 002 | Control | | | |
| | | Cell line- | Unedited | 213778 | 213523 | 0.267675659 |
| | | 003 | Control | | | |
| | 5637-OT27 | Cell line- | | 201455 | 200740 | 0.161394775 |
| | | 001 | | | | |
| | | Cell line- | | 188673 | 187926 | 0.157962500 |
| | | 002 | | | | |
| | | Cell line- | | 230750 | 229935 | 0.164832737 |
| | | 003 | | | | |
| | | Cell line- | Unedited | 212295 | 211477 | 0.151533373 |
| | | 001 | Control | | | |
| | | Cell line- | Unedited | 227789 | 226891 | 0.159583290 |
| | | 002 | Control | | | |
| | | Cell line- | Unedited | 262650 | 261836 | 0.146569574 |
| | | 003 | Control | | | |
| | 5637-OT28 | Cell line- | | 114432 | 113204 | 0 |
| | | 001 | | | | |
| | | Cell line- | | 118251 | 117543 | 0 |
| | | 002 | | | | |
| | | Cell line- | | 118255 | 117560 | 0 |
| | | 003 | | | | |
| | | Cell line- | Unedited | 113443 | 111274 | 0 |
| | | 001 | Control | | | |
| | | Cell line- | Unedited | 106477 | 106098 | 0 |
| | | 002 | Control | | | |
| | | Cell line- | Unedited | 143718 | 143096 | 0 |
| | | 003 | Control | | | |
| | 5637-OT30 | Cell line- | | 160587 | 152576 | 0.674088738 |
| | | 001 | | | | |
| | | Cell line- | | 121390 | 117492 | 0.379001840 |
| | | 002 | | | | |
| | | Cell line- | | 139404 | 126882 | 0.761009798 |
| | | 003 | | | | |
| | | Cell line- | Unedited | 141971 | 134386 | 0.724111794 |
| | | 001 | Control | | | |
| | | Cell line- | Unedited | 123163 | 115676 | 0.614580053 |
| | | 002 | Control | | | |
| | | Cell line- | Unedited | 133546 | 122424 | 0.627498331 |
| | | 003 | Control | | | |
| | 5637-OT31 | Cell line- | | 121550 | 79698 | 0 |
| | | 001 | | | | |
| | | Cell line- | | 102402 | 89013 | 0 |
| | | 002 | | | | |
| | | Cell line- | | 171221 | 107099 | 0 |
| | | 003 | | | | |
| | | Cell line- | Unedited | 116431 | 75090 | 0 |
| | | 001 | Control | | | |
| | | Cell line- | Unedited | 93988 | 80603 | 0 |
| | | 002 | Control | | | |
| | | Cell line- | Unedited | 105722 | 71745 | 0 |
| | | 003 | Control | | | |
| | 5637-OT32 | Cell line- | | 264592 | 260696 | 0 |
| | | 001 | | | | |
| | | Cell line- | | 188657 | 187004 | 0 |
| | | 002 | | | | |
| | | Cell line- | | 240995 | 238179 | 0 |
| | | 003 | | | | |
| | | Cell line- | Unedited | 220874 | 219104 | 0 |
| | | 001 | Control | | | |
| | | Cell line- | Unedited | 203165 | 202384 | 0 |
| | | 002 | Control | | | |
| | | Cell line- | Unedited | 248971 | 247369 | 0 |
| | | 003 | Control | | | |
| | 5637-OT33 | Cell line- | | 225237 | 224556 | 0 |
| | | 001 | | | | |
| | | Cell line- | | 187102 | 186684 | 0 |
| | | 002 | | | | |
| | | Cell line- | | 178561 | 178113 | 0 |
| | | 003 | | | | |
| | | Cell line- | Unedited | 184826 | 184394 | 0 |
| | | 001 | Control | | | |
| | | Cell line- | Unedited | 168306 | 168050 | 0 |
| | | 002 | Control | | | |
| | | Cell line- | Unedited | 255116 | 254649 | 0 |
| | | 003 | Control | | | |
| | 5637-OT34 | Cell line- | | 171527 | 171022 | 0 |
| | | 001 | | | | |
| | | Cell line- | | 156297 | 155944 | 0 |
| | | 002 | | | | |
| | | Cell line- | | 159002 | 158643 | 0 |
| | | 003 | | | | |
| | | Cell line- | Unedited | 162722 | 162342 | 0 |
| | | 001 | Control | | | |
| | | Cell line- | Unedited | 144254 | 143889 | 0 |
| | | 002 | Control | | | |
| | | Cell line- | Unedited | 147099 | 146895 | 0 |
| | | 003 | Control | | | |
| | 5637-OT35 | Cell line- | | 131706 | 117991 | 0 |
| | | 001 | | | | |
| | | Cell line- | | 81445 | 74829 | 0 |
| | | 002 | | | | |
| | | Cell line- | | 134157 | 126492 | 0 |
| | | 003 | | | | |
| | | Cell line- | Unedited | 83125 | 81203 | 0 |
| | | 001 | Control | | | |
| | | Cell line- | Unedited | 74808 | 73320 | 0 |
| | | 002 | Control | | | |
| | | Cell line- | Unedited | 90238 | 87975 | 0 |
| | | 003 | Control | | | |
| | 5637-OT36 | Cell line- | | 137315 | 137142 | 0 |
| | | 001 | | | | |
| | | Cell line- | | 116658 | 116494 | 0 |
| | | 002 | | | | |
| | | Cell line- | | 131894 | 131716 | 0 |
| | | 003 | | | | |
| | | Cell line- | Unedited | 114734 | 114569 | 0 |
| | | 001 | Control | | | |
| | | Cell line- | Unedited | 114146 | 113929 | 0 |
| | | 002 | Control | | | |
| | | Cell line- | Unedited | 112432 | 112269 | 0 |
| | | 003 | Control | | | |
| | 5637-OT37 | Cell line- | | 109283 | 109133 | 0 |
| | | 001 | | | | |
| | | Cell line- | | 99732 | 99578 | 0 |
| | | 002 | | | | |
| | | Cell line- | | 105017 | 104902 | 0 |
| | | 003 | | | | |
| | | Cell line- | Unedited | 99862 | 99707 | 0 |
| | | 001 | Control | | | |
| | | Cell line- | Unedited | 94766 | 94586 | 0 |
| | | 002 | Control | | | |
| | | Cell line- | Unedited | 83899 | 83785 | 0 |
| | | 003 | Control | | | |
| SGN005641 | 5641-OT1 | Cell line- | | 161814 | 161393 | 0 |
| | | 001 | | | | |
| | | Cell line- | | 170999 | 170570 | 0 |
| | | 002 | | | | |
| | | Cell line- | | 170987 | 170096 | 0 |
| | | 003 | | | | |
| | | Cell line- | Unedited | 178384 | 177503 | 0 |
| | | 001 | Control | | | |
| | | Cell line- | Unedited | 167232 | 166338 | 0 |
| | | 002 | Control | | | |
| | | Cell line- | Unedited | 237295 | 235822 | 0 |
| | | 003 | Control | | | |
| | 5641-OT2 | Cell line- | | 220258 | 219817 | 0 |
| | | 001 | | | | |
| | | Cell line- | | 217857 | 217454 | 0 |
| | | 002 | | | | |
| | | Cell line- | | 207456 | 207034 | 0 |
| | | 003 | | | | |
| | | Cell line- | Unedited | 199297 | 198835 | 0 |
| | | 001 | Control | | | |
| | | Cell line- | Unedited | 227035 | 226547 | 0 |
| | | 002 | Control | | | |
| | | Cell line- | Unedited | 228922 | 228505 | 0 |
| | | 003 | Control | | | |
| | 5641-OT3 | Cell line- | | 209463 | 209128 | 0 |
| | | 001 | | | | |
| | | Cell line- | | 221280 | 220968 | 0 |
| | | 002 | | | | |
| | | Cell line- | | 232003 | 231561 | 0 |
| | | 003 | | | | |
| | | Cell line- | Unedited | 221347 | 220880 | 0 |
| | | 001 | Control | | | |
| | | Cell line- | Unedited | 255923 | 255453 | 0 |
| | | 002 | Control | | | |
| | | Cell line- | Unedited | 362498 | 362060 | 0 |
| | | 003 | Control | | | |
| | 5641-OT4 | Cell line- | | 104728 | 103119 | 0.694327513 |
| | | 001 | | | | |
| | | Cell line- | | 108748 | 107709 | 0.586902313 |
| | | 002 | | | | |
| | | Cell line- | | 107756 | 105156 | 0.791134638 |
| | | 003 | | | | |
| | | Cell line- | Unedited | 107392 | 105243 | 0.810624521 |
| | | 001 | Control | | | |
| | | Cell line- | Unedited | 112005 | 110179 | 1.051938294 |
| | | 002 | Control | | | |
| | | Cell line- | Unedited | 102562 | 98685 | 1.684915599 |
| | | 003 | Control | | | |
| | 5641-OT5 | Cell line- | | 104658 | 104396 | 0 |
| | | 001 | | | | |
| | | Cell line- | | 93256 | 93121 | 0 |
| | | 002 | | | | |
| | | Cell line- | | 112485 | 112330 | 0 |
| | | 003 | | | | |
| | | Cell line- | Unedited | 125010 | 124813 | 0 |
| | | 001 | Control | | | |
| | | Cell line- | Unedited | 119046 | 118866 | 0 |
| | | 002 | Control | | | |
| | | Cell line- | Unedited | 130707 | 130470 | 0 |
| | | 003 | Control | | | |
| | 5641-OT6 | Cell line- | | 108372 | 108112 | 0 |
| | | 001 | | | | |
| | | Cell line- | | 100107 | 99952 | 0 |
| | | 002 | | | | |
| | | Cell line- | | 111609 | 111467 | 0 |
| | | 003 | | | | |
| | | Cell line- | Unedited | 110970 | 110848 | 0 |
| | | 001 | Control | | | |
| | | Cell line- | Unedited | 120451 | 120302 | 0 |
| | | 002 | Control | | | |
| | | Cell line- | Unedited | 115563 | 115399 | 0 |
| | | 003 | Control | | | |
| | 5641-OT7 | Cell line- | | 106744 | 106549 | 0 |
| | | 001 | | | | |
| | | Cell line- | | 106641 | 106525 | 0 |
| | | 002 | | | | |
| | | Cell line- | | 113067 | 112948 | 0 |
| | | 003 | | | | |
| | | Cell line- | Unedited | 111962 | 111776 | 0 |
| | | 001 | Control | | | |
| | | Cell line- | Unedited | 108443 | 108285 | 0 |
| | | 002 | Control | | | |
| | | Cell line- | Unedited | 116442 | 116032 | 0 |
| | | 003 | Control | | | |
| | 5641-OT8 | Cell line- | | 160201 | 159217 | 0 |
| | | 001 | | | | |
| | | Cell line- | | 132503 | 132094 | 0 |
| | | 002 | | | | |
| | | Cell line- | | 126263 | 125785 | 0 |
| | | 003 | | | | |
| | | Cell line- | Unedited | 123871 | 123624 | 0 |
| | | 001 | Control | | | |
| | | Cell line- | Unedited | 150319 | 150105 | 0 |
| | | 002 | Control | | | |
| | | Cell line- | Unedited | 118333 | 118076 | 0 |
| | | 003 | Control | | | |
| | 5641-OT9 | Cell line- | | 86077 | 85971 | 0 |
| | | 001 | | | | |
| | | Cell line- | | 87583 | 87444 | 0 |
| | | 002 | | | | |
| | | Cell line- | | 88293 | 88197 | 0 |
| | | 003 | | | | |
| | | Cell line- | Unedited | 88761 | 88685 | 0 |
| | | 001 | Control | | | |
| | | Cell line- | Unedited | 85529 | 85432 | 0 |
| | | 002 | Control | | | |
| | | Cell line- | Unedited | 76426 | 76368 | 0 |
| | | 003 | Control | | | |
| | 5641-OT10 | Cell line- | | 113386 | 113278 | 0 |
| | | 001 | | | | |
| | | Cell line- | | 101691 | 101580 | 0 |
| | | 002 | | | | |
| | | Cell line- | | 92347 | 92273 | 0 |
| | | 003 | | | | |
| | | Cell line- | Unedited | 80647 | 80562 | 0 |
| | | 001 | Control | | | |
| | | Cell line- | Unedited | 89253 | 89152 | 0 |
| | | 002 | Control | | | |
| | | Cell line- | Unedited | 88972 | 88892 | 0 |
| | | 003 | Control | | | |
| | 5641-OT11 | Cell line- | | 92824 | 92595 | 0 |
| | | 001 | | | | |
| | | Cell line- | | 73260 | 73127 | 0 |
| | | 002 | | | | |
| | | Cell line- | | 105510 | 105309 | 0 |
| | | 003 | | | | |
| | | Cell line- | Unedited | 104065 | 103865 | 0 |
| | | 001 | Control | | | |
| | | Cell line- | Unedited | 91648 | 91491 | 0 |
| | | 002 | Control | | | |
| | | Cell line- | Unedited | 91447 | 91293 | 0 |
| | | 003 | Control | | | |
| | 5641-OT12 | Cell line- | | 97585 | 97247 | 0 |
| | | 001 | | | | |
| | | Cell line- | | 97426 | 97211 | 0 |
| | | 002 | | | | |
| | | Cell line- | | 78779 | 78423 | 0 |
| | | 003 | | | | |
| | | Cell line- | Unedited | 64718 | 64486 | 0 |
| | | 001 | Control | | | |
| | | Cell line- | Unedited | 59815 | 59550 | 0 |
| | | 002 | Control | | | |
| | | Cell line- | Unedited | 61452 | 61247 | 0 |
| | | 003 | Control | | | |
| | 5641-OT13 | Cell line- | | 103291 | 103096 | 0 |
| | | 001 | | | | |
| | | Cell line- | | 86305 | 86216 | 0 |
| | | 002 | | | | |
| | | Cell line- | | 100756 | 100595 | 0 |
| | | 003 | | | | |
| | | Cell line- | Unedited | 103997 | 103856 | 0 |
| | | 001 | Control | | | |
| | | Cell line- | Unedited | 95807 | 95689 | 0 |
| | | 002 | Control | | | |
| | | Cell line- | Unedited | 89112 | 89004 | 0 |
| | | 003 | Control | | | |
| | 5641-OT14 | Cell line- | | 139205 | 135459 | 0 |
| | | 001 | | | | |
| | | Cell line- | | 151153 | 149870 | 0 |
| | | 002 | | | | |
| | | Cell line- | | 160920 | 158217 | 0 |
| | | 003 | | | | |
| | | Cell line- | Unedited | 64735 | 63470 | 0 |
| | | 001 | Control | | | |
| | | Cell line- | Unedited | 115417 | 112882 | 0 |
| | | 002 | Control | | | |
| | | Cell line- | Unedited | 95081 | 91021 | 0 |
| | | 003 | Control | | | |
| | 5641-OT15 | Cell line- | | 108578 | 108423 | 0.157012281 |
| | | 001 | | | | |
| | | Cell line- | | 97611 | 97471 | 0.170412627 |
| | | 002 | | | | |
| | | Cell line- | | 96956 | 96809 | 0.151697442 |
| | | 003 | | | | |
| | | Cell line- | Unedited | 95638 | 95497 | 0.145173254 |
| | | 001 | Control | | | |
| | | Cell line- | Unedited | 101008 | 100846 | 0.164551247 |
| | | 002 | Control | | | |
| | | Cell line- | Unedited | 91599 | 91488 | 0.167022465 |
| | | 003 | Control | | | |
| SGN005649 | 5649-OT1 | Cell line- | | 76048 | 75857 | 0 |
| | | 001 | | | | |
| | | Cell line- | | 70780 | 70601 | 0 |
| | | 002 | | | | |
| | | Cell line- | | 126558 | 126185 | 0 |
| | | 003 | | | | |
| | | Cell line- | Unedited | 122480 | 121976 | 0 |
| | | 001 | Control | | | |
| | | Cell line- | Unedited | 70885 | 70713 | 0 |
| | | 002 | Control | | | |
| | | Cell line- | Unedited | 72553 | 72379 | 0 |
| | | 003 | Control | | | |
| SGN005707 | 5707-OT1 | Cell line- | | 166967 | 166438 | 0 |
| | | 001 | | | | |
| | | Cell line- | | 134812 | 134372 | 0 |
| | | 002 | | | | |
| | | Cell line- | | 172446 | 171903 | 0 |
| | | 003 | | | | |
| | | Cell line- | Unedited | 177752 | 177153 | 0 |
| | | 001 | Control | | | |
| | | Cell line- | Unedited | 177980 | 177366 | 0 |
| | | 002 | Control | | | |
| | | Cell line- | Unedited | 183603 | 182862 | 0 |
| | | 003 | Control | | | |
| | 5707-OT2 | Cell line- | | 173825 | 173493 | 0 |
| | | 001 | | | | |
| | | Cell line- | | 141583 | 141402 | 0 |
| | | 002 | | | | |
| | | Cell line- | | 193808 | 193520 | 0 |
| | | 003 | | | | |
| | | Cell line- | Unedited | 178156 | 177903 | 0 |
| | | 001 | Control | | | |
| | | Cell line- | Unedited | 156895 | 156606 | 0 |
| | | 002 | Control | | | |
| | | Cell line- | Unedited | 213569 | 213252 | 0 |
| | | 003 | Control | | | |
| | 5707-OT3 | Cell line- | | 297933 | 296808 | 0 |
| | | 001 | | | | |
| | | Cell line- | | 143932 | 143346 | 0 |
| | | 002 | | | | |
| | | Cell line- | | 158384 | 157864 | 0 |
| | | 003 | | | | |
| | | Cell line- | Unedited | 148416 | 147807 | 0 |
| | | 001 | Control | | | |
| | | Cell line- | Unedited | 211383 | 210595 | 0 |
| | | 002 | Control | | | |
| | | Cell line- | Unedited | 169108 | 164614 | 0 |
| | | 003 | Control | | | |
| | 5707-OT4 | Cell line- | | 190978 | 190587 | 0 |
| | | 001 | | | | |
| | | Cell line- | | 166723 | 166385 | 0 |
| | | 002 | | | | |
| | | Cell line- | | 170839 | 170417 | 0 |
| | | 003 | | | | |
| | | Cell line- | Unedited | 157686 | 156844 | 0 |
| | | 001 | Control | | | |
| | | Cell line- | Unedited | 165612 | 164849 | 0 |
| | | 002 | Control | | | |
| | | Cell line- | Unedited | 166668 | 164906 | 0 |
| | | 003 | Control | | | |
| | 5707-OT5 | Cell line- | | 133048 | 132815 | 0 |
| | | 001 | | | | |
| | | Cell line- | | 106862 | 106724 | 0 |
| | | 002 | | | | |
| | | Cell line- | | 114604 | 114428 | 0 |
| | | 003 | | | | |
| | | Cell line- | Unedited | 110364 | 110079 | 0 |
| | | 001 | Control | | | |
| | | Cell line- | Unedited | 83804 | 83585 | 0 |
| | | 002 | Control | | | |
| | | Cell line- | Unedited | 75486 | 75257 | 0 |
| | | 003 | Control | | | |
| | 5707-OT6 | Cell line- | | 159466 | 159232 | 0 |
| | | 001 | | | | |
| | | Cell line- | | 136713 | 136550 | 0 |
| | | 002 | | | | |
| | | Cell line- | | 167055 | 166883 | 0 |
| | | 003 | | | | |
| | | Cell line- | Unedited | 174724 | 174509 | 0 |
| | | 001 | Control | | | |
| | | Cell line- | Unedited | 155684 | 155500 | 0 |
| | | 002 | Control | | | |
| | | Cell line- | Unedited | 150805 | 150622 | 0 |
| | | 003 | Control | | | |
| SGN006271 | 6271-OT1 | Cell line- | | 104474 | 104319 | 0 |
| | | 001 | | | | |
| | | Cell line- | | 101355 | 101261 | 0 |
| | | 002 | | | | |
| | | Cell line- | | 118131 | 117996 | 0 |
| | | 003 | | | | |
| | | Cell line- | Unedited | 129690 | 129537 | 0 |
| | | 001 | Control | | | |
| | | Cell line- | Unedited | 112070 | 111949 | 0 |
| | | 002 | Control | | | |
| | | Cell line- | Unedited | 120508 | 120384 | 0 |
| | | 003 | Control | | | |
| | 6271-OT2 | Cell line- | | 95376 | 95179 | 0 |
| | | 001 | | | | |
| | | Cell line- | | 82366 | 82262 | 0 |
| | | 002 | | | | |
| | | Cell line- | | 87630 | 87508 | 0 |
| | | 003 | | | | |
| | | Cell line- | Unedited | 89539 | 89431 | 0 |
| | | 001 | Control | | | |
| | | Cell line- | Unedited | 74460 | 74352 | 0 |
| | | 002 | Control | | | |
| | | Cell line- | Unedited | 94074 | 93667 | 0 |
| | | 003 | Control | | | |
| | 6271-OT3 | Cell line- | | 92660 | 92486 | 0 |
| | | 001 | | | | |
| | | Cell line- | | 98365 | 98222 | 0 |
| | | 002 | | | | |
| | | Cell line- | | 105675 | 105534 | 0 |
| | | 003 | | | | |
| | | Cell line- | Unedited | 113144 | 112976 | 0 |
| | | 001 | Control | | | |
| | | Cell line- | Unedited | 103875 | 103738 | 0 |
| | | 002 | Control | | | |
| | | Cell line- | Unedited | 123977 | 123803 | 0 |
| | | 003 | Control | | | |
| | 6271-OT4 | Cell line- | | 73006 | 72901 | 0 |
| | | 001 | | | | |
| | | Cell line- | | 116835 | 116762 | 0 |
| | | 002 | | | | |
| | | Cell line- | | 84427 | 84322 | 0 |
| | | 003 | | | | |
| | | Cell line- | Unedited | 115250 | 115150 | 0 |
| | | 001 | Control | | | |
| | | Cell line- | Unedited | 113690 | 113575 | 0 |
| | | 002 | Control | | | |
| | | Cell line- | Unedited | 71217 | 71100 | 0 |
| | | 003 | Control | | | |
| | 6271-OT5 | Cell line- | | 72047 | 71959 | 0 |
| | | 001 | | | | |
| | | Cell line- | | 77719 | 77550 | 0 |
| | | 002 | | | | |
| | | Cell line- | | 82175 | 82062 | 0 |
| | | 003 | | | | |
| | | Cell line- | Unedited | 76732 | 76618 | 0 |
| | | 001 | Control | | | |
| | | Cell line- | Unedited | 82590 | 82470 | 0 |
| | | 002 | Control | | | |
| | | Cell line- | Unedited | 96132 | 95905 | 0 |
| | | 003 | Control | | | |
| | 6271-OT6 | Cell line- | | 101919 | 101790 | 0 |
| | | 001 | | | | |
| | | Cell line- | | 101910 | 101802 | 0 |
| | | 002 | | | | |
| | | Cell line- | | 94901 | 94816 | 0 |
| | | 003 | | | | |
| | | Cell line- | Unedited | 107256 | 107148 | 0 |
| | | 001 | Control | | | |
| | | Cell line- | Unedited | 97718 | 97610 | 0 |
| | | 002 | Control | | | |
| | | Cell line- | Unedited | 112595 | 112492 | 0 |
| | | 003 | Control | | | |
| | 6271-OT7 | Cell line- | | 87913 | 87824 | 0 |
| | | 001 | | | | |
| | | Cell line- | | 93468 | 93360 | 0 |
| | | 002 | | | | |
| | | Cell line- | | 105762 | 105660 | 0 |
| | | 003 | | | | |
| | | Cell line- | Unedited | 93347 | 93211 | 0 |
| | | 001 | Control | | | |
| | | Cell line- | Unedited | 80444 | 80330 | 0 |
| | | 002 | Control | | | |
| | | Cell line- | Unedited | 107802 | 107695 | 0 |
| | | 003 | Control | | | |
| | 6271-OT8 | Cell line- | | 175435 | 175036 | 0.063654095 |
| | | 001 | | | | |
| | | Cell line- | | 141145 | 140891 | 0 |
| | | 002 | | | | |
| | | Cell line- | | 158861 | 158578 | 0 |
| | | 003 | | | | |
| | | Cell line- | Unedited | 152576 | 152316 | 0 |
| | | 001 | Control | | | |
| | | Cell line- | Unedited | 144367 | 144095 | 0 |
| | | 002 | Control | | | |
| | | Cell line- | Unedited | 144801 | 144532 | 0.074236425 |
| | | 003 | Control | | | |
| | 6271-OT9 | Cell line- | | 162750 | 162305 | 0 |
| | | 001 | | | | |
| | | Cell line- | | 138905 | 138526 | 0 |
| | | 002 | | | | |
| | | Cell line- | | 158137 | 157758 | 0 |
| | | 003 | | | | |
| | | Cell line- | Unedited | 144593 | 144254 | 0 |
| | | 001 | Control | | | |
| | | Cell line- | Unedited | 142405 | 142079 | 0 |
| | | 002 | Control | | | |
| | | Cell line- | Unedited | 142660 | 142295 | 0 |
| | | 003 | Control | | | |
| | 6271-OT10 | Cell line- | | 155507 | 155136 | 0 |
| | | 001 | | | | |
| | | Cell line- | | 125587 | 125379 | 0 |
| | | 002 | | | | |
| | | Cell line- | | 169473 | 169177 | 0 |
| | | 003 | | | | |
| | | Cell line- | Unedited | 148437 | 148146 | 0 |
| | | 001 | Control | | | |
| | | Cell line- | Unedited | 135334 | 135055 | 0 |
| | | 002 | Control | | | |
| | | Cell line- | Unedited | 132203 | 131957 | 0 |
| | | 003 | Control | | | |
| | 6271-OT11 | Cell line- | | 230065 | 229074 | 0 |
| | | 001 | | | | |
| | | Cell line- | | 178881 | 178373 | 0 |
| | | 002 | | | | |
| | | Cell line- | | 179163 | 178461 | 0 |
| | | 003 | | | | |
| | | Cell line- | Unedited | 186342 | 185739 | 0 |
| | | 001 | Control | | | |
| | | Cell line- | Unedited | 170231 | 169676 | 0 |
| | | 002 | Control | | | |
| | | Cell line- | Unedited | 149920 | 149193 | 0 |
| | | 003 | Control | | | |
| | 6271-OT12 | Cell line- | | 201631 | 201029 | 0 |
| | | 001 | | | | |
| | | Cell line- | | 169157 | 168387 | 0 |
| | | 002 | | | | |
| | | Cell line- | | 194148 | 193574 | 0 |
| | | 003 | | | | |
| | | Cell line- | Unedited | 195198 | 194614 | 0 |
| | | 001 | Control | | | |
| | | Cell line- | Unedited | 193695 | 192547 | 0 |
| | | 002 | Control | | | |
| | | Cell line- | Unedited | 204027 | 203298 | 0 |
| | | 003 | Control | | | |
| | 6271-OT13 | Cell line- | | 199418 | 195446 | 0 |
| | | 001 | | | | |
| | | Cell line- | | 212671 | 207564 | 0 |
| | | 002 | | | | |
| | | Cell line- | | 183122 | 181065 | 0 |
| | | 003 | | | | |
| | | Cell line- | Unedited | 182743 | 176875 | 0 |
| | | 001 | Control | | | |
| | | Cell line- | Unedited | 199049 | 193649 | 0 |
| | | 002 | Control | | | |
| | | Cell line- | Unedited | 200690 | 199042 | 0 |
| | | 003 | Control | | | |
| | 6271-OT14 | Cell line- | | 257180 | 256506 | 0 |
| | | 001 | | | | |
| | | Cell line- | | 166763 | 166325 | 0 |
| | | 002 | | | | |
| | | Cell line- | | 209061 | 208421 | 0 |
| | | 003 | | | | |
| | | Cell line- | Unedited | 195205 | 194698 | 0 |
| | | 001 | Control | | | |
| | | Cell line- | Unedited | 178902 | 178454 | 0 |
| | | 002 | Control | | | |
| | | Cell line- | Unedited | 163505 | 163052 | 0 |
| | | 003 | Control | | | |
| | 6271-OT15 | Cell line- | | 156149 | 155612 | 0 |
| | | 001 | | | | |
| | | Cell line- | | 143876 | 143394 | 0 |
| | | 002 | | | | |
| | | Cell line- | | 134749 | 134159 | 0 |
| | | 003 | | | | |
| | | Cell line- | Unedited | 128772 | 128158 | 0 |
| | | 001 | Control | | | |
| | | Cell line- | Unedited | 130433 | 129871 | 0 |
| | | 002 | Control | | | |
| | | Cell line- | Unedited | 127795 | 127261 | 0 |
| | | 003 | Control | | | |
| | 6271-OT16 | Cell line- | | 218883 | 217525 | 0.205181847 |
| | | 001 | | | | |
| | | Cell line- | | 221905 | 220641 | 0 |
| | | 002 | | | | |
| | | Cell line- | | 218335 | 217139 | 0 |
| | | 003 | | | | |
| | | Cell line- | Unedited | 186209 | 184963 | 0.508526331 |
| | | 001 | Control | | | |
| | | Cell line- | Unedited | 157542 | 156499 | 0 |
| | | 002 | Control | | | |
| | | Cell line- | Unedited | 188754 | 187277 | 0 |
| | | 003 | Control | | | |
| | 6271-OT17 | Cell line- | | 161453 | 161278 | 0 |
| | | 001 | | | | |
| | | Cell line- | | 178504 | 178281 | 0 |
| | | 002 | | | | |
| | | Cell line- | | 206030 | 205835 | 0 |
| | | 003 | | | | |
| | | Cell line- | Unedited | 197133 | 196908 | 0 |
| | | 001 | Control | | | |
| | | Cell line- | Unedited | 202015 | 201758 | 0 |
| | | 002 | Control | | | |
| | | Cell line- | Unedited | 161561 | 161262 | 0 |
| | | 003 | Control | | | |
| | 6271-OT18 | Cell line- | | 183539 | 183229 | 0 |
| | | 001 | | | | |
| | | Cell line- | | 166137 | 165905 | 0 |
| | | 002 | | | | |
| | | Cell line- | | 179771 | 179484 | 0 |
| | | 003 | | | | |
| | | Cell line- | Unedited | 189315 | 188984 | 0 |
| | | 001 | Control | | | |
| | | Cell line- | Unedited | 189606 | 189309 | 0 |
| | | 002 | Control | | | |
| | | Cell line- | Unedited | 216196 | 215840 | 0 |
| | | 003 | Control | | | |
| | 6271-OT19 | Cell line- | | 257542 | 257055 | 0.129616344 |
| | | 001 | | | | |
| | | Cell line- | | 241668 | 241322 | 0 |
| | | 002 | | | | |
| | | Cell line- | | 279941 | 279535 | 0 |
| | | 003 | | | | |
| | | Cell line- | Unedited | 262690 | 262280 | 0 |
| | | 001 | Control | | | |
| | | Cell line- | Unedited | 183889 | 183619 | 0 |
| | | 002 | Control | | | |
| | | Cell line- | Unedited | 320822 | 320290 | 0 |
| | | 003 | Control | | | |
| | 6271-OT20 | Cell line- | | 186375 | 186047 | 0 |
| | | 001 | | | | |
| | | Cell line- | | 183917 | 183648 | 0 |
| | | 002 | | | | |
| | | Cell line- | | 177736 | 177472 | 0 |
| | | 003 | | | | |
| | | Cell line- | Unedited | 190109 | 189814 | 0 |
| | | 001 | Control | | | |
| | | Cell line- | Unedited | 154204 | 153934 | 0 |
| | | 002 | Control | | | |
| | | Cell line- | Unedited | 163757 | 163115 | 0 |
| | | 003 | Control | | | |
| | 6271-OT21 | Cell line- | | 168503 | 167900 | 0 |
| | | 001 | | | | |
| | | Cell line- | | 176993 | 176544 | 0 |
| | | 002 | | | | |
| | | Cell line- | | 171154 | 164588 | 0 |
| | | 003 | | | | |
| | | Cell line- | Unedited | 181941 | 181390 | 0 |
| | | 001 | Control | | | |
| | | Cell line- | Unedited | 178876 | 178288 | 0 |
| | | 002 | Control | | | |
| | | Cell line- | Unedited | 233701 | 233053 | 0 |
| | | 003 | Control | | | |
| | 6271-OT22 | Cell line- | | 188272 | 187328 | 0 |
| | | 001 | | | | |
| | | Cell line- | | 185845 | 184925 | 0 |
| | | 002 | | | | |
| | | Cell line- | | 179229 | 178181 | 0 |
| | | 003 | | | | |
| | | Cell line- | Unedited | 189825 | 189033 | 0 |
| | | 001 | Control | | | |
| | | Cell line- | Unedited | 188151 | 187304 | 0 |
| | | 002 | Control | | | |
| | | Cell line- | Unedited | 205668 | 204481 | 0 |
| | | 003 | Control | | | |
| | 6271-OT23 | Cell line- | | 200089 | 199664 | 0 |
| | | 001 | | | | |
| | | Cell line- | | 180170 | 179674 | 0 |
| | | 002 | | | | |
| | | Cell line- | | 186611 | 186174 | 0 |
| | | 003 | | | | |
| | | Cell line- | Unedited | 171180 | 170783 | 0 |
| | | 001 | Control | | | |
| | | Cell line- | Unedited | 169326 | 168821 | 0 |
| | | 002 | Control | | | |
| | | Cell line- | Unedited | 221074 | 219473 | 0 |
| | | 003 | Control | | | |
| | 6271-OT24 | Cell line- | | 312973 | 312435 | 0 |
| | | 001 | | | | |
| | | Cell line- | | 234579 | 234243 | 0 |
| | | 002 | | | | |
| | | Cell line- | | 363235 | 362788 | 0 |
| | | 003 | | | | |
| | | Cell line- | Unedited | 358661 | 358169 | 0 |
| | | 001 | Control | | | |
| | | Cell line- | Unedited | 319366 | 318906 | 0 |
| | | 002 | Control | | | |
| | | Cell line- | Unedited | 210693 | 210341 | 0 |
| | | 003 | Control | | | |
| | 6271-OT25 | Cell line- | | 279203 | 278711 | 0 |
| | | 001 | | | | |
| | | Cell line- | | 238603 | 238250 | 0 |
| | | 002 | | | | |
| | | Cell line- | | 293612 | 293248 | 0 |
| | | 003 | | | | |
| | | Cell line- | Unedited | 214910 | 214580 | 0 |
| | | 001 | Control | | | |
| | | Cell line- | Unedited | 242210 | 241854 | 0 |
| | | 002 | Control | | | |
| | | Cell line- | Unedited | 246040 | 245675 | 0 |
| | | 003 | Control | | | |
| | 6271-OT26 | Cell line- | | 201955 | 201397 | 0 |
| | | 001 | | | | |
| | | Cell line- | | 192004 | 191423 | 0 |
| | | 002 | | | | |
| | | Cell line- | | 221068 | 220789 | 0 |
| | | 003 | | | | |
| | | Cell line- | Unedited | 72423 | 76514 | 0 |
| | | 001 | Control | | | |
| | | Cell line- | Unedited | 147010 | 150138 | 0 |
| | | 002 | Control | | | |
| | | Cell line- | Unedited | 180906 | 185409 | 0 |
| | | 003 | Control | | | |
| | 6271-OT27 | Cell line- | | 231554 | 230840 | 0 |
| | | 001 | | | | |
| | | Cell line- | | 287122 | 286324 | 0 |
| | | 002 | | | | |
| | | Cell line- | | 257135 | 256252 | 0 |
| | | 003 | | | | |
| | | Cell line- | Unedited | 308252 | 307539 | 0 |
| | | 001 | Control | | | |
| | | Cell line- | Unedited | 263362 | 262487 | 0 |
| | | 002 | Control | | | |
| | | Cell line- | Unedited | 220037 | 219165 | 0 |
| | | 003 | Control | | | |
| | 6271-OT28 | Cell line- | | 210649 | 210376 | 0 |
| | | 001 | | | | |
| | | Cell line- | | 323418 | 323118 | 0 |
| | | 002 | | | | |
| | | Cell line- | | 155433 | 155227 | 0 |
| | | 003 | | | | |
| | | Cell line- | Unedited | 197948 | 197705 | 0 |
| | | 001 | Control | | | |
| | | Cell line- | Unedited | 134533 | 134363 | 0 |
| | | 002 | Control | | | |
| | | Cell line- | Unedited | 212419 | 212189 | 0 |
| | | 003 | Control | | | |
| | 6271-OT29 | Cell line- | | 146183 | 145932 | 0 |
| | | 001 | | | | |
| | | Cell line- | | 131424 | 131240 | 0 |
| | | 002 | | | | |
| | | Cell line- | | 143192 | 143006 | 0 |
| | | 003 | | | | |
| | | Cell line- | Unedited | 330908 | 330528 | 0.057864524 |
| | | 001 | Control | | | |
| | | Cell line- | Unedited | 286415 | 286065 | 0.049588241 |
| | | 002 | Control | | | |
| | | Cell line- | Unedited | 259549 | 259254 | 0.055883606 |
| | | 003 | Control | | | |
| | 6271-OT30 | Cell line- | | 281060 | 280848 | 0 |
| | | 001 | | | | |
| | | Cell line- | | 221334 | 221187 | 0 |
| | | 002 | | | | |
| | | Cell line- | | 207473 | 207332 | 0 |
| | | 003 | | | | |
| | | Cell line- | Unedited | 209029 | 208851 | 0 |
| | | 001 | Control | | | |
| | | Cell line- | Unedited | 122708 | 122610 | 0 |
| | | 002 | Control | | | |
| | | Cell line- | Unedited | 124513 | 124358 | 0 |
| | | 003 | Control | | | |
| SGN006272 | 6272-OT1 | Cell line- | | 161319 | 160734 | 0 |
| | | 001 | | | | |
| | | Cell line- | | 143745 | 143201 | 0 |
| | | 002 | | | | |
| | | Cell line- | | 155430 | 154909 | 0 |
| | | 003 | | | | |
| | | Cell line- | Unedited | 164122 | 163466 | 0 |
| | | 001 | Control | | | |
| | | Cell line- | Unedited | 142671 | 142102 | 0 |
| | | 002 | Control | | | |
| | | Cell line- | Unedited | 128436 | 127857 | 0 |
| | | 003 | Control | | | |
| | 6272-OT2 | Cell line- | | 158097 | 157759 | 0 |
| | | 001 | | | | |
| | | Cell line- | | 165435 | 165162 | 0 |
| | | 002 | | | | |
| | | Cell line- | | 144895 | 144640 | 0 |
| | | 003 | | | | |
| | | Cell line- | Unedited | 192143 | 191778 | 0 |
| | | 001 | Control | | | |
| | | Cell line- | Unedited | 158232 | 157934 | 0 |
| | | 002 | Control | | | |
| | | Cell line- | Unedited | 167450 | 167128 | 0 |
| | | 003 | Control | | | |
| | 6272-OT3 | Cell line- | | 212526 | 212196 | 0 |
| | | 001 | | | | |
| | | Cell line- | | 99341 | 99176 | 0 |
| | | 002 | | | | |
| | | Cell line- | | 165306 | 165058 | 0 |
| | | 003 | | | | |
| | | Cell line- | Unedited | 112049 | 111865 | 0 |
| | | 001 | Control | | | |
| | | Cell line- | Unedited | 165296 | 165036 | 0 |
| | | 002 | Control | | | |
| | | Cell line- | Unedited | 108413 | 108241 | 0 |
| | | 003 | Control | | | |
| | 6272-OT4 | Cell line- | | 130088 | 129914 | 0 |
| | | 001 | | | | |
| | | Cell line- | | 120296 | 120177 | 0 |
| | | 002 | | | | |
| | | Cell line- | | 101671 | 101533 | 0 |
| | | 003 | | | | |
| | | Cell line- | Unedited | 109505 | 109337 | 0 |
| | | 001 | Control | | | |
| | | Cell line- | Unedited | 163962 | 163736 | 0 |
| | | 002 | Control | | | |
| | | Cell line- | Unedited | 78803 | 78682 | 0 |
| | | 003 | Control | | | |
| | 6272-OT5 | Cell line- | | 216728 | 216472 | 0 |
| | | 001 | | | | |
| | | Cell line- | | 179400 | 179219 | 0 |
| | | 002 | | | | |
| | | Cell line- | | 137480 | 137349 | 0 |
| | | 003 | | | | |
| | | Cell line- | Unedited | 134021 | 133860 | 0 |
| | | 001 | Control | | | |
| | | Cell line- | Unedited | 105594 | 105450 | 0 |
| | | 002 | Control | | | |
| | | Cell line- | Unedited | 101639 | 101524 | 0 |
| | | 003 | Control | | | |
| | 6272-OT6 | Cell line- | | 126751 | 126681 | 0 |
| | | 001 | | | | |
| | | Cell line- | | 112856 | 112761 | 0 |
| | | 002 | | | | |
| | | Cell line- | | 145660 | 145577 | 0 |
| | | 003 | | | | |
| | | Cell line- | Unedited | 129954 | 129858 | 0 |
| | | 001 | Control | | | |
| | | Cell line- | Unedited | 112629 | 112557 | 0 |
| | | 002 | Control | | | |
| | | Cell line- | Unedited | 128767 | 128514 | 0 |
| | | 003 | Control | | | |
| | 6272-OT7 | Cell line- | | 191577 | 191199 | 0 |
| | | 001 | | | | |
| | | Cell line- | | 161504 | 161206 | 0 |
| | | 002 | | | | |
| | | Cell line- | | 232045 | 231661 | 0 |
| | | 003 | | | | |
| | | Cell line- | Unedited | 198473 | 198150 | 0 |
| | | 001 | Control | | | |
| | | Cell line- | Unedited | 210432 | 210107 | 0 |
| | | 002 | Control | | | |
| | | Cell line- | Unedited | 157126 | 156707 | 0 |
| | | 003 | Control | | | |
| | 6272-OT8 | Cell line- | | 411307 | 410981 | 0 |
| | | 001 | | | | |
| | | Cell line- | | 182008 | 181862 | 0 |
| | | 002 | | | | |
| | | Cell line- | | 418721 | 418363 | 0 |
| | | 003 | | | | |
| | | Cell line- | Unedited | 322326 | 322044 | 0 |
| | | 001 | Control | | | |
| | | Cell line- | Unedited | 123729 | 123662 | 0 |
| | | 002 | Control | | | |
| | | Cell line- | Unedited | 252434 | 252252 | 0 |
| | | 003 | Control | | | |
| | 6272-OT9 | Cell line- | | 207447 | 206904 | 0 |
| | | 001 | | | | |
| | | Cell line- | | 183068 | 182709 | 0.060866293 |
| | | 002 | | | | |
| | | Cell line- | | 217309 | 216928 | 0.050805429 |
| | | 003 | | | | |
| | | Cell line- | Unedited | 189090 | 188769 | 0.055674220 |
| | | 001 | Control | | | |
| | | Cell line- | Unedited | 210561 | 210214 | 0.059167328 |
| | | 002 | Control | | | |
| | | Cell line- | Unedited | 408748 | 408171 | 0.086875167 |
| | | 003 | Control | | | |
| | 6272-OT10 | Cell line- | | 169209 | 168961 | 0 |
| | | 001 | | | | |
| | | Cell line- | | 156585 | 156410 | 0 |
| | | 002 | | | | |
| | | Cell line- | | 178619 | 178407 | 0 |
| | | 003 | | | | |
| | | Cell line- | Unedited | 159449 | 159225 | 0 |
| | | 001 | Control | | | |
| | | Cell line- | Unedited | 166621 | 166417 | 0 |
| | | 002 | Control | | | |
| | | Cell line- | Unedited | 176498 | 176261 | 0 |
| | | 003 | Control | | | |
| | 6272-OT11 | Cell line- | | 189155 | 188853 | 0 |
| | | 001 | | | | |
| | | Cell line- | | 171792 | 171533 | 0 |
| | | 002 | | | | |
| | | Cell line- | | 202078 | 201810 | 0 |
| | | 003 | | | | |
| | | Cell line- | Unedited | 203956 | 203704 | 0 |
| | | 001 | Control | | | |
| | | Cell line- | Unedited | 177280 | 177035 | 0 |
| | | 002 | Control | | | |
| | | Cell line- | Unedited | 204899 | 204633 | 0 |
| | | 003 | Control | | | |
| | 6272-OT12 | Cell line- | | 152236 | 152073 | 0 |
| | | 001 | | | | |
| | | Cell line- | | 92112 | 92014 | 0 |
| | | 002 | | | | |
| | | Cell line- | | 199569 | 199423 | 0 |
| | | 003 | | | | |
| | | Cell line- | Unedited | 139415 | 139263 | 0 |
| | | 001 | Control | | | |
| | | Cell line- | Unedited | 207342 | 207127 | 0 |
| | | 002 | Control | | | |
| | | Cell line- | Unedited | 244685 | 244454 | 0 |
| | | 003 | Control | | | |
| | 6272-OT13 | Cell line- | | 175809 | 175516 | 0 |
| | | 001 | | | | |
| | | Cell line- | | 131510 | 131202 | 0 |
| | | 002 | | | | |
| | | Cell line- | | 148836 | 148581 | 0 |
| | | 003 | | | | |
| | | Cell line- | Unedited | 113258 | 112987 | 0 |
| | | 001 | Control | | | |
| | | Cell line- | Unedited | 179908 | 179492 | 0 |
| | | 002 | Control | | | |
| | | Cell line- | Unedited | 260234 | 259820 | 0 |
| | | 003 | Control | | | |
| | 6272-OT14 | Cell line- | | 118433 | 118314 | 0 |
| | | 001 | | | | |
| | | Cell line- | | 193904 | 193734 | 0 |
| | | 002 | | | | |
| | | Cell line- | | 121126 | 121000 | 0 |
| | | 003 | | | | |
| | | Cell line- | Unedited | 136048 | 135917 | 0 |
| | | 001 | Control | | | |
| | | Cell line- | Unedited | 111992 | 111863 | 0 |
| | | 002 | Control | | | |
| | | Cell line- | Unedited | 201265 | 201038 | 0 |
| | | 003 | Control | | | |
| | 6272-OT15 | Cell line- | | 126465 | 126306 | 0 |
| | | 001 | | | | |
| | | Cell line- | | 98514 | 98390 | 0 |
| | | 002 | | | | |
| | | Cell line- | | 115381 | 115230 | 0 |
| | | 003 | | | | |
| | | Cell line- | Unedited | 112070 | 111937 | 0 |
| | | 001 | Control | | | |
| | | Cell line- | Unedited | 158874 | 158692 | 0 |
| | | 002 | Control | | | |
| | | Cell line- | Unedited | 213771 | 213342 | 0 |
| | | 003 | Control | | | |
| | 6272-OT16 | Cell line- | | 136275 | 135960 | 0 |
| | | 001 | | | | |
| | | Cell line- | | 116080 | 115861 | 0 |
| | | 002 | | | | |
| | | Cell line- | | 120286 | 120052 | 0 |
| | | 003 | | | | |
| | | Cell line- | Unedited | 112610 | 112409 | 0 |
| | | 001 | Control | | | |
| | | Cell line- | Unedited | 105762 | 105551 | 0 |
| | | 002 | Control | | | |
| | | Cell line- | Unedited | 105739 | 105521 | 0 |
| | | 003 | Control | | | |
| | 6272-OT17 | Cell line- | | 159809 | 159483 | 0 |
| | | 001 | | | | |
| | | Cell line- | | 138626 | 138353 | 0 |
| | | 002 | | | | |
| | | Cell line- | | 163538 | 163258 | 0 |
| | | 003 | | | | |
| | | Cell line- | Unedited | 170278 | 169941 | 0 |
| | | 001 | Control | | | |
| | | Cell line- | Unedited | 121795 | 121504 | 0 |
| | | 002 | Control | | | |
| | | Cell line- | Unedited | 123971 | 123746 | 0 |
| | | 003 | Control | | | |
| | 6272-OT18 | Cell line- | | 140493 | 140322 | 0 |
| | | 001 | | | | |
| | | Cell line- | | 129868 | 129716 | 0 |
| | | 002 | | | | |
| | | Cell line- | | 123585 | 123426 | 0 |
| | | 003 | | | | |
| | | Cell line- | Unedited | 140062 | 139862 | 0 |
| | | 001 | Control | | | |
| | | Cell line- | Unedited | 130705 | 130552 | 0 |
| | | 002 | Control | | | |
| | | Cell line- | Unedited | 147735 | 147562 | 0 |
| | | 003 | Control | | | |
| | 6272-OT19 | Cell line- | | 117945 | 117786 | 0 |
| | | 001 | | | | |
| | | Cell line- | | 116130 | 115944 | 0 |
| | | 002 | | | | |
| | | Cell line- | | 105551 | 105398 | 0 |
| | | 003 | | | | |
| | | Cell line- | Unedited | 114475 | 114286 | 0 |
| | | 001 | Control | | | |
| | | Cell line- | Unedited | 88553 | 88381 | 0 |
| | | 002 | Control | | | |
| | | Cell line- | Unedited | 138950 | 138724 | 0 |
| | | 003 | Control | | | |

TABLE 9
Primer sequences for amplification of the off-target sites.

| | Off- | | Left | Right |
|---|---|---|---|---|
| Off- | Target | | Primer | Primer |
| Target | SEQ | | SEQ | SEQ ID |
| Number | ID NO: | Chromosome | ID NO: | NO: |
| 5637- | 221 | 17 | 329 | 437 |
| OT1 | | | | |
| 5637- | 222 | 16 | 330 | 438 |
| OT2 | | | | |
| 5637- | 223 | 8 | 331 | 439 |
| OT3 | | | | |
| 5637- | 224 | 22 | 332 | 440 |
| OT4 | | | | |
| 5637- | 225 | 8 | 333 | 441 |
| OT5 | | | | |
| 5637- | 226 | 21 | 334 | 442 |
| OT6 | | | | |
| 5637- | 227 | 12 | 335 | 443 |
| OT7 | | | | |
| 5637- | 228 | 12 | 336 | 444 |
| OT8 | | | | |
| 5637- | 229 | 8 | 337 | 445 |
| OT9 | | | | |
| 5637- | 230 | 19 | 338 | 446 |
| OT10 | | | | |
| 5637- | 231 | 18 | 339 | 447 |
| OT11 | | | | |
| 5637- | 232 | 22 | 340 | 448 |
| OT12 | | | | |
| 5637- | 233 | 21 | 341 | 449 |
| OT13 | | | | |
| 5637- | 234 | 17 | 342 | 450 |
| OT14 | | | | |
| 5637- | 235 | 18 | 343 | 451 |
| OT15 | | | | |
| 5637- | 236 | 4 | 344 | 452 |
| OT16 | | | | |
| 5637- | 237 | 17 | 345 | 453 |
| OT17 | | | | |
| 5637- | 238 | 17 | 346 | 454 |
| OT18 | | | | |
| 5637- | 239 | 17 | 347 | 455 |
| OT19 | | | | |
| 5637- | 240 | 11 | 348 | 456 |
| OT20 | | | | |
| 5637- | 241 | 15 | 349 | 457 |
| OT21 | | | | |
| 5637- | 242 | 22 | 350 | 458 |
| OT22 | | | | |
| 5637- | 243 | 7 | 351 | 459 |
| OT23 | | | | |
| 5637- | 244 | 16 | 352 | 460 |
| OT24 | | | | |
| 5637- | 245 | 8 | 353 | 461 |
| OT25 | | | | |
| 5637- | 246 | 2 | 354 | 462 |
| OT26 | | | | |
| 5637- | 247 | 9 | 355 | 463 |
| OT27 | | | | |
| 5637- | 248 | 19 | 356 | 464 |
| OT28 | | | | |
| 5637- | 249 | 8 | 357 | 465 |
| OT29 | | | | |
| 5637- | 250 | 2 | 358 | 466 |
| OT30 | | | | |
| 5637- | 25 | 2 | 359 | 467 |
| OT31 | | | | |
| 5637- | 252 | 4 | 360 | 468 |
| OT32 | | | | |
| 5637- | 253 | 17 | 361 | 469 |
| OT33 | | | | |
| 5637- | 254 | 6 | 362 | 470 |
| OT34 | | | | |
| 5637- | 25 | 2 | 363 | 471 |
| OT35 | | | | |
| 5637- | 256 | 14 | 364 | 472 |
| OT36 | | | | |
| 5637- | 257 | 8 | 365 | 473 |
| OT37 | | | | |
| 5641- | 258 | 1 | 366 | 474 |
| OT1 | | | | |
| 5641- | 259 | 17 | 367 | 475 |
| OT2 | | | | |
| 5641- | 260 | 20 | 368 | 476 |
| OT3 | | | | |
| 5641- | 261 | 20 | 369 | 47 |
| OT4 | | | | |
| 5641- | 262 | 13 | 370 | 478 |
| OT5 | | | | |
| 5641- | 263 | 8 | 371 | 479 |
| OT6 | | | | |
| 5641- | 264 | 20 | 372 | 480 |
| OT7 | | | | |
| 5641- | 265 | 17 | 373 | 481 |
| OT8 | | | | |
| 5641- | 266 | 21 | 374 | 482 |
| OT9 | | | | |
| 5641- | 267 | 8 | 375 | 483 |
| OT10 | | | | |
| 5641- | 268 | 5 | 376 | 484 |
| OT11 | | | | |
| 5641- | 269 | 22 | 377 | 485 |
| OT12 | | | | |
| 5641- | 270 | 10 | 378 | 486 |
| OT13 | | | | |
| 5641- | 27 | 19 | 379 | 487 |
| OT14 | | | | |
| 5641- | 272 | 1 | 380 | 488 |
| OT15 | | | | |
| 5649- | 273 | 14 | 381 | 489 |
| OT1 | | | | |
| 5707- | 274 | 12 | 382 | 490 |
| OT1 | | | | |
| 5707- | 275 | 22 | 383 | 49 |
| OT2 | | | | |
| 5707- | 276 | 1 | 384 | 492 |
| OT3 | | | | |
| 5707- | 277 | 6 | 385 | 493 |
| OT4 | | | | |
| 5707- | 278 | 10 | 386 | 494 |
| OT5 | | | | |
| 5707- | 279 | 14 | 387 | 495 |
| OT6 | | | | |
| 6271- | 280 | 1 | 388 | 496 |
| OT1 | | | | |
| 6271- | 281 | 9 | 389 | 497 |
| OT2 | | | | |
| 6271- | 282 | 15 | 390 | 498 |
| OT3 | | | | |
| 6271- | 283 | 10 | 391 | 499 |
| OT4 | | | | |
| 6271- | 284 | 8 | 392 | 500 |
| OT5 | | | | |
| 6271- | 285 | 13 | 393 | 501 |
| OT6 | | | | |
| 6271- | 286 | 13 | 394 | 502 |
| OT7 | | | | |
| 6271- | 287 | 3 | 395 | 503 |
| OT8 | | | | |
| 6271- | 288 | 17 | 396 | 504 |
| OT9 | | | | |
| 6271- | 289 | 1 | 397 | 505 |
| OT10 | | | | |
| 6271- | 290 | 2 | 398 | 506 |
| OT11 | | | | |
| 6271- | 291 | 13 | 399 | 507 |
| OT12 | | | | |
| 6271- | 292 | 14 | 400 | 508 |
| OT13 | | | | |
| 6271- | 293 | 21 | 40 | 509 |
| OT14 | | | | |
| 6271- | 294 | 14 | 402 | 510 |
| OT15 | | | | |
| 6271- | 295 | 15 | 403 | 511 |
| OT16 | | | | |
| 6271- | 296 | 17 | 404 | 512 |
| OT17 | | | | |
| 6271- | 297 | 10 | 405 | 513 |
| OT18 | | | | |
| 6271- | 298 | 10 | 406 | 514 |
| OT19 | | | | |
| 6271- | 299 | 3 | 407 | 515 |
| OT20 | | | | |
| 6271- | 300 | 20 | 408 | 516 |
| OT21 | | | | |
| 6271- | 301 | 15 | 409 | 517 |
| OT22 | | | | |
| 6271- | 302 | 2 | 410 | 518 |
| OT23 | | | | |
| 6271- | 303 | 1 | 411 | 519 |
| OT24 | | | | |
| 6271- | 304 | 19 | 412 | 520 |
| OT25 | | | | |
| 6271- | 305 | 22 | 413 | 521 |
| OT26 | | | | |
| 6271- | 306 | 18 | 414 | 522 |
| OT27 | | | | |
| 6271- | 307 | 19 | 415 | 523 |
| OT28 | | | | |
| 6271- | 308 | 17 | 416 | 524 |
| OT29 | | | | |
| 6271- | 309 | 3 | 417 | 525 |
| OT30 | | | | |
| 6272- | 310 | 11 | 418 | 526 |
| OT1 | | | | |
| 6272- | 311 | 1 | 419 | 527 |
| OT2 | | | | |
| 6272- | 312 | 20 | 420 | 528 |
| OT3 | | | | |
| 6272- | 313 | 9 | 421 | 529 |
| OT4 | | | | |
| 6272- | 314 | 5 | 422 | 530 |
| OT5 | | | | |
| 6272- | 315 | 3 | 423 | 531 |
| OT6 | | | | |
| 6272- | 316 | 15 | 424 | 532 |
| OT7 | | | | |
| 6272- | 317 | 17 | 425 | 533 |
| OT8 | | | | |
| 6272- | 318 | 15 | 426 | 534 |
| OT9 | | | | |
| 6272- | 319 | 10 | 427 | 535 |
| OT10 | | | | |
| 6272- | 320 | 15 | 428 | 536 |
| OT11 | | | | |
| 6272- | 321 | 10 | 429 | 537 |
| OT12 | | | | |
| 6272- | 322 | 8 | 430 | 538 |
| OT13 | | | | |
| 6272- | 323 | 13 | 431 | 539 |
| OT14 | | | | |
| 6272- | 324 | 13 | 432 | 540 |
| OT15 | | | | |
| 6272- | 325 | 3 | 433 | 541 |
| OT16 | | | | |
| 6272- | 326 | 17 | 434 | 542 |
| OT17 | | | | |
| 6272- | 327 | 13 | 435 | 543 |
| OT18 | | | | |
| 6272- | 328 | 1 | 436 | 544 |
| OT19 | | | | |

TABLE 10
Description of Sequence Identifiers of the application

| SEQ ID NO | Description |
|---|---|
| 1 | SGN002770 spacer |
| 2 | SGN002770 target |
| 3 | SGN002771 spacer |
| 4 | SGN002771 target |
| 5 | SGN002772 spacer |
| 6 | SGN002772 target |
| 7 | SGN002773 spacer |
| 8 | SGN002773 target |
| 9 | SGN002774 spacer |
| 10 | SGN002774 target |
| 11 | SGN002775 spacer |
| 12 | SGN002775 target |
| 13 | SGN002776 spacer |
| 14 | SGN002776 target |
| 15 | SGN002777 spacer |
| 16 | SGN002777 target |
| 17 | SGN002778 spacer |
| 18 | SGN002778 target |
| 19 | SGN002779 spacer |
| 20 | SGN002779 target |
| 21 | SGN002780 spacer |
| 22 | SGN002780 target |
| 23 | SGN002781 spacer |
| 24 | SGN002781 target |
| 25 | SGN002782 spacer |
| 26 | SGN002782 target |
| 27 | SGN002783 spacer |
| 28 | SGN002783 target |
| 29 | SGN002784 spacer |
| 30 | SGN002784 target |
| 31 | SGN002785 spacer |
| 32 | SGN002785 target |
| 33 | SGN002786 spacer |
| 34 | SGN002786 target |
| 35 | SGN002787 spacer |
| 36 | SGN002787 target |
| 37 | SGN002788 spacer |
| 38 | SGN002788 target |
| 39 | SGN002789 spacer |
| 40 | SGN002789 target |
| 41 | SGN002790 spacer |
| 42 | SGN002790 target |
| 43 | SGN002791 spacer |
| 44 | SGN002791 target |
| 45 | SGN002792 spacer |
| 46 | SGN002792 target |
| 47 | SGN002793 spacer |
| 48 | SGN002793 target |
| 49 | SGN002794 spacer |
| 50 | SGN002794 target |
| 51 | SGN002795 spacer |
| 52 | SGN002795 target |
| 53 | SGN002796 spacer |
| 54 | SGN002796 target |
| 55 | SGN002797 spacer |
| 56 | SGN002797 target |
| 57 | SGN002798 spacer |
| 58 | SGN002798 target |
| 59 | SGN002799 spacer |
| 60 | SGN002799 target |
| 61 | SGN002800 spacer |
| 62 | SGN002800 target |
| 63 | SGN002801 spacer |
| 64 | SGN002801 target |
| 65 | SGN002802 spacer |
| 66 | SGN002802 target |
| 67 | SGN002803 spacer |
| 68 | SGN002803 target |
| 69 | SGN003378 spacer |
| 70 | SGN003378 target |
| 71 | SGN003379 spacer |
| 72 | SGN003379 target |
| 73 | SGN003380 spacer |
| 74 | SGN003380 target |
| 75 | SGN003381 spacer |
| 76 | SGN003381 target |
| 77 | SGN003382 spacer |
| 78 | SGN003382 target |
| 79 | SGN003383 spacer |
| 80 | SGN003383 target |
| 81 | SGN003384 spacer |
| 82 | SGN003384 target |
| 83 | SGN005042 spacer |
| 84 | SGN005042 target |
| 85 | SGN005043 spacer |
| 86 | SGN005043 target |
| 87 | SGN005044 spacer |
| 88 | SGN005044 target |
| 89 | SGN005050 spacer |
| 90 | SGN005050 target |
| 91 | SGN005051 spacer |
| 92 | SGN005051 target |
| 93 | SGN005052 spacer |
| 94 | SGN005052 target |
| 95 | SGN005053 spacer |
| 96 | SGN005053 target |
| 97 | SGN005054 spacer |
| 98 | SGN005054 target |
| 99 | SGN005055 spacer |
| 100 | SGN005055 target |
| 101 | SGN005056 spacer |
| 102 | SGN005056 target |
| 103 | SGN005057 spacer |
| 104 | SGN005057 target |
| 105 | SGN005058 spacer |
| 106 | SGN005058 target |
| 107 | SGN005059 spacer |
| 108 | SGN005059 target |
| 109 | SGN005060 spacer |
| 110 | SGN005060 target |
| 111 | SGN005061 spacer |
| 112 | SGN005061 target |
| 113 | SGN005062 spacer |
| 114 | SGN005062 target |
| 115 | SGN005063 spacer |
| 116 | SGN005063 target |
| 117 | SGN005064 spacer |
| 118 | SGN005064 target |
| 119 | SGN005065 spacer |
| 120 | SGN005065 target |
| 121 | SGN005066 spacer |
| 122 | SGN005066 target |
| 123 | SGN005067 spacer |
| 124 | SGN005067 target |
| 125 | SGN005068 spacer |
| 126 | SGN005068 target |
| 127 | SGN005069 spacer |
| 128 | SGN005069 target |
| 129 | SGN005070 spacer |
| 130 | SGN005070 target |
| 131 | SGN005071 spacer |
| 132 | SGN005071 target |
| 133 | SGN005072 spacer |
| 134 | SGN005072 target |
| 135 | SGN005073 spacer |
| 136 | SGN005073 target |
| 137 | SGN005074 spacer |
| 138 | SGN005074 target |
| 139 | SGN005075 spacer |
| 140 | SGN005075 target |
| 141 | SGN005076 spacer |
| 142 | SGN005076 target |
| 143 | SGN005077 spacer |
| 144 | SGN005077 target |
| 145 | SGN005078 spacer |
| 146 | SGN005078 target |
| 147 | SGN005079 spacer |
| 148 | SGN005079 target |
| 149 | SGN005104 spacer |
| 150 | SGN005104 target |
| 151 | SGN005635 spacer |
| 152 | SGN005635 target |
| 153 | SGN005636 spacer |
| 154 | SGN005636 target |
| 155 | SGN005637 spacer |
| 156 | SGN005637 target |
| 157 | SGN005638 spacer |
| 158 | SGN005638 target |
| 159 | SGN005639 spacer |
| 160 | SGN005639 target |
| 161 | SGN005640 spacer |
| 162 | SGN005640 target |
| 163 | SGN005641 spacer |
| 164 | SGN005641 target |
| 165 | SGN005642 spacer |
| 166 | SGN005642 target |
| 167 | SGN005643 spacer |
| 168 | SGN005643 target |
| 169 | SGN005644 spacer |
| 170 | SGN005644 target |
| 171 | SGN005645 spacer |
| 172 | SGN005645 target |
| 173 | SGN005646 spacer |
| 174 | SGN005646 target |
| 175 | SGN005647 spacer |
| 176 | SGN005647 target |
| 177 | SGN005648 spacer |
| 178 | SGN005648 target |
| 179 | SGN005649 spacer |
| 180 | SGN005649 target |
| 181 | SGN005650 spacer |
| 182 | SGN005650 target |
| 183 | SGN005651 spacer |
| 184 | SGN005651 target |
| 185 | SGN005683, SGN005684, |
| | SGN005685, SGN005686, |
| | SGN005687, SGN005688, |
| | SGN005689, SGN005690, and |
| | SGN005691 spacer |
| 186 | SGN005683, SGN005684, |
| | SGN005685, SGN005686, |
| | SGN005687, SGN005688, |
| | SGN005689, SGN005690, and |
| | SGN005691 target |
| 187 | SGN005692, SGN005693, |
| | SGN005694, SGN005695, |
| | SGN005696, SGN005697, |
| | SGN005698, SGN005699, and |
| | SGN005700 spacer |
| 188 | SGN005692, SGN005693, |
| | SGN005694, SGN005695, |
| | SGN005696, SGN005697, |
| | SGN005698, SGN005699, and |
| | SGN005700 target |
| 189 | SGN005701, SGN005702, |
| | SGN005703, SGN005704, |
| | SGN005705, SGN005706, |
| | SGN005707, SGN005708, and |
| | SGN005709 spacer |
| 190 | SGN005701, SGN005702, |
| | SGN005703, SGN005704, |
| | SGN005705, SGN005706, |
| | SGN005707, SGN005708, and |
| | SGN005709 target |
| 191 | SGN005710, SGN005711, |
| | SGN005712, SGN005713, |
| | SGN005714, SGN005715, |
| | SGN005716, SGN005717, and |
| | SGN005718 spacer |
| 192 | SGN005710, SGN005711, |
| | SGN005712, SGN005713, |
| | SGN005714, SGN005715, |
| | SGN005716, SGN005717, and |
| | SGN005718 target |
| 193 | SGN006269 and SGN006272 spacer |
| 194 | SGN006269 and SGN006272 target |
| 195 | SGN006270 and SGN006273 spacer |
| 196 | SGN006270 and SGN006273 target |
| 197 | SGN006271 and SGN006274 spacer |
| 198 | SGN006271 and SGN006274 target |
| 199 | SGN006275 spacer |
| 200 | SGN006275 target |
| 201 | SGN006276 spacer |
| 202 | SGN006276 target |
| 203 | SGN006277 spacer |
| 204 | SGN006277 target |
| 205 | SGN006278 spacer |
| 206 | SGN006278 target |
| 207 | SGN006279 spacer |
| 208 | SGN006279 target |
| 209 | SGN006280 spacer |
| 210 | SGN006280 target |
| 211 | SGN006281 spacer |
| 212 | SGN006281 target |
| 213 | SGN006282 spacer |
| 214 | SGN006282 target |
| 215 | FOXP3-1 left primer |
| 216 | FOXP3-1 right primer |
| 217 | FOXP3-2 left primer |
| 218 | FOXP3-2 right primer |
| 219 | FOXP3-Exonl left primer |
| 220 | FOXP3-Exon1 right primer |
| 221 | 5637-OT1 off-target sequence |
| 222 | 5637-OT2 off-target sequence |
| 223 | 5637-OT3 off-target sequence |
| 224 | 5637-OT4 off-target sequence |
| 225 | 5637-OT5 off-target sequence |
| 226 | 5637-OT6 off-target sequence |
| 227 | 5637-OT7 off-target sequence |
| 228 | 5637-OT8 off-target sequence |
| 229 | 5637-OT9 off-target sequence |
| 230 | 5637-OT10 off-target sequence |
| 231 | 5637-OT11 off-target sequence |
| 232 | 5637-OT12 off-target sequence |
| 233 | 5637-OT13 off-target sequence |
| 234 | 5637-OT14 off-target sequence |
| 235 | 5637-OT15 off-target sequence |
| 236 | 5637-OT16 off-target sequence |
| 237 | 5637-OT17 off-target sequence |
| 238 | 5637-OT18 off-target sequence |
| 239 | 5637-OT19 off-target sequence |
| 240 | 5637-OT20 off-target sequence |
| 241 | 5637-OT21 off-target sequence |
| 242 | 5637-OT22 off-target sequence |
| 243 | 5637-OT23 off-target sequence |
| 244 | 5637-OT24 off-target sequence |
| 245 | 5637-OT25 off-target sequence |
| 246 | 5637-OT26 off-target sequence |
| 247 | 5637-OT27 off-target sequence |
| 248 | 5637-OT28 off-target sequence |
| 249 | 5637-OT29 off-target sequence |
| 250 | 5637-OT30 off-target sequence |
| 251 | 5637-OT31 off-target sequence |
| 252 | 5637-OT32 off-target sequence |
| 253 | 5637-OT33 off-target sequence |
| 254 | 5637-OT34 off-target sequence |
| 255 | 5637-OT35 off-target sequence |
| 256 | 5637-OT36 off-target sequence |
| 257 | 5637-OT37 off-target sequence |
| 258 | 5641-OT1 off-target sequence |
| 259 | 5641-OT2 off-target sequence |
| 260 | 5641-OT3 off-target sequence |
| 261 | 5641-OT4 off-target sequence |
| 262 | 5641-OT5 off-target sequence |
| 263 | 5641-OT6 off-target sequence |
| 264 | 5641-OT7 off-target sequence |
| 265 | 5641-OT8 off-target sequence |
| 266 | 5641-OT9 off-target sequence |
| 267 | 5641-OT10 off-target sequence |
| 268 | 5641-OT11 off-target sequence |
| 269 | 5641-OT12 off-target sequence |
| 270 | 5641-OT13 off-target sequence |
| 271 | 5641-OT14 off-target sequence |
| 272 | 5641-OT15 off-target sequence |
| 273 | 5649-OT1 off-target sequence |
| 274 | 5707-OT1 off-target sequence |
| 275 | 5707-OT2 off-target sequence |
| 276 | 5707-OT3 off-target sequence |
| 277 | 5707-OT4 off-target sequence |
| 278 | 5707-OT5 off-target sequence |
| 279 | 5707-OT6 off-target sequence |
| 280 | 6271-OT1 off-target sequence |
| 281 | 6271-OT2 off-target sequence |
| 282 | 6271-OT3 off-target sequence |
| 283 | 6271-OT4 off-target sequence |
| 284 | 6271-OT5 off-target sequence |
| 285 | 6271-OT6 off-target sequence |
| 286 | 6271-OT7 off-target sequence |
| 287 | 6271-OT8 off-target sequence |
| 288 | 6271-OT9 off-target sequence |
| 289 | 6271-OT10 off-target sequence |
| 290 | 6271-OT11 off-target sequence |
| 291 | 6271-OT12 off-target sequence |
| 292 | 6271-OT13 off-target sequence |
| 293 | 6271-OT14 off-target sequence |
| 294 | 6271-OT15 off-target sequence |
| 295 | 6271-OT16 off-target sequence |
| 296 | 6271-OT17 off-target sequence |
| 297 | 6271-OT18 off-target sequence |
| 298 | 6271-OT19 off-target sequence |
| 299 | 6271-OT20 off-target sequence |
| 300 | 6271-OT21 off-target sequence |
| 301 | 6271-OT22 off-target sequence |
| 302 | 6271-OT23 off-target sequence |
| 303 | 6271-OT24 off-target sequence |
| 304 | 6271-OT25 off-target sequence |
| 305 | 6271-OT26 off-target sequence |
| 306 | 6271-OT27 off-target sequence |
| 307 | 6271-OT28 off-target sequence |
| 308 | 6271-OT29 off-target sequence |
| 309 | 6271-OT30 off-target sequence |
| 310 | 6272-OT1 off-target sequence |
| 311 | 6272-OT2 off-target sequence |
| 312 | 6272-OT3 off-target sequence |
| 313 | 6272-OT4 off-target sequence |
| 314 | 6272-OT5 off-target sequence |
| 315 | 6272-OT6 off-target sequence |
| 316 | 6272-OT7 off-target sequence |
| 317 | 6272-OT8 off-target sequence |
| 318 | 6272-OT9 off-target sequence |
| 319 | 6272-OT10 off-target sequence |
| 320 | 6272-OT11 off-target sequence |
| 321 | 6272-OT12 off-target sequence |
| 322 | 6272-OT13 off-target sequence |
| 323 | 6272-OT14 off-target sequence |
| 324 | 6272-OT15 off-target sequence |
| 325 | 6272-OT16 off-target sequence |
| 326 | 6272-OT17 off-target sequence |
| 327 | 6272-OT18 off-target sequence |
| 328 | 6272-OT19 off-target sequence |
| 329 | 5637-OT1 left primer sequence |
| 330 | 5637-OT2 left primer sequence |
| 331 | 5637-OT3 left primer sequence |
| 332 | 5637-OT4 left primer sequence |
| 333 | 5637-OT5 left primer sequence |
| 334 | 5637-OT6 left primer sequence |
| 335 | 5637-OT7 left primer sequence |
| 336 | 5637-OT8 left primer sequence |
| 337 | 5637-OT9 left primer sequence |
| 338 | 5637-OT10 left primer sequence |
| 339 | 5637-OT11 left primer sequence |
| 340 | 5637-OT12 left primer sequence |
| 341 | 5637-OT13 left primer sequence |
| 342 | 5637-OT14 left primer sequence |
| 343 | 5637-OT15 left primer sequence |
| 344 | 5637-OT16 left primer sequence |
| 345 | 5637-OT17 left primer sequence |
| 346 | 5637-OT18 left primer sequence |
| 347 | 5637-OT19 left primer sequence |
| 348 | 5637-OT20 left primer sequence |
| 349 | 5637-OT21 left primer sequence |
| 350 | 5637-OT22 left primer sequence |
| 351 | 5637-OT23 left primer sequence |
| 352 | 5637-OT24 left primer sequence |
| 353 | 5637-OT25 left primer sequence |
| 354 | 5637-OT26 left primer sequence |
| 355 | 5637-OT27 left primer sequence |
| 356 | 5637-OT28 left primer sequence |
| 357 | 5637-OT29 left primer sequence |
| 358 | 5637-OT30 left primer sequence |
| 359 | 5637-OT31 left primer sequence |
| 360 | 5637-OT32 left primer sequence |
| 361 | 5637-OT33 left primer sequence |
| 362 | 5637-OT34 left primer sequence |
| 363 | 5637-OT35 left primer sequence |
| 364 | 5637-OT36 left primer sequence |
| 365 | 5637-OT37 left primer sequence |
| 366 | 5641-OT1 left primer sequence |
| 367 | 5641-OT2 left primer sequence |
| 368 | 5641-OT3 left primer sequence |
| 369 | 5641-OT4 left primer sequence |
| 370 | 5641-OT5 left primer sequence |
| 371 | 5641-OT6 left primer sequence |
| 372 | 5641-OT7 left primer sequence |
| 373 | 5641-OT8 left primer sequence |
| 374 | 5641-OT9 left primer sequence |
| 375 | 5641-OT10 left primer sequence |
| 376 | 5641-OT11 left primer sequence |
| 377 | 5641-OT12 left primer sequence |
| 378 | 5641-OT13 left primer sequence |
| 379 | 5641-OT14 left primer sequence |
| 380 | 5641-OT15 left primer sequence |
| 381 | 5649-OT1 left primer sequence |
| 382 | 5707-OT1 left primer sequence |
| 383 | 5707-OT2 left primer sequence |
| 384 | 5707-OT3 left primer sequence |
| 385 | 5707-OT4 left primer sequence |
| 386 | 5707-OT5 left primer sequence |
| 387 | 5707-OT6 left primer sequence |
| 388 | 6271-OT1 left primer sequence |
| 389 | 6271-OT2 left primer sequence |
| 390 | 6271-OT3 left primer sequence |
| 391 | 6271-OT4 left primer sequence |
| 392 | 6271-OT5 left primer sequence |
| 393 | 6271-OT6 left primer sequence |
| 394 | 6271-OT7 left primer sequence |
| 395 | 6271-OT8 left primer sequence |
| 396 | 6271-OT9 left primer sequence |
| 397 | 6271-OT10 left primer sequence |
| 398 | 6271-OT11 left primer sequence |
| 399 | 6271-OT12 left primer sequence |
| 400 | 6271-OT13 left primer sequence |
| 401 | 6271-OT14 left primer sequence |
| 402 | 6271-OT15 left primer sequence |
| 403 | 6271-OT16 left primer sequence |
| 404 | 6271-OT17 left primer sequence |
| 405 | 6271-OT18 left primer sequence |
| 406 | 6271-OT19 left primer sequence |
| 407 | 6271-OT20 left primer sequence |
| 408 | 6271-OT21 left primer sequence |
| 409 | 6271-OT22 left primer sequence |
| 410 | 6271-OT23 left primer sequence |
| 411 | 6271-OT24 left primer sequence |
| 412 | 6271-OT25 left primer sequence |
| 413 | 6271-OT26 left primer sequence |
| 414 | 6271-OT27 left primer sequence |
| 415 | 6271-OT28 left primer sequence |
| 416 | 6271-OT29 left primer sequence |
| 417 | 6271-OT30 left primer sequence |
| 418 | 6272-OT1 left primer sequence |
| 419 | 6272-OT2 left primer sequence |
| 420 | 6272-OT3 left primer sequence |
| 421 | 6272-OT4 left primer sequence |
| 422 | 6272-OT5 left primer sequence |
| 423 | 6272-OT6 left primer sequence |
| 424 | 6272-OT7 left primer sequence |
| 425 | 6272-OT8 left primer sequence |
| 426 | 6272-OT9 left primer sequence |
| 427 | 6272-OT10 left primer sequence |
| 428 | 6272-OT11 left primer sequence |
| 429 | 6272-OT12 left primer sequence |
| 430 | 6272-OT13 left primer sequence |
| 431 | 6272-OT14 left primer sequence |
| 432 | 6272-OT15 left primer sequence |
| 433 | 6272-OT16 left primer sequence |
| 434 | 6272-OT17 left primer sequence |
| 435 | 6272-OT18 left primer sequence |
| 436 | 6272-OT19 left primer sequence |
| 437 | 5637-OT1 right primer sequence |
| 438 | 5637-OT2 right primer sequence |
| 439 | 5637-OT3 right primer sequence |
| 440 | 5637-OT4 right primer sequence |
| 441 | 5637-OT5 right primer sequence |
| 442 | 5637-OT6 right primer sequence |
| 443 | 5637-OT7 right primer sequence |
| 444 | 5637-OT8 right primer sequence |
| 445 | 5637-OT9 right primer sequence |
| 446 | 5637-OT10 right primer sequence |
| 447 | 5637-OT11 right primer sequence |
| 448 | 5637-OT12 right primer sequence |
| 449 | 5637-OT13 right primer sequence |
| 450 | 5637-OT14 right primer sequence |
| 451 | 5637-OT15 right primer sequence |
| 452 | 5637-OT16 right primer sequence |
| 453 | 5637-OT17 right primer sequence |
| 454 | 5637-OT18 right primer sequence |
| 455 | 5637-OT19 right primer sequence |
| 456 | 5637-OT20 right primer sequence |
| 457 | 5637-OT21 right primer sequence |
| 458 | 5637-OT22 right primer sequence |
| 459 | 5637-OT23 right primer sequence |
| 460 | 5637-OT24 right primer sequence |
| 461 | 5637-OT25 right primer sequence |
| 462 | 5637-OT26 right primer sequence |
| 463 | 5637-OT27 right primer sequence |
| 464 | 5637-OT28 right primer sequence |
| 465 | 5637-OT29 right primer sequence |
| 466 | 5637-OT30 right primer sequence |
| 467 | 5637-OT31 right primer sequence |
| 468 | 5637-OT32 right primer sequence |
| 469 | 5637-OT33 right primer sequence |
| 470 | 5637-OT34 right primer sequence |
| 471 | 5637-OT35 right primer sequence |
| 472 | 5637-OT36 right primer sequence |
| 473 | 5637-OT37 right primer sequence |
| 474 | 5641-OT1 right primer sequence |
| 475 | 5641-OT2 right primer sequence |
| 476 | 5641-OT3 right primer sequence |
| 477 | 5641-OT4 right primer sequence |
| 478 | 5641-OT5 right primer sequence |
| 479 | 5641-OT6 right primer sequence |
| 480 | 5641-OT7 right primer sequence |
| 481 | 5641-OT8 right primer sequence |
| 482 | 5641-OT9 right primer sequence |
| 483 | 5641-OT10 right primer sequence |
| 484 | 5641-OT11 right primer sequence |
| 485 | 5641-OT12 right primer sequence |
| 486 | 5641-OT13 right primer sequence |
| 487 | 5641-OT14 right primer sequence |
| 488 | 5641-OT15 right primer sequence |
| 489 | 5649-OT1 right primer sequence |
| 490 | 5707-OT1 right primer sequence |
| 491 | 5707-OT2 right primer sequence |
| 492 | 5707-OT3 right primer sequence |
| 493 | 5707-OT4 right primer sequence |
| 494 | 5707-OT5 right primer sequence |
| 495 | 5707-OT6 right primer sequence |
| 496 | 6271-OT1 right primer sequence |
| 497 | 6271-OT2 right primer sequence |
| 498 | 6271-OT3 right primer sequence |
| 499 | 6271-OT4 right primer sequence |
| 500 | 6271-OT5 right primer sequence |
| 501 | 6271-OT6 right primer sequence |
| 502 | 6271-OT7 right primer sequence |
| 503 | 6271-OT8 right primer sequence |
| 504 | 6271-OT9 right primer sequence |
| 505 | 6271-OT10 right primer sequence |
| 506 | 6271-OT11 right primer sequence |
| 507 | 6271-OT12 right primer sequence |
| 508 | 6271-OT13 right primer sequence |
| 509 | 6271-OT14 right primer sequence |
| 510 | 6271-OT15 right primer sequence |
| 511 | 6271-OT16 right primer sequence |
| 512 | 6271-OT17 right primer sequence |
| 513 | 6271-OT18 right primer sequence |
| 514 | 6271-OT19 right primer sequence |
| 515 | 6271-OT20 right primer sequence |
| 516 | 6271-OT21 right primer sequence |
| 517 | 6271-OT22 right primer sequence |
| 518 | 6271-OT23 right primer sequence |
| 519 | 6271-OT24 right primer sequence |
| 520 | 6271-OT25 right primer sequence |
| 521 | 6271-OT26 right primer sequence |
| 522 | 6271-OT27 right primer sequence |
| 523 | 6271-OT28 right primer sequence |
| 524 | 6271-OT29 right primer sequence |
| 525 | 6271-OT30 right primer sequence |
| 526 | 6272-OT1 right primer sequence |
| 527 | 6272-OT2 right primer sequence |
| 528 | 6272-OT3 right primer sequence |
| 529 | 6272-OT4 right primer sequence |
| 530 | 6272-OT5 right primer sequence |
| 531 | 6272-OT6 right primer sequence |
| 532 | 6272-OT7 right primer sequence |
| 533 | 6272-OT8 right primer sequence |
| 534 | 6272-OT9 right primer sequence |
| 535 | 6272-OT10 right primer sequence |
| 536 | 6272-OT11 right primer sequence |
| 537 | 6272-OT12 right primer sequence |
| 538 | 6272-OT13 right primer sequence |
| 539 | 6272-OT14 right primer sequence |
| 540 | 6272-OT15 right primer sequence |
| 541 | 6272-OT16 right primer sequence |
| 542 | 6272-OT17 right primer sequence |
| 543 | 6272-OT18 right primer sequence |
| 544 | 6272-OT19 right primer sequence |
| 545 | APG07433.1 RGN |
| 546 | APG07433.1 crRNA repeat |
| 547 | APG07433.1 tracrRNA |
| 548 | APG07433.1 coding sequence, mammalian |
| | codon optimized |
| 549 | APG07433.1 crRNA repeat truncated by 5 |
| | nt at 3′ end |
| 550 | APG07433.1 crRNA repeat truncated by 6 |
| | nt at 3′ end |
| 551 | APG07433.1 crRNA repeat truncated by 7 |
| | nt at 3′ end |
| 552 | APG07433.1 crRNA repeat truncated by 8 |
| | nt at 3′ end |
| 553 | APG07433.1 tracrRNA truncated by |
| | 5 nt at 5′ end, -4 tail, -2SL3 |
| 554 | APG07433.1 tracrRNA truncated by |
| | 6 nt at 5′ end (98 bb) |
| 555 | APG07433.1 tracrRNA 98bb_-6 tail |
| 556 | APG07433.1 tracrRNA 98bb_-6 |
| | tail_-2SL3 |
| 557 | APG07433.1 tracrRNA truncated by |
| | 7 nt at 5′ end (96 bb) |
| 558 | APG07433.1 tracrRNA 96bb_-6 tail |
| 559 | APG07433.1 tracrRNA 96bb_-6 |
| | tail_-2SL3 |
| 560 | APG07433.1 tracrRNA truncated by |
| | 8 nt at 5′ end (94 bb) |
| 561 | APG07433.1 tracrRNA 94bb_-6 tail |
| 562 | APG07433.1 tracrRNA 94bb_-6tail-2SL3 |
| 563 | APG07433.1 M backbone |
| 564 | APG07433.1 98 bb backbone |
| 565 | APG07433.1 98bb_-6 tail backbone |
| 566 | APG07433.1 98bb_-6 tail_-2SL3 backbone |
| 567 | APG07433.1 96 bb backbone |
| 568 | APG07433.1 96bb_-6 tail backbone |
| 569 | APG07433.1 96bb_-6 tail_-2SL3 backbone |
| 570 | APG07433.1 94 bb backbone |
| 571 | APG07433.1 94bb_-6 tail backbone |
| 572 | APG07433.1 94bb_-6tail-2SL3 backbone |
| 573 | APG07433.1 backbone |
| 574 | SGN005637 crRNA |
| 575 | SGN005641 crRNA |
| 576 | SGN005649 crRNA |
| 577 | SGN005707 crRNA |
| 578 | SGN006271 crRNA |
| 579 | SGN006272 crRNA |
| 580 | SGN002770 crRNA |
| 581 | SGN002771 crRNA |
| 582 | SGN002772 crRNA |
| 583 | SGN002773 crRNA |
| 584 | SGN002774 crRNA |
| 585 | SGN002775 crRNA |
| 586 | SGN002776 crRNA |
| 587 | SGN002777 crRNA |
| 588 | SGN002778 crRNA |
| 589 | SGN002779 crRNA |
| 590 | SGN002780 crRNA |
| 591 | SGN002781 crRNA |
| 592 | SGN002782 crRNA |
| 593 | SGN002783 crRNA |
| 594 | SGN002784 crRNA |
| 595 | SGN002785 crRNA |
| 596 | SGN002786 crRNA |
| 597 | SGN002787 crRNA |
| 598 | SGN002788 crRNA |
| 599 | SGN002789 crRNA |
| 600 | SGN002790 crRNA |
| 601 | SGN002791 crRNA |
| 602 | SGN002792 crRNA |
| 603 | SGN002793 crRNA |
| 604 | SGN002794 crRNA |
| 605 | SGN002795 crRNA |
| 606 | SGN002796 crRNA |
| 607 | SGN002797 crRNA |
| 608 | SGN002798 crRNA |
| 609 | SGN002799 crRNA |
| 610 | SGN002800 crRNA |
| 611 | SGN002801 crRNA |
| 612 | SGN002802 crRNA |
| 613 | SGN002803 crRNA |
| 614 | SGN003378 crRNA |
| 615 | SGN003379 crRNA |
| 616 | SGN003380 crRNA |
| 617 | SGN003381 crRNA |
| 618 | SGN003382 crRNA |
| 619 | SGN003383 crRNA |
| 620 | SGN003384 crRNA |
| 621 | SGN005042 crRNA |
| 622 | SGN005043 crRNA |
| 623 | SGN005044 crRNA |
| 624 | SGN005050 crRNA |
| 625 | SGN005051 crRNA |
| 626 | SGN005052 crRNA |
| 627 | SGN005053 crRNA |
| 628 | SGN005054 crRNA |
| 629 | SGN005055 crRNA |
| 630 | SGN005056 crRNA |
| 631 | SGN005057 crRNA |
| 632 | SGN005058 crRNA |
| 633 | SGN005059 crRNA |
| 634 | SGN005060 crRNA |
| 635 | SGN005061 crRNA |
| 636 | SGN005062 crRNA |
| 637 | SGN005063 crRNA |
| 638 | SGN005064 crRNA |
| 639 | SGN005065 crRNA |
| 640 | SGN005066 crRNA |
| 641 | SGN005067 crRNA |
| 642 | SGN005068 crRNA |
| 643 | SGN005069 crRNA |
| 644 | SGN005070 crRNA |
| 645 | SGN005071 crRNA |
| 646 | SGN005072 crRNA |
| 647 | SGN005073 crRNA |
| 648 | SGN005074 crRNA |
| 649 | SGN005075 crRNA |
| 650 | SGN005076 crRNA |
| 651 | SGN005077 crRNA |
| 652 | SGN005078 crRNA |
| 653 | SGN005079 crRNA |
| 654 | SGN005104 crRNA |
| 655 | SGN005635 crRNA |
| 656 | SGN005636 crRNA |
| 657 | SGN005638 crRNA |
| 658 | SGN005639 crRNA |
| 659 | SGN005640 crRNA |
| 660 | SGN005642 crRNA |
| 661 | SGN005643 crRNA |
| 662 | SGN005644 crRNA |
| 663 | SGN005645 crRNA |
| 664 | SGN005646 crRNA |
| 665 | SGN005647 crRNA |
| 666 | SGN005648 crRNA |
| 667 | SGN005650 crRNA |
| 668 | SGN005651 crRNA |
| 669 | SGN005683 SGN005684 SGN005685 crRNAs |
| 670 | SGN005686 SGN005687 SGN005688 crRNAs |
| 671 | SGN005689 SGN005690 SGN005691 crRNAs |
| 672 | SGN005692 SGN005693 SGN005694 crRNAs |
| 673 | SGN005695 SGN005696 SGN005697 crRNAs |
| 674 | SGN005698 SGN005699 SGN005700 crRNAs |
| 675 | SGN005701 SGN005702 SGN005703 crRNAs |
| 676 | SGN005704 SGN005705 SGN005706 crRNAs |
| 677 | SGN005708 SGN005709 crRNAs |
| 678 | SGN005710 SGN005711 SGN005712 crRNAs |
| 679 | SGN005713 SGN005714 SGN005715 crRNAs |
| 680 | SGN005716 SGN005717 SGN005718 crRNAs |
| 681 | SGN006269 crRNA |
| 682 | SGN006270 crRNA |
| 683 | SGN006273 crRNA |
| 684 | SGN006274 crRNA |
| 685 | SGN006275 crRNA |
| 686 | SGN006276 crRNA |
| 687 | SGN006277 crRNA |
| 688 | SGN006278 crRNA |
| 689 | SGN006279 crRNA |
| 690 | SGN006280 crRNA |
| 691 | SGN006281 crRNA |
| 692 | SGN006282 crRNA |
| 693 | SGN005637 guide RNA |
| 694 | SGN005641 guide RNA |
| 695 | SGN005649 guide RNA |
| 696 | SGN005707 guide RNA |
| 697 | SGN006271 guide RNA |
| 698 | SGN006272 guide RNA |
| 699 | SGN002770 guide RNA |
| 700 | SGN002771 guide RNA |
| 701 | SGN002772 guide RNA |
| 702 | SGN002773 guide RNA |
| 703 | SGN002774 guide RNA |
| 704 | SGN002775 guide RNA |
| 705 | SGN002776 guide RNA |
| 706 | SGN002777 guide RNA |
| 707 | SGN002778 guide RNA |
| 708 | SGN002779 guide RNA |
| 709 | SGN002780 guide RNA |
| 710 | SGN002781 guide RNA |
| 711 | SGN002782 guide RNA |
| 712 | SGN002783 guide RNA |
| 713 | SGN002784 guide RNA |
| 714 | SGN002785 guide RNA |
| 715 | SGN002786 guide RNA |
| 716 | SGN002787 guide RNA |
| 717 | SGN002788 guide RNA |
| 718 | SGN002789 guide RNA |
| 719 | SGN002790 guide RNA |
| 720 | SGN002791 guide RNA |
| 721 | SGN002792 guide RNA |
| 722 | SGN002793 guide RNA |
| 723 | SGN002794 guide RNA |
| 724 | SGN002795 guide RNA |
| 725 | SGN002796 guide RNA |
| 726 | SGN002797 guide RNA |
| 727 | SGN002798 guide RNA |
| 728 | SGN002799 guide RNA |
| 729 | SGN002800 guide RNA |
| 730 | SGN002801 guide RNA |
| 731 | SGN002802 guide RNA |
| 732 | SGN002803 guide RNA |
| 733 | SGN003378 guide RNA |
| 734 | SGN003379 guide RNA |
| 735 | SGN003380 guide RNA |
| 736 | SGN003381 guide RNA |
| 737 | SGN003382 guide RNA |
| 738 | SGN003383 guide RNA |
| 739 | SGN003384 guide RNA |
| 740 | SGN005042 guide RNA |
| 741 | SGN005043 guide RNA |
| 742 | SGN005044 guide RNA |
| 743 | SGN005050 guide RNA |
| 744 | SGN005051 guide RNA |
| 745 | SGN005052 guide RNA |
| 746 | SGN005053 guide RNA |
| 747 | SGN005054 guide RNA |
| 748 | SGN005055 guide RNA |
| 749 | SGN005056 guide RNA |
| 750 | SGN005057 guide RNA |
| 751 | SGN005058 guide RNA |
| 752 | SGN005059 guide RNA |
| 753 | SGN005060 guide RNA |
| 754 | SGN005061 guide RNA |
| 755 | SGN005062 guide RNA |
| 756 | SGN005063 guide RNA |
| 757 | SGN005064 guide RNA |
| 758 | SGN005065 guide RNA |
| 759 | SGN005066 guide RNA |
| 760 | SGN005067 guide RNA |
| 761 | SGN005068 guide RNA |
| 762 | SGN005069 guide RNA |
| 763 | SGN005070 guide RNA |
| 764 | SGN005071 guide RNA |
| 765 | SGN005072 guide RNA |
| 766 | SGN005073 guide RNA |
| 767 | SGN005074 guide RNA |
| 768 | SGN005075 guide RNA |
| 769 | SGN005076 guide RNA |
| 770 | SGN005077 guide RNA |
| 771 | SGN005078 guide RNA |
| 772 | SGN005079 guide RNA |
| 773 | SGN005104 guide RNA |
| 774 | SGN005635 guide RNA |
| 775 | SGN005636 guide RNA |
| 776 | SGN005638 guide RNA |
| 777 | SGN005639 guide RNA |
| 778 | SGN005640 guide RNA |
| 779 | SGN005642 guide RNA |
| 780 | SGN005643 guide RNA |
| 781 | SGN005644 guide RNA |
| 782 | SGN005645 guide RNA |
| 783 | SGN005646 guide RNA |
| 784 | SGN005647 guide RNA |
| 785 | SGN005648 guide RNA |
| 786 | SGN005650 guide RNA |
| 787 | SGN005651 guide RNA |
| 788 | SGN005683 guide RNA |
| 789 | SGN005684 guide RNA |
| 790 | SGN005685 guide RNA |
| 791 | SGN005686 guide RNA |
| 792 | SGN005687 guide RNA |
| 793 | SGN005688 guide RNA |
| 794 | SGN005689 guide RNA |
| 795 | SGN005690 guide RNA |
| 796 | SGN005691 guide RNA |
| 797 | SGN005692 guide RNA |
| 798 | SGN005693 guide RNA |
| 799 | SGN005694 guide RNA |
| 800 | SGN005695 guide RNA |
| 801 | SGN005696 guide RNA |
| 802 | SGN005697 guide RNA |
| 803 | SGN005698 guide RNA |
| 804 | SGN005699 guide RNA |
| 805 | SGN005700 guide RNA |
| 806 | SGN005701 guide RNA |
| 807 | SGN005702 guide RNA |
| 808 | SGN005703 guide RNA |
| 809 | SGN005704 guide RNA |
| 810 | SGN005705 guide RNA |
| 811 | SGN005706 guide RNA |
| 812 | SGN005708 guide RNA |
| 813 | SGN005709 guide RNA |
| 814 | SGN005710 guide RNA |
| 815 | SGN005711 guide RNA |
| 816 | SGN005712 guide RNA |
| 817 | SGN005713 guide RNA |
| 818 | SGN005714 guide RNA |
| 819 | SGN005715 guide RNA |
| 820 | SGN005716 guide RNA |
| 821 | SGN005717 guide RNA |
| 822 | SGN005718 guide RNA |
| 823 | SGN006269 guide RNA |
| 824 | SGN006270 guide RNA |
| 825 | SGN006273 guide RNA |
| 826 | SGN006274 guide RNA |
| 827 | SGN006275 guide RNA |
| 828 | SGN006276 guide RNA |
| 829 | SGN006277 guide RNA |
| 830 | SGN006278 guide RNA |
| 831 | SGN006279 guide RNA |
| 832 | SGN006280 guide RNA |
| 833 | SGN006281 guide RNA |
| 834 | SGN006282 guide RNA |
| 835 | Streptococcus pyogenes Cas9 |
| 836 | Streptococcus pyogenes |
| | Cas9 D10A nickase |
| 837 | Neisseria meningitidis Cas9 (Nme2Cas9) |
| 838 | APG05083.1 RGN |
| 839 | APG05083.1 crRNA repeat |
| 840 | APG05083.1 tracrRNA |
| 841 | APG07513.1 RGN |
| 842 | APG07513.1 crRNA repeat |
| 843 | APG07513.1 tracrRNA |
| 844 | APG08290.1 RGN |
| 845 | APG08290.1 crRNA repeat sequence |
| 846 | APG08290.1 tracrRNA |
| 847 | APG05459.1 RGN |
| 848 | APG05459.1 crRNA repeat |
| 849 | APG05459.1 tracrRNA |
| 850 | APG01688.1 RGN |
| 851 | APG01688.1 crRNA repeat |
| 852 | APG01688.1 tracrRNA |
| 853 | APG03128 RGN |
| 854 | APG03128 crRNA repeat |
| 855 | APG03128 tracrRNA |
| 856 | APG05733.1 RGN |
| 857 | APG05733.1 crRNA repeat |
| 858 | APG05733.1 tracrRNA |
| 859 | APG01658.1 RGN |
| 860 | APG01658.1 crRNA repeat |
| 861 | APG01658.1 tracrRNA |
| 862 | APG06498.1 RGN |
| 863 | APG06498.1 crRNA repeat |
| 864 | APG06498.1 tracrRNA |
| 865 | APG06877.1 RGN |
| 866 | APG06877.1 crRNA repeat |
| 867 | APG06877.1 tracrRNA |
| 868 | APG09053.1 RGN |
| 869 | APG09053.1 crRNA repeat |
| 870 | APG09053.1 tracrRNA |
| 871 | APG04293.1 RGN |
| 872 | APG04293.1 crRNA repeat |
| 873 | APG04293.1 tracrRNA |
| 874 | APG06646.1 RGN |
| 875 | APG06646.1 crRNA repeat |
| 876 | APG06646.1 tracrRNA |
| 877 | APG02874 RGN |
| 878 | APG02874 crRNA repeat |
| 879 | APG02874 tracrRNA |
| 880 | APG03031 RGN |
| 881 | APG03031 crRNA repeat |
| 882 | APG03031 tracrRNA |
| 883 | APG09208 RGN |
| 884 | APG09208 crRNA repeat |
| 885 | APG09208 tracrRNA |
| 886 | APG09344 RGN |
| 887 | APG09344 crRNA repeat |
| 888 | APG09344 tracrRNA |
| 889 | APG07991 RGN |
| 890 | APG07991 crRNA repeat |
| 891 | APG07991 tracrRNA |
| 892 | APG01868 RGN |
| 893 | APG01868 crRNA repeat |
| 894 | APG01868 tracrRNA |
| 895 | APG02998 RGN |
| 896 | APG02998 crRNA repeat |
| 897 | APG02998 tracrRNA |
| 898 | APG07433.1 deletion variant RGN |
| 899 | APG08290.1 deletion variant RGN |
| 900 | LPG10134 RGN |
| 901 | LPG10134 crRNA repeat |
| 902 | LPG10134 tracrRNA |
| 903 | LPG10136 RGN |
| 904 | LPG10136 crRNA repeat |
| 905 | LPG10136 tracrRNA |
| 906 | LPG10138 RGN |
| 907 | LPG10138 crRNA repeat |
| 908 | LPG10138 tracrRNA |
| 909 | LPG10139 RGN |
| 910 | LPG10139 crRNA repeat |
| 911 | LPG10139 tracrRNA |
| 912 | LPG10141 RGN |
| 913 | LPG10141 crRNA repeat |
| 914 | LPG10141 tracrRNA |
| 915 | LPG10145 RGN |
| 916 | LPG10145 crRNA repeat |
| 917 | LPG10145 tracrRNA |
| 918 | SpyCas9 crRNA repeat |
| 919 | SpyCas9 tracrRNA |
| 920 | SpyCas9 sgRNA |
| 921 | Conserved amino acid motif in a meganuclease |
| 922 | SV40 nuclear localization signal |
| 923 | nucleoplasmin nuclear localization signal |
| 924 | Human U6 promoter |
| 925 | NC_041760 promoter |
| 926 | NW_004848155 promoter |
| 927 | LR738627 promoter |
| 928 | NC_044556 promoter |
| 929 | XM_030845548 promoter |
| 930 | Mini NC_041760 promoter |
| 931 | Mini NW_004848155 promoter |
| 932 | Mini LR738627 promoter |
| 933 | Mini NC_044556 promoter |
| 934 | Mini XM_030845548 promoter |
| 935 | CSB/ERCC6, a member of a nucleotide excision repair |
| | (NER) pathway |
| 936 | 3xFLAG tag |
| 937 | Homo sapiens FOXP3 protein (isoform a; NCBI |
| | Reference Sequence NP_054728.2) |
| 938 | Homo sapiens FOXP3 protein (isoform b; NCBI |
| | Reference Sequence NP_001107849.1) |
| 939 | Homo sapiens FOXP3 gene |
| 940 | MS modified APG07433.1 crRNA repeat |
| 941 | MS modified APG07433.1 tracrRNA |
| 942 | MS modified APG07433.1 crRNA repeat truncated by |
| | 5 nt at 3′ end |
| 943 | MS modified APG07433.1 crRNA repeat truncated by |
| | 6 nt at 3′ end |
| 944 | MS modified APG07433.1 crRNA repeat truncated by |
| | 7 nt at 3′ end |
| 945 | MS modified APG07433.1 crRNA repeat truncated by |
| | 8 nt at 3′ end |
| 946 | MS modified APG07433.1 tracrRNA truncated by 5 nt |
| | at 5′ end, -4 tail, -2SL3 |
| 947 | MS modified APG07433.1 tracrRNA truncated by 6 nt |
| | at 5′ end (98 bb) |
| 948 | MS modified APG07433.1 tracrRNA 98bb_-6 tail |
| 949 | MS modified APG07433.1 tracrRNA 98bb_- 6 tail_-2SL3 |
| 950 | MS modified APG07433.1 tracrRNA truncated by 7 nt |
| | at 5′ end (96 bb) |
| 951 | MS modified APG07433.1 tracrRNA 96bb_-6 tail |
| 952 | MS modified APG07433.1 tracrRNA 96bb_- 6 tail_-2SL3 |
| 953 | MS modified APG07433.1 tracrRNA truncated by 8 nt |
| | at 5′ end (94 bb) |
| 954 | MS modified APG07433.1 tracrRNA 94bb_-6 tail |
| 955 | MS modified APG07433.1 tracrRNA 94bb_-6tail-2SL3 |
| 956 | MS modified APG07433.1 M backbone |
| 957 | MS modified APG07433.1 98 bb backbone |
| 958 | MS modified APG07433.1 98bb_-6 tail backbone |
| 959 | MS modified APG07433.1 98bb_-6 tail_-2SL3 |
| | backbone |
| 960 | MS modified APG07433.1 96 bb backbone |
| 961 | MS modified APG07433.1 96bb_-6 tail backbone |
| 962 | MS modified APG07433.1 96bb_-6 tail_-2SL3 |
| | backbone |
| 963 | MS modified APG07433.1 94 bb backbone |
| 964 | MS modified APG07433.1 94bb_-6 tail backbone |
| 965 | MS modified APG07433.1 94bb_-6tail-2SL3 backbone |
| 966 | MS modified APG07433.1 backbone |
| 967 | MS modified SGN005637 crRNA |
| 968 | MS modified SGN005641 crRNA |
| 969 | MS modified SGN005649 crRNA |
| 970 | MS modified SGN005707 crRNA |
| 971 | MS modified SGN006271 crRNA |
| 972 | MS modified SGN006272 crRNA |
| 973 | MS modified SGN002770 crRNA |
| 974 | MS modified SGN002771 crRNA |
| 975 | MS modified SGN002772 crRNA |
| 976 | MS modified SGN002773 crRNA |
| 977 | MS modified SGN002774 crRNA |
| 978 | MS modified SGN002775 crRNA |
| 979 | MS modified SGN002776 crRNA |
| 980 | MS modified SGN002777 crRNA |
| 981 | MS modified SGN002778 crRNA |
| 982 | MS modified SGN002779 crRNA |
| 983 | MS modified SGN002780 crRNA |
| 984 | MS modified SGN002781 crRNA |
| 985 | MS modified SGN002782 crRNA |
| 986 | MS modified SGN002783 crRNA |
| 987 | MS modified SGN002784 crRNA |
| 988 | MS modified SGN002785 crRNA |
| 989 | MS modified SGN002786 crRNA |
| 990 | MS modified SGN002787 crRNA |
| 991 | MS modified SGN002788 crRNA |
| 992 | MS modified SGN002789 crRNA |
| 993 | MS modified SGN002790 crRNA |
| 994 | MS modified SGN002791 crRNA |
| 995 | MS modified SGN002792 crRNA |
| 996 | MS modified SGN002793 crRNA |
| 997 | MS modified SGN002794 crRNA |
| 998 | MS modified SGN002795 crRNA |
| 999 | MS modified SGN002796 crRNA |
| 1000 | MS modified SGN002797 crRNA |
| 1001 | MS modified SGN002798 crRNA |
| 1002 | MS modified SGN002799 crRNA |
| 1003 | MS modified SGN002800 crRNA |
| 1004 | MS modified SGN002801 crRNA |
| 1005 | MS modified SGN002802 crRNA |
| 1006 | MS modified SGN002803 crRNA |
| 1007 | MS modified SGN003378 crRNA |
| 1008 | MS modified SGN003379 crRNA |
| 1009 | MS modified SGN003380 crRNA |
| 1010 | MS modified SGN003381 crRNA |
| 1011 | MS modified SGN003382 crRNA |
| 1012 | MS modified SGN003383 crRNA |
| 1013 | MS modified SGN003384 crRNA |
| 1014 | MS modified SGN005042 crRNA |
| 1015 | MS modified SGN005043 crRNA |
| 1016 | MS modified SGN005044 crRNA |
| 1017 | MS modified SGN005050 crRNA |
| 1018 | MS modified SGN005051 crRNA |
| 1019 | MS modified SGN005052 crRNA |
| 1020 | MS modified SGN005053 crRNA |
| 1021 | MS modified SGN005054 crRNA |
| 1022 | MS modified SGN005055 crRNA |
| 1023 | MS modified SGN005056 crRNA |
| 1024 | MS modified SGN005057 crRNA |
| 1025 | MS modified SGN005058 crRNA |
| 1026 | MS modified SGN005059 crRNA |
| 1027 | MS modified SGN005060 crRNA |
| 1028 | MS modified SGN005061 crRNA |
| 1029 | MS modified SGN005062 crRNA |
| 1030 | MS modified SGN005063 crRNA |
| 1031 | MS modified SGN005064 crRNA |
| 1032 | MS modified SGN005065 crRNA |
| 1033 | MS modified SGN005066 crRNA |
| 1034 | MS modified SGN005067 crRNA |
| 1035 | MS modified SGN005068 crRNA |
| 1036 | MS modified SGN005069 crRNA |
| 1037 | MS modified SGN005070 crRNA |
| 1038 | MS modified SGN005071 crRNA |
| 1039 | MS modified SGN005072 crRNA |
| 1040 | MS modified SGN005073 crRNA |
| 1041 | MS modified SGN005074 crRNA |
| 1042 | MS modified SGN005075 crRNA |
| 1043 | MS modified SGN005076 CrRNA |
| 1044 | MS modified SGN005077 crRNA |
| 1045 | MS modified SGN005078 crRNA |
| 1046 | MS modified SGN005079 crRNA |
| 1047 | MS modified SGN005104 crRNA |
| 1048 | MS modified SGN005635 crRNA |
| 1049 | MS modified SGN005636 crRNA |
| 1050 | MS modified SGN005638 crRNA |
| 1051 | MS modified SGN005639 crRNA |
| 1052 | MS modified SGN005640 crRNA |
| 1053 | MS modified SGN005642 crRNA |
| 1054 | MS modified SGN005643 crRNA |
| 1055 | MS modified SGN005644 crRNA |
| 1056 | MS modified SGN005645 crRNA |
| 1057 | MS modified SGN005646 crRNA |
| 1058 | MS modified SGN005647 crRNA |
| 1059 | MS modified SGN005648 crRNA |
| 1060 | MS modified SGN005650 crRNA |
| 1061 | MS modified SGN005651 crRNA |
| 1062 | SGN005683 SGN005684 |
| | SGN005685 MS modified crRNAs |
| 1063 | SGN005686 SGN005687 |
| | SGN005688 MS modified crRNAs |
| 1064 | SGN005689 SGN005690 |
| | SGN005691 MS modified crRNAs |
| 1065 | SGN005692 SGN005693 |
| | SGN005694 MS modified crRNAs |
| 1066 | SGN005695 SGN005696 |
| | SGN005697 MS modified crRNAs |
| 1067 | SGN005698 SGN005699 |
| | SGN005700 MS modified crRNAs |
| 1068 | SGN005701 SGN005702 |
| | SGN005703 MS modified crRNAs |
| 1069 | SGN005704 SGN005705 |
| | SGN005706 MS modified crRNAs |
| 1070 | SGN005708 SGN005709 MS modified crRNAs |
| 1071 | SGN005710 SGN005711 |
| | SGN005712 MS modified crRNAs |
| 1072 | SGN005713 SGN005714 |
| | SGN005715 MS modified crRNAs |
| 1073 | SGN005716 SGN005717 |
| | SGN005718 MS modified crRNAs |
| 1074 | MS modified SGN006269 crRNA |
| 1075 | MS modified SGN006270 crRNA |
| 1076 | MS modified SGN006273 crRNA |
| 1077 | MS modified SGN006274 crRNA |
| 1078 | MS modified SGN006275 crRNA |
| 1079 | MS modified SGN006276 crRNA |
| 1080 | MS modified SGN006277 crRNA |
| 1081 | MS modified SGN006278 crRNA |
| 1082 | MS modified SGN006279 crRNA |
| 1083 | MS modified SGN006280 crRNA |
| 1084 | MS modified SGN006281 crRNA |
| 1085 | MS modified SGN006282 crRNA |
| 1086 | MS modified SGN005637 guide RNA |
| 1087 | MS modified SGN005641 guide RNA |
| 1088 | MS modified SGN005649 guide RNA |
| 1089 | MS modified SGN005707 guide RNA |
| 1090 | MS modified SGN006271 guide RNA |
| 1091 | MS modified SGN006272 guide RNA |
| 1092 | MS modified SGN002770 guide RNA |
| 1093 | MS modified SGN002771 guide RNA |
| 1094 | MS modified SGN002772 guide RNA |
| 1095 | MS modified SGN002773 guide RNA |
| 1096 | MS modified SGN002774 guide RNA |
| 1097 | MS modified SGN002775 guide RNA |
| 1098 | MS modified SGN002776 guide RNA |
| 1099 | MS modified SGN002777 guide RNA |
| 1100 | MS modified SGN002778 guide RNA |
| 1101 | MS modified SGN002779 guide RNA |
| 1102 | MS modified SGN002780 guide RNA |
| 1103 | MS modified SGN002781 guide RNA |
| 1104 | MS modified SGN002782 guide RNA |
| 1105 | MS modified SGN002783 guide RNA |
| 1106 | MS modified SGN002784 guide RNA |
| 1107 | MS modified SGN002785 guide RNA |
| 1108 | MS modified SGN002786 guide RNA |
| 1109 | MS modified SGN002787 guide RNA |
| 1110 | MS modified SGN002788 guide RNA |
| 1111 | MS modified SGN002789 guide RNA |
| 1112 | MS modified SGN002790 guide RNA |
| 1113 | MS modified SGN002791 guide RNA |
| 1114 | MS modified SGN002792 guide RNA |
| 1115 | MS modified SGN002793 guide RNA |
| 1116 | MS modified SGN002794 guide RNA |
| 1117 | MS modified SGN002795 guide RNA |
| 1118 | MS modified SGN002796 guide RNA |
| 1119 | MS modified SGN002797 guide RNA |
| 1120 | MS modified SGN002798 guide RNA |
| 1121 | MS modified SGN002799 guide RNA |
| 1122 | MS modified SGN002800 guide RNA |
| 1123 | MS modified SGN002801 guide RNA |
| 1124 | MS modified SGN002802 guide RNA |
| 1125 | MS modified SGN002803 guide RNA |
| 1126 | MS modified SGN003378 guide RNA |
| 1127 | MS modified SGN003379 guide RNA |
| 1128 | MS modified SGN003380 guide RNA |
| 1129 | MS modified SGN003381 guide RNA |
| 1130 | MS modified SGN003382 guide RNA |
| 1131 | MS modified SGN003383 guide RNA |
| 1132 | MS modified SGN003384 guide RNA |
| 1133 | MS modified SGN005042 guide RNA |
| 1134 | MS modified SGN005043 guide RNA |
| 1135 | MS modified SGN005044 guide RNA |
| 1136 | MS modified SGN005050 guide RNA |
| 1137 | MS modified SGN005051 guide RNA |
| 1138 | MS modified SGN005052 guide RNA |
| 1139 | MS modified SGN005053 guide RNA |
| 1140 | MS modified SGN005054 guide RNA |
| 1141 | MS modified SGN005055 guide RNA |
| 1142 | MS modified SGN005056 guide RNA |
| 1143 | MS modified SGN005057 guide RNA |
| 1144 | MS modified SGN005058 guide RNA |
| 1145 | MS modified SGN005059 guide RNA |
| 1146 | MS modified SGN005060 guide RNA |
| 1147 | MS modified SGN005061 guide RNA |
| 1148 | MS modified SGN005062 guide RNA |
| 1149 | MS modified SGN005063 guide RNA |
| 1150 | MS modified SGN005064 guide RNA |
| 1151 | MS modified SGN005065 guide RNA |
| 1152 | MS modified SGN005066 guide RNA |
| 1153 | MS modified SGN005067 guide RNA |
| 1154 | MS modified SGN005068 guide RNA |
| 1155 | MS modified SGN005069 guide RNA |
| 1156 | MS modified SGN005070 guide RNA |
| 1157 | MS modified SGN005071 guide RNA |
| 1158 | MS modified SGN005072 guide RNA |
| 1159 | MS modified SGN005073 guide RNA |
| 1160 | MS modified SGN005074 guide RNA |
| 1161 | MS modified SGN005075 guide RNA |
| 1162 | MS modified SGN005076 guide RNA |
| 1163 | MS modified SGN005077 guide RNA |
| 1164 | MS modified SGN005078 guide RNA |
| 1165 | MS modified SGN005079 guide RNA |
| 1166 | MS modified SGN005104 guide RNA |
| 1167 | MS modified SGN005635 guide RNA |
| 1168 | MS modified SGN005636 guide RNA |
| 1169 | MS modified SGN005638 guide RNA |
| 1170 | MS modified SGN005639 guide RNA |
| 1171 | MS modified SGN005640 guide RNA |
| 1172 | MS modified SGN005642 guide RNA |
| 1173 | MS modified SGN005643 guide RNA |
| 1174 | MS modified SGN005644 guide RNA |
| 1175 | MS modified SGN005645 guide RNA |
| 1176 | MS modified SGN005646 guide RNA |
| 1177 | MS modified SGN005647 guide RNA |
| 1178 | MS modified SGN005648 guide RNA |
| 1179 | MS modified SGN005650 guide RNA |
| 1180 | MS modified SGN005651 guide RNA |
| 1181 | MS modified SGN005683 guide RNA |
| 1182 | MS modified SGN005684 guide RNA |
| 1183 | MS modified SGN005685 guide RNA |
| 1184 | MS modified SGN005686 guide RNA |
| 1185 | MS modified SGN005687 guide RNA |
| 1186 | MS modified SGN005688 guide RNA |
| 1187 | MS modified SGN005689 guide RNA |
| 1188 | MS modified SGN005690 guide RNA |
| 1189 | MS modified SGN005691 guide RNA |
| 1190 | MS modified SGN005692 guide RNA |
| 1191 | MS modified SGN005693 guide RNA |
| 1192 | MS modified SGN005694 guide RNA |
| 1193 | MS modified SGN005695 guide RNA |
| 1194 | MS modified SGN005696 guide RNA |
| 1195 | MS modified SGN005697 guide RNA |
| 1196 | MS modified SGN005698 guide RNA |
| 1197 | MS modified SGN005699 guide RNA |
| 1198 | MS modified SGN005700 guide RNA |
| 1199 | MS modified SGN005701 guide RNA |
| 1200 | MS modified SGN005702 guide RNA |
| 1201 | MS modified SGN005703 guide RNA |
| 1202 | MS modified SGN005704 guide RNA |
| 1203 | MS modified SGN005705 guide RNA |
| 1204 | MS modified SGN005706 guide RNA |
| 1205 | MS modified SGN005708 guide RNA |
| 1206 | MS modified SGN005709 guide RNA |
| 1207 | MS modified SGN005710 guide RNA |
| 1208 | MS modified SGN005711 guide RNA |
| 1209 | MS modified SGN005712 guide RNA |
| 1210 | MS modified SGN005713 guide RNA |
| 1211 | MS modified SGN005714 guide RNA |
| 1212 | MS modified SGN005715 guide RNA |
| 1213 | MS modified SGN005716 guide RNA |
| 1214 | MS modified SGN005717 guide RNA |
| 1215 | MS modified SGN005718 guide RNA |
| 1216 | MS modified SGN006269 guide RNA |
| 1217 | MS modified SGN006270 guide RNA |
| 1218 | MS modified SGN006273 guide RNA |
| 1219 | MS modified SGN006274 guide RNA |
| 1220 | MS modified SGN006275 guide RNA |
| 1221 | MS modified SGN006276 guide RNA |
| 1222 | MS modified SGN006277 guide RNA |
| 1223 | MS modified SGN006278 guide RNA |
| 1224 | MS modified SGN006279 guide RNA |
| 1225 | MS modified SGN006280 guide RNA |
| 1226 | MS modified SGN006281 guide RNA |
| 1227 | MS modified SGN006282 guide RNA |
| 1228 | MS modified APG05083.1 crRNA repeat |
| 1229 | MS modified APG05083.1 tracrRNA |
| 1230 | MS modified APG07513.1 crRNA repeat |
| 1231 | MS modified APG07513.1 tracrRNA |
| 1232 | MS modified APG08290.1 crRNA repeat sequence |
| 1233 | MS modified APG08290.1 tracrRNA |

That which is claimed:

1. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
wherein the tracrRNA comprises:
(iii) an anti-repeat; and
(iv) a tail,
wherein the spacer is capable of hybridizing to a target sequence in a forkhead box P3 (FOXP3) gene,
wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

2. The gRNA of claim 1, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by lto 5 nucleotides.

3. The gRNA of claim 1, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

4. The gRNA of any one of claims 1-3, wherein the crRNA repeat has the nucleotide sequence set forth as SEQ ID NO: 546 or a nucleotide sequence that differs in length and/or sequence from SEQ ID NO: 546 by 1 to 8 nucleotides.

5. The gRNA of claim 4, wherein the crRNA repeat has the nucleotide sequence set forth as any one of SEQ ID NOs: 546, 549-552, 839, 842, and 845.

6. The gRNA of any one of claims 1-5, wherein the crRNA has the nucleotide sequence set forth as any one of SEQ ID NOs: 574-692.

7. The gRNA of any one of claims 1-6, wherein the tracrRNA has a nucleotide sequence having at least 80% sequence identity, at least 90% sequence identity, or at least 95% sequence identity to SEQ ID NO: 547.

8. The gRNA of any one of claims 1-6, wherein the tracrRNA has a nucleotide sequence that differs in length from SEQ ID NO: 547 by 1 to 16 nucleotides.

9. The gRNA of claim 8, wherein the tracrRNA has a nucleotide sequence that is 8 nucleotides shorter than SEQ ID NO: 547.

10. The gRNA of claim 8, wherein the tracrRNA has a nucleotide sequence that is 11 nucleotides shorter than SEQ ID NO: 547.

11. The gRNA of claim 7 or 8, wherein the tracrRNA has the nucleotide sequence set forth as any one of SEQ ID NOs: 547, 553-562, 840, 842, and 846.

12. The gRNA of any one of claims 1-3, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.

13. The gRNA of claim 12, wherein the linker has a nucleotide sequence set forth as AAAG, GAAA, ACUU, or CAAAGG.

14. The gRNA of claim 13, wherein the linker has a nucleotide sequence set forth as AAAG.

15. The gRNA of any one of claims 12-14, wherein the backbone of the sgRNA comprises a total length of at least 85, 90, 95, 100, 105, 110, 115, or 120 nucleotides.

16. The gRNA of any one of claims 12-14, wherein the backbone of the sgRNA comprises a total length of at most 85, 90, 95, 100, 105, 110, 115, or 120 nucleotides.

17. The gRNA of any one of claims 12-14, wherein the backbone of the sgRNA comprises a total length of 86 to 98 nucleotides.

18. The gRNA of any one of claims 12-14, wherein the backbone of the sgRNA comprises a total length of 94 nucleotides.

19. The gRNA of any one of claims 12-14, wherein the backbone of the sgRNA has a nucleotide sequence having at least 80% sequence identity, at least 90% sequence identity, at least 95% sequence identity, or 100% sequence identity to any one of SEQ ID NOs: 563-573.

20. The gRNA of any one of claims 1-3, wherein the gRNA comprises a first stem loop formed by hybridization of the crRNA repeat and the anti-repeat, wherein the first stem loop comprises a first stem and a second stem, and wherein the first stem of the first stem loop comprises a total length of at least 3, 4, 5, 6, 7, 8, 9, 10, or 11 base pairs (bp).

21. The gRNA of any one of claims 1-3, wherein the gRNA comprises a first stem loop formed by hybridization of the crRNA repeat and the anti-repeat, wherein the first stem loop comprises a first stem and a second stem, and wherein the first stem of the first stem loop comprises a total length of at most 3, 4, 5, 6, 7, 8, 9, 10, or 11 bp.

22. The gRNA of claim 20 or 21, wherein the first stem of the first stem loop comprises a total length of 6 bp.

23. The gRNA of claim 20 or 21, wherein the first stem of the first stem loop comprises a total length of 3 bp.

24. The gRNA of any one of claims 1-3, wherein the tail of the tracrRNA comprises a total length of at least 1, 2, 3, 4, 5, 6, or 7 nucleotides.

25. The gRNA of any one of claims 1-3, wherein the tail of the tracrRNA comprises a total length of at most 1, 2, 3, 4, 5, 6, or 7 nucleotides.

26. The gRNA of claim 24 or 25, wherein the tail of the tracrRNA comprises a total length of 3 nucleotides.

27. The gRNA of claim 24 or 25, wherein the tail of the tracrRNA comprises a total length of 1 nucleotide.

28. The gRNA of claim 20 or 21, wherein the gRNA further comprises a second stem loop most proximal to the tail, wherein the second stem loop comprises a first stem and a second stem.

29. The gRNA of claim 28, wherein the first stem of the second stem loop comprises a total length of at least 1, 2, 3, 4, 5, or 6 bp.

30. The gRNA of claim 28, wherein the first stem of the second stem loop comprises a total length of at most 1, 2, 3, 4, 5, or 6 bp.

31. The gRNA of claim 29 or 30, wherein the first stem of the second stem loop comprises a total length of 5 bp.

32. The gRNA of any one of claims 28-31, wherein the first stem of the first stem loop comprises a total length of 6 bp, the tail of the tracrRNA comprises a total length of 3 nucleotides, and the first stem of the second stem loop comprises a total length of 5 bp.

33. The gRNA of any one of claims 1-3, wherein the gRNA is a dual guide RNA (dgRNA).

34. The gRNA of claim 33, wherein the crRNA repeat of the dgRNA comprises a total length of at least 10, 11, 12, 13, 14, 15, 16, 17, 18, 19, 20, or 21 nucleotides.

35. The gRNA of claim 33, wherein the crRNA repeat of the dgRNA comprises a total length of at most 10, 11, 12, 13, 14, 15, 16, 17, 18, 19, 20, or 21 nucleotides.

36. The gRNA of claim 34 or 35, wherein the crRNA repeat of the dgRNA comprises a total length of 13 nucleotides.

37. The gRNA of claim 34 or 35, wherein the crRNA repeat of the dgRNA comprises a total length of 16 nucleotides.

38. The gRNA of claim 34 or 35, wherein the crRNA repeat of the dgRNA comprises a total length of 21 nucleotides.

39. The gRNA of claim 33, wherein the tracrRNA of the dgRNA comprises a total length of at least 65, 70, 75, 80, or 85 nucleotides.

40. The gRNA of claim 33, wherein the tracrRNA of the dgRNA comprises a total length of at most 65, 70, 75, 80, or 85 nucleotides.

41. The gRNA of claim 39 or 40, wherein the tracrRNA of the dgRNA comprises a total length of 74 nucleotides.

42. The gRNA of claim 39 or 40, wherein the tracrRNA of the dgRNA comprises a total length of 77 nucleotides.

43. The gRNA of any one of claims 1-42, wherein the gRNA comprises a total length of at least 85, 90, 95, 100, 105, 110, 115, 120, 125, 130, or 135 nucleotides.

44. The gRNA of any one of claims 1-42, wherein the gRNA comprises a total length of at most 85, 90, 95, 100, 105, 110, 115, 120, 125, 130, or 135 nucleotides.

45. The gRNA of any one of claims 1-42, wherein the gRNA comprises a total length of 106 to 135 nucleotides.

46. The gRNA of claim 45, wherein the gRNA comprises a total length of 117 to 119 nucleotides.

47. The gRNA of any one of claims 1-46, wherein the gRNA is capable of targeting a bound RNA-guided nuclease (RGN) polypeptide to the target sequence.

48. The gRNA of claim 47, wherein the RGN polypeptide is capable of recognizing a consensus protospacer adjacent motif (PAM) having the nucleotide sequence set forth as NNNNCC.

49. The gRNA of claim 48, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.

50. The gRNA of any one of claims 47-49, wherein the RGN polypeptide has an amino acid sequence having at least 80% sequence identity to SEQ ID NO: 545; and wherein the target sequence and the spacer are selected from the group consisting of:
a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides;
b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides;
c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides;
d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides;
e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and
f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides.

51. The gRNA of claim 50, wherein the spacer has the nucleotide sequence set forth as any one of SEQ ID NOs: 155, 163, 189, 179, 197, and 193.

52. The gRNA of claim 50 or 51, wherein the RGN polypeptide has the amino acid sequence set forth as SEQ ID NO: 545.

53. The gRNA of any one of claims 47-52, wherein the gRNA has a nucleotide sequence set forth as any one of SEQ ID NOs: 693-834.

54. The gRNA of claim 53, wherein the gRNA has a nucleotide sequence set forth as any one of SEQ ID NOs: 693, 694, 695, 696, 697, and 698.

55. The gRNA of any one of claims 1-54, wherein the gRNA comprises at least one chemical modification.

56. The gRNA of claim 55, wherein the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.

57. The gRNA of claim 56, wherein the at least one chemical modification comprises MS modifications at the 3 terminal nucleotides at the 5′ region and at the 3 terminal nucleotides at the 3′ region of the gRNA.

58. The gRNA of claim 57, wherein the crRNA repeat has the nucleotide sequence set forth as any one of SEQ ID NOs: 940, 942-945, 1228, 1230, and 1232.

59. The gRNA of claim 57 or 58, wherein the crRNA has the nucleotide sequence set forth as any one of SEQ ID NOs: 967-1085.

60. The gRNA of any one of claims 57-59, wherein the tracrRNA has the nucleotide sequence set forth as any one of SEQ ID NOs: 941, 946-955, 1229, 1231, and 1233.

61. The gRNA of any one of claims 57-60, wherein the gRNA has the nucleotide sequence set forth as any one of SEQ ID NOs: 1086-1227.

62. The gRNA of claim 56, wherein the BNA comprises a 2′,4′ BNA modification.

63. The gRNA of claim 62, wherein the 2′,4′ BNA modification is selected from the group consisting of: locked nucleic acid (LNA) modification, BNA^{NC}[N-Me]modification, 2′-0,4′-C-ethylene bridged nucleic acid (2′,4′-ENA) modification, and S-constrained ethyl (cEt) modification.

64. The gRNA of claim 63, wherein the 2′,4′ BNA is a LNA modification.

65. The gRNA of claim 63, wherein the 2′,4′ BNA is a cEt modification.

66. The gRNA of claim 56, wherein the at least one chemical modification comprises a BNA modification, 2′-O-Me modification, PS modification, or a combination thereof.

67. The gRNA of any one of claims 1-66, wherein the gRNA further comprises an extension comprising an edit template for reverse transcriptase (RT) editing.

68. A guide RNA (gRNA) comprising a CRISPR RNA (crRNA) and a trans-activating CRISPR RNA (tracrRNA), wherein the crRNA comprises
(i) a crRNA repeat; and
(ii) a spacer,
wherein the tracrRNA comprises:
(iii) an anti-repeat; and
(iv) a tail,
wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.

69. The gRNA of claim 68, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

70. The gRNA of claim 68 or 69, wherein the spacer is capable of hybridizing to a target sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

71. A nucleic acid molecule comprising a CRISPR RNA (crRNA) or encoding a crRNA, wherein the crRNA comprises a spacer and a crRNA repeat, wherein the spacer is capable of hybridizing to a target sequence in a forkhead box P3 (FOXP3) gene, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

72. The nucleic acid molecule of claim 71, wherein the spacer has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.

73. The nucleic acid molecule of claim 71, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

74. The nucleic acid molecule of any one of claims 71-73, wherein the crRNA repeat has the nucleotide sequence set forth as SEQ ID NO: 546 or a nucleotide sequence that differs in length and/or sequence from SEQ ID NO: 546 by 1 to 8 nucleotides.

75. The nucleic acid molecule of claim 74, wherein the crRNA repeat has the nucleotide sequence set forth as any one of SEQ ID NOs: 546, 549-552, 839, 842, and 845.

76. The nucleic acid molecule of any one of claims 71-75, wherein the crRNA has the nucleotide sequence set forth as any one of SEQ ID NOs: 574-692.

77. The nucleic acid molecule of any one of claims 71-76, wherein the crRNA is capable of binding a trans-activating CRISPR RNA (tracrRNA) to form a guide RNA (gRNA), wherein the tracrRNA comprises an anti-repeat and a tail.

78. The nucleic acid molecule of claim 77, wherein the tracrRNA has a nucleotide sequence having at least 80% sequence identity, at least 90% sequence identity, or at least 95% sequence identity to SEQ ID NO: 547.

79. The nucleic acid molecule of claim 77, wherein the tracrRNA has a nucleotide sequence that differs in length from SEQ ID NO: 547 by 1 to 16 nucleotides.

80. The nucleic acid molecule of claim 79, wherein the tracrRNA has a nucleotide sequence that is 8 nucleotides shorter than SEQ ID NO: 547.

81. The nucleic acid molecule of claim 79, wherein the tracrRNA has a nucleotide sequence that is 11 nucleotides shorter than SEQ ID NO: 547.

82. The nucleic acid molecule of any one of claims 77-81, wherein the tracrRNA has the nucleotide sequence set forth as any one of SEQ ID NOs: 547, 553-562, 840, 842, and 846.

83. The nucleic acid molecule of claim 77, wherein the gRNA is a single guide RNA (sgRNA) comprising the crRNA and the tracrRNA linked by a linker, wherein the sgRNA comprises a backbone and the spacer, and wherein the backbone of the sgRNA comprises the crRNA repeat, the linker, and the tracrRNA.

84. The nucleic acid molecule of claim 83, wherein the backbone of the sgRNA comprises a total length of 86 to 98 nucleotides.

85. The nucleic acid molecule of claim 83, wherein the backbone of the sgRNA comprises a total length of 94 nucleotides.

86. The nucleic acid molecule of any one of claims 83-85, wherein the backbone of the sgRNA has a nucleotide sequence having at least 80% sequence identity, at least 90% sequence identity, at least 95% sequence identity, or 100% sequence identity to any one of SEQ ID NOs: 563-573.

87. The nucleic acid molecule of any one of claims 77-86, wherein the gRNA comprises a first stem loop comprising a first stem and a second stem formed by hybridization of the crRNA repeat and the anti-repeat, wherein the first stem of the first stem loop comprises a total length of at least 3, 4, 5, 6, 7, 8, 9, 10, or 11 bp.

88. The nucleic acid molecule of any one of claims 77-86, wherein the gRNA comprises a first stem loop comprising a first stem and a second stem formed by hybridization of the crRNA repeat and the anti-repeat, wherein the first stem of the first stem loop comprises a total length of at most 3, 4, 5, 6, 7, 8, 9, 10, or 11 bp.

89. The nucleic acid molecule of claim 87 or 88, wherein the first stem of the first stem loop comprises a total length of 6 bp.

90. The nucleic acid molecule of claim 87 or 88, wherein the first stem of the first stem loop comprises a total length of 3 bp.

91. The nucleic acid molecule of any one of claims 77-90, wherein the tail of the tracrRNA comprises a total length of at least 1, 2, 3, 4, 5, 6, or 7 nucleotides.

92. The nucleic acid molecule of any one of claims 77-90, wherein the tail of the tracrRNA comprises a total length of at most 1, 2, 3, 4, 5, 6, or 7 nucleotides.

93. The nucleic acid molecule of claim 91 or 92, wherein the tail of the tracrRNA comprises a total length of 3 nucleotides.

94. The nucleic acid molecule of claim 91 or 92, wherein the tail of the tracrRNA comprises a total length of 1 nucleotide.

95. The nucleic acid molecule of any one of claims 87-94, wherein the gRNA further comprises a second stem loop most proximal to the tail, wherein the second stem loop comprises a first stem and a second stem.

96. The nucleic acid molecule of claim 95, wherein the first stem of the second stem loop comprises a total length of at least 1, 2, 3, 4, 5, or 6 bp.

97. The nucleic acid molecule of claim 95, wherein the first stem of the second stem loop comprises a total length of at most 1, 2, 3, 4, 5, or 6 bp.

98. The nucleic acid molecule of claim 96 or 97, wherein the first stem of the second stem loop comprises a total length of 5 bp.

99. The nucleic acid molecule of any one of claims 95-98, wherein the first stem of the first stem loop comprises a total length of 6 bp, the tail of the tracrRNA comprises a total length of 3 nucleotides, and the first stem of the second stem loop comprises a total length of 5 bp.

100. The nucleic acid molecule of claim 77, wherein the gRNA is a dual guide RNA (dgRNA).

101. The nucleic acid molecule of claim 100, wherein the crRNA repeat comprises a total length of at least 10, 11, 12, 13, 14, 15, 16, 17, 18, 19, 20, or 21 nucleotides.

102. The nucleic acid molecule of claim 100, wherein the crRNA repeat comprises a total length of at most 10, 11, 12, 13, 14, 15, 16, 17, 18, 19, 20, or 21 nucleotides.

103. The nucleic acid molecule of claim 101 or 102, wherein the crRNA repeat comprises a total length of 13 nucleotides.

104. The nucleic acid molecule of claim 101 or 102, wherein the crRNA repeat comprises a total length of 16 nucleotides.

105. The nucleic acid molecule of claim 101 or 102, wherein the crRNA repeat of the dgRNA comprises a total length of 21 nucleotides.

106. The nucleic acid molecule of any one of claims 100-105, wherein the tracrRNA comprises a total length of at least 65, 70, 75, 80, or 85 nucleotides.

107. The nucleic acid molecule of any one of claims 100-105, wherein the tracrRNA comprises a total length of at most 65, 70, 75, 80, or 85 nucleotides.

108. The nucleic acid molecule of claim 106 or 107, wherein the tracrRNA comprises a total length of 74 nucleotides.

109. The nucleic acid molecule of claim 106 or 107, wherein the tracrRNA comprises a total length of 77 nucleotides.

110. The nucleic acid molecule of any one of claims 77-109, wherein the gRNA comprises a total length of at least 85, 90, 95, 100, 105, 110, 115, 120, 125, 130, or 135 nucleotides.

111. The nucleic acid molecule of any one of claims 77-109, wherein the gRNA comprises a total length of at most 85, 90, 95, 100, 105, 110, 115, 120, 125, 130, or 135 nucleotides.

112. The nucleic acid molecule of any one of claims 77-109, wherein the gRNA comprises a total length of 106 to 135 nucleotides.

113. The nucleic acid molecule of claim 112, wherein the gRNA comprises a total length of 117 to 119 nucleotides.

114. The nucleic acid molecule of any one of claims 77-113, wherein the gRNA is capable of targeting a bound RNA-guided nuclease (RGN) polypeptide to a target sequence.

115. The nucleic acid molecule of claim 114, wherein the RGN polypeptide is capable of recognizing a consensus protospacer adjacent motif (PAM) having the nucleotide sequence set forth as NNNNCC.

116. The nucleic acid molecule of claim 115, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.

117. The nucleic acid molecule of claim any one of claims 114-116, wherein the RGN polypeptide comprises an amino acid sequence having at least 80% sequence identity to SEQ ID NO: 545; and wherein the target sequence and the spacer are selected from the group consisting of:
a) a target sequence having the nucleotide sequence set forth as SEQ ID NO: 156 and a spacer having the nucleotide sequence set forth as SEQ ID NO: 155 or a nucleotide sequence that differs in length and/or sequence from SEQ ID NO: 155 by 1 to 5 nucleotides;
b) a target sequence having the nucleotide sequence set forth as SEQ ID NO: 164 and a spacer having the nucleotide sequence set forth as SEQ ID NO: 163 or a nucleotide sequence that differs in length and/or sequence from SEQ ID NO: 163 by 1 to 5 nucleotides;
c) a target sequence having the nucleotide sequence set forth as SEQ ID NO: 190 and a spacer having the nucleotide sequence set forth as SEQ ID NO: 189 or a nucleotide sequence that differs in length and/or sequence from SEQ ID NO: 189 by 1 to 5 nucleotides;
d) a target sequence having the nucleotide sequence set forth as SEQ ID NO: 180 and a spacer having the nucleotide sequence set forth as SEQ ID NO: 179 or a nucleotide sequence that differs in length and/or sequence from SEQ ID NO: 179 by 1 to 5 nucleotides;
e) a target sequence having the nucleotide sequence set forth as SEQ ID NO: 198 and a spacer having the nucleotide sequence set forth as SEQ ID NO: 197 or a nucleotide sequence that differs in length and/or sequence from SEQ ID NO: 197 by 1 to 5 nucleotides; and
f) a target sequence having the nucleotide sequence set forth as SEQ ID NO: 194 and a spacer having the nucleotide sequence set forth as SEQ ID NO: 193 or a nucleotide sequence that differs in length and/or sequence from SEQ ID NO: 193 by 1 to 5 nucleotides.

118. The nucleic acid molecule of claim 117, wherein the spacer has the nucleotide sequence set forth as any one of SEQ ID NOs: 155, 163, 189, 179, 197, and 193.

119. The nucleic acid molecule of claim 117 or 118, wherein the RGN polypeptide comprises an amino acid sequence set forth as SEQ ID NO: 545.

120. The nucleic acid molecule of any one of claims 77-119, wherein the gRNA has a nucleotide sequence set forth as any one of SEQ ID NOs: 693-834.

121. The nucleic acid molecule of claim 120, wherein the gRNA has a nucleotide sequence set forth as any one of SEQ ID NOs: 693, 694, 695, 696, 697, and 698.

122. The nucleic acid molecule of any one of claims 77-119, wherein the gRNA comprises at least one chemical modification.

123. The nucleic acid molecule of claim 122, wherein the at least one chemical modification comprises a bridged nucleic acid (BNA) modification; 2′-O-methyl (2′-O-Me) modification; 2′-O-methoxy-ethyl (2′MOE) modification; 2′-fluoro (2′-F) modification; 2′F-4′Cα-OMe modification; 2′,4′-di-Cα-OMe modification; 2′-O-methyl 3′phosphorothioate (MS) modification; 2′-O-methyl 3′thiophosphonoacetate (MSP) modification; 2′-O-methyl 3′phosphonoacetate (MP) modification; phosphorothioate (PS) modification; or a combination thereof.

124. The nucleic acid molecule of claim 123, wherein the at least one chemical modification comprises MS modifications at the 3 terminal nucleotides at the 5′ region and at the 3 terminal nucleotides at the 3′ region of the gRNA.

125. The nucleic acid molecule of claim 124, wherein the crRNA repeat has the nucleotide sequence set forth as any one of SEQ ID NOs: 940, 942-945, 1228, 1230, and 1232.

126. The nucleic acid molecule of claim 124 or 125, wherein the crRNA has the nucleotide sequence set forth as any one of SEQ ID NOs: 967-1085.

127. The nucleic acid molecule of any one of claims 124-126, wherein the tracrRNA has the nucleotide sequence set forth as any one of SEQ ID NOs: 941, 946-955, 1229, 1231, and 1233.

128. The nucleic acid molecule of any one of claims 124-127, wherein the gRNA has the nucleotide sequence set forth as any one of SEQ ID NOs: 1086-1227.

129. The nucleic acid molecule of claim 123, wherein the BNA comprises a 2′,4′ BNA modification.

130. The nucleic acid molecule of claim 129, wherein the 2′,4′ BNA modification is selected from the group consisting of: locked nucleic acid (LNA) modification, BNA^{NC}[N-Me]modification, 2′-O,4′-C-ethylene bridged nucleic acid (2′,4′-ENA) modification, and S-constrained ethyl (cEt) modification.

131. The nucleic acid molecule of claim 130, wherein the 2′,4′ BNA is a LNA modification.

132. The nucleic acid molecule of claim 130, wherein the 2′,4′ BNA is a cEt modification.

133. The nucleic acid molecule of claim 123, wherein the at least one chemical modification comprises a BNA modification, 2′-O-Me modification, PS modification, or a combination thereof.

134. The nucleic acid molecule of any one of claims 77-133, wherein the gRNA further comprises an extension comprising an edit template for reverse transcriptase editing.

135. A nucleic acid molecule comprising a CRISPR RNA (crRNA) or encoding a crRNA, wherein the crRNA comprises a spacer and a crRNA repeat, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213, or has a nucleotide sequence that differs in length and/or sequence from any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213 by 1 to 5 nucleotides.

136. The nucleic acid molecule of claim 135, wherein the spacer has the nucleotide sequence set forth as any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, and 213.

137. The nucleic acid molecule of claim 135 or 136, wherein the spacer is capable of hybridizing to a target sequence, and wherein the target sequence has the nucleotide sequence set forth as any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, and 214.

138. A vector comprising the nucleic acid molecule of any one of claims 71-76, wherein the nucleic acid molecule comprises a polynucleotide encoding the crRNA.

139. The vector of claim 138, wherein the nucleic acid molecule further comprises a heterologous promoter operably linked to the polynucleotide encoding the crRNA.

140. The vector of claim 139, wherein the heterologous promoter is an RNA polymerase III (pol III) promoter.

141. The vector of any one of claims 138-140, wherein the vector further comprises a nucleic acid molecule encoding an RGN polypeptide.

142. The vector of claim 141, wherein the crRNA is capable of binding a tracrRNA to form a guide RNA, and wherein the guide RNA is capable of binding to the RGN polypeptide.

143. The vector of claim 141 or 142, wherein the vector further comprises a promoter operably linked to the nucleic acid molecule encoding the RGN polypeptide.

144. A vector comprising the nucleic acid molecule of any one of claims 77-134, wherein the nucleic acid molecule comprises a polynucleotide encoding the crRNA, and wherein the vector further comprises a polynucleotide encoding the tracrRNA.

145. The vector of claim 144, wherein the polynucleotide encoding the crRNA and the polynucleotide encoding the tracrRNA are operably linked to the same promoter and are encoded as a sgRNA.

146. The vector of claim 144, wherein the polynucleotide encoding the crRNA and the polynucleotide encoding the tracrRNA are operably linked to separate promoters.

147. The vector of any one of claims 144-146, wherein the vector further comprises a nucleic acid molecule encoding an RGN polypeptide.

148. The vector of claim 147, wherein the crRNA is capable of binding the tracrRNA to form a guide RNA, and wherein the guide RNA is capable of binding to the RGN polypeptide.

149. The vector of claim 147 or 148, wherein the vector further comprises a promoter operably linked to the nucleic acid molecule encoding the RGN polypeptide.

150. A cell comprising the gRNA of any one of claims 1-70, the nucleic acid molecule of any one of claims 71-137, or the vector of any one of claims 138-149.

151. An RNA-guided nuclease (RGN) system for binding a target sequence within a forkhead box P3 (FOXP3) gene, wherein the RGN system comprises:
a) one or more guide RNA (gRNA) of any one of claims 1-70, or one or more polynucleotides comprising one or more nucleotide sequences encoding the one or more gRNA of any one of claims 1-70; and
b) an RGN polypeptide, or a polynucleotide comprising a nucleotide sequence encoding the RGN polypeptide.

152. The RGN system of claim 151, wherein the one or more gRNA is capable of forming a complex with the RGN polypeptide to direct the RGN polypeptide to bind to the target sequence.

153. The RGN system of claim 151 or 152, wherein the RGN polypeptide is capable of recognizing a consensus protospacer adjacent motif (PAM) having the nucleotide sequence set forth as NNNNCC.

154. The RGN system of claim 153, wherein the RGN polypeptide is capable of recognizing a full PAM having a nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.

155. The RGN system of any one of claims 151-154, wherein the RGN polypeptide comprises an amino acid sequence set forth as SEQ ID NO: 545.

156. The RGN system of any one of claims 151-155, wherein the polynucleotide comprising a nucleotide sequence encoding the RGN polypeptide is codon optimized for expression in a mammalian cell.

157. The RGN system of any one of claims 151-156, wherein at least one of the one or more nucleotide sequences encoding the one or more gRNAs and the nucleotide sequence encoding the RGN polypeptide is operably linked to a promoter heterologous to the nucleotide sequence.

158. The RGN system of any one of claims 151-157, wherein the one or more nucleotide sequences encoding the one or more gRNAs and the nucleotide sequence encoding the RGN polypeptide are located on one vector.

159. The RGN system of any one of claims 151-155, wherein the polynucleotide comprising a nucleotide sequence encoding the RGN polypeptide comprises an mRNA.

160. The RGN system of any one of claims 151-159, wherein the RGN polypeptide is nuclease inactive or is a nickase.

161. The RGN system of any one of claims 151-160, wherein the RGN polypeptide is fused to a base-editing polypeptide.

162. The RGN system of claim 161, wherein the base-editing polypeptide comprises a deaminase.

163. The RGN system of any one of claims 151-160, wherein the RGN polypeptide is fused to a reverse transcriptase (RT) editing polypeptide.

164. The RGN system of claim 163, wherein the RT editing polypeptide comprises a DNA polymerase.

165. The RGN system of claim 164, wherein the DNA polymerase comprises a reverse transcriptase.

166. The RGN system of any one of claims 163-165, wherein the gRNA further comprises an extension comprising an edit template for RT editing.

167. The RGN system of any one of claims 151-166, wherein the RGN polypeptide comprises one or more nuclear localization signals.

168. A ribonucleoprotein (RNP) complex comprising the one or more gRNA and the RGN polypeptide of the RGN system of any one of claims 151-167.

169. A cell comprising the RGN system of any one of claims 151-167 or the RNP complex of claim 168.

170. The cell of claim 169, wherein the cell is a eukaryotic cell.

171. The cell of claim 170, wherein the eukaryotic cell is a mammalian cell.

172. The cell of claim 171, wherein the mammalian cell is a human cell.

173. The cell of claim 171 or 172, wherein the mammalian cell or human cell is a T cell or an induced pluripotent stem cell.

174. A method for binding a target sequence within a FOXP3 gene, comprising delivering the RGN system of any one of claims 151-167 or the RNP complex of claim 168 to the target sequence or a cell comprising the target sequence.

175. The method of claim 174, wherein cleavage or modification of the target sequence occurs.

176. A method for assembling an RNA-guided nuclease (RGN) ribonucleoprotein complex, the method comprising combining under conditions suitable for formation of the complex:
a) the guide RNA of any one of claims 1-70; and
b) an RGN polypeptide that binds the guide RNA.

177. The method of claim 176, wherein the RGN polypeptide is capable of recognizing a consensus protospacer adjacent motif (PAM) having the nucleotide sequence set forth as NNNNCC.

178. The method of claim 176 or 177, wherein the complex directs cleavage of the target sequence.

179. The method of claim 178, wherein the cleavage generates a double-stranded break.

180. The method of claim 178, wherein the cleavage generates a single-stranded break.

181. A method for binding a target sequence within a FOXP3 gene, the method comprising:
a) combining under conditions suitable for formation of a ribonucleoprotein (RNP) complex:
i) the guide RNA of any one of claims 1-70; and
ii) an RGN polypeptide that binds the guide RNA;
thereby assembling an RNP complex; and
b) contacting the target sequence or a cell comprising the target sequence with the assembled RNP complex.

182. The method of claim 181, wherein the guide RNA hybridizes to the target sequence, thereby directing binding of the RNP complex to the target sequence.

183. The method of claim 181 or 182, wherein the RGN polypeptide is capable of recognizing a consensus protospacer adjacent motif (PAM) having the nucleotide sequence set forth as NNNNCC.

184. The method of claim 183, wherein the RGN polypeptide is capable of recognizing a full protospacer adjacent motif (PAM) having the nucleotide sequence set forth as any one of CAACCCCA, AACCCCAG, TTGTCCAA, CAGGCCTG, GGGTCCTT, CAAGCCCT, ATGCCCAA, CCAACCCC, CATGCCAC, GCCACCAT, GGACCCGA, CCTTCCTT, TTGGCCCT, GGGGCCGA, CTCGCCCA, GCACCCAA, CCCTCCAG, CAGCCCTC, GGCCCCCA, GGGCCCCC, GGCCCCGG, AGGGCCGA, TCCCCCTG, TTCCCCCT, GTTCCCCC, GGTTCCCC, GGGGCCCA, CGGCCCTG, GGGCCCAT, TCGGCCCT, CATGCCTC, GCCTCCTC, TCTTCCTT, TGGCCC, CAGACC, TCGGCC, CTTGCC, GGCCCC, GCAGCC, AAGCCC, GCCTCC, GCCACC, ATCCCC, AAAGCC, CCATCC, CCTTCC, TGGGCC, GGGCCC, CGGGCC, AACCCC, TCGCCC, CATGCC, AGGGCC, TGAACC, CCCGCC, TCTTCC, and GGCTCC.

185. The method of any one of claims 181-184, wherein the RGN polypeptide comprises an amino acid sequence set forth as SEQ ID NO: 545.

186. The method of any one of claims 181-185, wherein the method is performed in vitro or ex vivo.

187. The method of any one of claims 181-186, wherein the RGN polypeptide is capable of cleaving the target sequence, thereby allowing for the cleaving and/or modifying of the target sequence.

188. The method of claim 187, wherein the cleaving generates a single-stranded break.

189. The method of claim 187, wherein the cleaving generates a double-stranded break.

190. The method of claim 187, wherein the cleaving results in insertion of a heterologous sequence within the target sequence.

191. The method of any one of claims 181-186, wherein the RGN polypeptide is nuclease inactive or is a nickase.

192. The method of claim 191, wherein the RGN polypeptide is fused to a base-editing polypeptide.

193. The method of claim 192, wherein the base-editing polypeptide comprises a deaminase.

194. The method of any one of claims 181-186, wherein the RGN is fused to a reverse transcriptase (RT) editing polypeptide.

195. The method of claim 194, wherein the RT editing polypeptide comprises a DNA polymerase.

196. The method of claim 195, wherein the DNA polymerase comprises a reverse transcriptase.

197. The method of any one of claims 194-196, wherein the gRNA further comprises an extension comprising an edit template for RT editing.

198. A method for modulating expression of a forkhead box P3 (FOXP3) gene in a population of cells, comprising delivering the RGN system of any one of claims 151-167 or the RNP complex of claim 168 to the population of cells, wherein the population of cells comprises the target sequence, and wherein FOXP3 gene expression is modulated as compared to FOXP3 gene expression in a control population of cells.

199. The method of claim 198, wherein cleavage or modification of the target sequence occurs.

200. The method of claim 199, wherein cleavage or modification of the target sequence is detected by sequencing.

201. The method of any one of claims 198-200, wherein FOXP3 gene expression is measured by quantitative PCR, microarray, RNA-seq, flow cytometry, immunoblot, enzyme-linked immunosorbent assay (ELISA), protein immunoprecipitation, immunostaining, high performance liquid chromatography (HPLC), liquid chromatography-mass spectrometry (LC/MS), mass spectrometry, or a combination thereof.

202. The method of any one of claims 198-201, wherein FOXP3 gene expression is decreased.

203. The method of claim 202, wherein the decrease in FOXP3 gene expression comprises decrease in FOXP3 mRNA and/or Foxp3 protein level.

204. The method of any one of claims 199-203, wherein cleavage or modification of the target sequence occurs at a rate of 40% to 100%.

205. The method of any one of claims 199-204, wherein cleavage or modification of the target sequence occurs at a rate of 80% to 100%.

206. The method of any one of claims 198-205, wherein the control population of cells has not been subjected to the delivering.

207. The method of any one of claims 198-206, wherein the population of cells comprises T cells.
